(19) United States
(12) Patent Application Publication
(10) Pub. No.: US 2026/0216087 A1
(43) Pub. Date: Jul. 30, 2026

(54) NOVEL IONIZABLE LIPID COMPOUNDS FOR NUCLEIC ACID DELIVERY

(71) Applicant: CELON PHARMA S.A., Kielpin/Lomianki (PL)

(72) Inventors: Maciej WIECZOREK, Lomianki (PL); Michal MROCZKIEWICZ, Warszawa (PL); Mateusz MACH, Warszawa (PL); Krzysztof DUBIEL, Babice Nowe (PL); Tadeusz LEMEK, Krakow (PL); Bartosz SETNER, Wroclaw (PL); Ewelina JUSZCZYK, Warszawa (PL); Pawel ZERO, Warsaw (PL); Kinga GALAZKA, Warszawa (PL); Gabriela MAREK, Warszawa (PL); Jakub PIATKOWSKI, Lidzbark (PL)

(73) Assignee: CELON PHARMA S.A., Kielpin/Lomianki (PL)

(21) Appl. No.: 19/145,426

(22) PCT Filed: Dec. 26, 2023
(86) PCT No.: PCT/IB2023/063252

§ 371 Date: Jul. 2, 2025

Related U.S. Application Data
(60) Provisional application No. 63/478,560, filed on Jan. 5, 2023.

(51) Int. Cl. A61K 9/51; A61K 31/7105; A61K 48/00; C07C 227/18; C07C 229/12; C07C 229/16; C07D 233/90

(52) U.S. Cl. CPC A61K 9/5123; A61K 31/7105; A61K 48/0033; C07C 227/18; C07C 229/12; C07C 229/16; C07D 233/90; C07C 2601/04

(57) ABSTRACT

Novel ionizable lipid compounds, compositions comprising such ionizable lipid compounds, and related methods of their use are disclosed. Nanoparticle compositions include a novel lipid as well as additional lipids such as phospholipids, structural lipids, and PEG lipids. Nanoparticle compositions further including biologically active agents, such as siRNA or mRNA, are useful in the delivery of said biologically active agents to subjects in need thereof.

### CROSS-REFERENCE TO RELATED APPLICATION

This application claims priority benefit of U.S. provisional application No. 63/478,560 filed Jan. 5, 2023, the disclosures of which are incorporated herein by reference in their entirety.

### TECHNICAL FIELD

The present disclosure provides novel ionizable lipid compounds, compositions comprising such ionizable lipid compounds, and related methods of their use.

### BACKGROUND

Treatment and prevention of diseases using biologically active substances such as small molecule drugs, proteins, and nucleic acids, including DNA and mRNA, have the potential to revolutionize modern medicine. In particular, therapeutic, diagnostic, and/or prophylactic nucleic acids have the potential of achieving long-lasting or even curative effects via gene or RNA inhibition, addition, replacement or editing. However nucleic acid delivery to cells is made difficult by the relative instability and low cell permeability of such molecules as well as short expression windows and needs for frequent re-dosing of subjects.

Thus, there exists a need to develop compounds, compositions, and methods that improve expression profiles, stability, facilitate internalization, increase target affinity, and reduce needs for frequent dosing of nucleic acid therapeutics and prophylactics.

### SUMMARY OF THE INVENTION

The present disclosure provides novel compounds and compositions and methods involving the same.

In one aspect, the present disclosure provides compounds of Formula (I):

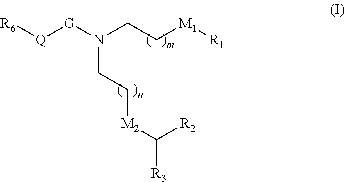

or a salt or isomer thereof, wherein
each m is independently an integer from 4-13, e.g., 4, 5, 6, 7, 8, 9, 10, 11, 12, or 13 or any subrange selected from within the range of 4-13, e.g., 4-9, 6-8, 4-7, 4-5, 5-9, 6-13 etc.; each n is independently an integer from 1-3, e.g., 1, 2 or 3, or any subrange selected from the range of 1-3, e.g., 1-2, 2-3, etc.;
each R¹ is independently selected from C1-C5 alkyl, C2-C5 alkenyl, C2-C5 alkynyl, wherein the alkyl, the alkenyl, or the alkynyl is linear or branched;

each R₂ and R₃ is independently selected from C₁ to C₁₄ alkyl;

each R₆ is independently selected from H,

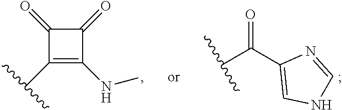

each M₁ and M₂ is independently selected from —C(O)O—, and —OC(O)—, wherein at least one of M₁ or M₂ is —C(O)O—;
Q is —O— or —NH—;
and each G is —(CR₄R₅)ₖ—;
wherein each k is selected from an integer from 2-5, e.g. 2, 3, 4, and 5; or any subrange selected from the range of 2-5, e.g. 2, -3, 3-4, and 2-5 etc.;
and each R₄ and R₅ is independently selected from H, and C₁-C₃ alkyl.

In certain aspects, compounds of Formula I may include, for example, the following compounds:

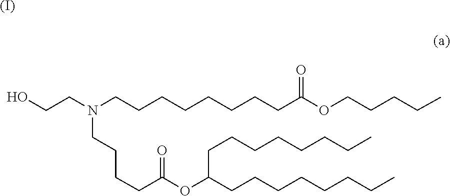 [SMILES: "(I)" ; CCCCCCCCC(CCCCCCCC)OC(=O)CCCCN(CCO)CCCCCCCCC(=O)OCCCCC]

(also referred to as Compound 1)

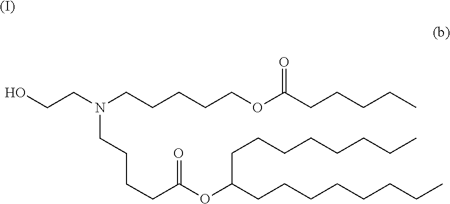 [SMILES: "(I)" ; CCCCCCCCC(CCCCCCCC)OC(=O)CCCCN(CCO)CCCCCOC(=O)CCCCC]

(also referred to as Compound 68)

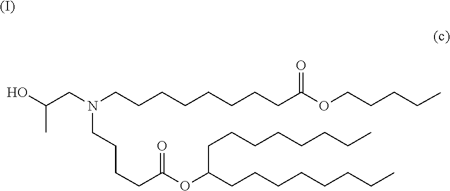 [SMILES: "(I)" ; CCCCCCCCC(CCCCCCCC)OC(=O)CCCCN(CCCCCCCCC(=O)OCCCCC)CC(C)O]

(also referred to as Compound 50)

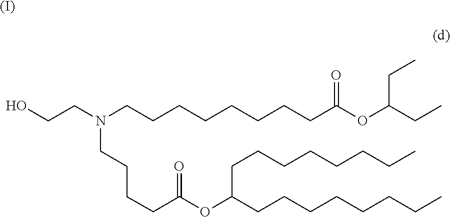 [SMILES: "(I)" ; CCCCCCCCC(CCCCCCCC)OC(=O)CCCCN(CCO)CCCCCCCCC(=O)OC(CC)CC]

(also referred to as Compound 48)

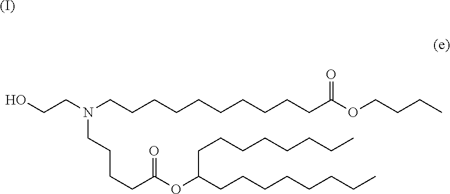 [SMILES: "(I)" ; CCCCCCCCC(CCCCCCCC)OC(=O)CCCCN(CCO)CCCCCCCCCCC(=O)OCCCC]

(also referred to as Compound 35)

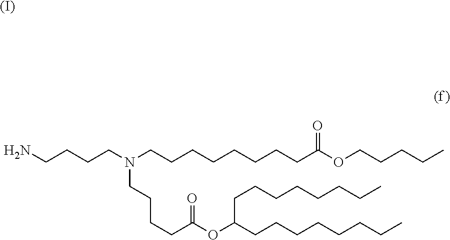 [SMILES: "(I)" ; CCCCCCCCC(CCCCCCCC)OC(=O)CCCCN(CCCCN)CCCCCCCCC(=O)OCCCCC]

(also referred to as Compound 53)

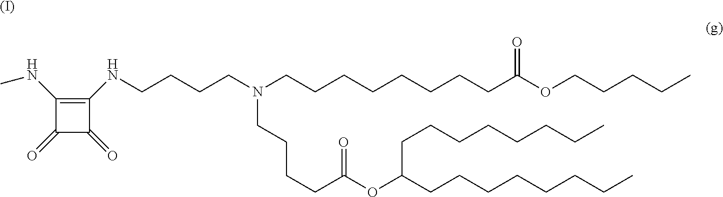 [SMILES: "(I)" ; CCCCCCCCC(CCCCCCCC)OC(=O)CCCCN(CCCCCCCCC(=O)OCCCCC)CCCCNc1c(NC)c(=O)c1=O]

(also referred to as Compound 54)

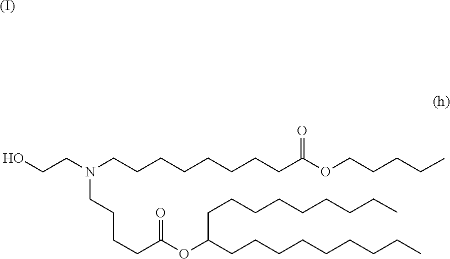 [SMILES: "(I)" ; CCCCCCCCCC(CCCCCCCCC)OC(=O)CCCCN(CCO)CCCCCCCCC(=O)OCCCCC]

(also referred to as Compound 42)

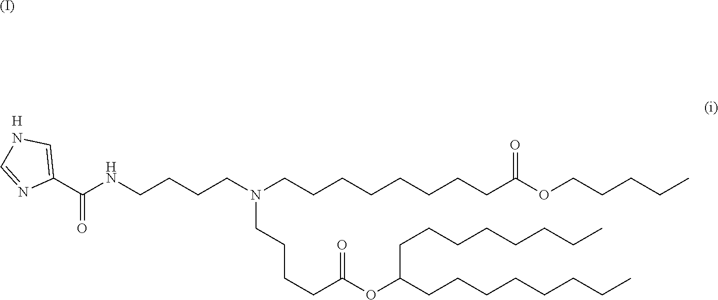 [SMILES: "(I)" ; CCCCCCCCC(CCCCCCCC)OC(=O)CCCCN(CCCCCCCCC(=O)OCCCCC)CCCCNC(=O)c1c[nH]cn1]

(also referred to as Compound 65)

In some aspects, compounds of Formula I may include, for example:

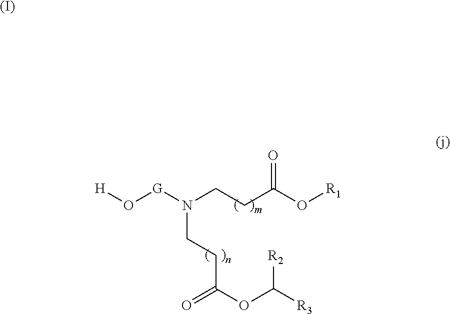

or a salt or isomer thereof, wherein
each m is independently an integer from 4-13;
each n is independently an integer from 1-3;
each R¹ is independently selected from C1-C5 alkyl, C2-C5 alkenyl, C2-C5 alkynyl, wherein the alkyl, the alkenyl, or the alkynyl is linear or branched;
each R₂ and R₃ is independently selected from C₁ to C₁₄ alkyl;
and each G is —(CR₄R₅)ₖ—;
wherein each k is selected from an integer from 2-5;
and each R₄ and R₅ is independently selected from H, and C₁-C₃ alkyl.

In one aspect, the present disclosure provides the general synthesis route for the synthesis of compounds of Formula (I) (j)

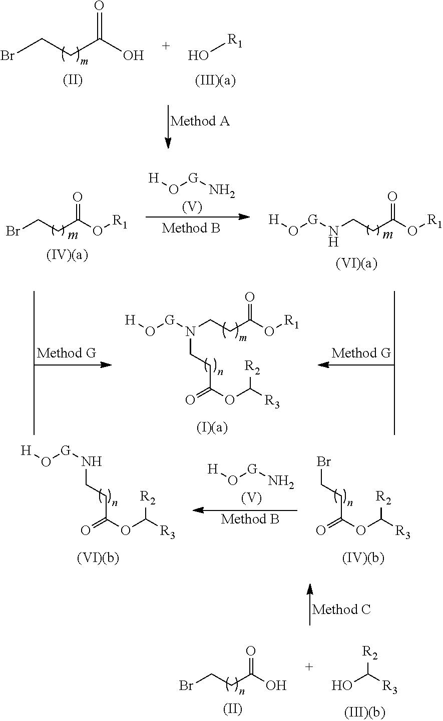

In some aspects, compounds of Formula I may include, for example:

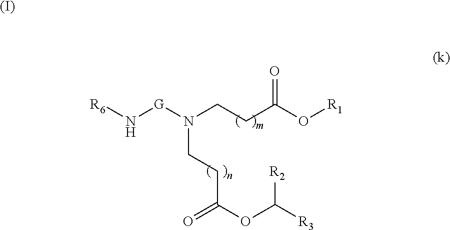

or a salt or isomer thereof, wherein
each m is independently an integer from 4-13;
each n is independently an integer from 1-3;
each R¹ is independently selected from C1-C5 alkyl, C2-C5 alkenyl, C2-C5 alkynyl, wherein the
alkyl, the alkenyl, or the alkynyl is linear or branched;
each R₂ and R₃ is independently selected from C₁ to C₁₄ alkyl;
each R₆ is H,
and each G is —(CR₄R₅)ₖ—;
wherein each k is selected from an integer from 2-5;
and each R₄ and R₅ is independently selected from H, and C₁-C₃ alkyl.
In one aspect, the present disclosure provides the general synthesis route for the synthesis of compounds of Formula (I)(k):

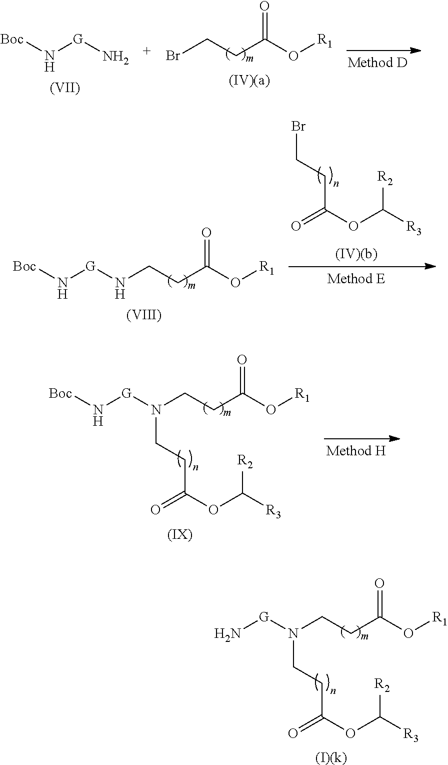

In some aspects, compounds of Formula I may include, for example:

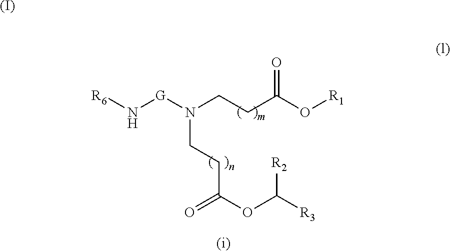

or a salt or isomer thereof, wherein
each m is independently an integer from 4-13;
each n is independently an integer from 1-3;
each R¹ is independently selected from C1-C5 alkyl, C2-C5 alkenyl, C2-C5 alkynyl, wherein the
alkyl, the alkenyl, or the alkynyl is linear or branched;
each R₂ and R₃ is independently selected from C₁ to C₁₄ alkyl;
each R₆ is

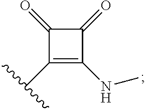 [SMILES: *c1c(NC)c(=O)c1=O]

and each G is —(CR₄R₅)ₖ—;
wherein each k is selected from an integer from 2-5;
and each R₄ and R₅ is independently selected from H, and C₁-C₃ alkyl.

In some aspects, compounds of Formula I may include, for example:

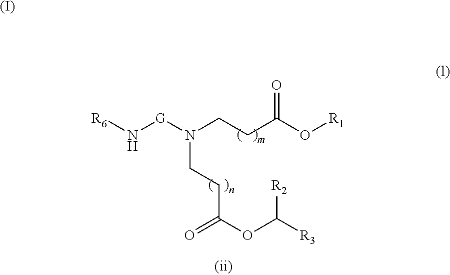

or a salt or isomer thereof, wherein
each m is independently an integer from 4-13;
each n is independently an integer from 1-3;
each R¹ is independently selected from C1-C5 alkyl, C2-C5 alkenyl, C2-C5 alkynyl, wherein the alkyl, the alkenyl, or the alkynyl is linear or branched;
each R₂ and R₃ is independently selected from C₁ to C₁₄ alkyl;
each R₆ is

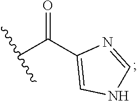 [SMILES: *C(=O)c1c[nH]cn1]

and each G is —(CR₄R₅)ₖ—;
wherein each k is selected from an integer from 2-5;
and each R₄ and R₅ is independently selected from H, and C₁-C₃ alkyl.

In one aspect, the present disclosure provides the general synthesis route for the synthesis of compounds of Formula (I) (1) (i) and (I) (1) (ii):

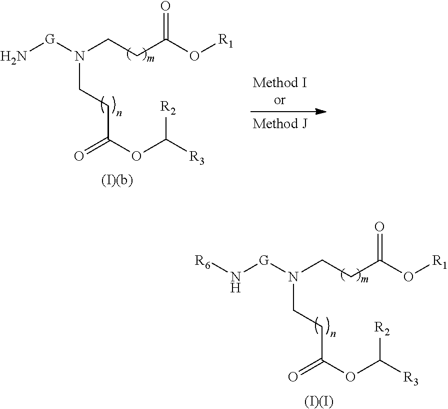

In some aspects, compounds of Formula I may include, for example:

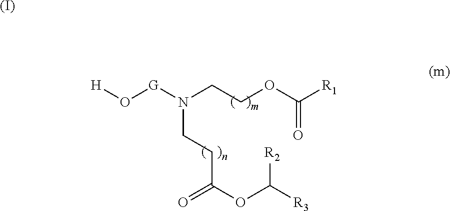

or a salt or isomer thereof, wherein
each m is independently an integer from 4-13;
each n is independently an integer from 1-3;
each R¹ is independently selected from C1-C5 alkyl, C2-C5 alkenyl, C2-C5 alkynyl, wherein the alkyl, the alkenyl, or the alkynyl is linear or branched;
each R₂ and R₃ is independently selected from C₁ to C₁₄ alkyl;
and each G is —(CR₄R₅)ₖ—;
wherein each k is selected from an integer from 2-5;
and each R₄ and R₅ is independently selected from H, and C₁-C₃ alkyl.

In one aspect, the present disclosure provides the general synthesis route for the synthesis of compounds of Formula (I) (m):

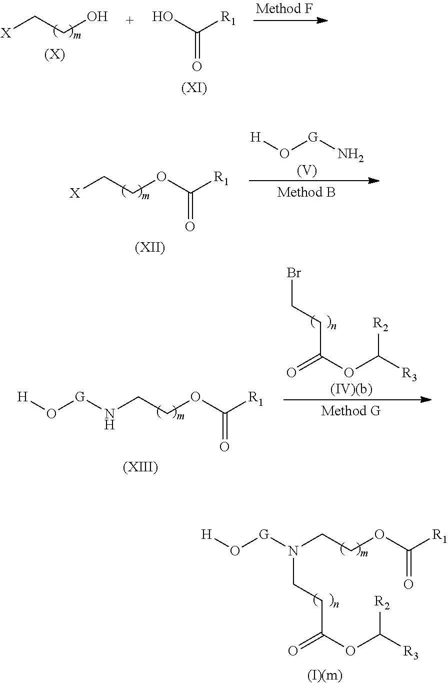

wherein
X is Cl, Br;
each m is independently an integer from 4-13, e.g., 4, 5, 6, 7, 8, 9, 10, 11, 12, or 13 or any subrange selected from within the range of 4-13, e.g., 4-9, 6-8, 4-7, 4-5, 5-9, 6-13 etc.;
each n is independently an integer from 1-3, e.g., 1, 2 or 3, or any subrange selected from the range of 1-3, e.g., 1-2, 2-3, etc.;
each R₁ is C1-C5 alkyl, wherein the alkyl is linear or branched;
each R₂ and R₃ is independently selected from C₁ to C₁₄ alkyl;
and each G is —(CR₄R₅)ₖ—;
wherein each k is selected from an integer from 2-5, e.g. 2, 3, 4, and 5; or any subrange selected from the range of 2-5, e.g. 2, -3, 3-4, and 2-5 etc.;
and each R₄ and R₅ is independently selected from H, and C₁-C₃ alkyl.

In one aspect, method Fis:

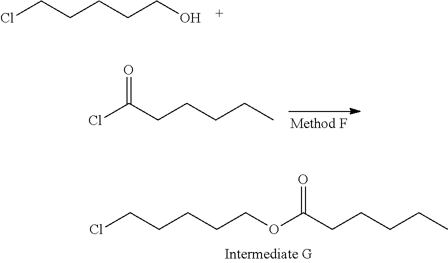

In one aspect, the present disclosure provides the Representative Procedure 1 for the synthesis of Compound 1/(I) (a):

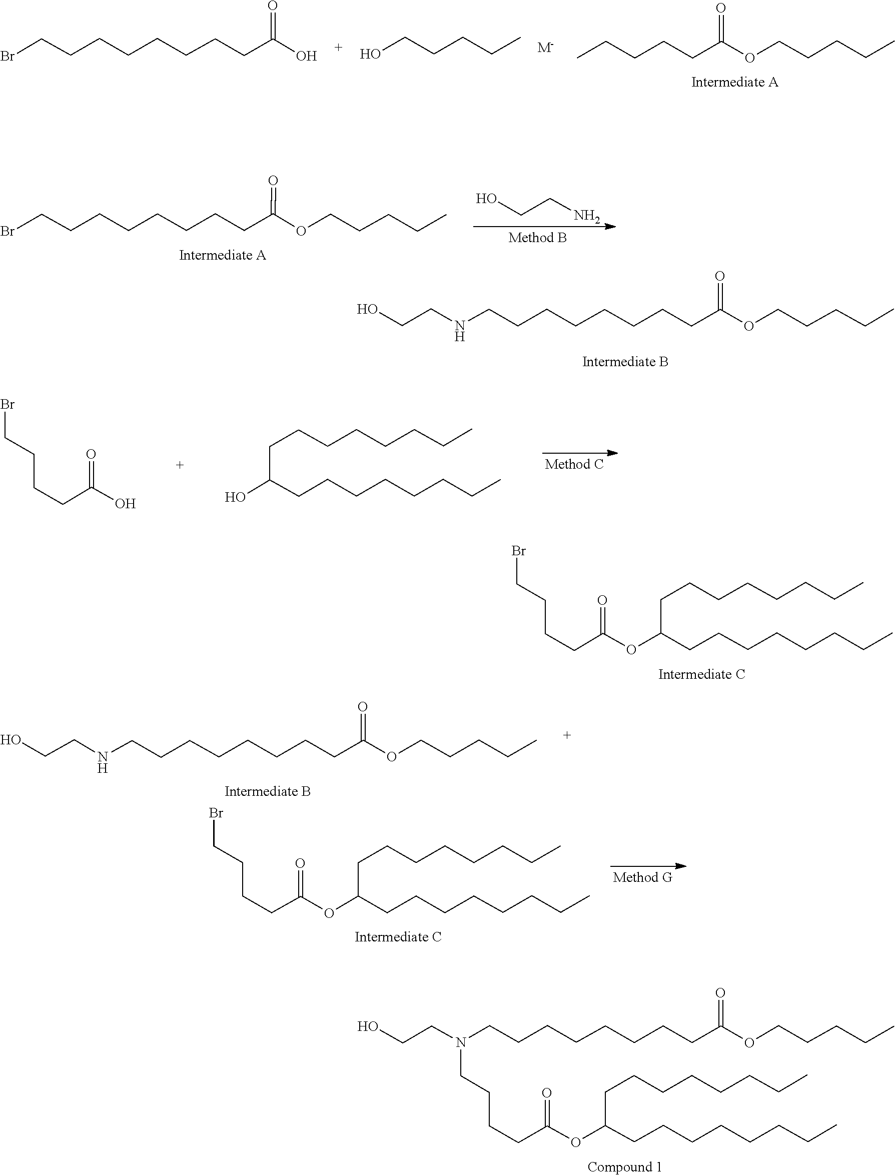

In one aspect, the present disclosure provides the Representative Procedure 2 for the synthesis of Compound 53/(I) (f):

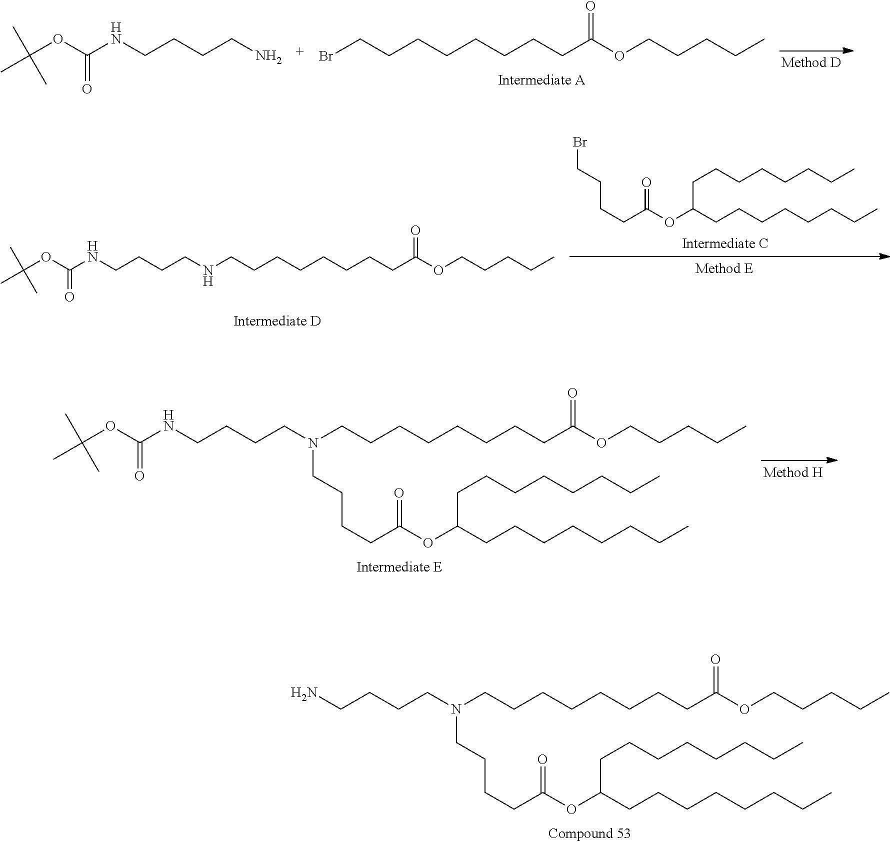

In one aspect, the present disclosure provides the Representative Procedure 3 for the synthesis of Compound 54 (I) (g):

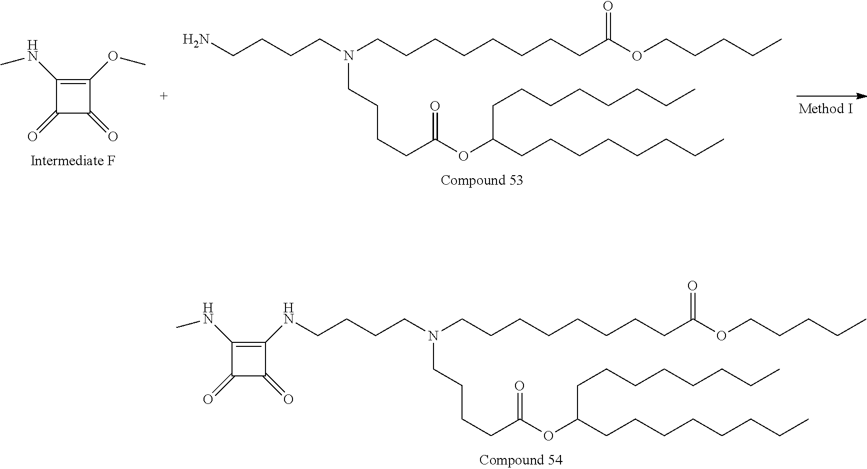

wherein the synthesis route for intermediate F is:

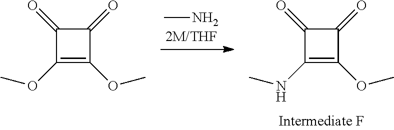

In one aspect, the present disclosure provides the Representative Procedure 4 for the synthesis of Compound 65/(1) (i):

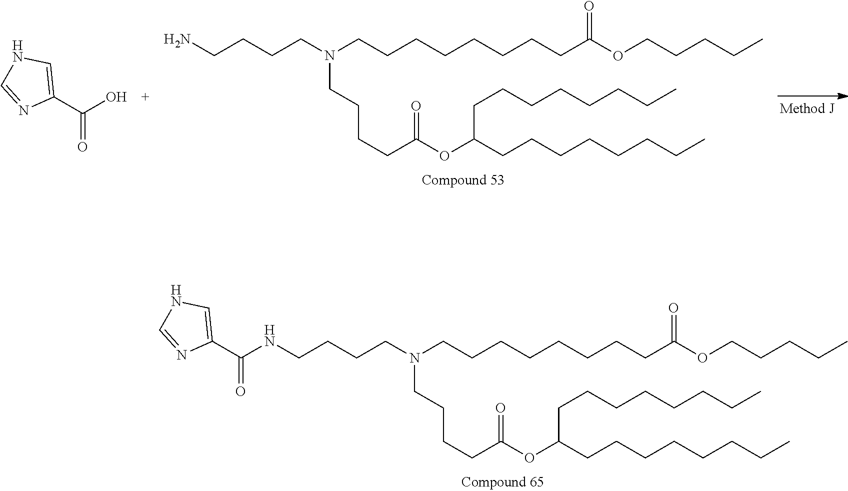 [SMILES: O=C(O)c1c[nH]cn1 ; CCCCCCCCC(CCCCCCCC)OC(=O)CCCCN(CCCCN)CCCCCCCCC(=O)OCCCCC ; CCCCCCCCC(CCCCCCCC)OC(=O)CCCCN(CCCCCCCCC(=O)OCCCCC)CCCCNC(=O)c1c[nH]cn1]

In another aspect, the present disclosure provides a method of delivering a payload (e.g. a therapeutic, diagnostic and/or prophylactic nucleic acid) to a cell (e.g., a mammalian cell) by administering a nanoparticle composition comprising (i) a compound of Formula (I), and (ii) a payload to a subject in need thereof, wherein upon administration to a subject in a therapeutically effective amount, provides a therapeutic benefit to the subject.

In another aspect, the present disclosure provides a method of producing a polypeptide of interest in a cell (e.g., a mammalian cell) by contacting the cell with a nanoparticle composition comprising (i) a compound of Formulae (I), and (ii) an mRNA encoding the polypeptide of interest, whereby the mRNA is capable of being translated in the cell to produce the polypeptide.

In another aspect, the present disclosure provides a method of introducing a gene to a cell (e.g., a mammalian cell) by contacting the cell with a nanoparticle composition comprising (i) a compound of Formula (I), and (ii) a DNA encoding the gene of interest, whereby the cell becomes capable of expressing the introduced gene.

In another aspect, the present disclosure provides a method of decreasing the expression of a gene in a cell by contacting the cell with a nanoparticle composition comprising (i) a compound of Formula (I), and (ii) a siRNA capable of decreasing the expression of a gene of interest, whereby the cell decreases the expression of the gene of interest.

In another aspect, the present disclosure provides a nanoparticle composition comprising (i) a compound of Formula (I), (ii) a phospholipid moiety and (ii) a payload. The phospholipid moiety may be selected from a phospholipid known in the art, such as phosphatidyl choline, phosphatidyl ethanolamine, phosphatidyl glycerol, phosphatidyl serine, phosphatidic acid, 2-lysophosphatidyl choline, and a sphingomyelin. A fatty acid moiety may be selected from the non-limiting group consisting of lauric acid, myristic acid, myristoleic acid, palmitic acid, palmitoleic acid, stearic acid, oleic acid, linoleic acid, alpha-linolenic acid, erucic acid, arachidic acid, arachidonic acid, phytanic acid, eicosapentaenoic acid, behenic acid, docosapentaenoic acid, and docosahexaenoic acid. For example, in certain aspects, a phospholipid is independently selected from 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-dioleoyl-sn-glycero-3-phosphoethanol amine (DOPE), 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), and sphingomyelin. In certain aspects, the phospholipid is DOPE. In other aspects, the phospholipid is DSPC. Non-natural species including natural species with modifications and substitutions including branching, oxidation, cyclization, and alkynes are also contemplated.

In another aspect, the present disclosure provides a nanoparticle composition comprising (i) a compound of Formula (I), (ii) a structural lipid and (iii) a payload. The structural lipid may be selected from a structural lipid known in the art, such as cholesterol, fecosterol, sitosterol, ergosterol, campesterol, stigmasterol, brassicasterol, tomatidine, ursolic acid, and alpha-tocopherol.

In another aspect, the present disclosure provides a nanoparticle composition comprising (i) a compound of Formula (I), (ii) PEG lipid and (iii) a payload. The PEG lipid may be selected from a PEG lipid known in the art, such as PEG-modified phosphatidylethanolamine, a PEG-modified phosphatidic acid, a PEG-modified ceramide, a PEG-modified dialkylamine, a PEG-modified diacylglycerol, and a PEG-modified dialkylglycerol.

In another aspect, the present disclosure provides a nanoparticle composition comprising (i) a compound of Formula (I), (ii) a phospholipid moiety, (iii) a structural lipid, (iv) PEG lipid, (v) a payload, or any combination thereof.

In some aspects, the nanoparticle composition of the present invention are employed with another therapeutic compound separate from the nanoparticle for treatment of the same indication in the individual. In particular cases, the nanoparticles and the therapeutic agent are delivered separately or together. When delivered together, they may or may not be in the same formulation, and they may or may not be delivered by the same route.

In another aspect, the present disclosure provides methods of synthesizing a compound of Formula (I).

In another aspect, the present disclosure provides methods of making a nanoparticle composition including a lipid component comprising the compound of Formula (I).

### BRIEF DESCRIPTION OF THE DRAWINGS

FIG. 1 shows Formula I lipids formulated in LNPs transfected into HEK293 cells and expressing Fluc-mRNA reporter compared to transfection of naked mRNA.

FIG. 2 shows in vivo expression of Fluc-mRNA reporter payload in BALB mice over 144 hours after subcutaneous dosing of LNP formulations comprising Formula I lipids compared to in vivo expression of the naked mRNA and a FDA approved LNP (LNP156/SM-102).

FIG. 3 shows a scaled graph of in vivo expression of Fluc-mRNA reporter payload in BALB mice over 144 hours after subcutaneous dosing of LNP formulations comprising Formula I lipids compared to in vivo expression of the naked mRNA and a FDA approved LNP (LNP156/SM-102).

FIG. 4 shows body weight fluctuation of BALB mice after subcutaneous dosing of LNP formulations comprising Formula I lipids and a Fluc-mRNA reporter payload compared to in vivo expression of the naked mRNA and a FDA approved LNP (LNP156/SM-102).

FIG. 5 shows total flux AUC values of in vivo expression of Fluc-mRNA reporter payload in BALB mice after subcutaneous dosing of LNP formulations comprising Formula I lipids compared to in vivo expression of the naked mRNA and a FDA approved LNP (LNP156/SM-102).

FIG. 6 shows a scaled graph of total flux AUC values of in vivo expression of Fluc-mRNA reporter payload in BALB mice after subcutaneous dosing of LNP formulations comprising Formula I lipids compared to in vivo expression of the naked mRNA and a FDA approved LNP (LNP156/SM-102).

FIG. 7 shows 2D in vivo expression of Fluc-mRNA reporter payload in BALB mice over 144 hours after intravenous dosing of LNP formulations comprising Formula I lipids compared to in vivo expression of the naked mRNA and a FDA approved LNP (LNP167/D-LIN-MC3 Onpattro®).

FIG. 8 shows a scaled graph of 2D in vivo expression of Fluc-mRNA reporter payload in BALB mice over 144 hours after intravenous dosing of LNP formulations comprising Formula I lipids compared to in vivo expression of the naked mRNA and a FDA approved LNP (LNP167/D-LIN-MC3 Onpattro®).

FIG. 9 shows 2D body weight fluctuation of BALB mice after intravenous dosing of LNP formulations comprising Formula I lipids and a Fluc-mRNA reporter payload compared to in vivo expression of the naked mRNA and a FDA approved LNP (LNP167/D-LIN-MC3 Onpattro®).

FIG. 10 shows total flux AUC values of in vivo expression of Fluc-mRNA reporter payload in BALB mice after intravenous dosing of LNP formulations comprising Formula I lipids compared to in vivo expression of the naked mRNA and a FDA approved LNP (LNP167/D-LIN-MC3 Onpattro®).

FIG. 11 shows a scaled graph of total flux AUC values of in vivo expression of Fluc-mRNA reporter payload in BALB mice after intravenous dosing of LNP formulations comprising Formula I lipids compared to in vivo expression of the naked mRNA and a FDA approved LNP (LNP167/D-LIN-MC3 Onpattro®).

FIG. 12 shows 3D in vivo expression of Fluc-mRNA reporter payload in BALB mice over 144 hours after intravenous dosing of LNP formulations comprising Formula I lipids compared to in vivo expression of the naked mRNA and a FDA approved LNP (LNP167/D-LIN-MC3 Onpattro®).

FIG. 13 shows a scaled graph of 3D in vivo expression of Fluc-mRNA reporter payload in BALB mice over 144 hours after intravenous dosing of LNP formulations comprising Formula I lipids compared to in vivo expression of the naked mRNA and a FDA approved LNP (LNP167/D-LIN-MC3 Onpattro®).

FIG. 14 shows 3D total flux AUC values of in vivo expression of Fluc-mRNA reporter payload in BALB mice after intravenous dosing of LNP formulations comprising Formula I lipids compared to in vivo expression of the naked mRNA and a FDA approved LNP (LNP167/D-LIN-MC3 Onpattro®).

FIG. 15 shows a scaled graph of 3D total flux AUC values of in vivo expression of Fluc-mRNA reporter payload in BALB mice after intravenous dosing of LNP formulations comprising Formula I lipids compared to in vivo expression of the naked mRNA and a FDA approved LNP (LNP167/D-LIN-MC3 Onpattro®).

### DETAILED DESCRIPTION

The disclosure relates to novel ionizable lipids and lipid nanoparticle compositions including a novel ionizable lipid. The disclosure also provides methods of delivering a therapeutic, diagnostic and/or prophylactic agent to a cell, and treating a disease or disorder in a subject need thereof. For example, a method of delivering a therapeutic, diagnostic and/or prophylactic agent to a cell involves contacting a nanoparticle composition of the disclosure comprising a nucleic acid (e.g. DNA or RNA) with a cell, whereby the nucleic acid provides a therapeutic benefit to the subject. A method of delivering a therapeutic, diagnostic and/or prophylactic agent to a target cell or organ may involve administration of a nanoparticle composition comprising one or more ionizable lipids of the disclosure and a payload to the subject.

Certain compounds of the present invention may exist in particular geometric or stereoisomeric forms. The compounds of the present disclosure contemplates all such compounds, including cis- and trans-isomers, R- and S-enantiomers, diastereomers, (D)-isomers, (L)-isomers, the racemic mixtures thereof, and other mixtures thereof. Additional asymmetric carbon atoms may be present in a substituent such as an alkyl group. All such isomers, as well as mixtures thereof, are intended to be included in this invention.

As used herein, the term “isomer” means any geometric isomer, tautomer, zwitterion, stereoisomer, enantiomer, or diastereomer of a compound. Compounds may include one or more chiral centers and/or double bonds and may thus exist as stereoisomers, such as double-bond isomers (i.e., geometric E/Z isomers) or diastereomers (e.g., enantiomers (i.e., (+) or (−)) or cis/trans isomers). The present disclosure encompasses any and all isomers of the compounds described herein, including stereomerically pure forms (e.g., geometrically pure, enantiomerically pure, or diastereomerically pure) and enantiomeric and stereoisomeric mixtures, e.g., racemates.

A person of ordinary skill in the art will recognize that the reactions can be optimized to give one isomer preferentially, new schemes may be devised to produce a single isomer, or isomeric mixtures containing any of a variety of isomer ratios may be utilized. For example, where only two isomers are combined, mixtures containing 50:50, 60:40, 70:30, 80:20, 90:10, 95:5, 96:4, 97:3, 98:2, 99:1, or 100:0 isomer ratios are all contemplated by the present invention. Those of ordinary skill in the art will readily appreciate that analogous ratios are contemplated for more complex isomer mixtures. If one isomer is preferred, techniques such as preparative thin layer chromatography, preparative HPLC, preparative chiral HPLC, or preparative SFC may be used to separate the isomers.

One of ordinary skill in the art will appreciate that the synthetic methods, as described herein, utilize a variety of protecting groups. By the term “protecting group”, as used herein, it is meant that a particular functional moiety, e.g., O, S, or N, is temporarily blocked so that a reaction can be carried out selectively at another reactive site in a multifunctional compound. In preferred embodiments, a protecting group reacts selectively in good yield to give a protected substrate that is stable to the projected reactions; the protecting group should be selectively removable in good yield by readily available, preferably non-toxic reagents that do not attack the other functional groups; the protecting group forms an easily separable derivative (more preferably without the generation of new stereogenic centers); and the protecting group has a minimum of additional functionality to avoid further sites of reaction. As detailed herein, oxygen, sulfur, nitrogen, and carbon protecting groups may be utilized. Hydroxyl protecting groups include methyl, methoxylmethyl (MOM), methylthiomethyl (MTM), t-butylthiomethyl, (phenyldimethylsilyl) methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl) ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl, p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri (p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl) xanthenyl, 9-(9-phenyl-10-oxo) anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio) pentanoate (levulinoyldithioacetal), pivaloate, adamantoate, crotonate, 4-methoxycrotonate, benzoate, p-phenylbenzoate, 2,4,6-trimethylbenzoate (mesitoate), alkyl methyl carbonate, 9-fluorenylmethyl carbonate (Fmoc), alkyl ethyl carbonate, alkyl 2,2,2-trichloroethyl carbonate (Troc), 2-(trimethylsilyl)ethyl carbonate (TMSEC), 2-(phenylsulfonyl)ethyl carbonate (Psec), 2-(triphenylphosphonio)ethyl carbonate (Peoc), alkyl isobutyl carbonate, alkyl vinyl carbonate alkyl allyl carbonate, alkyl p-nitrophenyl carbonate, alkyl benzyl carbonate, alkyl p-methoxybenzyl carbonate, alkyl 3,4-dimethoxybenzyl carbonate, alkyl o-nitrobenzyl carbonate, alkyl p-nitrobenzyl carbonate, alkyl S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy) butyrate, 2-(methylthiomethoxymethyl)benzoate, 2,6-dichloro-4-methylphenoxyacetate, 2,6-dichloro-4-(1,1,3,3-tetramethylbutyl) phenoxyacetate, 2,4-bis(1,1-dimethylpropyl) phenoxyacetate, chlorodiphenylacetate, isobutyrate, monosuccinoate, (E)-2-methyl-2-butenoate, o-(methoxycarbonyl)benzoate, «-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts). For protecting 1,2- or 1,3-diols, the protecting groups include methylene acetal, ethylidene acetal, 1-t-butylethylidene ketal, 1-phenylethylidene ketal, (4-methoxyphenyl)ethylidene acetal, 2,2,2-trichloroethylidene acetal, acetonide, cyclopentylidene ketal, cyclohexylidene ketal, cycloheptylidene ketal, benzylidene acetal, p-methoxybenzylidene acetal, 2,4-dimethoxybenzylidene ketal, 3,4-dimethoxybenzylidene acetal, 2-nitrobenzylidene acetal, methoxymethylene acetal, ethoxymethylene acetal, dimethoxymethylene ortho ester, 1-methoxyethylidene ortho ester, 1-ethoxyethylidine ortho ester, 1,2-dimethoxyethylidene ortho ester, α-methoxybenzylidene ortho ester, 1-(N,N-dimethylamino)ethylidene derivative, α-(N,N′-dimethylamino)benzylidene derivative, 2-oxacyclopentylidene ortho ester, di-t-butylsilylene group (DTBS), 1,3-(1,1,3,3-tetraisopropyldisiloxanylidene) derivative (TIPDS), tetra-t-butoxydisiloxane-1,3-diylidene derivative (TBDS), cyclic carbonates, cyclic boronates, ethyl boronate, and phenyl boronate. Amino-protecting groups include methyl carbamate, ethyl carbamante, 9-fluorenylmethyl carbamate (Fmoc), 9-(2-sulfo) fluorenylmethyl carbamate, 9-(2,7-dibromo) fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BQC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl) methyl carbamate, phenothiazinyl-(10)-carbonyl derivative, N′-p-toluenesulfonylaminocarbonyl derivative, N′-phenylaminothiocarbonyl derivative, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxycarbonylvinyl carbamate, o-(N,N-dimethylcarboxamido)benzyl carbamate, 1,1-dimethyl-3-(N,N-dimethylcarboxamido) propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, 2,4,6-trimethylbenzyl carbamate, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxycarbonylamino) acetamide, 3-(p-hydroxyphenyl) propanamide, 3-(o-nitrophenyl) propanamide, 2-methyl-2-(o-nitrophenoxy) propanamide, 2-methyl-2-(o-phenylazophenoxy) propanamide, 4-chlorobutanamide, 3-methyl-3-nitrobutanamide, o-nitrocinnamide, N-acetylmethionine derivative, o-nitrobenzamide, o-(benzoyloxymethyl)benzamide, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl) ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethylJamine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl) mesityl]methyleneamine, N—(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentacarbonylchromium- or tungsten) carbonyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfonamide, 2-nitro-4-methoxybenzenesulfenamide, triphenylmethylsulfenamide, 3-nitropyridinesulfenamide (Npys), p-toluenesulfonamide (Ts), benzenesulfonamide, 2,3,6,-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide. Exemplary protecting groups are detailed herein, however, it will be appreciated that the present invention is not intended to be limited to these protecting groups; rather, a variety of additional equivalent protecting groups can be readily identified using the above criteria and utilized in the method of the present invention. Additionally, a variety of protecting groups are described in Protective Groups in Organic Synthesis, Third Ed. Greene, T. W. and Wuts, P. G., Eds., John Wiley & Sons, New York: 1999, the entire contents of which are hereby incorporated by reference.

It will be appreciated that the compounds, as described herein, may be substituted with any number of substituents or functional moieties. In general, the term “substituted” whether preceded by the term “optionally” or not, and substituents contained in formulas of this invention, refer to the replacement of hydrogen radicals in a given structure with the radical of a specified substituent. When more than one position in any given structure may be substituted with more than one substituent selected from a specified group, the substituent may be either the same or different at every position. As used herein, the term “substituted” is contemplated to include all permissible substituents of organic compounds. In a broad aspect, the permissible substituents include acyclic and cyclic, branched and unbranched, carbocyclic and heterocyclic, aromatic and nonaromatic substituents of organic compounds. For purposes of this invention, heteroatoms such as nitrogen may have hydrogen substituents and/or any permissible substituents of organic compounds described herein which satisfy the valencies of the heteroatoms. Furthermore, this invention is not intended to be limited in any manner by the permissible substituents of organic compounds. Combinations of substituents and variables envisioned by this invention are preferably those that result in the formation of stable compounds useful in the treatment, for example, of infectious diseases or proliferative disorders. The term “stable”, as used herein, preferably refers to compounds which possess stability sufficient to allow manufacture and which maintain the integrity of the compound for a sufficient period of time to be detected and preferably for a sufficient period of time to be useful for the purposes detailed herein.

As used herein, the term “aliphatic” includes both saturated and unsaturated, straight chain (i.e., unbranched), branched, acyclic, cyclic, or polycyclic aliphatic hydrocarbons, which are optionally substituted with one or more functional groups. As will be appreciated by one of ordinary skill in the art, “aliphatic” is intended herein to include, but is not limited to, alkyl, alkenyl, alkynyl, cycloalkyl, cycloalkenyl, and cycloalkynyl moieties.

As used herein, the term “alkyl” refers to saturated, straight- or branched-chain hydrocarbon radicals derived from a hydrocarbon moiety containing between one and twenty carbon atoms by removal of a single hydrogen atom. Examples of alkyl radicals include, but are not limited to, methyl, ethyl, propyl, isopropyl, n-butyl, tert-butyl, n-pentyl, neopentyl, n-hexyl, n-heptyl, n-octyl, n-decyl, n-undecyl, and dodecyl.

As used herein, the term “alkenyl” refers to a monovalent group derived from a hydrocarbon moiety having at least one carbon-carbon double bond by the removal of a single hydrogen atom. Alkenyl groups include, for example, ethenyl, propenyl, butenyl, 1-methyl-2-buten-1-yl, and the like.

As used herein, the term “alkynyl” refers to a monovalent group derived form a hydrocarbon having at least one carbon-carbon triple bond by the removal of a single hydrogen atom. Representative alkynyl groups include ethynyl, 2-propynyl (propargyl), 1-propynyl, and the like.

As used herein, the term “carboxylic acid” refers to a group of formula —CO2H.

Alkyl, alkenyl, and cyclyl (e.g., carbocyclyl and heterocyclyl) groups may be optionally substituted unless otherwise specified.

As used herein, the terms “aryl” and “heteroaryl” refer to stable mono- or polycyclic, heterocyclic, polycyclic, and polyheterocyclic unsaturated moieties having 3-14 carbon atoms, each of which may be substituted or unsubstituted. Substituents include, but are not limited to, any of the previously mentioned substitutents.

In certain aspects of the present disclosure, “aryl” refers to a mono- or bicyclic carbocyclic ring system having one or two aromatic rings including, but not limited to, phenyl, naphthyl, tetrahydronaphthyl, indanyl, indenyl, and the like.

In certain aspects of the present disclosure, the term “heteroaryl”, as used herein, an aromatic ring containing the indicated number of atoms (e.g., 5 to 12, or 5 to 10 membered heteroaryl) made up of one or more heteroatoms (e.g., 1, 2, 3 or 4 heteroatoms) selected from N, O and S and with the remaining ring atoms being carbon. Heteroaryl groups do not contain adjacent S and O atoms. In some embodiments, the total number of S and O atoms in the heteroaryl group is not more than 2. In some embodiments, the total number of S and O atoms in heteroaryl group is not more than 1. Heteroaryl groups may be bound to the parent structure by a carbon or nitrogen atom, as valency permits. For example, “pyridyl” includes 2-pyridyl, 3-pyridyl and 4-pyridyl groups, and “pyrrolyl” includes 1-pyrrolyl, 2-pyrrolyl and 3-pyrrolyl groups. When nitrogen is present in a heteroaryl ring, it may, where the nature of the adjacent atoms and groups permits, exist in an oxidized state (i.e., N+-O−). Additionally, when sulfur is present in a heteroaryl ring, it may, where the nature of the adjacent atoms and groups permits, exist in an oxidized state (i.e., S+-O− or SO2). Heteroaryl groups may be monocyclic or polycyclic (e.g., bicyclic, tricyclic). Any of the aryl or heteroaryl substituents described above and herein may be substituted or unsubstituted. In some instances, a heteroaryl group is monocyclic. Examples include pyrrole, pyrazole, imidazole, triazole (e.g., 1,2,3-triazole, 1,2,4-triazole, 1,2,4-triazole), tetrazole, furan, isoxazole, oxazole, oxadiazole (e.g., 1,2,3-oxadiazole, 1,2,4-oxadiazole, 1,3,4-oxadiazole), thiophene, isothiazole, thiazole, thiadiazole (e.g., 1,2,3-thiadiazole, 1,2,4-thiadiazole, 1,3,4-thiadiazole), pyridine, pyridazine, pyrimidine, pyrazine, triazine (e.g., 1,2,4-triazine, 1,3,5-triazine) and tetrazine. In some instances, both rings of a polycyclic heteroaryl group are aromatic. Examples include indole, isoindole, indazole, benzoimidazole, benzotriazole, benzofuran, benzoxazole, benzoisoxazole, benzoxadiazole, benzothiophene, benzothiazole, benzoisothiazole, benzothiadiazole, 1H-pyrrolo[2,3-b]pyridine, 1H-pyrazolo[3,4-b]pyridine, 3H-imidazo[4,5-b]pyridine, 3H-[1,2,3]triazolo[4,5-b]pyridine, 1H-pyrrolo[3,2-b]pyridine, 1H-pyrazolo[4,3-b]pyridine, 1H-imidazo[4,5-b]pyridine, 1H-[1,2,3]triazolo[4,5-b]pyridine, 1H-pyrrolo[2,3-c]pyridine, 1H-pyrazolo[3,4-c]pyridine, 3H-imidazo[4,5-c]pyridine, 3H-[1,2,3]triazolo[4,5-c]pyridine, 1H-pyrrolo[3,2-c]pyridine, 1H-pyrazolo[4,3-c]pyridine, 1H-imidazo[4,5-c]pyridine, 1H-[1,2,3]triazolo[4,5-c]pyridine, furo[2,3-b]pyridine, oxazolo[5,4-b]pyridine, isoxazolo[5,4-b]pyridine, [1,2,3]oxadiazolo[5,4-b]pyridine, furo[3,2-b]pyridine, oxazolo[4,5-b]pyridine, isoxazolo[4,5-b]pyridine, [1,2,3]oxadiazolo[4,5-b]pyridine, furo[2,3-c]pyridine, oxazolo[5,4-c]pyridine, isoxazolo[5,4-c]pyridine, [1,2,3]oxadiazolo[5,4-c]pyridine, furo[3,2-c]pyridine, oxazolo[4,5-c]pyridine, isoxazolo[4,5-c]pyridine, [1,2,3]oxadiazolo[4,5-c]pyridine, thieno[2,3-b]pyridine, thiazolo[5,4-b]pyridine, isothiazolo[5,4-b]pyridine, [1,2,3]thiadiazolo[5,4-b]pyridine, thieno[3,2-b]pyridine, thiazolo[4,5-b]pyridine, isothiazolo[4,5-b]pyridine, [1,2,3]thiadiazolo[4,5-b]pyridine, thieno[2,3-c]pyridine, thiazolo[5,4-c]pyridine, isothiazolo[5,4-c]pyridine, [1,2,3]thiadiazolo[5,4-c]pyridine, thieno[3,2-c]pyridine, thiazolo[4,5-c]pyridine, isothiazolo[4,5-c]pyridine, [1,2,3]thiadiazolo[4,5-c]pyridine, quinoline, isoquinoline, cinnoline, quinazoline, quinoxaline, phthalazine, naphthyridine (e.g., 1,8-naphthyridine, 1,7-naphthyridine, 1,6-naphthyridine, 1,5-naphthyridine, 2,7-naphthyridine, 2,6-naphthyridine), imidazo[1,2-a]pyridine, 1H-pyrazolo[3,4-d]thiazole, 1H-pyrazolo[4,3-d]thiazole and imidazo[2,1-b]thiazole.

As used herein, the term “cycloalkyl”, as used herein, refers specifically to groups having three to seven, preferably three to ten carbon atoms. Suitable cycloalkyls include, but are not limited to cyclopropyl, cyclobutyl, cyclopentyl, cyclohexyl, cycloheptyl and the like, which, as in the case of other aliphatic, heteroaliphatic, or heterocyclic moieties, may optionally be substituted with substituents including, but not limited to aliphatic; heteroaliphatic; aryl; heteroaryl; arylalkyl; heteroarylalkyl; alkoxy; aryloxy; heteroalkoxy; heteroaryloxy; alkylthio; arylthio; heteroalkylthio; heteroarylthio; —F; —Cl; —Br; —I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓ; —OCO₂Rₓ; —OCON(Rₓ)₂; —N(Rₓ)₂; —S(O)₂Rₓ; —NRₓ(CO)Rₓ, wherein each occurrence of Rₓ independently includes, but is not limited to, aliphatic, heteroaliphatic, aryl, heteroaryl, arylalkyl, or heteroarylalkyl, wherein any of the aliphatic, heteroaliphatic, arylalkyl, or heteroarylalkyl substituents described above and herein may be substituted or unsubstituted, branched or unbranched, cyclic or acyclic, and wherein any of the aryl or heteroaryl substituents described above and herein may be substituted or unsubstituted.

As used herein, the term “heteroaliphatic” refers to aliphatic moieties that contain one or more oxygen, sulfur, nitrogen, phosphorus, or silicon atoms, e.g., in place of carbon atoms. Heteroaliphatic moieties may be branched, unbranched, cyclic or acyclic and include saturated and unsaturated heterocycles such as morpholino, pyrrolidinyl, etc. In certain embodiments, heteroaliphatic moieties are substituted by independent replacement of one or more of the hydrogen atoms thereon with one or more moieties including, but not limited to aliphatic; heteroaliphatic; aryl; heteroaryl; arylalkyl; heteroarylalkyl; alkoxy; aryloxy; heteroalkoxy; heteroaryloxy; alkylthio; arylthio; heteroalkylthio; heteroarylthio; —F; —Cl; —Br; —I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓ; —OCO₂Rₓ; —OCON(Rₓ)₂; —N(Rₓ)₂; —S(O)₂Rₓ; —NRₓ(CO)Rₓ, wherein each occurrence of Rₓ independently includes, but is not limited to, aliphatic, heteroaliphatic, aryl, heteroaryl, arylalkyl, or heteroarylalkyl, wherein any of the aliphatic, heteroaliphatic, arylalkyl, or heteroarylalkyl substituents described above and herein may be substituted or unsubstituted, branched or unbranched, cyclic or acyclic, and wherein any of the aryl or heteroaryl substituents described above and herein may be substituted or unsubstituted.

As used herein, the terms “halo” and “halogen” refer to an atom selected from fluorine, chlorine, bromine, and iodine.

As used herein, the term “haloalkyl” denotes an alkyl group, as defined above, having one, two, or three halogen atoms attached thereto and is exemplified by such groups as chloromethyl, bromoethyl, trifluoromethyl, and the like.

As used herein, the term “heterocycloalkyl” or “heterocycle” refers to a non-aromatic 5-, 6-, or 7-membered ring or a polycyclic group, including, but not limited to a bi- or tri-cyclic group comprising fused six-membered rings having between one and three heteroatoms independently selected from oxygen, sulfur and nitrogen, wherein (i) each 5-membered ring has 0 to 1 double bonds and each 6-membered ring has 0 to 2 double bonds, (ii) the nitrogen and sulfur heteroatoms may be optionally be oxidized, (iii) the nitrogen heteroatom may optionally be quaternized, and (iv) any of the above heterocyclic rings may be fused to a benzene ring. Representative heterocycles include, but are not limited to, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, and tetrahydrofuryl. In certain embodiments, a “substituted heterocycloalkyl or heterocycle” group is utilized and as used herein, refers to a heterocycloalkyl or heterocycle group, as defined above, substituted by the independent replacement of one, two or three of the hydrogen atoms thereon with but are not limited to aliphatic; heteroaliphatic; aryl; heteroaryl; arylalkyl; heteroarylalkyl; alkoxy; aryloxy; heteroalkoxy; heteroaryloxy; alkylthio; arylthio; heteroalkylthio; heteroarylthio; —F; —Cl; —Br; —I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓ; —OCO₂Rₓ; —OCON(Rₓ)₂; —N(Rₓ)₂; —S(O)₂Rₓ; —NRₓ (CO)Rₓ, wherein each occurrence of Rₓ independently includes, but is not limited to, aliphatic, heteroaliphatic, aryl, heteroaryl, arylalkyl, or heteroarylalkyl, wherein any of the aliphatic, heteroaliphatic, arylalkyl, or heteroarylalkyl substituents described above and herein may be substituted or unsubstituted, branched or unbranched, cyclic or acyclic, and wherein any of the aryl or heteroaryl substituents described above and herein may be substituted or unsubstituted.

As used herein, the terms halo and halogen refer to an atom selected from fluorine, chlorine, bromine, and iodine.

As used herein the term “heterocyclic” refers to a non-aromatic partially unsaturated or fully saturated 3- to 10-membered ring system, which includes single rings of 3 to 8 atoms in size and bi- and tri-cyclic ring systems which may include aromatic six-membered aryl or aromatic heterocyclic groups fused to a non-aromatic ring. These heterocyclic rings include those having from one to three heteroatoms independently selected from oxygen, sulfur, and nitrogen, in which the nitrogen and sulfur heteroatoms may optionally be oxidized and the nitrogen heteroatom may optionally be quaternized.

As used herein the term “heteroaryl” refers to a cyclic aromatic radical having from five to ten ring atoms of which one ring atom is selected from sulfur, oxygen, and nitrogen; zero, one, or two ring atoms are additional heteroatoms independently selected from sulfur, oxygen, and nitrogen; and the remaining ring atoms are carbon, the radical being joined to the rest of the molecule via any of the ring atoms, such as, for example, pyridyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl, isoquinolinyl, and the like.

As used herein, the term “squaramide” refers to a conformationally rigid cyclobutene ring composed of two carbonyl hydrogen-bond acceptors in close proximity to two NH hydrogen-bond donors. These cyclobutene rings comprise a delocalized nitrogen lone pair, which confers the four-membered ring with aromatic character (Hückel's rule: [4n+2]π electrons, n=0). A description of the chemical and physical properties of squaramides are are found in L. A. Marchetti, L. K. Kumawat, N. Mao, J. C. Stephens, R. B. Elmes, The versatility of squaramides: from supramolecular chemistry to chemical biology Chem, 5 (2019), pp. 1398-1485, which is incorporated herein by reference in its entirety.

As used herein, a “peptide” or “protein” comprises a string of at least three amino acids linked together by peptide bonds. The terms “protein” and “peptide” may be used interchangeably. Peptide may refer to an individual peptide or a collection of peptides. Inventive peptides preferably contain only natural amino acids, although non-natural amino acids (i.e., compounds that do not occur in nature but that can be incorporated into a polypeptide chain) and/or amino acid analogs as are known in the art may alternatively be employed. Also, one or more of the amino acids in an inventive peptide may be modified, for example, by the addition of a chemical entity such as a carbohydrate group, a phosphate group, a farnesyl group, an isofarnesyl group, a fatty acid group, a linker for conjugation, functionalization, or other modification, etc. In a preferred embodiment, the modifications of the peptide lead to a more stable peptide (e.g., greater half-life in vivo). These modifications may include cyclization of the peptide, the incorporation of D-amino acids, etc. None of the modifications should substantially interfere with the desired biological activity of the peptide.

As used herein, the terms “polynucleotide” or “oligonucleotide” refers to a polymer of nucleotides. The polymer may include natural nucleosides (i.e., adenosine, thymidine, guanosine, cytidine, uridine, deoxyadenosine, deoxythymidine, deoxyguanosine, and deoxycytidine), nucleoside analogs (e.g., 2-aminoadenosine, 2-thiothymidine, inosine, pyrrolo-pyrimidine, 3-methyl adenosine, C5-propynylcytidine, C5-propynyluridine, C5-bromouridine, C5-fluorouridine, C5-iodouridine, C5-methylcytidine, 7-deazaadenosine, 7-deazaguanosine, 8-oxoadenosine, 8-oxoguanosine, O (6)-methylguanine, and 2-thiocytidine), chemically modified bases, biologically modified bases (e.g., methylated bases), intercalated bases, modified sugars (e.g., 2% fluororibose, ribose, 2′-deoxyribose, arabinose, and hexose), or modified phosphate groups (e.g., phosphorothioates and 5′-N-phosphoramidite linkages).

As used herein, the terms “approximately” and “about,” as applied to one or more values of interest, refer to a value that is similar to a stated reference value. In certain aspects, the term “approximately” or “about” refers to a range of values that fall within 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). For example, when used in the context of an amount of a given compound in a lipid component of a nanoparticle composition, “about” may mean±/−10% of the recited value.

As used herein, the term “compound,” includes all isomers and isotopes of the structure depicted. “Isotopes” refers to atoms having the same atomic number but different mass numbers resulting from a different number of neutrons in the nuclei. For example, isotopes of hydrogen include tritium and deuterium. Further, a compound, salt, or complex of the present disclosure can be prepared in combination with solvent or water molecules to form solvates and hydrates by routine methods.

As used herein, the term “contacting” means establishing a physical connection between two or more entities. For example, contacting a cell with a nanoparticle composition means that the cell and a nanoparticle are made to share a physical connection. Methods of contacting cells with external entities both in vivo and ex vivo are well known in the biological arts, including intravenous, intramuscular, intradermal, and subcutaneous methods of administration, and may involve varied amounts of nanoparticle compositions.

As used herein, the term “delivering” means providing an entity to a destination. For example, delivering an effective amount of a biologically active agent to a subject may involve administering a nanoparticle composition including the biologically active agent to the subject (e.g., by an intravenous, intramuscular, intradermal, or subcutaneous route).

As used herein, “encapsulation efficiency” refers to the amount of a therapeutic and/or prophylactic that becomes part of a nanoparticle composition, relative to the initial total amount of therapeutic and/or prophylactic used in the preparation of a nanoparticle composition. For example, if 97 mg of therapeutic, diagnostic and/or prophylactic are encapsulated in a nanoparticle composition out of a total 100 mg of therapeutic and/or prophylactic initially provided to the composition, the encapsulation efficiency may be given as 97%. As used herein, “encapsulation” may refer to complete, substantial, or partial enclosure, confinement, surrounding, or encasement.

As used herein, “expression” of a nucleic acid sequence refers to translation of an mRNA into a polypeptide or protein and/or post-translational modification of a polypeptide or protein.

As used herein, the term “effective amount” of an active agent or drug delivery device refers to the amount necessary to elicit the desired biological response. As will be appreciated by those of ordinary skill in this art, the effective amount of an agent or device may vary depending on such factors as the desired biological effect, the agent to be delivered, the composition of the encapsulating matrix, the target tissue, etc. For example, the effective amount of microparticles containing an antigen to be delivered to immunize an individual is the amount that results in an immune response sufficient to prevent infection with an organism having the administered antigen.

As used herein, the term “subject” or “patient” refers to any organism to which a composition in accordance with the disclosure may be administered, e.g., for experimental, diagnostic, prophylactic, and/or therapeutic purposes. Typical subjects include animals (e.g., mammals such as mice, rats, rabbits, non-human primates, and humans) and/or plants.

As used herein, “methods of administration” may include intravenous, intramuscular, intradermal, subcutaneous, or other methods of delivering a composition to a subject. A method of administration may be selected to target delivery (e.g., to specifically deliver) to a specific region or system of a body.

As used herein, “modified” means non-natural. For example, an RNA may be a modified RNA. That is, an RNA may include one or more nucleobases, nucleosides, nucleotides, or linkers that are non-naturally occurring.

As used herein, a “nanoparticle composition” is a composition comprising one or more lipids. Nanoparticle compositions are typically sized on the order of micrometers or smaller and may include a lipid bilayer. Nanoparticle compositions encompass lipid nanoparticles (LNPs), liposomes (e.g., lipid vesicles), and lipoplexes. For example, a nanoparticle composition may be a liposome having a lipid bilayer with a diameter of 500 nm or less.

As used herein, “naturally occurring” means existing in nature without artificial modification.

As used herein, a “PEG lipid” or “PEGylated lipid” refers to a lipid comprising a polyethylene glycol component, such as PEG-modified phosphatidylethanolamine, a PEG-modified phosphatidic acid, a PEG-modified ceramide, a PEG-modified dialkylamine, a PEG-modified diacylglycerol, and a PEG-modified dialkylglycerol.

The phrase “pharmaceutically acceptable” is used herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable excipient,” as used herein, refers to any ingredient other than the compounds described herein (for example, a vehicle capable of suspending, complexing, or dissolving the active compound) and having the properties of being substantially nontoxic and non-inflammatory in a patient. Excipients may include, for example: anti-adherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspending or dispersing agents, sweeteners, and waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E (alpha-tocopherol), vitamin C, xylitol, and other species disclosed herein.

As used herein, “pharmaceutically acceptable salts” refers to derivatives of the disclosed compounds wherein the parent compound is altered by converting an existing acid or base moiety to its salt form (e.g., by reacting a free base group with a suitable organic acid). Examples of pharmaceutically acceptable salts include, but are not limited to, mineral or organic acid salts of basic residues such as amines; alkali or organic salts of acidic residues such as carboxylic acids; and the like. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17^{th} ed., Mack Publishing Company, Easton, Pa., 1985, p. 1418, Pharmaceutical Salts: Properties, Selection, and Use, P. H. Stahl and C. G. Wermuth (eds.), Wiley-VCH, 2008, and Berge et al., Journal of Pharmaceutical Science, 66, 1-19 (1977), each of which is incorporated herein by reference in its entirety.

As used herein, an “RNA” refers to a ribonucleic acid that may be naturally or non-naturally occurring. For example, an RNA may include modified and/or non-naturally occurring components such as one or more nucleobases, nucleosides, nucleotides, or linkers. An RNA may include a cap structure, a chain terminating nucleoside, a stem loop, a polyA sequence, and/or a polyadenylation signal. An RNA may have a nucleotide sequence encoding a polypeptide of interest. For example, an RNA may be a messenger RNA (mRNA). Translation of an mRNA encoding a particular polypeptide, for example, in vivo translation of an mRNA inside a mammalian cell, may produce the encoded polypeptide. RNAs may be selected from the non-liming group consisting of small interfering RNA (siRNA), asymmetrical interfering RNA (aiRNA), microRNA (miRNA), Dicer-substrate RNA (dsRNA), small hairpin RNA (shRNA), mRNA, and mixtures thereof.

As used herein, the term “therapeutic agent” refers to any agent that, when administered to a subject, has a therapeutic and/or diagnostic effect and/or elicits a desired biological and/or pharmacological effect. The term “prophylactic agent” refers to any agent that, when administered to a subject, has a prophylactic effect. Therapeutic and/or prophylactic agents are also referred to as “biologically active agents” or “agents.” Such agents include, but are not limited to, small molecules, organometallic compounds, nucleic acids, proteins, peptides, polynucleotides, metals, isotopically labeled chemical compounds, drugs, vaccines, immunological agents, etc. As used herein, “small molecules” include, but is not limited to, antineoplastic agents (e.g., vincristine, doxorubicin, mitoxantrone, camptothecin, cisplatin, bleomycin, cyclophosphamide, methotrexate, and streptozotocin), antitumor agents (e.g., actinomycin D, vincristine, vinblastine, cystine arabinoside, anthracyclines, alkylative agents, platinum compounds, antimetabolites, and nucleoside analogs, such as methotrexate and purine and pyrimidine analogs), anti-infective agents, local anesthetics (e.g., dibucaine and chlorpromazine), beta-adrenergic blockers (e.g., propranolol, timolol, and labetolol), antihypertensive agents (e.g., clonidine and hydralazine), anti-depressants (e.g., imipramine, amitriptyline, and doxepim), anti-conversants (e.g., phenytoin), antihistamines (e.g., diphenhydramine, chlorphenirimine, and promethazine), antibiotic/antibacterial agents (e.g., gentamycin, ciprofloxacin, and cefoxitin), antifungal agents (e.g., miconazole, terconazole, econazole, isoconazole, butaconazole, clotrimazole, itraconazole, nystatin, naftifine, and amphotericin B), antiparasitic agents, hormones, hormone antagonists, immunomodulators, neurotransmitter antagonists, antiglaucoma agents, vitamins, narcotics, and imaging agents.

As used herein, the term “therapeutically effective amount” means an amount of an agent to be delivered (e.g., nucleic acid, drug, composition, therapeutic agent, diagnostic agent, prophylactic agent, etc.) that is sufficient, when administered to a subject suffering from or susceptible to an infection, disease, disorder, and/or condition, to treat, improve symptoms of, diagnose, prevent, and/or delay the onset of the infection, disease, disorder, and/or condition.

### Nanoparticle Compositions

The present disclosure provides novel ionizable lipids and delivery systems based on the use of novel ionizable lipids, such as nanoparticle compositions. Nanoparticle compositions comprising a lipid component comprising a compound according to Formula (I) are described herein.

In one aspect, the present disclosure provides compounds of Formula (I):

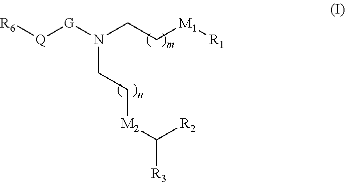

or a salt or isomer thereof, wherein
each m is independently an integer from 4-13, e.g., 4, 5, 6, 7, 8, 9, 10, 11, 12, or 13 or any subrange selected from within the range of 4-13, e.g., 4-9, 6-8, 4-7, 4-5, 5-9, 6-13, etc.; each n is independently an integer from 1-3, e.g., 1, 2 or 3, or any subrange selected from the range of 1-3, e.g., 1-2, 2-3, etc.;
each R¹ is independently selected from C1-C5 alkyl, C2-C5 alkenyl, C2-C5 alkynyl, wherein the alkyl, the alkenyl, or the alkynyl is linear or branched;
each R₂ and R₃ is independently selected from C₁ to C₁₄ alkyl;
each R₆ is independently selected from H,

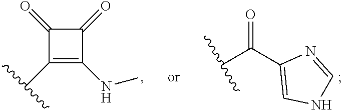

each M₁ and M₂ is independently selected from —C(O)O—, and —OC(O)—, wherein at least one of M₁ or M₂ is —C(O)O—;
each Q is selected from —O— or —NH—;
and each G is —(CR₄R₅)ₖ—;
wherein each k is selected from an integer from 2-5, e.g. 2, 3, 4, and 5; or any subrange selected from the range of 2-5, e.g. 2, -3, 3-4, and 2-5 etc.;
and each R₄ and R₅ is independently selected from H, and C₁-C₃ alkyl

### Synthesis Scheme 1.

In one aspect, the present disclosure provides a method for synthesizing the compound:

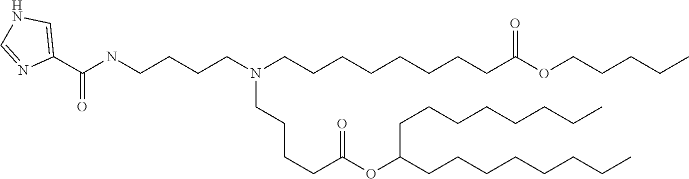

(I) (i) (also referred to as Compound 65)
comprising performing the following reaction:

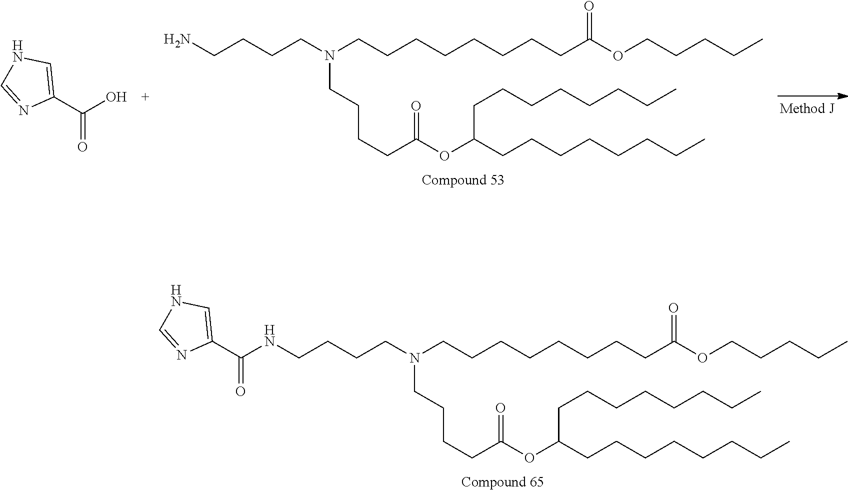 [SMILES: O=C(O)c1c[nH]cn1 ; CCCCCCCCC(CCCCCCCC)OC(=O)CCCCN(CCCCN)CCCCCCCCC(=O)OCCCCC ; CCCCCCCCC(CCCCCCCC)OC(=O)CCCCN(CCCCCCCCC(=O)OCCCCC)CCCCNC(=O)c1c[nH]cn1]

### Synthesis Scheme 2

In one aspect, the present disclosure provides a method for synthesizing the compound

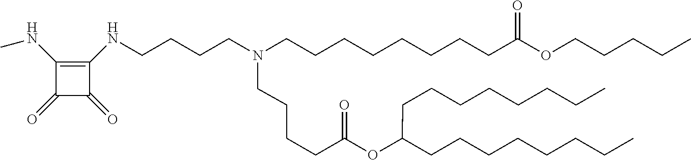 [SMILES: CCCCCCCCC(CCCCCCCC)OC(=O)CCCCN(CCCCCCCCC(=O)OCCCCC)CCCCNc1c(NC)c(=O)c1=O]

(I) (g) (also referred to as Compound 54)
comprising performing the following reaction:

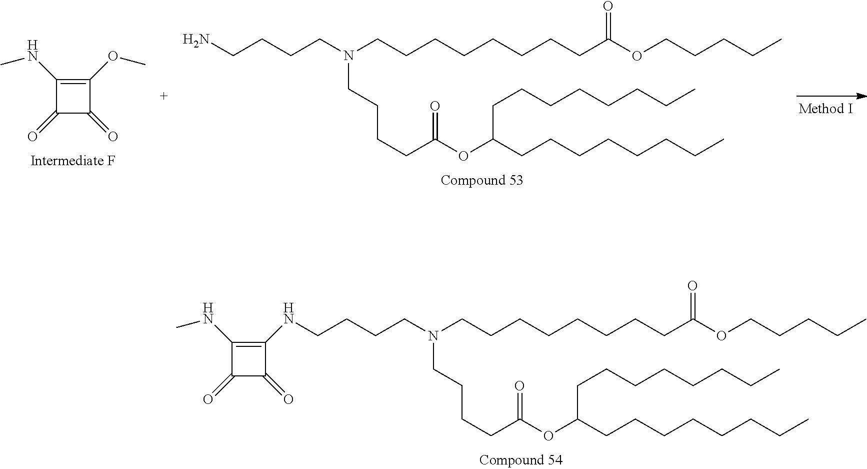

wherein the synthesis route for intermediate F is:

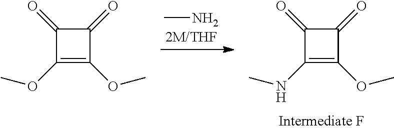

### Synthesis Scheme 3

In one aspect, the present disclosure provides a method for synthesizing the compound

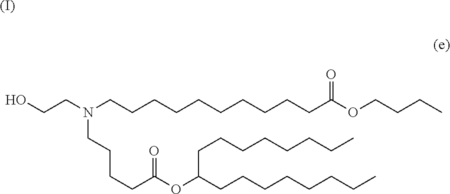 [SMILES: "(I)" ; CCCCCCCCC(CCCCCCCC)OC(=O)CCCCN(CCO)CCCCCCCCCCC(=O)OCCCC]

(also referred to as Compound 35)
comprising performing the following reaction:

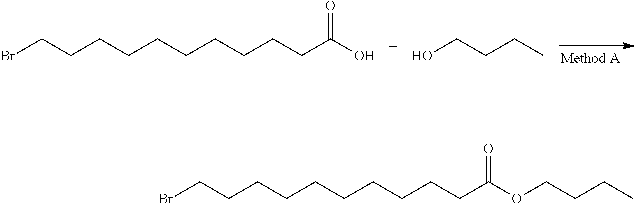

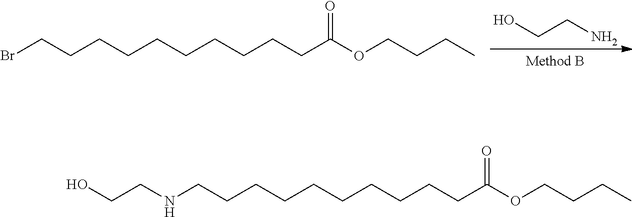

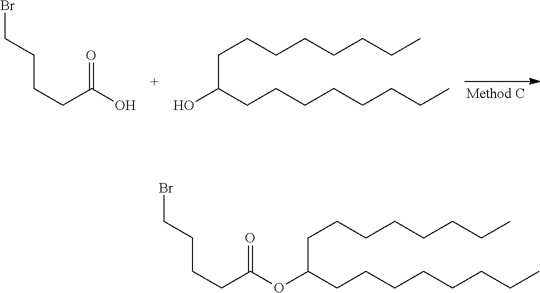 [SMILES: O=C(O)CCCCBr ; CCCCCCCCC(O)CCCCCCCC ; CCCCCCCCC(CCCCCCCC)OC(=O)CCCCBr]

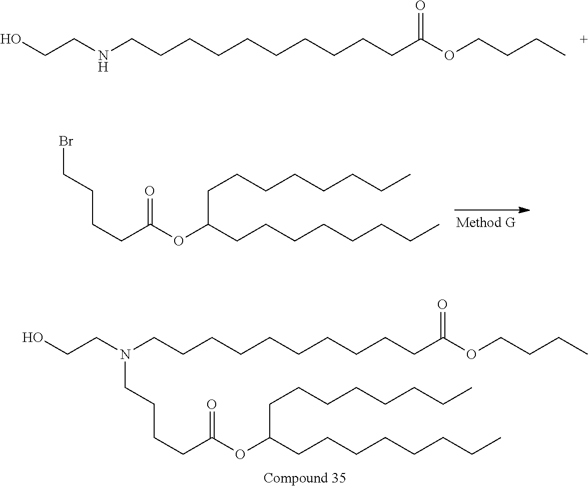 [SMILES: CCCCOC(=O)CCCCCCCCCCNCCO ; CCCCCCCCC(CCCCCCCC)OC(=O)CCCCBr ; CCCCCCCCC(CCCCCCCC)OC(=O)CCCCN(CCO)CCCCCCCCCCC(=O)OCCCC]

### Synthesis Scheme 4

In one aspect, the present disclosure provides a method for synthesizing the compound

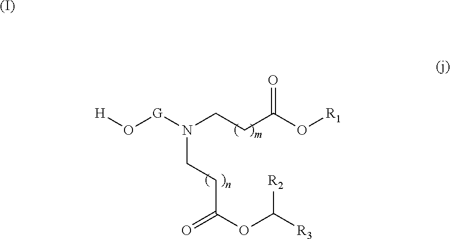

or a salt or isomer thereof, wherein
each m is independently an integer from 4-13;
each n is independently an integer from 1-3;
each R¹ is independently selected from C1-C5 alkyl, C2-C5 alkenyl, C2-C5 alkynyl, wherein the alkyl, the alkenyl, or the alkynyl is linear or branched;
each R₂ and R₃ is independently selected from C₁ to C₁₄ alkyl;
and each G is —(CR₄R₅)ₖ—;
wherein each k is selected from an integer from 2-5;
and each R₄ and R₅ is independently selected from H, and C₁-C₃ alkyl, comprising performing the following reaction:

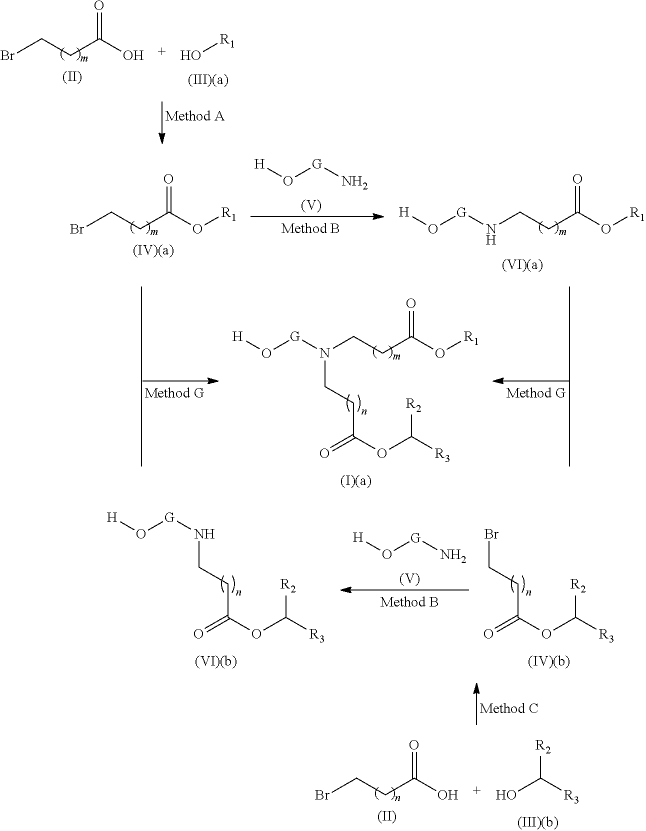

### Synthesis Scheme 5

In one aspect, the present disclosure provides a method for synthesizing the compound

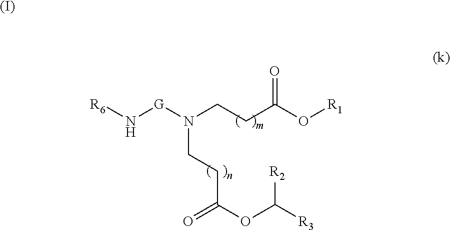

or a salt or isomer thereof, wherein
each m is independently an integer from 4-13;
each n is independently an integer from 1-3;
each R¹ is independently selected from C1-C5 alkyl, C2-C5 alkenyl, C2-C5 alkynyl, wherein the alkyl, the alkenyl, or the alkynyl is linear or branched;
each R₂ and R₃ is independently selected from C₁ to C₁₄ alkyl;
each R₆ is H,
and each G is —(CR₄R₅)ₖ—;
wherein each k is selected from an integer from 2-5;
and each R₄ and R₅ is independently selected from H, and C₁-C₃ alky, comprising performing the following reaction:

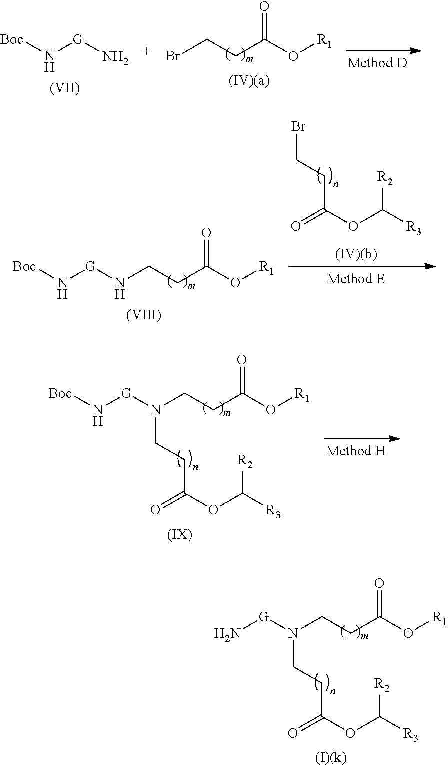

### Synthesis Scheme 6

In one aspect, the present disclosure provides a method for synthesizing the compound

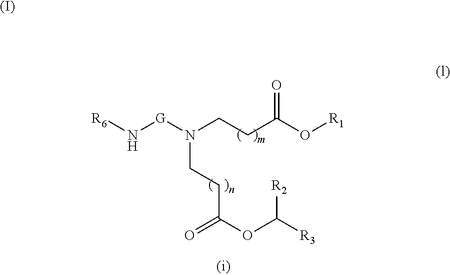

or a salt or isomer thereof, wherein
each m is independently an integer from 4-13;
each n is independently an integer from 1-3;
each R¹ is independently selected from C1-C5 alkyl, C2-C5 alkenyl, C2-C5 alkynyl, wherein the alkyl, the alkenyl, or the alkynyl is linear or branched;
each R₂ and R₃ is independently selected from C₁ to C₁₄ alkyl;
each R₆ is

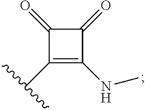 [SMILES: *c1c(NC)c(=O)c1=O]

and each G is —(CR₄R₅)ₖ—;
wherein each k is selected from an integer from 2-5;
and each R₄ and R₅ is independently selected from H, and C₁-C₃ alkyl, comprising performing the following reaction:

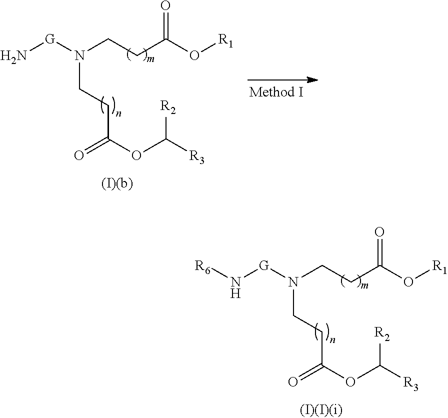

### Synthesis Scheme 7

In one aspect, the present disclosure provides a method for synthesizing the compound

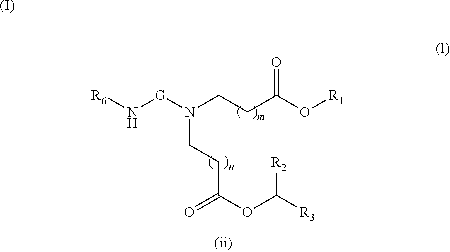

or a salt or isomer thereof, wherein
each m is independently an integer from 4-13;
each n is independently an integer from 1-3;
each R¹ is independently selected from C1-C5 alkyl, C2-C5 alkenyl, C2-C5 alkynyl, wherein the alkyl, the alkenyl, or the alkynyl is linear or branched;
each R₂ and R₃ is independently selected from C₁ to C₁₄ alkyl;
each R₆ is

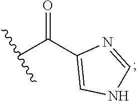 [SMILES: *C(=O)c1c[nH]cn1]

and each G is —(CR₄R₅)ₖ—;
wherein each k is selected from an integer from 2-5;
and each R₄ and R₅ is independently selected from H, and C₁-C₃ alkyl, comprising performing the following reaction:

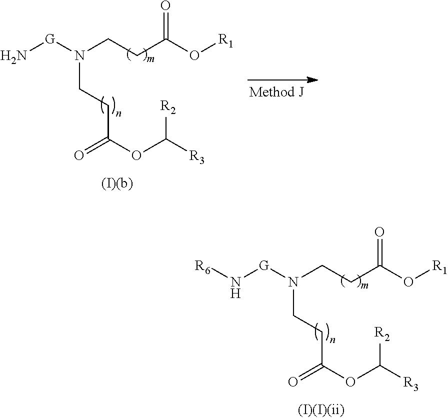

### Synthesis Scheme 8

In one aspect, the present disclosure provides a method for synthesizing the compound

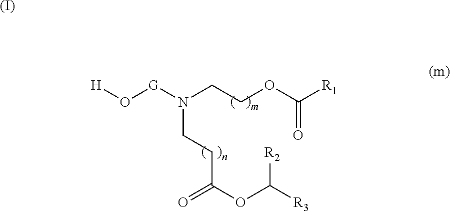

or a salt or isomer thereof, wherein
each m is independently an integer from 4-13;
each n is independently an integer from 1-3;
each R¹ is independently selected from C1-C5 alkyl, C2-C5 alkenyl, C2-C5 alkynyl, wherein the alkyl, the alkenyl, or the alkynyl is linear or branched;
each R₂ and R₃ is independently selected from C₁ to C₁₄ alkyl;
and each G is —(CR₄R₅)ₖ—;
wherein each k is selected from an integer from 2-5;
and each R₄ and R₅ is independently selected from H, and C₁-C₃ alkyl, comprising performing the following reaction:

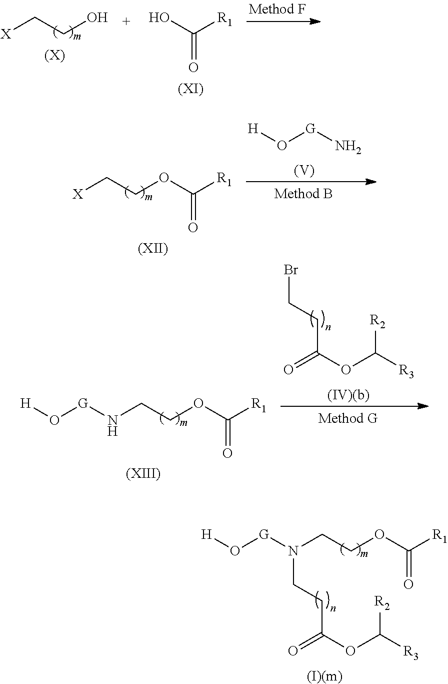

wherein
X is Cl, Br;
each m is independently an integer from 4-13, e.g., 4, 5, 6, 7, 8, 9, 10, 11, 12, or 13 or any subrange selected from within the range of 4-13, e.g., 4-9, 6-8, 4-7, 4-5, 5-9, 6-13 etc.;
each n is independently an integer from 1-3, e.g., 1, 2 or 3, or any subrange selected from the range of 1-3, e.g., 1-2, 2-3, etc.;
each R₁ is Ri is C₁-C₅ alkyl, wherein the alkyl is linear or branched;
each R₂ and R₃ is independently selected from C₁ to C₁₄ alkyl;
and each G is —(CR₄R₅)ₖ—;
wherein each k is selected from an integer from 2-5, e.g. 2, 3, 4, and 5; or any subrange selected from the range of 2-5, e.g. 2, -3, 3-4, and 2-5 etc.;
and each R₄ and R₅ is independently selected from H, and C₁-C₃ alkyl.

In one aspect, method F is:

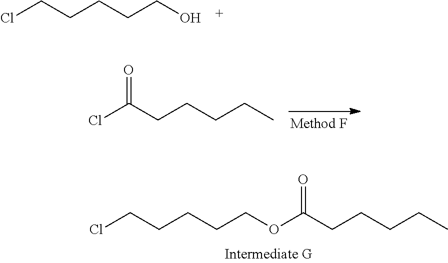

### Synthesis Scheme 9

In one aspect, the present disclosure provides a method for synthesizing the compound

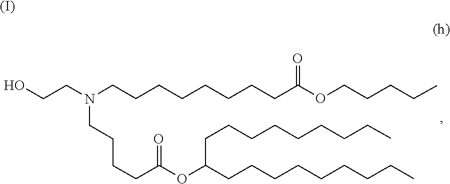 [SMILES: "(I)" ; CCCCCCCCCC(CCCCCCCCC)OC(=O)CCCCN(CCO)CCCCCCCCC(=O)OCCCCC]

comprising performing the following reaction:

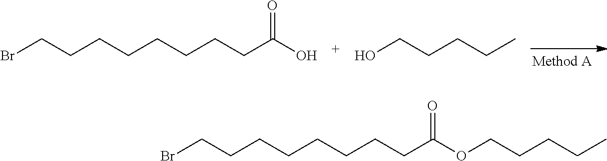

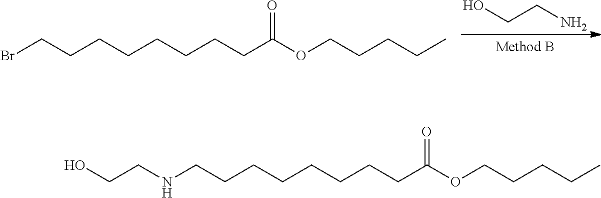

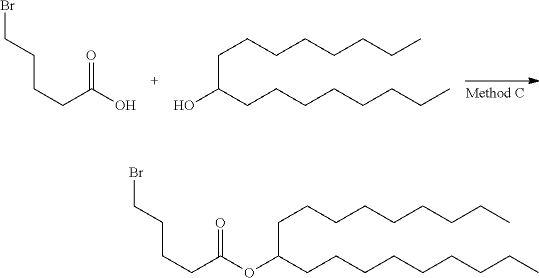 [SMILES: O=C(O)CCCCBr ; CCCCCCCCC(O)CCCCCCCC ; CCCCCCCCCC(CCCCCCCCC)OC(=O)CCCCBr]

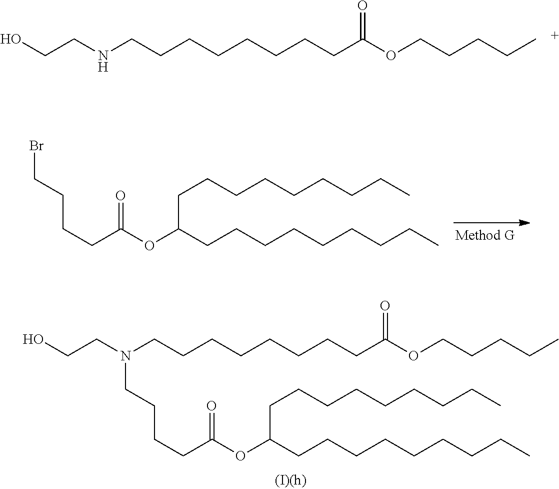 [SMILES: CCCCCOC(=O)CCCCCCCCNCCO ; CCCCCCCCCC(CCCCCCCCC)OC(=O)CCCCBr ; CCCCCCCCCC(CCCCCCCCC)OC(=O)CCCCN(CCO)CCCCCCCCC(=O)OCCCCC]

### Synthesis Scheme 10

In one aspect, the present disclosure provides a method for synthesizing the compound

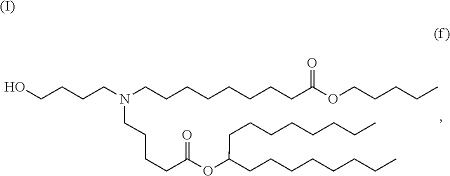 [SMILES: "(I)" ; CCCCCCCCC(CCCCCCCC)OC(=O)CCCCN(CCCCO)CCCCCCCCC(=O)OCCCCC]

comprising performing the following reaction:

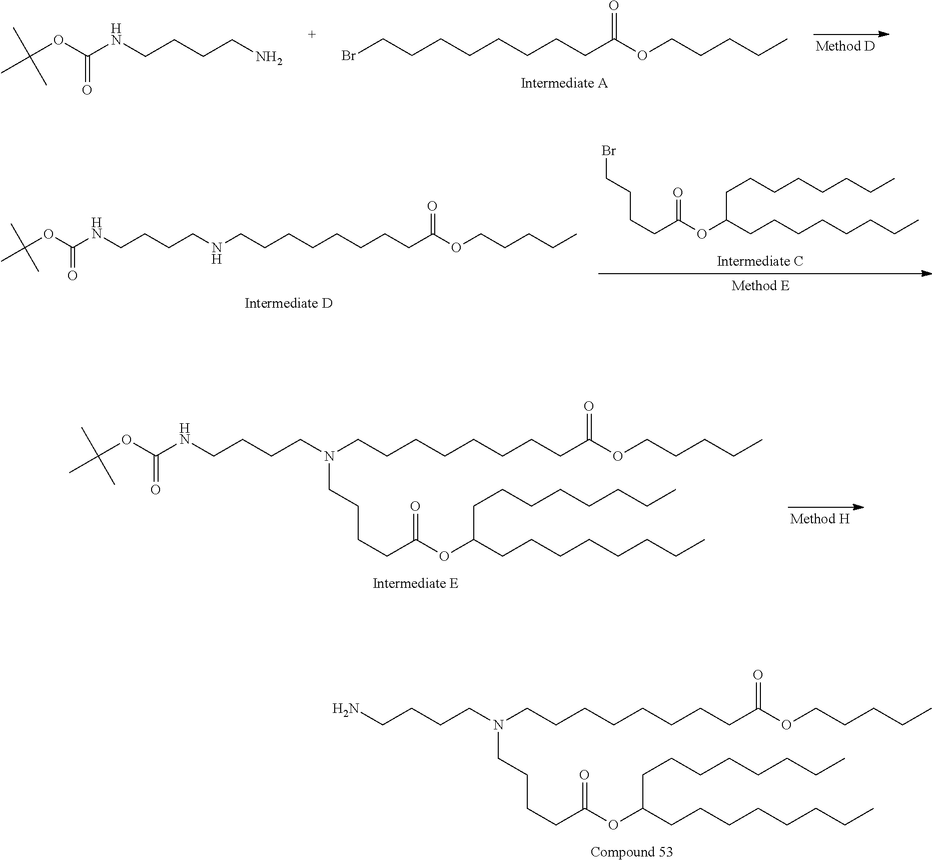

In certain aspects, compounds of Formula I may include, for example, the following compounds:

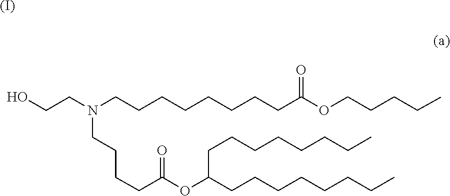 [SMILES: "(I)" ; CCCCCCCCC(CCCCCCCC)OC(=O)CCCCN(CCO)CCCCCCCCC(=O)OCCCCC]

(also referred to as Compound 1)

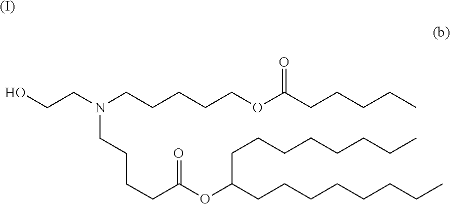 [SMILES: "(I)" ; CCCCCCCCC(CCCCCCCC)OC(=O)CCCCN(CCO)CCCCCOC(=O)CCCCC]

(also referred to as Compound 68)

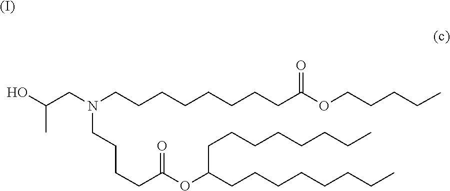 [SMILES: "(I)" ; CCCCCCCCC(CCCCCCCC)OC(=O)CCCCN(CCCCCCCCC(=O)OCCCCC)CC(C)O]

(also referred to as Compound 50)

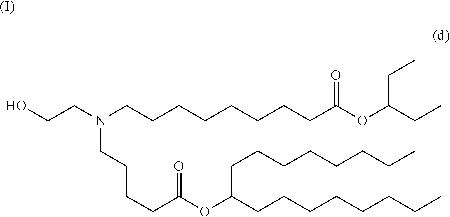 [SMILES: "(I)" ; CCCCCCCCC(CCCCCCCC)OC(=O)CCCCN(CCO)CCCCCCCCC(=O)OC(CC)CC]

(also referred to as Compound 48)

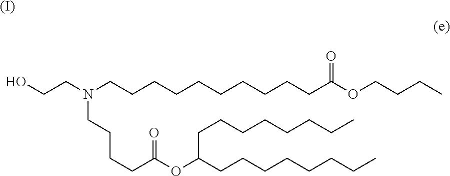 [SMILES: "(I)" ; CCCCCCCCC(CCCCCCCC)OC(=O)CCCCN(CCO)CCCCCCCCCCC(=O)OCCCC]

(also referred to as Compound 35)

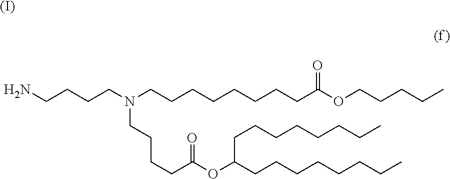 [SMILES: "(I)" ; CCCCCCCCC(CCCCCCCC)OC(=O)CCCCN(CCCCN)CCCCCCCCC(=O)OCCCCC]

(also referred to as Compound 53)

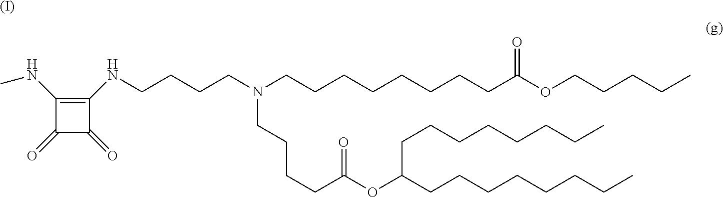 [SMILES: "(I)" ; CCCCCCCCC(CCCCCCCC)OC(=O)CCCCN(CCCCCCCCC(=O)OCCCCC)CCCCNc1c(NC)c(=O)c1=O]

(also referred to as Compound 54)

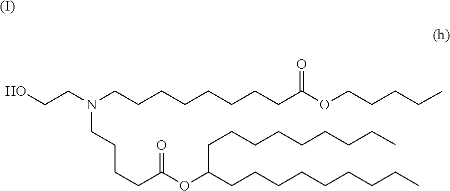 [SMILES: "(I)" ; CCCCCCCCCC(CCCCCCCCC)OC(=O)CCCCN(CCO)CCCCCCCCC(=O)OCCCCC]

(also referred to as Compound 42)

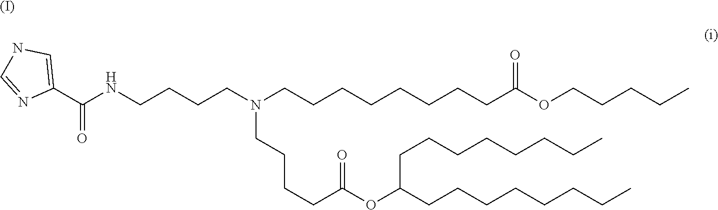

(also referred to as Compound 65).

In certain aspects, compounds of Formula I may include, for example, Compound 1 to 103 or a salt or isomer thereof.

### Lipid Nanoparticles

In some aspects, the dimension of a nanoparticle composition is 1 μm or shorter (e.g., 1 μm, 900 nm, 800 nm, 700 nm, 600 nm, 500 nm, 400 nm, 300 nm, 200 nm, 175 nm, 150 nm, 125 nm, 100 nm, 75 nm, 50 nm, or shorter), e.g., when measured by dynamic light scattering (DLS), transmission electron microscopy, scanning electron microscopy, or another method. Nanoparticle compositions include, for example, lipid nanoparticles (LNPs), liposomes, lipid vesicles, and lipoplexes. In some aspects, nanoparticle compositions are vesicles including one or more lipid bilayers. In certain aspects, a nanoparticle composition includes two or more concentric bilayers separated by aqueous compartments. Lipid bilayers may be functionalized and/or crosslinked to one another. Lipid bilayers may include one or more ligands, proteins, or channels.

Nanoparticle compositions comprise a lipid component including at least one compound according to Formula (I). For example, the lipid component of a nanoparticle composition may include one or more of Compounds (I) (a)-(I) (i). Nanoparticle compositions may also include a variety of other components. For example, the lipid component of a nanoparticle composition may comprise (i) a compound of Formula (I), (ii) a phospholipid moiety, (iii) a structural lipid, (iv) PEG lipid, (v) a payload, or any combination thereof. The elements of the lipid component may be provided in specific fractions.

In some embodiments, a nanoparticle composition may target a particular type or class of cells (e.g., cells of a particular organ or system thereof). For example, a nanoparticle composition including a biologically active agent of interest may be specifically delivered to a mammalian liver, kidney, spleen, femur, or lung. Specific delivery to a particular class of cells, an organ, or a system or group thereof implies that a higher proportion of nanoparticle compositions including a biologically active agent are delivered to the destination (e.g., tissue) of interest relative to other destinations, e.g., upon administration of a nanoparticle composition to a mammal. In some embodiments, specific delivery may result in a greater than 2 fold, 5 fold, 10 fold, 15 fold, or 20 fold increase in the amount of the biologically active agent per 1 g of tissue of the targeted destination (e.g., tissue of interest, such as a liver) as compared to another destination (e.g., the spleen).

As another example of targeted or specific delivery, the biologically active agent is an mRNA that encodes a protein-binding partner (e.g., an antibody or functional fragment thereof, a scaffold protein, or a peptide) or a receptor on a cell surface may be included in a nanoparticle composition. An mRNA may additionally or instead be used to direct the synthesis and extracellular localization of lipids, carbohydrates, or other biological moieties. Alternatively, other payloads or elements (e.g., lipids or ligands) of a nanoparticle composition may be selected based on their affinity for particular receptors (e.g., low density lipoprotein receptors) such that a nanoparticle composition may more readily interact with a target cell population including the receptors. For example, ligands may include, but are not limited to, members of a specific binding pair, antibodies, monoclonal antibodies, Fv fragments, single chain Fv (scFv) fragments, Fab′ fragments, F(ab′)₂ fragments, single domain antibodies, camelized antibodies and fragments thereof, humanized antibodies and fragments thereof, and multivalent versions thereof; multivalent binding reagents including mono- or bi-specific antibodies such as disulfide stabilized Fv fragments, scFv tandems, diabodies, tribodies, or tetrabodies; and aptamers, receptors, and fusion proteins.

In some embodiments, a ligand may be a surface-bound antibody, which can permit tuning of cell targeting specificity. This is especially useful since highly specific antibodies can be raised against an epitope of interest for the desired targeting site. In one embodiment, multiple antibodies are expressed on the surface of a cell, and each antibody can have a different specificity for a desired target. Such approaches can increase the avidity and specificity of targeting interactions.

### Ionizable Lipids

A nanoparticle composition may include one or more ionizable lipids (e.g., lipids that may have a positive or partial positive charge at physiological pH) in addition to a lipid according to Formula (I).

The lipid component of a nanoparticle composition may include one or more phospholipid moieties, such as one or more (poly) unsaturated lipids. Phospholipids may assemble into one or more lipid bilayers. In general, phospholipids may include a phospholipid moiety and one or more fatty acid moieties.

Phospholipids useful in the compositions and methods may be selected from a phospholipid known in the art, such as phosphatidyl choline, phosphatidyl ethanolamine, phosphatidyl glycerol, phosphatidyl serine, phosphatidic acid, 2-lysophosphatidyl choline, and a sphingomyelin. A fatty acid moiety may be selected from the non-limiting group consisting of lauric acid, myristic acid, myristoleic acid, palmitic acid, palmitoleic acid, stearic acid, oleic acid, linoleic acid, alpha-linolenic acid, erucic acid, arachidic acid, arachidonic acid, phytanic acid, eicosapentaenoic acid, behenic acid, docosapentaenoic acid, and docosahexaenoic acid. For example, in certain embodiments, a phospholipid is selected from the group consisting of 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-dioleoyl-sn-glycero-3-phosphoethanol amine (DOPE), 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), and sphingomyelin. In certain embodiments, the phospholipid is DOPE. In other embodiments, the phospholipid is DSPC. Non-natural species including natural species with modifications and substitutions including branching, oxidation, cyclization, and alkynes are also contemplated.

The lipid component of a nanoparticle composition may include one or more structural lipids. Structural lipids may be selected from a structural lipid known in the art, such as cholesterol, fecosterol, sitosterol, ergosterol, campesterol, stigmasterol, brassicasterol, tomatidine, ursolic acid, and alpha-tocopherol. In some embodiments, the structural lipid includes cholesterol and a corticosteroid (such as prednisolone, dexamethasone, prednisone, and hydrocortisone), or a combination thereof.

The lipid component of a nanoparticle composition may include one or more PEG or PEG-modified lipids. Such species may be alternately referred to as PEGylated lipids. A PEG lipid is a lipid modified with polyethylene glycol. A PEG lipid may be selected from a PEG lipid known in the art, such as PEG-modified phosphatidylethanolamine, a PEG-modified phosphatidic acid, a PEG-modified ceramide, a PEG-modified dialkylamine, a PEG-modified diacylglycerol, and a PEG-modified dialkylglycerol.

### Adjuvants

In some aspects, the nanoparticle composition of the present disclosure may include one or more lipids described herein and further include one or more adjuvants, which may be selected from an adjuvant known in the art, such as, Glucopyranosyl Lipid Adjuvant (GLA), CpG oligodeoxynucleotides (e.g., Class A or B), poly(I: C), aluminum hydroxide, and Pam3CSK4.

### Polynucleotides and Nucleic Acids

In some aspects, the biologically active agent delivered by a nanoparticle composition of the present invention is a polynucleotide or nucleic acid (e.g., ribonucleic acid or deoxyribonucleic acid). Exemplary polynucleotides for use in accordance with the present disclosure include, but are not limited to, one or more of deoxyribonucleic acid (DNA), ribonucleic acid (RNA) including messenger mRNA (mRNA), hybrids thereof, RNAi-inducing agents, RNAi agents, siRNAs, shRNAs, miRNAs, antisense RNAs, ribozymes, catalytic DNA, RNAs that induce triple helix formation, aptamers, vectors, etc. In some embodiments, a therapeutic and/or prophylactic is an RNA. RNAs useful in the compositions and methods described herein can be selected from the group consisting of, but are not limited to, shortmers, antagomirs, antisense, ribozymes, small interfering RNA (siRNA), asymmetrical interfering RNA (aiRNA), microRNA (miRNA), Dicer-substrate RNA (dsRNA), small hairpin RNA (shRNA), transfer RNA (IRNA), messenger RNA (mRNA), and mixtures thereof.

In certain aspects, the biologically active agent is an mRNA. An mRNA may encode any polypeptide of interest, including any naturally or non-naturally occurring or otherwise modified polypeptide. A polypeptide encoded by an mRNA may be of any size and may have any secondary structure or activity, the polypeptide can be, for example, a functional polypeptide, protein or enzyme, and upon being expressed (i.e., translated) by one or more target cells a functional expression product (e.g., a polypeptide, protein or enzyme) is produced, and in some instances secreted by the target cell into the peripheral circulation (e.g., plasma) of a subject.

In other aspects, the biologically active agent is an siRNA or antisense RNA. An siRNA or antisense RNA is functional in its RNA form and is capable of modulating or otherwise decreasing or eliminating the expression of an endogenous nucleic acid or gene. In certain embodiments such encapsulated polynucleotides may be natural or recombinant in nature and may modulate the expression of a gene or nucleic acid of interest using either sense or antisense mechanisms of action. For example, an siRNA or antisense RNA could be selected to silence a gene associated with a particular disease, disorder, or condition upon administration to a subject in need thereof.

In some aspects, a biologically active agent is an shRNA or a vector or plasmid encoding the same. An shRNA may be produced in its functional form inside a target cell upon delivery of an appropriate construct to the nucleus and is capable of modulating the expression of an endogenous nucleic acid or gene. Constructs and mechanisms relating to shRNA are well known in the art.

Nucleic acids and polynucleotides useful in the disclosure comprise a first region of linked nucleosides encoding a polypeptide of interest (e.g., a coding region), a first flanking region located at the 5′-terminus of the first region (e.g., a 5′-UTR), a second flanking region located at the 3′-terminus of the first region (e.g., a 3′-UTR), at least one 5′-cap region, a poly-A region, one or more intronic nucleotide sequences capable of being excised from the polynucleotide, or any combination thereof. In some embodiments, a polynucleotide or nucleic acid may include a 5′ cap structure, a chain terminating nucleotide, a stem loop, and/or a polyadenylation signal. Any one of the regions of a nucleic acid may include one or more modified nucleosides.

The amount of the biologically active agent in a nanoparticle composition may depend on the size, composition, desired target and/or application, or other properties of the nanoparticle composition as well as on the properties of the biologically active agent. For example, the amount of a nucleic acid useful in a nanoparticle composition may depend on the size, sequence, and other characteristics of the nucleic acid. The relative amounts of other elements (e.g., lipids) in a nanoparticle composition may also vary. In some embodiments, the wt/wt ratio of the lipid component to a biologically active agent in a nanoparticle composition may be from about 5:1 to about 60:1, such as 5:1, 6:1, 7:1, 8:1, 9:1, 10:1, 11:1, 12:1, 13:1, 14:1, 15:1, 16:1, 17:1, 18:1, 19:1, 20:1, 25:1, 30:1, 35:1, 40:1, 45:1, 50:1, and 60:1. The amount of a biologically active agent in a nanoparticle composition may, for example, be measured using absorption spectroscopy (e.g., ultraviolet-visible spectroscopy).

### Pharmaceutical Compositions

Nanoparticle compositions may be formulated as pharmaceutical compositions. Pharmaceutical compositions may include one or more nanoparticle compositions. For example, a pharmaceutical composition may include one or more nanoparticle compositions comprising one or more biologically active agents and a solvent, a diluent, an adjuvant, at least one excipient, a carrier, a dispersing agent or a combination thereof. A pharmaceutically acceptable carrier may be selected from one or more carriers known in the art, including Tris, an acetate (e.g., sodium acetate), an citrate (e.g., sodium citrate), saline, PBS, or sucrose.

A “pharmaceutically acceptable” salt, solvent, diluent, carrier or excipient, as used herein, means approved by a regulatory agency of the federal or a state government, or as listed in the U.S. Pharmacopoeia or other generally recognized pharmacopoeia for use in mammals, and more particularly in humans. In some aspects, one or more excipients may make up greater than 50% of the total mass or volume of a pharmaceutical composition including a nanoparticle composition. For example, the one or more excipients or accessory ingredients may make up 50%, 60%, 70%, 80%, 90%, or more of a pharmaceutical convention.

Relative amounts of the one or more nanoparticle compositions, the one or more pharmaceutically acceptable excipients, and/or any additional ingredients in a pharmaceutical composition in accordance with the present disclosure will vary, depending upon the identity, size, and/or condition of the subject treated and further depending upon the route by which the composition is to be administered.

In certain embodiments, the pharmaceutical compositions of the disclosure are refrigerated or frozen for storage and/or shipment (e.g., being stored at a temperature of 4° C. or lower, such as a temperature between about −150° C. and about 0° C. or between about −80° C. and about −20° C.). In certain embodiments, the disclosure also relates to a method of increasing stability of the nanoparticle compositions and/or pharmaceutical compositions comprising a compound of any of Formula (I) by storing the nanoparticle compositions and/or pharmaceutical compositions at a temperature of 4° C. or lower. For example, the nanoparticle compositions and/or pharmaceutical compositions disclosed herein are stable for about at least 1 week, at least 2 weeks, at least 3 weeks, at least 4 weeks, at least 5 weeks, at least 6 weeks, at least 1 month, at least 2 months, at least 4 months, at least 6 months, at least 8 months, at least 10 months, at least 12 months, at least 14 months, at least 16 months, at least 18 months, at least 20 months, at least 22 months, or at least 24 months, e.g., at a temperature of 4° C. or lower (e.g., between about 4° C. and −20° C.).

A pharmaceutical composition including one or more nanoparticle compositions may be prepared by any method known or hereafter developed in the art of pharmacology. In general, such preparatory methods include bringing the active ingredient into association with an excipient and/or one or more other accessory ingredients, and then, if desirable or necessary, dividing, shaping, and/or packaging the product into a desired single- or multi-dose unit.

A pharmaceutical composition in accordance with the present disclosure may be prepared, packaged, and/or sold in bulk, as a single unit dose, and/or as a plurality of single unit doses. As used herein, a “unit dose” is discrete amount of the pharmaceutical composition comprising a predetermined amount of the active ingredient (e.g., nanoparticle composition). The amount of the active ingredient is generally equal to the dosage of the active ingredient which would be administered to a subject and/or a convenient fraction of such a dosage such as, for example, one-half or one-third of such a dosage.

Injectable preparations, for example, sterile injectable aqueous or oleaginous suspensions may be formulated according to the known art using suitable dispersing agents, wetting agents, and/or suspending agents. Injectables can be prepared in conventional forms, either as liquid solutions or suspensions, solid forms suitable for solution or suspension in liquid prior to injection, or as emulsions. Suitable excipients are, for example, water, saline, dextrose, mannitol, lactose, lecithin, albumin, sodium glutamate, cysteine hydrochloride, and the like. In addition, if desired, the injectable pharmaceutical formulations may contain minor amounts of nontoxic auxiliary substances, such as wetting agents, pH buffering agents, and the like. Physiologically compatible buffers include, but are not limited to, Hanks's solution, Ringer's solution, or physiological saline buffer. Sterile, fixed oils are conventionally employed as a solvent or suspending medium. For this purpose any bland fixed oil can be employed including synthetic mono- or diglycerides. Fatty acids such as oleic acid can be used in the preparation of injectables.

Injectable formulations can be sterilized, for example, by filtration through a bacterial-retaining filter, and/or by incorporating sterilizing agents in the form of sterile solid compositions which can be dissolved or dispersed in sterile water or other sterile injectable medium prior to use.

Pharmaceutical formulations for parenteral administration, e.g., by bolus injection or continuous infusion, include aqueous solutions of the active formulation (e.g., the formulation that can include a compound, a retinoid, a second lipid, a stabilizing agent, and/or a therapeutic agent) in water-soluble form. Additionally, suspensions of the active compounds may be prepared as appropriate oily injection suspensions. Aqueous injection suspensions may contain substances which increase the viscosity of the suspension, such as sodium carboxymethyl cellulose, sorbitol, or dextran. Optionally, the suspension may also contain suitable stabilizers or agents that increase the solubility of the compounds to allow for the preparation of highly concentrated solutions. Formulations for injection may be presented in unit dosage form, e.g., in ampoules or in multi-dose containers, with an added preservative. The formulations may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

In addition to the preparations described previously, the formulations may also be formulated as a depot preparation. Such long acting formulations may be administered by intramuscular injection. Thus, for example, the formulations (e.g., the formulation that can include a compound, a retinoid, a second lipid, a stabilizing agent, and/or a therapeutic agent) may be formulated with suitable polymeric or hydrophobic materials (for example as an emulsion in an acceptable oil) or ion exchange resins, or as sparingly soluble derivatives, for example, as a sparingly soluble salt.

The compositions and formulations of the description may also be formulated for topical delivery and may be applied to the subject's skin using any suitable process for application of topical delivery vehicle. For example, the formulation may be applied manually, using an applicator, or by a process that involves both. Following application, the formulation may be worked into the subject's skin, e.g., by rubbing. Application may be performed multiple times daily or on a once-daily basis. For example, the formulation may be applied to a subject's skin once a day, twice a day, or multiple times a day, or may be applied once every two days, once every three days, or about once every week, once every two weeks, or once every several weeks.

### Methods of Delivering a Biologically Active Agent to a Cell

In one aspect, the present disclosure provides methods of producing a polypeptide of interest in a mammalian cell. Methods of producing polypeptides involve contacting a cell with a nanoparticle composition including an mRNA encoding the polypeptide of interest. Upon contacting the cell with the nanoparticle composition, the mRNA may be taken up and translated in the cell to produce the polypeptide of interest. The step of contacting a mammalian cell with a nanoparticle composition including an mRNA encoding a polypeptide of interest may be performed in vivo, ex vivo, in culture, or in vitro. The amount of nanoparticle composition contacted with a cell, and/or the amount of mRNA therein, may depend on the type of cell or tissue being contacted, the means of administration, the physiochemical characteristics of the nanoparticle composition and the mRNA (e.g., size, charge, and chemical composition) therein, and other factors. In general, an effective amount of the nanoparticle composition will allow for efficient polypeptide production in the cell. Metrics for efficiency may include polypeptide translation (indicated by polypeptide expression), level of mRNA degradation, and immune response indicators.

In one aspect, the present disclosure provides methods of delivering a biologically active agent such as siRNA into a cell. Suitable cells for use according to the methods described herein include prokaryotes, yeast, or higher eukaryotic cells, including plant and animal cells (e.g., mammalian cells). In some aspects, the cells can be cancer cells. In other aspects, the cells can be stem cells (e.g. pHSC cell line). In certain aspects, the formulations described herein can be used to transfect a cell.

The step of contacting a nanoparticle composition including a nucleic acid with a cell may involve or cause transfection. A phospholipid including in the lipid component of a nanoparticle composition may facilitate transfection and/or increase transfection efficiency, for example, by interacting and/or fusing with a cellular or intracellular membrane.

### Methods of Administration to a Subject

The present disclosure provides methods of delivering a biologically active agent to a cell or organ. Although the descriptions provided herein of nanoparticle compositions and pharmaceutical compositions including nanoparticle compositions are principally directed to compositions which are suitable for administration to humans, it will be understood by the skilled artisan that such compositions are generally suitable for administration to any other mammal. Modification of compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the compositions is contemplated include, but are not limited to, humans, other primates, and other mammals, including commercially relevant mammals such as cattle, pigs, hoses, sheep, cats, dogs, mice, and/or rats.

Pharmaceutical compositions including one or more nanoparticle compositions may be administered to any patient or subject, including those patients or subjects that may benefit from a therapeutic effect provided by the delivery of a therapeutic and/or prophylactic to one or more particular cells, tissues, organs, or systems or groups thereof. Although the descriptions provided herein of nanoparticle compositions and pharmaceutical compositions including nanoparticle compositions are principally directed to compositions which are suitable for administration to humans, it will be understood by a person of ordinary skill in the art that such compositions are generally suitable for administration to any other mammal. As will be readily apparent to one skilled in the art, the therapeutically effective in vivo dosage to be administered to a human or non-human subject and the particular mode of administration will vary depending upon the age, weight and mammalian species treated, the particular compounds employed, and the specific use for which these compounds are employed.

The dose of a pharmaceutical composition can be tailored to achieve a desired effect, but will depend on such factors as weight, diet, concurrent medication and other factors which those skilled in the medical arts will recognize. The determination of effective dosage levels, that is the dosage levels necessary to achieve the desired result, can be accomplished by one skilled in the art using routine pharmacological methods. For example, acceptable in vitro studies can be used to establish useful doses and routes of administration of the compositions identified by the present methods using established pharmacological methods. The attending physician would know how to and when to terminate, interrupt, or adjust administration due to toxicity or organ dysfunctions. Conversely, the attending physician would also know to adjust treatment to higher levels if the clinical response were not adequate (precluding toxicity). Dosage amount and interval may be adjusted individually to provide plasma levels of the active moiety which are sufficient to maintain the modulating effects, or minimal effective concentration (MEC). The MEC will vary for each compound but can be estimated from in vitro data.

In certain aspects, compositions in accordance with the present disclosure may be administered at dosage levels sufficient to deliver from about 0.0001 mg/kg to about 10 mg/kg, from about 0.001 mg/kg to about 10 mg/kg, from about 0.005 mg/kg to about 10 mg/kg, from about 0.01 mg/kg to about 10 mg/kg, from about 0.05 mg/kg to about 10 mg/kg, from about 0.1 mg/kg to about 10 mg/kg, from about 1 mg/kg to about 10 mg/kg, from about 2 mg/kg to about 10 mg/kg, from about 5 mg/kg to about 10 mg/kg, from about 0.0001 mg/kg to about 5 mg/kg, from about 0.001 mg/kg to about 5 mg/kg, from about 0.005 mg/kg to about 5 mg/kg, from about 0.01 mg/kg to about 5 mg/kg, from about 0.05 mg/kg to about 5 mg/kg, from about 0.1 mg/kg to about 5 mg/kg, from about 1 mg/kg to about 5 mg/kg, from about 2 mg/kg to about 5 mg/kg, from about 0.0001 mg/kg to about 2.5 mg/kg, from about 0.001 mg/kg to about 2.5 mg/kg, from about 0.005 mg/kg to about 2.5 mg/kg, from about 0.01 mg/kg to about 2.5 mg/kg, from about 0.05 mg/kg to about 2.5 mg/kg, from about 0.1 mg/kg to about 2.5 mg/kg, from about 1 mg/kg to about 2.5 mg/kg, from about 2 mg/kg to about 2.5 mg/kg, from about 0.0001 mg/kg to about 1 mg/kg, from about 0.001 mg/kg to about 1 mg/kg, from about 0.005 mg/kg to about 1 mg/kg, from about 0.01 mg/kg to about 1 mg/kg, from about 0.05 mg/kg to about 1 mg/kg, from about 0.1 mg/kg to about 1 mg/kg, from about 0.0001 mg/kg to about 0.25 mg/kg, from about 0.001 mg/kg to about 0.25 mg/kg, from about 0.005 mg/kg to about 0.25 mg/kg, from about 0.01 mg/kg to about 0.25 mg/kg, from about 0.05 mg/kg to about 0.25 mg/kg, or from about 0.1 mg/kg to about 0.25 mg/kg of a therapeutic and/or prophylactic agent (e.g., an mRNA) in a given dose, where a dose of 1 mg/kg (mpk) provides 1 mg of a therapeutic and/or prophylactic agent per 1 kg of subject body weight. In certain embodiments, a dose of about 0.001 mg/kg to about 10 mg/kg of a biologically active agent of a nanoparticle composition may be administered. In other aspects, a dose of about 0.005 mg/kg to about 2.5 mg/kg of a biologically active agent may be administered. In certain aspects, a dose of about 0.1 mg/kg to about 1 mg/kg may be administered. In other aspects, a dose of about 0.05 mg/kg to about 0.25 mg/kg may be administered.

The desired dosage may be delivered, for example, three times a day, two times a day, once a day, every other day, every third day, every week, every two weeks, every three weeks, or every four weeks. In certain embodiments, the desired dosage may be delivered using multiple administrations (e.g., two, three, four, five, six, seven, eight, nine, ten, eleven, twelve, thirteen, fourteen, or more administrations). In some embodiments, a single dose may be administered, for example, prior to or after a surgical procedure or in the instance of an acute disease, disorder, or condition.

Nanoparticle compositions including one or more biologically active agents may be used in combination with one or more other biologically active or imaging agents. By “in combination with,” it is not intended to imply that the agents must be administered at the same time and/or formulated for delivery together, although these methods of delivery are within the scope of the present disclosure. For example, one or more nanoparticle compositions including one or more different biologically active or imaging agents may be administered in combination. Compositions can be administered concurrently with, prior to, or subsequent to, one or more other desired therapeutics or medical procedures. In general, each agent will be administered at a dose and/or on a time schedule determined for that agent. In some embodiments, the present disclosure encompasses the delivery of compositions, or imaging, biologically active compositions thereof in combination with agents that improve their bioavailability, reduce and/or modify their metabolism, inhibit their excretion, and/or modify their distribution within the body.

It will further be appreciated that biologically active or imaging active agents utilized in combination may be administered together in a single composition or administered separately in different compositions. In general, it is expected that agents utilized in combination will be utilized at levels that do not exceed the levels at which they are utilized individually. In some embodiments, the levels utilized in combination may be lower than those utilized individually.

The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, a composition useful for treating cancer may be administered concurrently with a chemotherapeutic agent), or they may achieve different effects (e.g., control of any adverse effects, such as infusion related reactions).

### EXAMPLES

The ionizable lipids of the present disclosure were synthesized and formulated into LNPs using commercially available firefly luciferase reporter mRNA and investigational lipid, together with DSPC, cholesterol and a PEG-lipid in a fixed relative molar proportion. Lipids dissolved in ethanol of total concentration 12.87 mM were mixed by microfluidization with an aqueous phase (pH=4.5) containing mRNA in a ratio 1:3, respectively. The total flow rate was 12 mL/min, N/P ratio was 6. Buffer exchange was performed using Amicon filters. The formulation was sterilized by filtration.

Transfection efficacy and cell cytotoxicity of the LNPs were assayed in HEK293 cells and BALB mice. The LNPs of the present disclosure were found to be effectively transfected in to HEK293 cells in vitro and expressed in BALB mice in vivo. The data demonstrated better or comparable transfection efficacies than LNPs currently approved by FDA.

### Example 1: Transfection Efficiency

Transfection efficiency of LNPs containing novel ionizable lipids of Formula I were determined by detecting expression of firefly luciferase (Fluc) in HEK293 cells. In vitro tests of LNP formulations containing novel lipids of the disclosure were carried out using firefly luciferase luminescence assay.

HEK293 cells on passage 10-19 grown in EMEM full medium (comprising MEM, 10% FBS, 1% AA, 1% NEAA), in a cell culture incubator at 37° C. with 5% CO₂ were placed in a 96-well plate. An equal number of human embryonic kidney 293 (HEK-293) cells were transfected with LNP formulations loaded with commercially available firefly luciferase mRNA in the presence of human apolipoprotein E3. The quantity of LNP added to cells corresponded to 0.1 μg/mL of total mRNA in LNP samples. 10 ml of cell suspension containing 1 million of cells in EMEM full medium were placed into each well at an amount of seeding 10⁴ cells per well. A positive control (eg. NA of known transfection efficiency transfected with Lipofectamine3000), negative control (cells treated with RNA with no Lipofectamine or LNP) and 3 wells of untreated cells were also placed into the plate as controls. After 22 hours, the growth medium was replaced with 100 μL Opti-MEM 2% FBS in all wells 2 hours before transfection.

The transfection agent comprised nucleic acid (NA) of choice encoding Fluc protein, Lipofectamine 3000 with P3000 reagent or LNP formulation. 10 μL of the transfecting agents were added to each well. The cells were incubated in a cell culture incubator overnight.

24 hours after transfection, the cells in the 96-well plate are washed with PBS then lysed using 20 μL of 1× Cell Lysis Buffer. A Luciferase Assay Reagent containing dissolving Luciferase Assay Substrate in 10 mL of Luciferase Assay Buffer are added to the wells. luminescence of expressed firefly luciferase was measured according to firefly luciferase luminescence assay protocol.

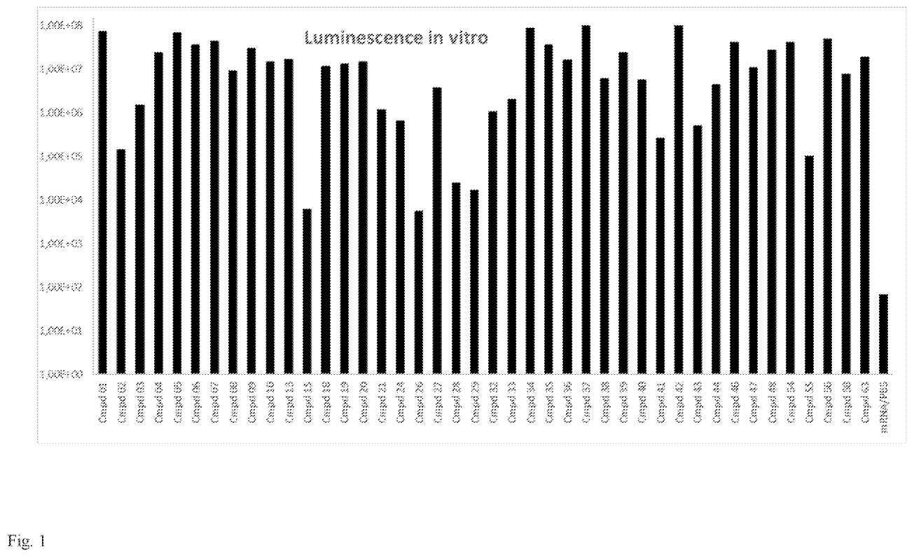

FIG. 1 shows Formula I lipids formulated in LNPs transfected into HEK293 cells and expressing Fluc-mRNA reporter compared to transfection of naked mRNA.

### Example 2: In Vivo BALB Mice, Subcutaneous Dosing Fluc-mRNA Reporter/IVIS

LNPs containing novel ionizable lipids of Formula I were tested in vivo in an experiment approved by the Local Ethics Committee for Animal Experiments in Warsaw and was conducted on 36 female mice from the BALB/cAnNRj strain (3 mice/group/route of administration).

To examine the duration and distribution of protein production from mRNA-LNPs in vivo, 1.0 g of luciferase mRNA-LNPs were administered into mice using subcutaneous or intravenous administration in a q1dx1 schedule (one dose of mRNA-LNP). Bioluminescence imaging was performed with the IVIS Spectrum CT imaging system. Mice were administered D-luciferin at a dose of 150 mg/kg intraperitoneally. 8 minutes (s.c. administration) or 5 minutes (i.v. administration) after receiving D-luciferin, mice were anesthetized in a chamber with 4% isoflurane (Aerrane, Baxter) and placed on the imaging platform while being maintained on 2% isoflurane via a nose cone. Mice were imaged at 13 and 15 minutes (s.c. administration) or 10 minutes (i.v. administration) post-administration of D-luciferin. Bioluminescence values were quantified by measuring photon flux (photons/second) in the region of interest where the bioluminescence signal emanated using a commercially available imaging software.

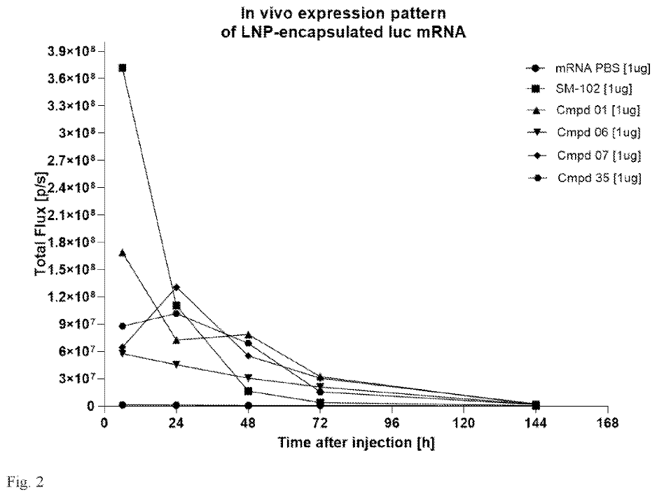

FIG. 2 shows in vivo expression of Fluc-mRNA reporter payload in BALB mice over 144 hours after subcutaneous dosing of LNP formulations comprising Formula I lipids compared to in vivo expression of the naked mRNA and a FDA approved LNP (LNP156/SM-102).

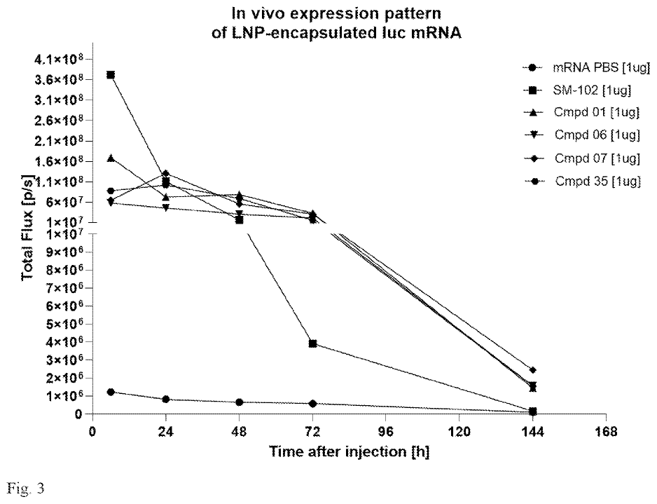

FIG. 3 shows a scaled graph of in vivo expression of Fluc-mRNA reporter payload in BALB mice over 144 hours after subcutaneous dosing of LNP formulations comprising Formula I lipids compared to in vivo expression of the naked mRNA and a FDA approved LNP (LNP156/SM-102).

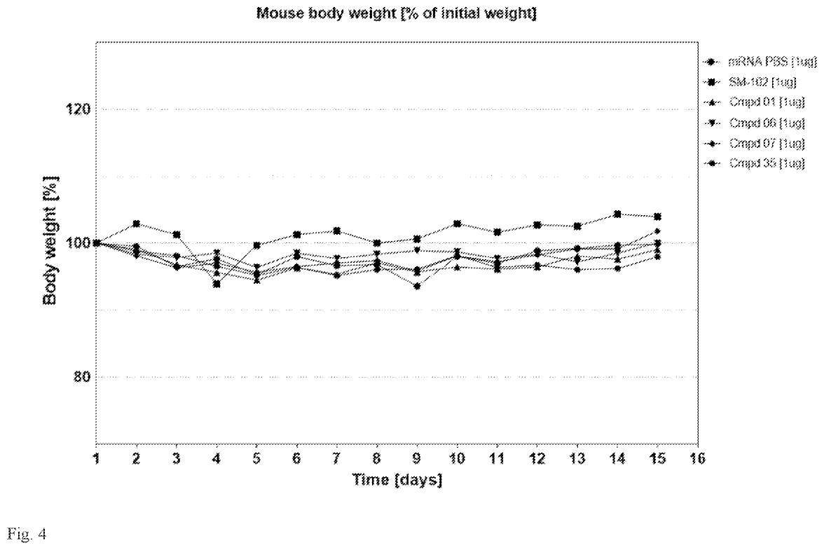

FIG. 4 shows body weight fluctuation of BALB mice after subcutaneous dosing of LNP formulations comprising Formula I lipids and a Fluc-mRNA reporter payload compared to in vivo expression of the naked mRNA and a FDA approved LNP (LNP156/SM-102).

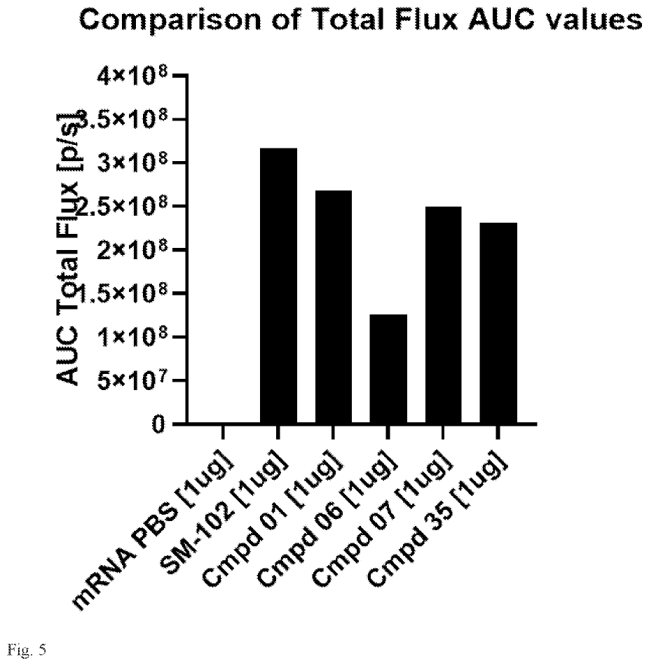

FIG. 5 shows total flux AUC values of in vivo expression of Fluc-mRNA reporter payload in BALB mice after subcutaneous dosing of LNP formulations comprising Formula I lipids compared to in vivo expression of the naked mRNA and a FDA approved LNP (LNP156/SM-102).

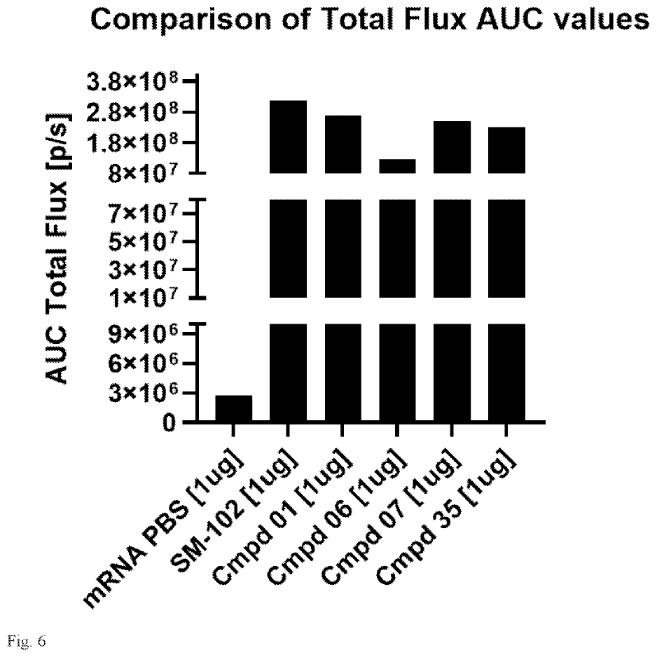

FIG. 6 shows a scaled graph of total flux AUC values of in vivo expression of Fluc-mRNA reporter payload in BALB mice after subcutaneous dosing of LNP formulations comprising Formula I lipids compared to in vivo expression of the naked mRNA and a FDA approved LNP (LNP156/SM-102).

### Example 3: In Vivo BALB Mice, Intravenous Dosing Fluc-mRNA Reporter/IVIS (2D)

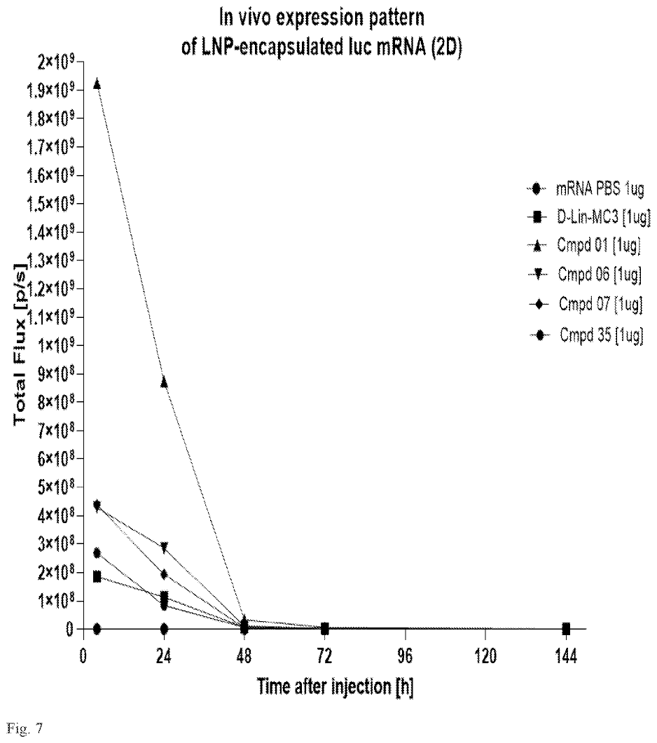

FIG. 7 shows 2D in vivo expression of Fluc-mRNA reporter payload in BALB mice over 144 hours after intravenous dosing of LNP formulations comprising Formula I lipids compared to in vivo expression of the naked mRNA and a FDA approved LNP (LNP167/D-LIN-MC3 Onpattro®).

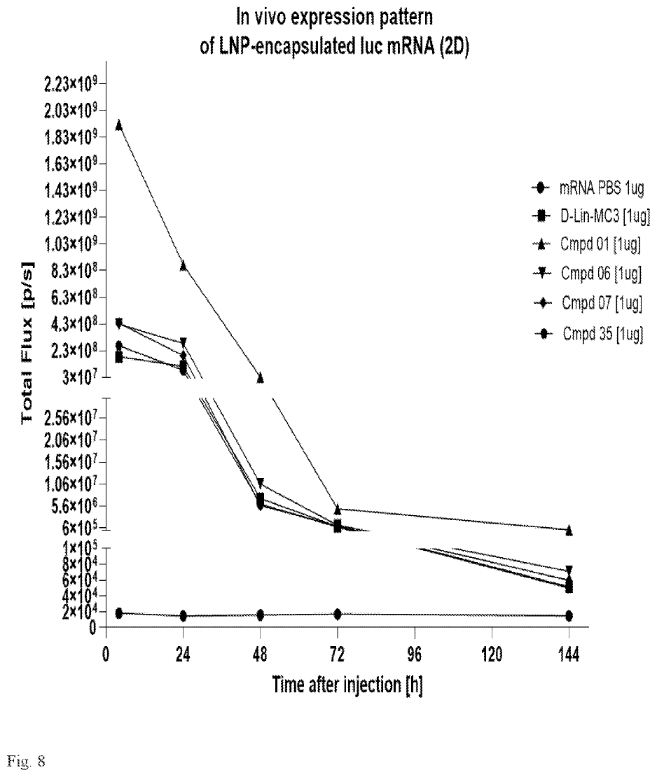

FIG. 8 shows a scaled graph of 2D in vivo expression of Fluc-mRNA reporter payload in BALB mice over 144 hours after intravenous dosing of LNP formulations comprising Formula I lipids compared to in vivo expression of the naked mRNA and a FDA approved LNP (LNP167/D-LIN-MC3 Onpattro®).

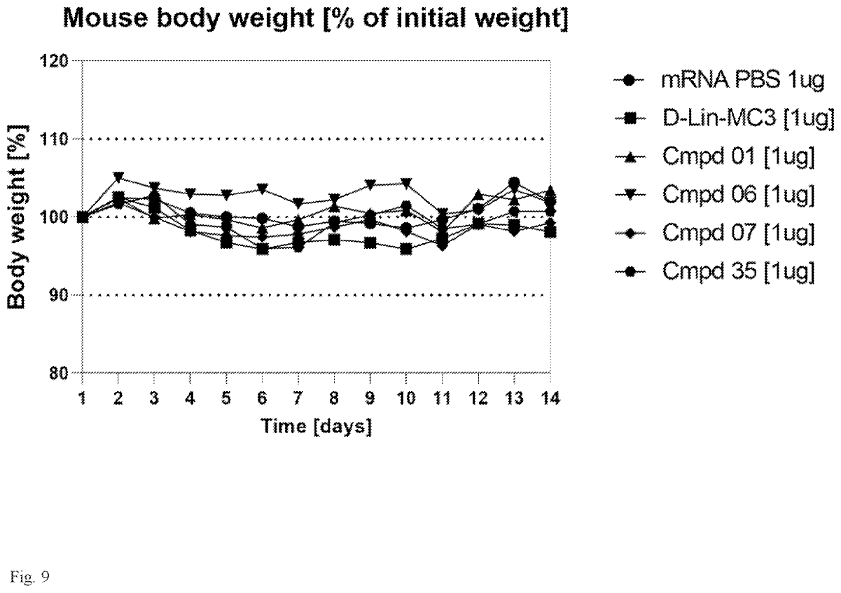

FIG. 9 shows 2D body weight fluctuation of BALB mice after intravenous dosing of LNP formulations comprising Formula I lipids and a Fluc-mRNA reporter payload compared to in vivo expression of the naked mRNA and a FDA approved LNP (LNP167/D-LIN-MC3 Onpattro®).

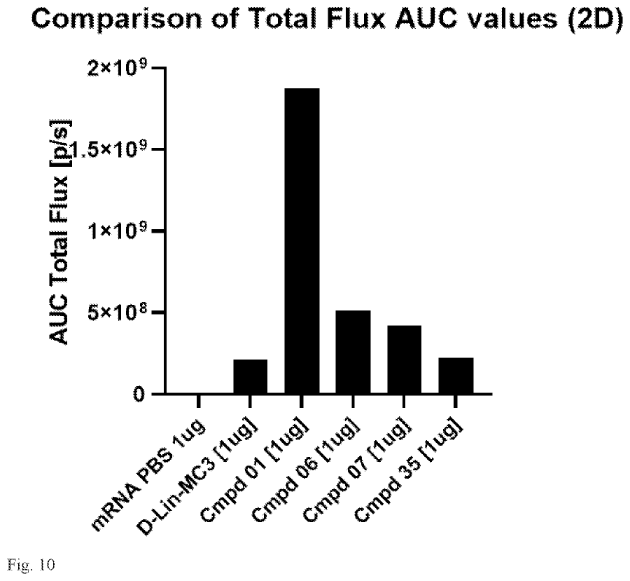

FIG. 10 shows total flux AUC values of in vivo expression of Fluc-mRNA reporter payload in BALB mice after intravenous dosing of LNP formulations comprising Formula I lipids compared to in vivo expression of the naked mRNA and a FDA approved LNP (LNP167/D-LIN-MC3 Onpattro®).

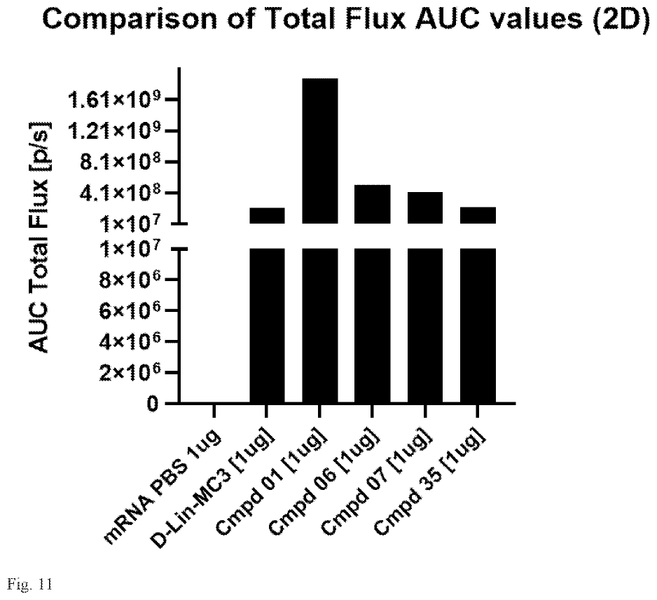

FIG. 11 shows a scaled graph of total flux AUC values of in vivo expression of Fluc-mRNA reporter payload in BALB mice after intravenous dosing of LNP formulations comprising Formula I lipids compared to in vivo expression of the naked mRNA and a FDA approved LNP (LNP167/D-LIN-MC3 Onpattro®).

### Example 4: In Vivo BALB Mice, Intravenous Dosing Fluc-mRNA Reporter/IVIS (3D)

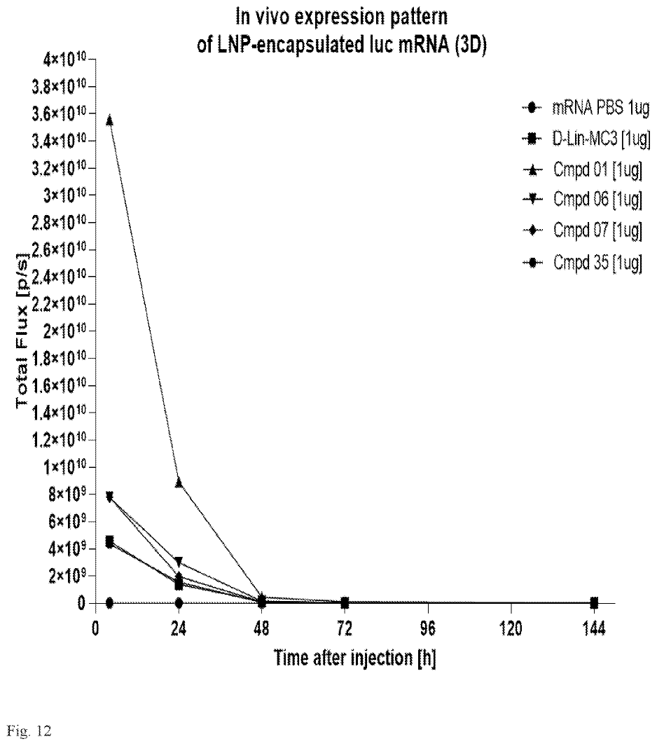

FIG. 12 shows 3D in vivo expression of Fluc-mRNA reporter payload in BALB mice over 144 hours after intravenous dosing of LNP formulations comprising Formula 1 lipids compared to in vivo expression of the naked mRNA and a FDA approved LNP (LNP167/D-LIN-MC3 Onpattro®).

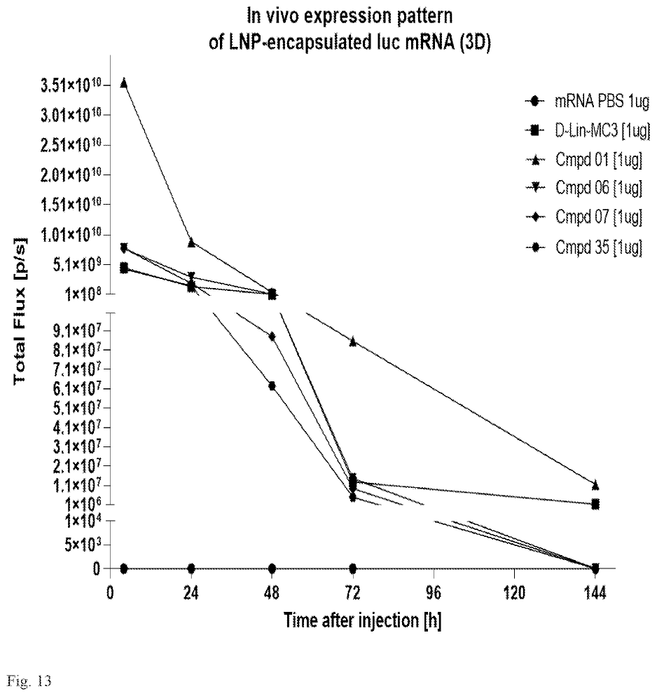

FIG. 13 shows a scaled graph of 3D in vivo expression of Fluc-mRNA reporter payload in BALB mice over 144 hours after intravenous dosing of LNP formulations comprising Formula I lipids compared to in vivo expression of the naked mRNA and a FDA approved LNP (LNP167/D-LIN-MC3 Onpattro®).

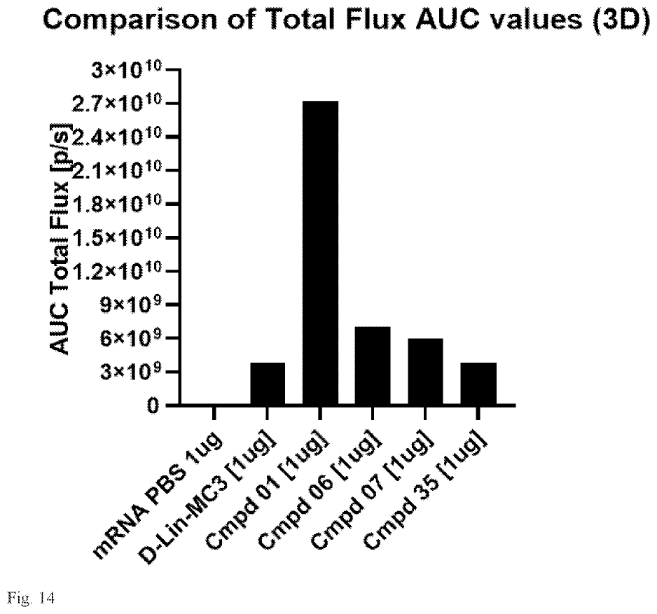

FIG. 14 shows 3D total flux AUC values of in vivo expression of Fluc-mRNA reporter payload in BALB mice after intravenous dosing of LNP formulations comprising Formula I lipids compared to in vivo expression of the naked mRNA and a FDA approved LNP (LNP167/D-LIN-MC3 Onpattro®).

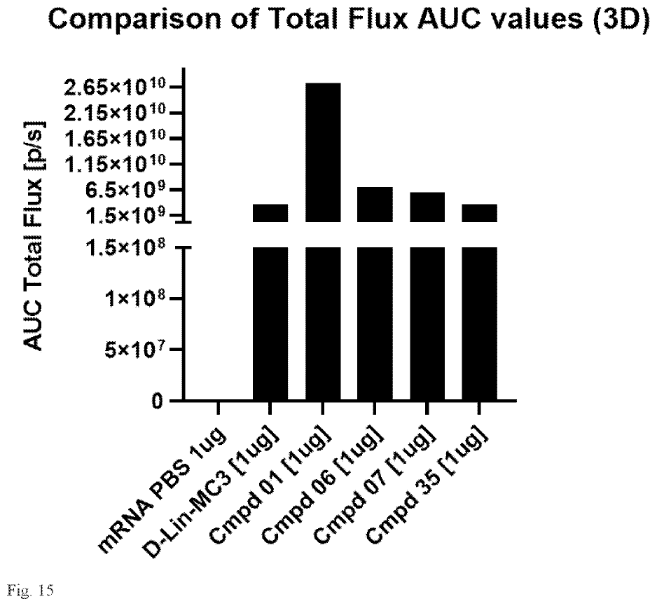

FIG. 15 shows a scaled graph of 3D total flux AUC values of in vivo expression of Fluc-mRNA reporter payload in BALB mice after intravenous dosing of LNP formulations comprising Formula I lipids compared to in vivo expression of the naked mRNA and a FDA approved LNP (LNP167/D-LIN-MC3 Onpattro®).

FIG. 9 shows 2D body weight fluctuation of BALB mice after intravenous dosing of LNP formulations comprising Formula I lipids and a Fluc-mRNA reporter payload compared to in vivo expression of the naked mRNA and a FDA approved LNP (LNP167/D-LIN-MC3 Onpattro®).

### Example 5: Representative Synthesis Procedure

Several compounds of Formula I can be synthesized using the Representative Procedure 1 described below:

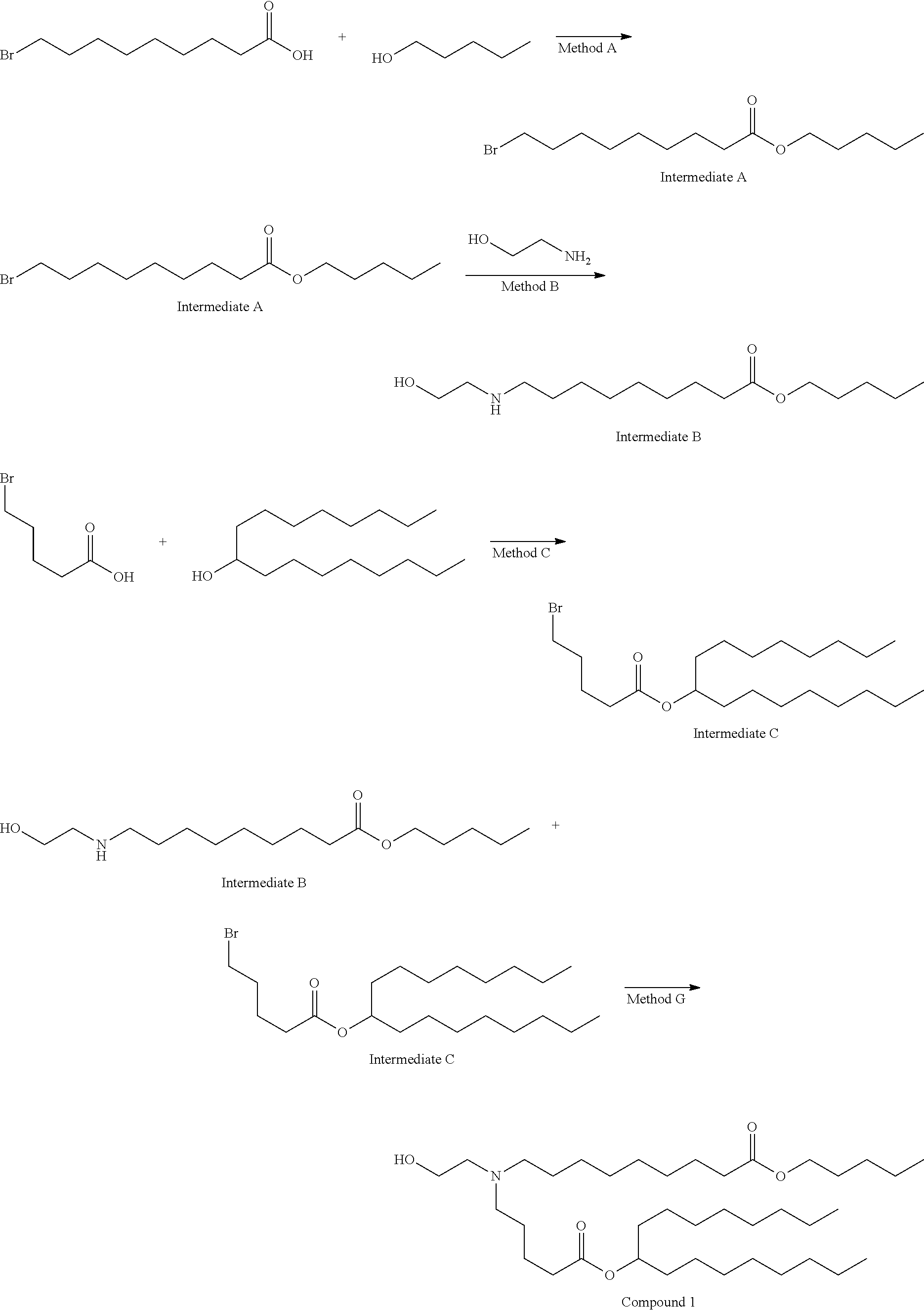

Several compounds of Formula I can be synthesized using the Representative Procedure 2 described below:

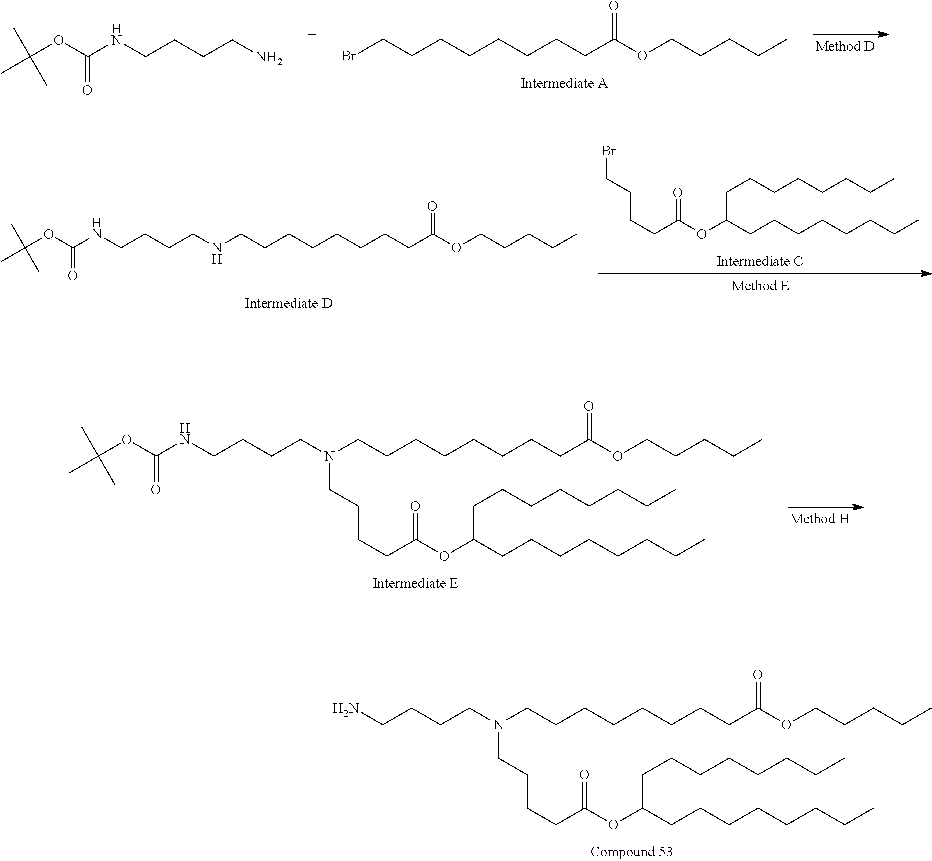

Several compounds of Formula I can be synthesized using the Representative Procedure 3 described below:

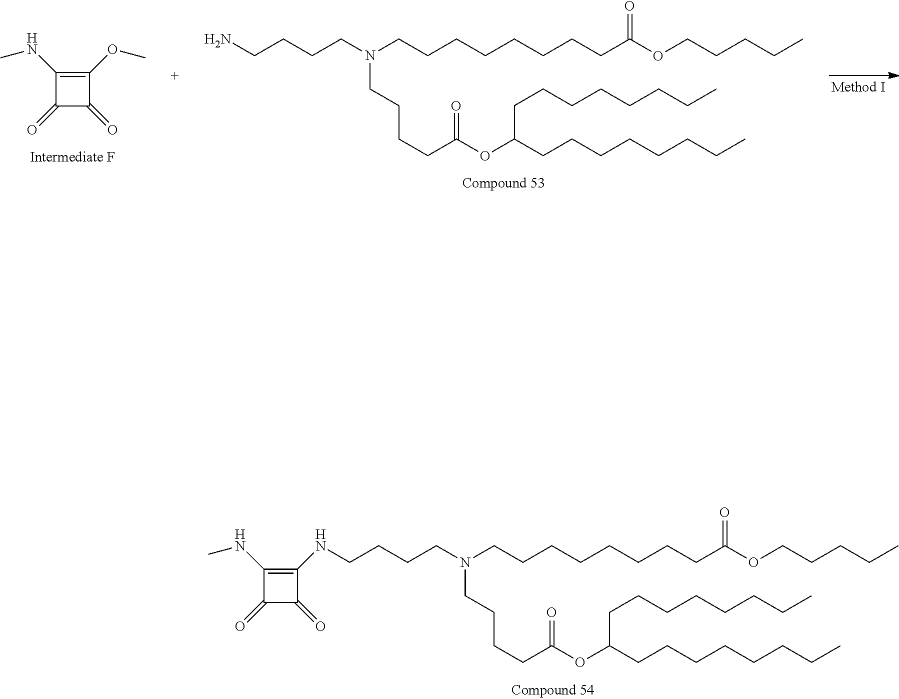

Several compounds of Formula I can be synthesized using the Representative Procedure 4 described below:

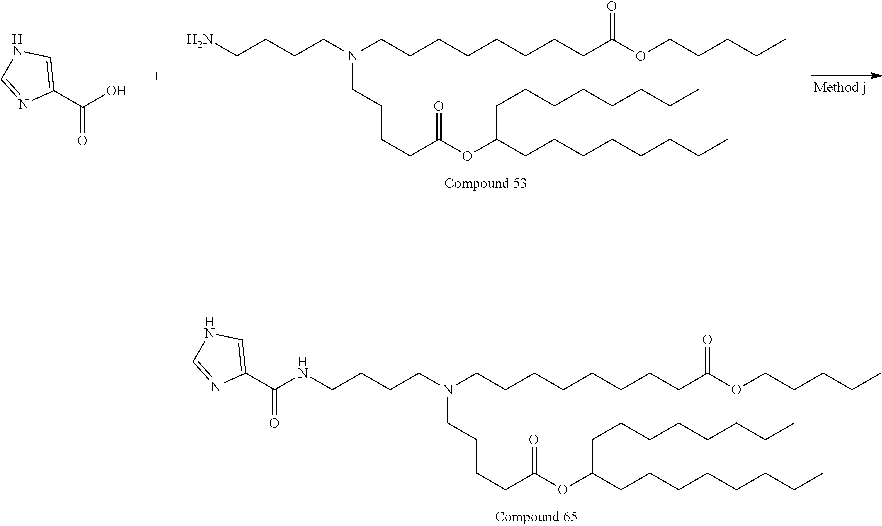 [SMILES: O=C(O)c1c[nH]cn1 ; CCCCCCCCC(CCCCCCCC)OC(=O)CCCCN(CCCCN)CCCCCCCCC(=O)OCCCCC ; CCCCCCCCC(CCCCCCCC)OC(=O)CCCCN(CCCCCCCCC(=O)OCCCCC)CCCCNC(=O)c1c[nH]cn1]

### Pentyl 9-bromononanoate (Intermediate A) (Method A)

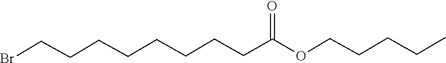 [SMILES: CCCCCOC(=O)CCCCCCCCBr]

To the crystals of 9-bromononanoic acid (15 g, 63.3 mmol, 1 eq) thionyl chloride (4.9 mL, 66.4 mmol, 1.05 eq) was added for 10 minutes upon stirring at room temperature under argon atmosphere. The mixture was stirred at room temperature for 1 hour and then at 60° C. for 5 hours. Then to the reaction mixture pantan-1-ol (8.26 mL, 75.9 mmol, 1.2 eq) was added for 30 minutes at 60° C. Next, the reaction mixture was stirred subsequently at 60° C. for 20 hours, at 80° C. for 7 hours and at 120° C. for 1 hour. Then, the reaction mixture was diluted with hexane (50 mL) and washed subsequently with saturated solution of sodium bicarbonate (100 mL), water (100 mL) and brine (100 mL). The organic layer was dried with sodium sulfate and the solvent was evaporated. The residual crude product (pentyl 9-bromononanoate, Intermediate A) (18.9 g, 61.5 mmol, 97%) was obtained as a dark yellow oil, and was used for the next reaction without further purification. ¹H NMR (600 MHz, CDCl₃) δ 4.06 (t, J=6.8 Hz, 2H), 3.40 (t, J=6.8 Hz, 2H), 2.29 (t, J=7.5 Hz, 2H), 1.85 (qu, J=7.0 Hz, 2H), 1.68-1.58 (m, 4H), 1.48-1.38 (m, 2H), 1.38-1.26 (m, 10H), 0.91 (t, J=7.0 Hz, 3H); ¹³C NMR (150 MHz, CDCl₃) δ 174.0, 64.5, 34.5, 34.1, 32.9, 29.2, 29.2, 28.7, 28.5, 28.2, 25.1, 22.5, 14.1.

### Pentyl 9-((2-hydroxyethyl)amino) nonanoate (Intermediate B) (Method B)

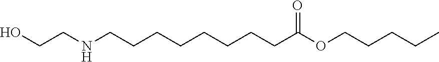 [SMILES: CCCCCOC(=O)CCCCCCCCNCCO]

The mixture of pentyl 9-bromononanoate (5.00 g, 16.3 mmol, 1.00 eq), ethanolamine (1.98 mL, 32.5 mmol, 2.00 eq), potassium carbonate (9.09 g, 65.1 mmol, 4 eq), and potassium iodide (2.99 g, 17.9 mmol, 1.10 eq) in a mixture of dioxane and acetonitrile (125 mL, 4:1, v/v) was stirred for 16 hours at 80° C. The reaction mixture was cooled and the solids were filtered off and washed with acetonitrile (50 mL). The filtrate was evaporated and the residue was purified by silica gel (Phase A: DCM 100%, Phase B: DCM/MeOH/NH₄OH, 80:20:1, v/v/v; Phase A: Phase B, from 100% to 0%) to obtain pentyl 9-((2-hydroxyethyl)amino) nonanoate (3.05 g, 10.6 mmol, 65%) as colourless oil. MS (ESI): m/z [M+H]⁺ 288.2 for C₁₆H₃₄NO₃; ¹H NMR (400 MHz, CDCl₃) δ 4.05 (t, J=6.7 Hz, 2H), 3.65-3.62 (m, 2H), 2.79-2.76 (m, 2H), 2.63-2.59 (m, 2H), 2.28 (t, J=7.5 Hz, 2H), 2.12 (bs, 2H), 1.65-1.58 (m, 4H), 1.50-1.45 (m, 2H), 1.34-1.30 (m, 12H), 0.90 (t, J=6.9 Hz, 3H); ¹³C NMR (100 MHz, CDCl₃) δ 174.1, 64.5, 60.9, 51.1, 49.6, 34.2, 30.2, 29.5, 29.3, 29.2, 28.5, 28.2, 27.3, 25.1, 22.5, 14.1

### Heptadecan-9-yl 5-bromopentanoate (Intermediate C) (Method C)

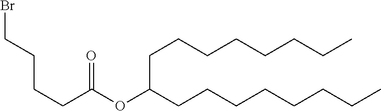 [SMILES: CCCCCCCCC(CCCCCCCC)OC(=O)CCCCBr]

To the crystals of 5-bromovaleric acid (10 g, 55.2 mmol, 1.00 eq) thionyl chloride (4.45 mL, 60.8 mmol, 1.1 eq) was added for 10 minutes upon stirring at room temperature under argon atmosphere. The mixture was heated to 80° C. and stirred for 1 hour. Then to the reaction mixture heptadecan-9-ol (14.2 g, 55.2 mmol, 1.0 eq) was added for 1 hour at 80° C. Next, the reaction mixture was stirred for 4 hours at 100° C. and for 16 hours at 50° C. Then, the reaction mixture was cooled to room temperature, diluted with EtOAc (100 mL) and washed subsequently with saturated solution of sodium bicarbonate (3×50 mL), water (50 mL), and brine (50 mL). The organic layer was dried with sodium sulfate and the solvent was evaporated. The residue (22.4 g) was purified by silica gel (hexane 100% to hexane:t-BuOMe, 90:10, v/v) to obtain heptadecan-9-yl 5-bromopentanoate (20.7 g, 48.9 mmol, 89%) as a pale yellow oil. ¹H NMR (600 MHz, CDCl₃) δ 4.89-4.85 (m, 1H), 3.41 (t, J=6.7 Hz, 2H), 2.33 (t, J=7.3 Hz, 2H), 1.93-1.88 (m, 2H), 1.81-1.77 (m, 2H), 1.51-1.49 (m, 4H), 1.30-1.26 (m, 24H), 0.88 (t, J=7.0 Hz, 6H); ¹³C NMR (150 MHz, CDCl₃) δ 173.1, 74.6, 34.3, 33.8, 33.2, 32.2, 32.0, 29.7, 29.6, 29.4, 25.5, 23.8, 22.8, 14.2.

### Pentyl 9-((4-((tert-butoxycarbonyl)amino)butyl)amino) nonanoate (Intermediate D) (Method D)

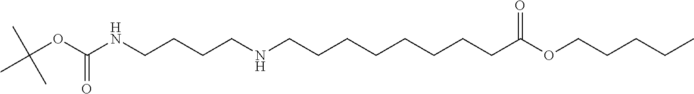 [SMILES: CCCCCOC(=O)CCCCCCCCNCCCCNC(=O)OC(C)(C)C]

To the mixture of pentyl 9-bromononanoate (Intermediate A) (0.50 g, 1.46 mmol, 1 eq) and tert-butyl (4-aminobutyl) carbamate (4.14 g, 22.0 mmol, 15 eq) a mixture of ethanol (5 mL) and acetonitrile (3 mL) was added. The reaction mixture was stirred for 20 hours at 80° C. Then the reaction mixture was cooled to room temperature, diluted with ethyl acetate (50 mL) and washed with water (2×100 mL) and brine (100 mL). The organic layer was dried with sodium sulfate and evaporated to dryness to give crude product as colorless oil (950 mg). Product was purified by silica gel chromatography (Phase A: t-BuOMe; Phase B t-BuOMe:MeOH:NH₄OH=50:50:1; 100% A to A:B=50:50) to obtain pentyl 9-((4-((tert-butoxycarbonyl)amino)butyl)amino) nonanoate as a beige wax (580 mg, 1.40 mmol, 96%); MS (ESI): m/z [M+H]⁺ 415.7 for C₂₃H₄₇N₂O₄; ¹H NMR (600 MHz, CDCl₃) δ 4.92 (bs, 1H), 4.06 (t, J=6.8 Hz, 2H), 3.12 (d, J=5.5 Hz, 2H, 2.61 (t, J=6.8 Hz, 2H), 2.57 (t, J=7.3 Hz, 2H), 2.29 (t, J=7.5 Hz, 2H) p, 1.64-1.58 (m, 4H), 1.54-1.48 (m, 4H), 1.48-1.40 (m, 12H), 1.36-1.30 (m, 12H), 1.30-1.27 (m, 8H), 0.91 (t, J=7.1 Hz, 3H); ¹³C NMR (150 MHz, CDCl₃) δ 173.9, 156.0, 78.9, 64.4, 50.1, 49.6, 40.5, 34.4, 30.1, 29.4, 29.2, 29.1, 28.4, 28.3, 28.1, 27.9, 27.6, 27.3, 25.0, 22.3, 13.9.

### Pentyl 9-((4-((tert-butoxycarbonyl)amino)butyl) (5-(heptadecan-9-yloxy)-5-oxopentyl)amino) nonanoate (Intermediate E) (Method E)

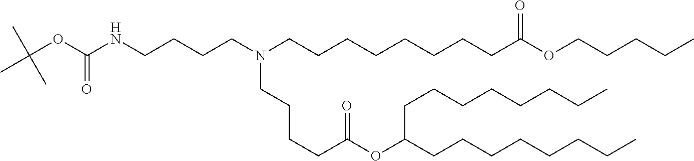 [SMILES: CCCCCCCCC(CCCCCCCC)OC(=O)CCCCN(CCCCCCCCC(=O)OCCCCC)CCCCNC(=O)OC(C)(C)C]

The mixture of heptadecan-9-yl 5-bromopentanoate (Intermediate C) (584 mg, 1.39 mmol, 1.05 eq) and pentyl 9-((4-((tert-butoxycarbonyl)amino)butyl)amino) nonanoate (Intermediate D) (550 mg, 1.33 mmol, 1.00 eq) was dissolved in a mixture of cyclopentyl methyl ether (2 mL) and acetonitrile (2 mL). Then, to the mixture potassium carbonate (741 mg, 5.31 mmol, 4.00 eq) and potassium iodide (221 mg, 1.33 mmol, 1.00 eq) were added. The reaction mixture were stirred for 20 hours at 85° C. Then, the reaction mixture was cooled to room temperature and solvents were evaporated to dryness. The residue was dissolved with ethyl acetate (50 mL) and water (100 mL) was added. The aqueous layer was extracted with ethyl acetate (2×20 mL). Combined organic layers were washed with brine (50 mL), dried with sodium sulfate, and evaporated to dryness to obtain crude product as pale yellow oil (1.28 g). Product was purified by silica gel chromatography (Phase A: t-BuOMe; Phase B t-BuOMe:MeOH:NH₄OH=50:50:1; 100% A to A:B=50:50) to obtain pentyl 9-((4-((tert-butoxycarbonyl)amino)butyl) (5-(heptadecan-9-yloxy)-5-oxopentyl)amino) nonanoate (0.985 g, 1.31 mmol, 99%) as colorless oil. MS (ESI): m/z [M+H]⁺ 754.0 for C₄₆H₈₉N₂O₆; ¹H NMR (600 MHz, CDCl₃) δ 5.03 (s, 1H), 4.89-4.83 (m, 1H), 4.06 (t, J=6.8 Hz, 2H), 3.11 (bd, J=5.7 Hz, 2H), 2.39 (bs, 6H), 2.29 (t, J=7.4, 2H), 2.28 (t, J=7.6, 2H), 1.65-1.58 (m, 6H), 1.51-1.44 (m, 20H), 1.35-1.26 (m, 36H), 0.90 (t, J=7.1 Hz, 3H), 0.89 (t, J=7.0 Hz, 6H); ¹³C NMR (150 MHz, CDCl₃) δ 173.9, 173.4, 156.0, 78.8, 74.2, 64.3, 54.0, 53.8, 53.6, 40.5, 34.6, 34.4, 34.1, 31.8, 29.5, 29.5, 29.4, 29.3, 29.2, 29.1, 28.4, 28.3, 28.1, 28.1, 27.5, 26.9, 26.4, 25.3, 25.0, 24.7, 23.2, 22.6, 22.3, 14.1, 13.9.

### 3-methoxy-4-(methylamino)cyclobut-3-ene-1,2-dione (Intermediate F)

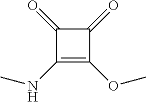

To the suspension of 3,4-dimethoxy-3-cyclobutene-1,2-dione (10.0 g, 70.4 mmol) in diethyl ether (300 mL) in a 2 M solution of methylamine in tetrahydrofuran (42 mL, 83.0 mmol, 1.2 eq) was added for 2 hours. The reaction mixture was stirred at room temperature for 20 hours. Then, the volatiles were evaporated under reduced pressure. The residue was washed with diethyl ether (3×20 mL), filtered off and dried with sodium sulfate to obtain the product as white solids (9.50 g, 67.3 mmol, 98%). Mp. 171-174° C.; ¹H NMR (400 MHz, DMSO-d₆) δ 8.21 (s, 1H), 4.29 (s, 3H), 3.39 (bs, 1H), 3.04 (d, J=4.7 Hz, 1H).

### 5-Chloropentyl hexanoate (Intermediate G) (Method F)

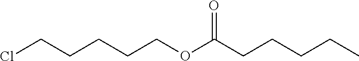

A solution of 5-chloro-1-pentanol (3.00 g, 23.2 mmol, 1.0 eq), DIPEA (9.98 mL, 58.1 mmol, 2.5 eq) and 4-dimethylaminopyridine (143 mg, 1.16 mmol, 0.05 eq) in dichloromethane (50 mL) was cooled to 0° C. and flushed with argon. To the solution hexanoyl chloride (3.32 mL, 23.2 mmol, 1 eq) was added for 10 minutes. Then, the reaction mixture was stirred at room temperature for 20 hours. The reaction mixture was subsequently washed with 1 M solution of hydrochloric acid (2×50 mL), water (50 mL), saturated solution of sodium bisulfate (50 mL) and brine (50 mL). The organic layer was dried with sodium sulfate and evaporated to dryness. Product was purified by silica gel chromatography (hexane 100% to hexane:ethyl acetate=70:30) to obtain 5-chloropentyl hexanoate (3.85 g, 17.3 mmol, 99%) as pale yellow oil. MS (APCI): m/z [M+H]⁺ 220.8 for C₁₁H₂₂ClO₂; ¹H NMR (600 MHz, CDCl₃) δ 4.08 (t, J=6.6 Hz, 2H), 3.54 (t, J=6.6 Hz, 2H), 2.30-2.28 (m, 2H), 1.83-1.78 (m, 2H), 1.67-1.61 (m, 4H), 1.53-1.50 (m, 2H), 1.35-1.28 (m, 4H), 0.89 (t, J=7.1 Hz, 3H); ¹³C NMR (150 MHz, CDCl₃) δ 174.1, 64.0, 44.9, 34.5, 32.3, 31.5, 28.1, 24.8, 23.5, 22.5, 14.0.

### 9-Bromononyl hexanoate (Intermediate H) (Method F)

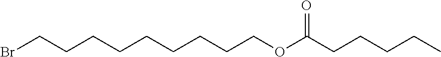 [SMILES: CCCCCC(=O)OCCCCCCCCCBr]

A solution of 9-bromo-1-nonanol (3.00 g, 13.0 mmol, 1.00 equiv), DIPEA (5.74 mL, 32.6 mmol, 2.50 equiv) and 4-dimethylaminopyridine (80 mg, 0.65 mmol, 0.05 equiv) in dichloromethane (50 mL) was cooled to 0° C. and flushed with argon. To the solution hexanoyl chloride (1.79 g, 13.0 mmol, 1.00 equiv), was added for 10 minutes. Then, the reaction mixture was stirred at room temperature for the weekend. The reaction mixture was subsequently washed with 1 M solution of hydrochloric acid (2×50 mL), water (50 mL), saturated aqueous solution of sodium bisulfate (50 mL) and brine (50 mL). The organic layer was dried over sodium sulfate and evaporated to dryness. Product was purified by silica gel chromatography (hexane 100% to hexane:ethyl acetate=70:30) to obtain 9-bromononyl hexanoate (2.52 g, 7.76 mmol, 59%) as colorless oil. ¹H NMR (400 MHz, CDCl₃) δ 4.04 (t, J=6.7 Hz, 2H), 3.39 (t, J=6.9 Hz, 2H), 2.29-2.25 (m, 2H), 1.87-1.80 (m, 2H), 1.65-1.58 (m, 4H), 1.44-1.37 (m, 2H), 1.34-1.25 (m, 12H), 0.88 (t, J=7.0 Hz, 3H); ¹³C NMR (100 MHz, CDCl₃) δ 174.1, 64.5, 34.5, 34.1, 32.9, 31.5, 29.4, 29.3, 28.8, 28.8, 28.3, 26.0, 24.9, 22.5, 14.1.

### 11-bromoundecyl hexanoate (Intermediate I) (Method F)

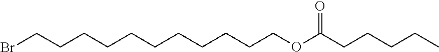 [SMILES: CCCCCC(=O)OCCCCCCCCCCCBr]

A solution of 11-bromo-1-undecanol (3.00 g, 11.6 mmol, 1.00 equiv), DIPEA (5.1 mL, 29.0 mmol, 2.50 equiv) and 4-dimethylaminopyridine (71 mg, 0.58 mmol, 0.05 equiv) in dichloromethane (50 mL) was cooled to 0° C. and flushed with argon. To the solution hexanoyl chloride (1.59 g, 11.6 mmol, 1.00 equiv), was added for 10 minutes. Then, the reaction mixture was stirred at room temperature for the weekend. The reaction mixture was subsequently washed with 1 M solution of hydrochloric acid (2×50 mL), water (50 mL), saturated solution of sodium bisulfate (50 mL) and brine (50 mL). The organic layer was dried over sodium sulfate and evaporated to dryness. Product was purified by silica gel chromatography (hexane 100% to hexane:ethyl acetate=70:30) to obtain 11-bromoundecyl hexanoate (2.52 g, 7.14 mmol, 61%) as pale yellow oil. 1H NMR (400 MHz, CDCl₃) δ 4.05 (t, J=6.7 Hz, 2H), 3.40 (t, J=6.9 Hz, 2H), 2.30-2.27 (m, 2H), 1.89-1.81 (m, 2H), 1.66-1.58 (m, 4H), 1.45-1.38 (m, 2H), 1.35-1.28 (m, 16H), 0.89 (t, J=7.0 Hz, 3H); ¹³C NMR (100 MHz, CDCl₃) δ 174.2, 64.5, 34.5, 34.2, 33.0, 31.5, 29.6, 29.6, 29.5, 29.4, 28.9, 28.8, 28.3, 26.1, 24.9, 22.5, 14.1.

### Pentyl 9-(3-hydroxypropylamino) nonanoate (Intermediate J)

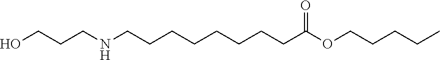 [SMILES: CCCCCOC(=O)CCCCCCCCNCCCO]

Intermediate J was synthesized according to Representative Procedure 1 and general Methods A and B starting from 3-hydroxy-1-aminopropane (7.00 equiv) instead of ethanolamine. Product (pentyl 9-(4-hydroxybutylamino) nonanoate) (220 mg, 0.73 mmol, 83%) was obtained as a colorless oil. MS (ESI): m/z [M+H]⁺ 302.4 for C₁₇H₃₅NO₃; ¹H NMR (600 MHz, CDCl₃) δ 4.04 (t, J=6.8 Hz, 2H), 3.79 (t, J=5.3 Hz, 2H), 3.00 (bs, 1H), 2.87-2.85 (zm, 2H), 2.58 (t, J=7.2 Hz, 2H), 2.27 (t, J=7.6 Hz, 2H), 1.67 (qu, J=5.5 Hz, 2H), 1.65-1.58 (m, 4H), 1.49-1.43 (m, 2H), 1.36-1.32 (m, 4H), 1.32-1.28 (m, 8H), 0.89 (t, J=7.1 Hz, 3H); ¹³C NMR (151 MHz, CDCl₃) δ 173.9, 64.5, 64.3, 50.1, 49.8, 34.3, 30.6, 29.8, 29.3, 29.1, 29.0, 28.3, 28.0, 27.1, 24.9, 22.3, 13.9.

### Pentyl 9-(4-hydroxybutylamino) nonanoate (Intermediate K)

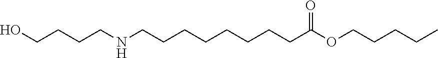 [SMILES: CCCCCOC(=O)CCCCCCCCNCCCCO]

Intermediate K was synthesized according to Representative Procedure 1 and general Methods A and B starting from 4-hydroxy-1-aminobutane (5.00 equiv) instead of ethanolamine. Product (pentyl 9-(4-hydroxybutylamino) nonanoate) (150 mg, 0.48 mmol, 81%) was obtained as a colorless oil. MS (ESI): m/z [M+H]⁺ 316.4 for C₁₈H₃₇NO₃; ¹H NMR (600 MHz, CDCl₃) δ 4.04 (t, J=6.8 Hz, 2H), 3.55 (t, J=5.2 Hz, 2H), 2.62 (t, J=5.5 Hz, 2H), 2.57 (t, J=7.3 Hz, 2H), 2.26 (t, J=7.5 Hz, 2H), 1.70-1.65 (m, 2H), 1.65-1.58 (m, 4H), 1.52-1.45 (m, 2H), 1.37-1.31 (m, 4H), 1.3-1.27 (m, 8H), 0.89 (t, J=7.1 Hz, 3H); ¹³C NMR (151 MHz, CDCl₃) δ 173.9, 64.3, 62.5, 49.6, 49.5, 34.3, 32.7, 29.7, 29.3, 29.1, 29.0, 28.9, 28.3, 28.0, 27.2, 24.9, 22.3, 13.9.

### Pentyl 9-((5-(heptadecan-9-yloxy)-5-oxopentyl) (2 hydroxyethyl)amino) nonanoate (Compound (1) (a)) (Compound 1) (Method G)

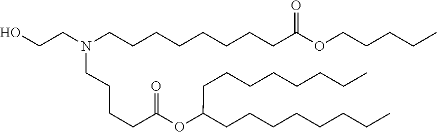 [SMILES: CCCCCCCCC(CCCCCCCC)OC(=O)CCCCN(CCO)CCCCCCCCC(=O)OCCCCC]

To the mixture of pentyl 9-((2-hydroxyethyl)amino) nonanoate (Intermediate B) (350 mg, 1.22 mmol, 1.00 eq) and heptadecan-9-yl 5-bromopentanoate (Intermediate C) (536 mg, 1.28 mmol, 1.05 eq) were added acetonitrile (10 mL), and cyclopentyl methyl ether (15 mL). Then potassium carbonate (680 mg, 4.87 mmol, 4.00 eq) and potassium iodide (223 mg, 1.34 mmol, 1.10 eq) were added. The reaction mixture was stirred for 4 hours at 80° C. and then for 2 days at 60° C. The reaction mixture was cooled and solids were filtered off, washed with acetonitrile (25 mL). The filtrate was evaporated and the residue was purified by silica gel (Phase A: DCM 100%, Phase B: DCM/MeOH/NH₄OH, 80:20:1, v/v/v; A:B, from 100% to 0%) to obtain pentyl 9-((5-(heptadecan-9-yloxy)-5-oxopentyl) (2-hydroxyethyl)amino) nonanoate (490 mg, 0.78 mmol, 64%) as colourless oil. MS (ESI): m/z [M+H]⁺ 626.6 for C₃₈H₇₆NO₅; ¹H NMR (400 MHz, CDCl₃) δ 4.86 (qu, J=6.3 Hz, 1H), 4.05 (t, J=6.8 Hz, 2H), 3.56 (t, J=5.1 Hz, 2H), 2.62 (t, J=4.9 Hz, 2H), 2.50 (dt, J=14.9, 7.5 Hz, 4H), 2.29 (q, J=7.5 Hz, 4H), 1.65-1.58 (m, 6H), 1.55-1.46 (m, 8H), 1.34-1.25 (m, 36H), 0.92-0.86 (m, 9H); ¹³C NMR (100 MHz, CDCl₃) δ 174.1, 173.4, 74.5, 64.5, 58.4, 55.9, 54.0, 53.7, 34.5, 34.3, 32.0, 29.7, 26.6, 29.5, 29.4, 29.3, 28.5, 28.2, 27.5, 26.9, 26.4, 25.5, 25.1, 23.1, 22.8, 22.5, 14.2, 14.1.

### Pentyl 6-{[5-(heptadecan-9-yloxy)-5-oxopentyl](2-hydroxyethyl)amino}hexanoate (Compound 2)

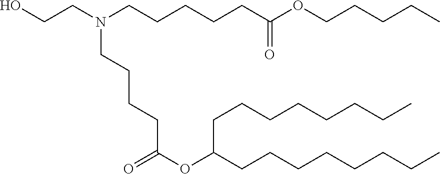 [SMILES: CCCCCCCCC(CCCCCCCC)OC(=O)CCCCN(CCO)CCCCCC(=O)OCCCCC]

Compound 2 was synthesized according to Representative Procedure 1 and general Methods A, B, C, and G. Product was obtained as a pale yellow oil (491 mg, 0.776 mmol, 65%). MS (ESI): m/z [M+H]⁺ 584.9 for C₃₅H₇NO₅; ¹H NMR (600 MHz, CDCl₃) δ 4.88-4.84 (m, 1H), 4.05 (t, J=6.8 Hz, 2H), 3.53 (t, J=5.3 Hz, 2H), 2.57 (t, J=5.3 Hz, 2H), 2.49-2.44 (m, 4H), 2.31-2.28 (m, 4H), 1.65-1.58 (m, 6H), 1.50-1.43 (m, 8H), 1.35-1.25 (m, 31H), 0.90 (t, J=7.1 Hz, 3H), 0.87 (t, J=7.0 Hz, 6H). ¹³C NMR (150 MHz, CDCl₃) δ 173.9, 173.4, 74.4, 64.6, 58.5, 55.7, 53.7, 53.6, 34.6, 34.4, 34.3, 32.0, 29.7, 29.6, 29.4, 28.5, 28.2, 27.1, 27.0, 26.7, 25.5, 25.0, 23.1, 22.8, 22.5, 14.2, 14.1.

### Pentyl 7-{[5-(heptadecan-9-yloxy)-5-oxopentyl](2-hydroxyethyl)amino}heptanoate (Compound 3)

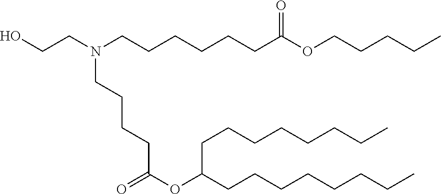 [SMILES: CCCCCCCCC(CCCCCCCC)OC(=O)CCCCN(CCO)CCCCCCC(=O)OCCCCC]

Compound 3 was synthesized according to Representative Procedure 1 and general Methods A, B, C, and G. Product was obtained as a pale yellow oil (246 mg, 0.411 mmol, 71%). MS (ESI): m/z [M+H]⁺ 598.5 for C₃₆H₇₂NO₅; ¹H NMR (400 MHz, CDCl₃-CD₃OD=2:1) δ 4.87 (p, J=6.2 Hz, 1H), 4.07 (t, J=6.7 Hz, 2H), 3.62 (t, J=5.8 Hz, 2H), 2.67 (t, J=5.8 Hz, 2H), 2.63-2.49 (m, 4H), 2.33 (dt, J=9.7, 7.4 Hz, 4H), 1.70-1.59 (m, 6H), 1.58-1.44 (m, 8H), 1.41-1.21 (m, 32H), 0.92 (t, J=7.0 Hz, 3H), 0.82 (t, J=6.9 Hz, 3H); ¹³C NMR (100 MHz, CDCl₃-CD₃OD=2:1) δ 174.9, 174.3, 75.1, 65.0, 58.9, 56.0, 54.4, 54.1, 34.7, 34.6, 34.4, 32.2, 29.8, 29.7, 29.6, 29.3, 28.6, 28.4, 27.4, 26.5, 26.1, 25.7, 25.2, 23.3, 23.0, 22.6, 14.2, 14.1.

### Pentyl 8-((5-(heptadecan-9-yloxy)-5-oxopentyl) (2-hydroxyethyl)amino) octanoate (Compound 4)

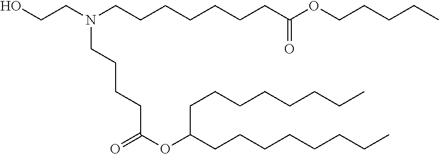 [SMILES: CCCCCCCCC(CCCCCCCC)OC(=O)CCCCN(CCO)CCCCCCCC(=O)OCCCCC]

Compound 4 was synthesized according to Representative Procedure 1 and general Methods A, B, C, and G. Product was obtained as a pale yellow oil (230 mg, 0.372 mmol, 68%). MS (ESI): m/z [M+H]⁺ 613.1 for C₃₇H₇₄NO₅; ¹H NMR (600 MHz, CDCl₃) δ 4.88-4.84 (m, 1H), 4.06 (t, J=6.8 Hz, 2H), 3.52 (t, J=5.4 Hz, 2H), 2.57 (t, J=5.4 Hz, 2H), 2.49-2.42 (m, 4H), 2.31-2.27 (m, 4H), 1.64-1.59 (m, 6H), 1.51-1.46 (m, 6H), 1.45-1.40 (m, 2H), 1.36-1.25 (m, 35H), 0.90 (t, J=7.1 Hz, 3H), 0.87 (t, J=7.1 Hz, 6H); ¹³C NMR (150 MHz, CDCl₃) δ 174.1, 173.5, 74.4, 64.6, 58.5, 55.7, 53.9, 53.6, 34.6, 34.5, 34.3, 32.0, 29.7, 29.7, 29.4, 29.4, 29.3, 28.5, 28.2, 27.4, 27.2, 26.8, 25.5, 25.1, 23.1, 22.8, 22.5, 14.2, 14.1.

### Pentyl 10-((5-(heptadecan-9-yloxy)-5-oxopentyl) (2-hydroxyethyl)amino) decanoate (Compound 5)

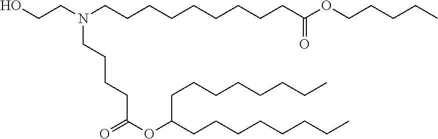 [SMILES: CCCCCCCCC(CCCCCCCC)OC(=O)CCCCN(CCO)CCCCCCCCCC(=O)OCCCCC]

Compound 5 was synthesized according to Representative Procedure 1 and general Methods A, B, C, and G. Product was obtained as a colorless oil (330 mg, 0.516 mmol, 78%). MS (ESI): m/z [M+H]⁺ 640.9 for C₃₉H₇₈NO₅; ¹H NMR (600 MHz, CDCl₃) δ 4.87 (qu, J=6.3 Hz, 1H), 4.06 (t, J=6.8 Hz, 2H), 3.52 (t, J=5.4 Hz, 2H), 2.90 (s, 1H), 2.57 (t, J=5.4 Hz, 2H), 2.47 (t, J=7.4 Hz, 2H), 2.43 (t, J=7.5 Hz, 2H), 2.31 (t, J=7.7 Hz, 2H), 2.30 (t, J=7.6 Hz, 2H), 1.65-1.59 (m, 6H), 1.51-1.46 (m, 6H), 1.45-1.40 (m, 2H), 1.38-1.34 (m, 6H), 1.30-1.26 (m, 32H), 0.91 (t, J=7.0 Hz, 3H), 0.89 (t, J=7.1 Hz, 6H); ¹³C NMR (150 MHz, CDCl₃) δ 173.9, 173.3, 74.2, 64.3, 58.4, 55.5, 53.8, 53.4, 34.4, 34.3, 34.1 (2×), 31.8 (2×), 29.5 (3×), 29.5 (2×), 29.4, 29.2 (3×), 29.1, 28.3, 28.1, 27.4, 27.2, 26.7, 25.3 (2×), 25.0, 23.0, 22.6 (2×), 22.3, 14.1 (2×), 13.9.

### Pentyl 11-((5-(heptadecan-9-yloxy)-5-oxopentyl) (2-hydroxyethyl)amino) undecanoate (Compound 6)

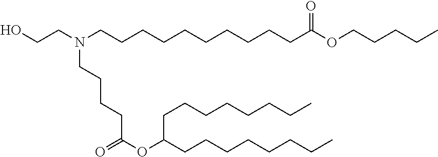 [SMILES: CCCCCCCCC(CCCCCCCC)OC(=O)CCCCN(CCO)CCCCCCCCCCC(=O)OCCCCC]

Compound 6 was synthesized according to Representative Procedure 1 and general Methods A, B, C, and G. Product was obtained as a pale yellow oil (337 mg, 0.52 mmol, 81%). MS (ESI): m/z [M+H]⁺ 655.1 for C₄₀H₈₀NO₅; ¹H NMR (600 MHz, CDCl₃) δ 4.86 (qu, J=6.2 Hz, 1H), 4.05 (t, J=6.8 Hz, 2H), 3.58 (t, J=6.1 Hz, 2H), 2.65 (t, J=5.1 Hz, 2H), 2.60-2.48 (m, 4H), 2.29 (dt, J=14.9, 7.4 Hz, 4H), 1.66-1.57 (m, 6H), 1.57-1.43 (m, 8H), 1.36-1.31 (m, 4H), 1.31-1.20 (m, 36H), 0.90 (t, J=7.0 Hz, 3H), 0.87 (t, J=7.0 Hz, 6H); ¹³C NMR (150 MHz, CDCl₃) δ 174.1, 173.2, 74.6, 64.5, 58.3, 55.9, 53.9, 53.2, 34.5, 34.2, 32.2, 31.9, 29.6, 29.6, 29.6, 29.5, 29.3, 29.2, 28.4, 28.2, 27.4, 25.4, 25.1, 22.7, 22.4, 14.2, 14.0.

### Pentyl 12-((5-(heptadecan-9-yloxy)-5-oxopentyl) (2-hydroxyethyl)amino) dodecanoate (Compound 7)

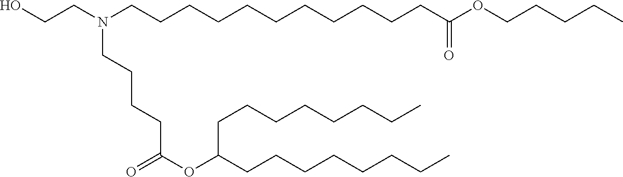 [SMILES: CCCCCCCCC(CCCCCCCC)OC(=O)CCCCN(CCO)CCCCCCCCCCCC(=O)OCCCCC]

Compound 7 was synthesized according to Representative Procedure 1 and general Methods A, B, C, and G. Product was obtained as a pale yellow oil (270 mg, 0.404 mmol, 67%). MS (ESI): m/z [M+H]⁺ 668.9 for C₄₁H₈₂NO₅; ¹H NMR (400 MHz, CDCl₃-CD₃OD=2:1) δ 4.87 (p, J=6.2 Hz, 1H), 4.07 (t, J=6.7 Hz, 2H), 3.61 (t, J=5.8 Hz, 2H), 2.65 (t, J=5.7 Hz, 2H), 2.60-2.48 (m, 4H), 2.33 (dt, J=13.2, 7.3 Hz, 4H), 1.70-1.58 (m, 6H), 1.57-1.42 (m, 8H), 1.39-1.21 (m, 42H), 0.92 (t, J=7.0 Hz, 3H), 0.88 (t, J=7.0 Hz, 6H); ¹³C NMR (100 MHz, CDCl₃-CD₃OD=2:1) δ 174.5, 173.7, 74.5, 64.4, 58.4, 55.4, 53.9, 53.5, 34.2, 34.1, 33.8, 31.6, 29.3, 29.3, 29.3, 29.2, 29.2, 29.0, 28.8, 28.0, 27.8, 27.2, 26.2, 25.6, 25.1, 24.7, 22.7, 22.4, 22.0, 13.6, 13.5

### Pentyl 13-((5-(heptadecan-9-yloxy)-5-oxopentyl) (2-hydroxyethyl)amino)tridecanoate (Compound 8)

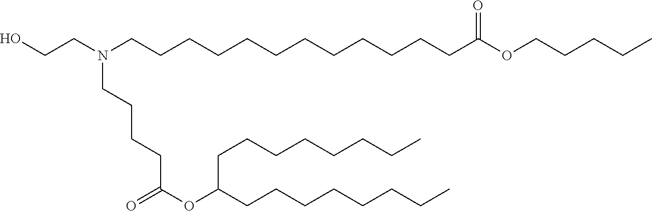 [SMILES: CCCCCCCCC(CCCCCCCC)OC(=O)CCCCN(CCO)CCCCCCCCCCCCC(=O)OCCCCC]

Compound 8 was synthesized according to Representative Procedure 1 and general Methods A, B, C, and G. Product was obtained as a pale yellow oil (160 mg, 0.235 mmol, 54%). MS (ESI): m/z [M+H]⁺ 682.8 for C₄₂H₈₄NO₅; ¹H NMR (400 MHz, CDCl₃-CD₃OD=2:1) δ 4.87 (qu, J=6.2 Hz, 1H), 4.07 (t, J=6.7 Hz, 2H), 3.61 (t, J=5.8 Hz, 2H), 2.64 (t, J=5.9 Hz, 2H), 2.58-2.46 (m, 4H), 2.33 (dt, J=12.5, 7.4 Hz, 4H), 1.69-1.58 (m, 6H), 1.58-1.39 (m, 10H), 1.39-1.17 (m, 42H), 0.91 (t, J=7.0 Hz, 3H), 0.87 (t, J=7.0 Hz, 6H).

### Pentyl 14-((5-(heptadecan-9-yloxy)-5-oxopentyl) (2-hydroxyethyl)amino)tetradecanoate (Compound 9)

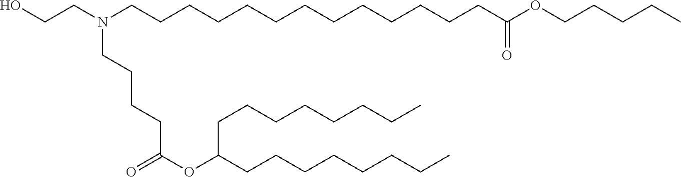 [SMILES: CCCCCCCCC(CCCCCCCC)OC(=O)CCCCN(CCO)CCCCCCCCCCCCCC(=O)OCCCCC]

Compound 9 was synthesized according to Representative Procedure 1 and general Methods A, B, C, and G. Product was obtained as a pale yellow oil (264 mg, 0.380 mmol, 68%). MS (ESI): m/z [M+H]⁺ 696.9 for C₄₃H₈₆NO₅; ¹H NMR (400 MHz, CDCl₃-CD₃OD=2:1) δ 4.87 (qu, J=6.2 Hz, 1H), 4.07 (t, J=6.7 Hz, 2H), 3.61 (t, J=5.9 Hz, 2H), 2.64 (t, J=5.9 Hz, 2H), 2.58-2.47 (m, 4H), 2.33 (dt, J=12.7, 7.3 Hz, 4H), 1.71-1.58 (m, 6H), 1.58-1.41 (m, 10H), 1.39-1.21 (m, 44H), 0.92 (t, J=7.0 Hz, 3H), 0.89 (t, J=7.0 Hz, 6H); ¹³C NMR (100 MHz, CDCl₃-CD₃OD=2:1) δ 175.2, 174.3, 75.1, 65.0, 59.1, 56.0, 54.5, 54.1, 34.8, 34.7, 34.4, 32.2, 30.0, 29.9, 29.9, 29.8, 29.8, 29.7, 29.6, 29.6, 29.6, 29.4, 28.6, 28.4, 27.8, 26.8, 26.3, 25.7, 25.3, 23.3, 23.0, 22.6, 14.2, 14.1

### Pentyl 15-((5-(heptadecan-9-yloxy)-5-oxopentyl) (2-hydroxyethyl)amino) pentadecanoate (Compound 10)

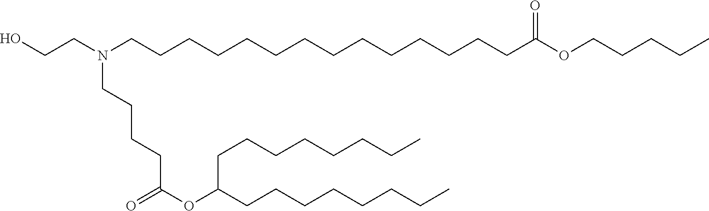 [SMILES: CCCCCCCCC(CCCCCCCC)OC(=O)CCCCN(CCO)CCCCCCCCCCCCCCC(=O)OCCCCC]

Compound 10 was synthesized according to Representative Procedure 1 and general Methods A, B, C, and G. Product was obtained as a pale yellow oil (315 mg, 0.444 mmol, 83%). MS (ESI): m/z [M+H]⁺ 710.7 for C₄₄H₈₈NO₅; ¹H NMR (400 MHz, CDCl₃-CD₃OD=2:1) δ 4.88 (p, J=6.3 Hz, 1H), 4.07 (t, J=6.7 Hz, 2H), 3.65 (t, J=5.8 Hz, 2H), 2.77-2.69 (m, 2H), 2.68-2.56 (m, 4H), 2.39-2.28 (m, 4H), 1.69-1.59 (m, 6H), 1.59-1.46 (m, 8H), 1.38-1.23 (m, 48H), 0.95-0.86 (m, 9H).

### Pentyl 9-((2-(heptadecan-9-yloxy)-2-oxoethyl) (2-hydroxyethyl)amino) nonanoate (Compound 11)

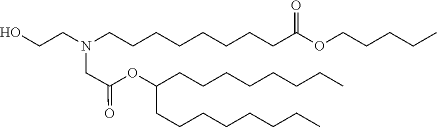 [SMILES: CCCCCCCCC(CCCCCCCC)OC(=O)CN(CCO)CCCCCCCCC(=O)OCCCCC]

Compound 11 was synthesized according to Representative Procedure 1 and general Methods A, B, C, and G. Product was obtained as a colorless oil (225 mg, 0,385 mmol, 73%). MS (ESI): m/z [M+H]⁺ 585.0 for C₃₅H₇₀NO₅; ¹H NMR (400 MHz, CDCl₃) δ 4.92 (qu, J=6.3 Hz, 1H), 4.06 (t, J=6.7 Hz, 2H), 3.57 (t, J=5.0 Hz, 2H), 3.38 (s, 2H), 2.81 (t, J=4.5 Hz, 2H), 2.66 (t, J=7.3 Hz, 2H), 2.28 (t, J=7.5 Hz, 2H), 1.66-1.57 (m, 4H), 1.53-1.47 (m, 6H), 1.35-1.26 (m, 36H), 0.92-0.86 (m, 9H); ¹³C NMR (100 MHz, CDCl₃) δ 173.9, 171.5, 75.2, 64.4, 58.8, 56.9, 55.0, 54.6, 34.4, 34.1 (2×), 31.8 (2×), 29.5 (2×), 29.3, 29.2, 29.2 (2×), 28.3, 28.1, 27.6, 27.1, 25.3 (2×), 25.0, 22.6 (2×), 22.3, 14.1 (2×), 13.9.

### Pentyl 9-((3-(heptadecan-9-yloxy)-3-oxopropyl) (2-hydroxyethyl)amino) nonanoate (Compound 12)

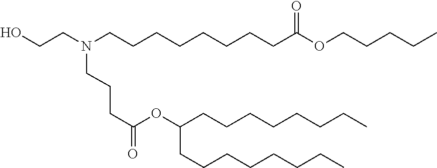 [SMILES: CCCCCCCCC(CCCCCCCC)OC(=O)CCCN(CCO)CCCCCCCCC(=O)OCCCCC]

Compound 12 was synthesized according to Representative Procedure 1 and general Methods A, B, C, and G. Product was obtained as a (mg, mmol, %). MS (ESI); m/z [M+H]⁺ 598.6 for C₃₆H₇₂NO₅; ¹H NMR (600 MHz, CDCl₃) δ 4.90-4.86 (m, 1H), 4.06 (t, J=6.8 Hz, 2H), 3.55 (t, J=5.2 Hz, 2H), 2.80 (t, J=6.9 Hz, 2H), 2.59-2.57 (m, 2H), 2.45-2.42 (m, 4H), 2.30-2.27 (m, 2H), 1.65-1.59 (m, 4H), 1.52-1.51 (m, 4H), 1.45-1.32 (m, 6H), 1.29-1.26 (m, 36H), 0.91 (t, J=7.1 Hz, 3H), 0.88 (t, J=7.1 Hz, 6H); ¹³C NMR (150 MHz, CDCl₃) δ 174.1, 172.7, 74.8, 64.5, 59.0, 55.8, 54.1, 49.5, 34.5, 34.2, 33.1, 32.0, 29.7, 29.6, 29.5, 29.4, 29.4, 29.3, 28.5, 28.2, 27.5, 27.3, 25.5, 25.1, 22.8, 22.5, 14.2, 14.1.

### Pentyl 9-((4-(heptadecan-9-yloxy)-4-oxobutyl) (2-hydroxyethyl)amino) nonanoate (Compound 13)

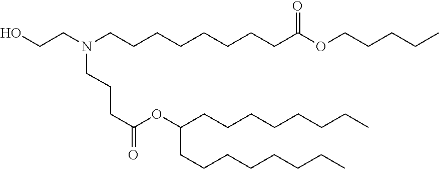 [SMILES: CCCCCCCCC(CCCCCCCC)OC(=O)CCCN(CCO)CCCCCCCCC(=O)OCCCCC]

Compound 13 was synthesized according to Representative Procedure 1 and general Methods A, B, C, and G. Product was obtained as a pale yellow oil (220 mg, 0.356 mmol, 47%). MS (ESI): m/z [M+H]⁺ 612.5 for C₃₇H₇₄NO₅; ¹H NMR (400 MHz, CDCl₃) δ 4.86 (qu, J=6.3 Hz, 1H), 4.05 (t, J=6.8 Hz, 2H), 3.57 (t, J=5.2 Hz, 2H), 2.63 (t, J=4.9 Hz, 2H), 2.56-2.48 (m, 4H), 2.30 (dt, J=10.0, 7.4 Hz, 4H), 1.84-1.76 (m, 2H), 1.64-1.58 (m, 4H), 1.51-1.46 (m, 6H), 1.34-1.25 (m, 36H), 0.92-0.86 (m, 9H). ¹³C NMR (100 MHz, CDCl₃) δ 174.1, 173.3, 74.7, 64.5, 58.5, 56.0, 54.0, 53.2, 34.5, 34.2, 32.3, 32.0, 29.7, 29.7, 29.5, 29.4, 29.3, 28.5, 28.2, 27.5, 26.9, 25.5, 25.1, 22.8, 22.5, 14.2, 14.1.

### Pentyl 10-((2-(heptadecan-9-yloxy)-2-oxoethyl) (2-hydroxyethyl)amino) decanoate (Compound 14)

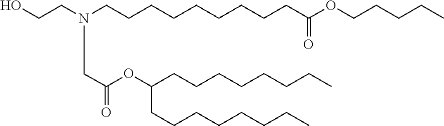 [SMILES: CCCCCCCCC(CCCCCCCC)OC(=O)CN(CCO)CCCCCCCCCC(=O)OCCCCC]

Compound 14 was synthesized according to Representative Procedure 1 and general Methods A, B, C, and G. Product was obtained as a colorless oil (290 mg, 0.485 mmol, 73%). MS (ESI): m/z [M+H]⁺ 598.9 for C₃₆H₇₂NO₅; ¹H NMR (400 MHz, CDCl₃) δ 4.92 (qu, J=6.3 Hz, 1H), 4.06 (t, J=6.8 Hz, 2H), 3.56 (bt, J=5.2 Hz, 2H), 3.36 (s, 2H), 3.22 (bs, 1H), 2.80 (t, J=5.2 Hz, 2H), 2.64 (t, J=7.5 Hz, 2H), 2.29 (t, J=7.5 Hz, 2H), 1.62-1.62 (m, 4H), 1.53-1.52 (m, 4H), 1.45-1.45 (m, 2H), 1.35-1.25 (m, 38H), 0.91 (t, J=6.9 Hz, 3H), 0.88 (t, J=7.0 Hz, 6H); ¹³C NMR (100 MHz, CDCl₃) δ 173.9, 171.7, 75.0, 64.3, 58.9, 56.9, 55.1, 54.6, 34.4, 34.0, 31.8 (2×), 29.5 (3×), 29.4, 29.2 (2×), 29.2 (2×), 29.1, 28.3, 28.1, 27.7, 27.1, 25.3 (2×), 25.0, 22.6 (2×), 22.3, 14.1 (2×), 13.9.

### Pentyl 10-((3-(heptadecan-9-yloxy)-3-oxopropyl) (2-hydroxyethyl)amino) decanoate (Compound 15)

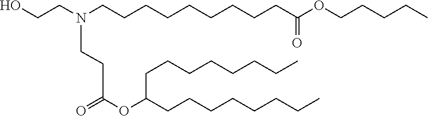 [SMILES: CCCCCCCCC(CCCCCCCC)OC(=O)CCN(CCO)CCCCCCCCCC(=O)OCCCCC]

Compound 15 was synthesized according to Representative Procedure 1 and general Methods A, B, C, and G. Product was obtained as a colorless oil (150 mg, 0,245 mmol, 36%). MS (ESI): m/z [M+H]⁺ 612.9 for C₃₇H₇₄NO₅; ¹H NMR (600 MHz, CDCl₃) δ 4.88 (qu, J=6.3 Hz, 1H), 4.06 (t, J=6.8 Hz, 2H), 3.58 (bs, 2H), 2.84 (bs, 2H), 2.62 (bs, 2H), 2.47 (bs, 4H), 2.29 (t, J=7.8 Hz, 2H), 1.57-1.68 (m, 4H), 1.47-1.57 (m, 4H), 1.39-1.47 (bs, 2H), 1.32-1.39 (m, 6H), 1.13-1.32 (m, 34H), 0.91 (t, J=7.1 Hz, 3H), 0.88 (t, J=7.1 Hz, 6H); ¹³C NMR (150 MHz, CDCl₃) δ 173.9, 172.4, 74.8, 64.4, 58.7, 55.8, 54.0, 49.4, 34.4, 34.0 (2×), 32.7, 31.8 (2×), 29.5 (2×), 29.5 (2×), 29.4, 29.2 (3×), 29.1, 28.3, 28.1, 27.3, 26.9, 25.3 (2×), 25.0, 22.6 (2×), 22.3, 14.1 (2×), 13.9.

### Pentyl 6-((4-(heptadecan-9-yloxy)-4-oxobutyl) (2-hydroxyethyl)amino) hexanoate (Compound 16)

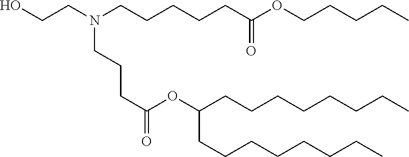 [SMILES: CCCCCCCCC(CCCCCCCC)OC(=O)CCCN(CCO)CCCCCC(=O)OCCCCC]

Compound 16 was synthesized according to Representative Procedure 1 and general Methods A, B, C, and G. Product was obtained as a pale yellow oil (110 mg, 0.191 mmol, 31%). MS (ESI): m/z [M+H]⁺ 570.9 for C₃₄H₆₈NO₅; ¹H NMR (600 MHz, CDCl₃) δ 4.88-4.84 (m, 1H), 4.06 (t, J=6.8 Hz, 2H), 3.61 (bs, 2H), 2.68 (bs, 2H), 2.60-2.56 (m, 3H), 2.32 (t, J=7.2 Hz, 2H), 2.30 (t, J=7.5 Hz, 2H), 1.83 (bs, 2H), 1.66-1.60 (m, 4H), 1.54-1.50 (m, 6H), 1.36-1.25 (m, 32H), 0.90 (t, J=7.1 Hz, 3H), 0.87 (t, J=7.0 Hz, 6H); ¹³C NMR (150 MHz, CDCl₃) δ 173.8, 173.2, 74.8, 64.6, 58.4, 56.2, 53.9, 53.3, 34.3, 34.2, 32.2, 32.0, 29.7, 29.4, 28.5, 28.2, 27.0, 25.5, 24.9, 22.8, 22.5, 14.2, 14.1.

### Pentyl 8-((4-(heptadecan-9-yloxy)-4-oxobutyl) (2-hydroxyethyl)amino) octanoate (Compound 17)

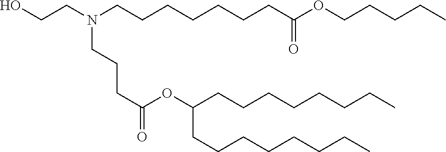 [SMILES: CCCCCCCCC(CCCCCCCC)OC(=O)CCCN(CCO)CCCCCCCC(=O)OCCCCC]

Compound 17 was synthesized according to Representative Procedure 1 and general Methods A, B, C, and G. Product was obtained as a pale yellow oil (200 mg, 0.331 mmol, 60%). MS (ESI): m/z [M+H]⁺ 598.9 for C₃₆H₇₂NO₅; ¹H NMR (600 MHz, CDCl₃) δ 4.87-4.83 (m, 1H), 4.04 (t, J=6.8 Hz, 2H), 3.61 (bs, 2H), 2.69-2.56 (m, 5H), 2.31 (t, J=7.1 Hz, 2H), 2.27 (t, J=7.5 Hz, 2H), 1.83 (bs, 2H), 1.63-1.57 (m, 4H), 1.50-1.49 (m, 6H), 1.34-1.24 (m, 36H), 0.89 (t, J=7.0 Hz, 3H), 0.86 (t, J=7.0 Hz, 6H); ¹³C NMR (150 MHz, CDCl₃) δ 174.0, 173.2, 74.8, 64.6, 58.3, 56.3, 54.1, 53.3, 34.5, 34.2, 32.0, 29.7, 29.4, 29.2, 28.5, 28.2, 27.3, 25.5, 25.1, 22.8, 22.5, 14.2, 14.1.

### Pentyl 10-((4-(heptadecan-9-yloxy)-4-oxobutyl) (2-hydroxyethyl)amino) decanoate (Compound 18)

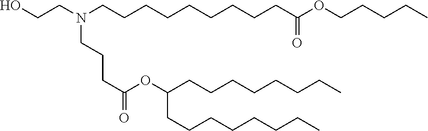 [SMILES: CCCCCCCCC(CCCCCCCC)OC(=O)CCCN(CCO)CCCCCCCCCC(=O)OCCCCC]

Compound 18 was synthesized according to Representative Procedure 1 and general Methods A, B, C, and G. Product was obtained as a colorless oil (330 mg, 0,527 mmol, 79%). MS (ESI): m/z [M+H]⁺ 627.1 for C₃₈H₇₆NO₅; ¹H NMR (600 MHz, CDCl₃) δ 4.87 (qu, J=6.3 Hz, 1H), 4.06 (t, J=6.8 Hz, 2H), 3.53 (t, J=5.4 Hz, 2H), 2.59 (t, J=5.4 Hz, 2H), 2.49 (t, J=7.3 Hz, 2H), 2.45 (t, J=7.5 Hz, 2H), 2.30 (t, J=7.6 Hz, 2H), 2.29 (t, J=7.7, 2H), 1.77 (qu, J=7.3 Hz, 2H), 1.65-1.59 (m, 4H), 1.51-1.50 (m, 4H), 1.42 (qu, J=7.2 Hz, 2H), 1.38-1.32 (m, 4H), 1.30-1.26 (m, 34H), 0.91 (t, J=7.0 Hz, 3H), 0.88 (t, J=7.0 Hz, 6H); ¹³C NMR (150 MHz, CDCl₃) 173.9, 173.3, 74.4, 64.3, 58.5, 55.6, 53.7, 53.0, 34.4, 34.1 (2×), 32.3, 31.8 (2×), 29.5 (3×), 29.5 (2×), 29.4, 29.2 (3×), 29.1, 28.3, 28.1, 27.4, 27.1, 25.3 (2×), 25.0, 22.6 (2×), 22.6, 22.3, 14.1 (2×), 13.9.

### Pentyl 11-((4-(heptadecan-9-yloxy)-4-oxobutyl) (2-hydroxyethyl)amino) undecanoate (Compound 19)

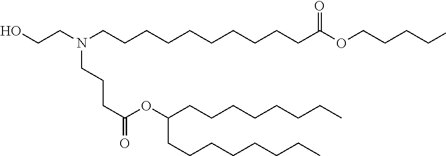 [SMILES: CCCCCCCCC(CCCCCCCC)OC(=O)CCCN(CCO)CCCCCCCCCCC(=O)OCCCCC]

Compound 19 was synthesized according to Representative Procedure 1 and general Methods A, B, C, and G. Product was obtained as a pale yellow oil (323 mg, 0.51 mmol, 80%). MS (ESI): m/z [M+H]⁺ 641.1 for C₃₉H₇₇NO₅; ¹H NMR (600 MHz, CDCl₃) δ 4.86 (p, J=6.3 Hz, 1H), 4.05 (t, J=6.8 Hz, 2H), 3.58 (t, J=4.5 Hz, 2H), 2.65 (t, J=5.1 Hz, 2H), 2.59-2.54 (m, 2H), 2.53-2.48 (m, 2H), 2.30 (dt, J=18.4, 7.4 Hz, 4H), 1.81 (p, J=7.2 Hz, 2H), 1.65-1.57 (m, 4H), 1.54-1.43 (m, 6H), 1.36-1.30 (m, 4H), 1.30-1.20 (m, 36H), 0.90 (t, J=7.1 Hz, 3H), 0.87 (t, J=7.0 Hz, 6H); ¹³C NMR (150 MHz, CDCl₃) δ 174.1, 173.2, 74.6, 64.5, 58.3, 55.9, 53.9, 53.2, 34.5, 34.2, 32.2, 31.9, 29.6, 29.6, 29.6, 29.5, 29.3, 29.2, 28.4, 28.2, 27.4, 25.4, 25.1, 22.7, 22.4, 14.2, 14.0

### Pentyl 12-((4-(heptadecan-9-yloxy)-4-oxobutyl) (2-hydroxyethyl)amino) dodecanoate (Compound 20)

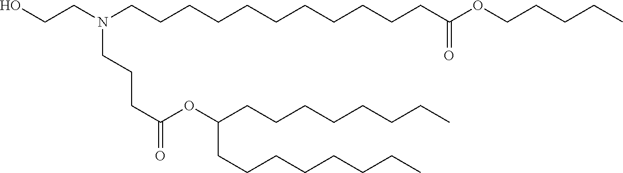 [SMILES: CCCCCCCCC(CCCCCCCC)OC(=O)CCCN(CCO)CCCCCCCCCCCC(=O)OCCCCC]

Compound 20 was synthesized according to Representative Procedure 1 and general Methods A, B, C, and G. Product was obtained as a pale yellow oil (264 mg, 0.404 mmol, 67%). MS (ESI): m/z [M+H]⁺ for C₄₀H₈₀NO₅; ¹H NMR (600 MHz, CDCl₃-CD₃OD=2:1) δ 4.88 (p, J=6.2 Hz, 1H), 4.07 (t, J=6.7 Hz, 2H), 3.62 (t, J=5.7 Hz, 2H), 2.68 (t, J=5.3 Hz, 2H), 2.62-2.51 (m, 4H), 2.38-2.29 (m, 4H), 1.86-1.78 (m, 2H), 1.68-1.59 (m, 4H), 1.58-1.51 (m, 4H), 1.50-1.46 (m, 2H), 1.39-1.22 (m, 42H), 0.92 (t, J=7.0 Hz, 3H), 0.88 (t, J=7.0 Hz, 6H); ¹³C NMR (150 MHz, CDCl₃-CD₃OD=2:1) δ 174.5, 173.5, 74.7, 64.4, 58.5, 55.5, 54.0, 53.1, 34.2, 33.8, 32.0, 31.6, 29.4, 29.3, 29.3, 29.2, 29.2, 29.2, 29.0, 28.9, 28.0, 27.8, 27.2, 26.2, 25.1, 24.8, 22.4, 22.0, 21.7, 13.6, 13.5.

### Pentyl 15-((4-(heptadecan-9-yloxy)-4-oxobutyl) (2-hydroxyethyl)amino) pentadecanoate (Compound 21)

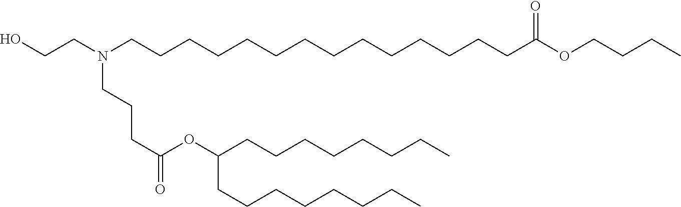 [SMILES: CCCCCCCCC(CCCCCCCC)OC(=O)CCCN(CCO)CCCCCCCCCCCCCCC(=O)OCCCC]

Compound 21 was synthesized according to Representative Procedure 1 and general Methods A, B, C, and G. Product was obtained as a pale yellow oil (270 mg, 0.388 mmol, 72%). MS (ESI): m/z [M+H]⁺ for C₄₃H₈₅NO₅; ¹H NMR (400 MHz, CDCl₃-CD₃OD=2:1) δ 4.88 (qu, J=6.3 Hz, 1H), 4.07 (t, J=6.7 Hz, 2H), 3.61 (t, J=5.9 Hz, 2H), 2.69-2.63 (m, 2H), 2.60-2.49 (m, 4H), 2.38-2.27 (m, 4H), 1.87-1.76 (m, 2H), 1.68-1.58 (m, 4H), 1.57-1.43 (m, 6H), 1.38-1.23 (m, 48H), 0.95-0.86 (m, 9H); ¹³C NMR (100 MHz, CDCl₃-CD₃OD=2:1) δ 174.5, 173.6, 74.6, 64.3, 58.5, 55.5, 54.0, 53.1, 34.1, 33.8, 32.0, 31.6, 29.4, 29.3, 29.3, 29.2, 29.2, 29.0, 28.8, 28.0, 27.8, 27.2, 26.2, 25.1, 24.7, 22.3, 22.0, 21.7, 13.6, 13.5.

### Methyl 9-((5-(heptadecan-9-yloxy)-5-oxopentyl) (2-hydroxyethyl)amino) nonanoate (Compound 22)

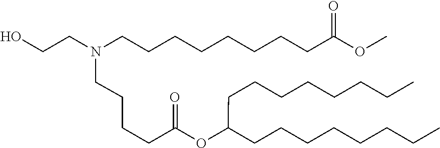 [SMILES: CCCCCCCCC(CCCCCCCC)OC(=O)CCCCN(CCO)CCCCCCCCC(=O)OC]

Compound 22 was synthesized according to Representative Procedure 1 and general Methods A, B, C, and G. Product was obtained as a pale yellow oil (441 mg, 0.77 mmol, 90%). MS (ESI): m/z [M+H]⁺ 571.0 for C₃₄H₆₈NO₅; ¹H NMR (400 MHz, CDCl₃) δ 4.86 (qu, J=6.3 Hz, 1H), 3.66 (s, 3H), 3.53 (t, J=5.4 Hz, 2H), 2.58 (t, J=5.4 Hz, 2H), 2.52-2.41 (m, 4H), 2.32-2.26 (m, 4H), 1.66-1.56 (m, 4H), 1.54-1.38 (m, 8H), 1.32-1.19 (m, 32H), 0.87 (t, J=6.9 Hz, 6H); ¹³C NMR (100 MHz, CDCl₃) δ 174.4, 173.4, 74.4, 58.4, 55.6, 53.9, 53.5, 51.5, 34.5, 34.2, 34.2, 31.9, 29.6, 29.6, 29.4, 29.3, 29.3, 29.2, 27.4, 27.1, 26.6, 25.4, 25.0, 23.0, 22.7, 14.2.

### Ethyl 9-((5-(heptadecan-9-yloxy)-5-oxopentyl) (2-hydroxyethyl)amino) nonanoate (Compound 23)

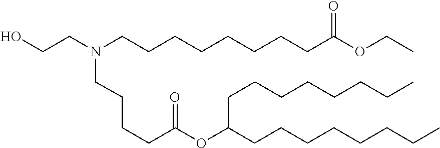 [SMILES: CCCCCCCCC(CCCCCCCC)OC(=O)CCCCN(CCO)CCCCCCCCC(=O)OCC]

Compound 23 was synthesized according to Representative Procedure 1 and general Methods A, B, C, and G. Product was obtained as a pale yellow oil (408 mg, 0.70 mmol, 91%). MS (ESI): m/z [M+H]⁺ 585.0 for C₃₅H₇₀NO₅; ¹H NMR (600 MHz, CDCl₃) δ 4.86 (qu, J=6.3 Hz, 1H), 4.12 (q, J=7.1 Hz, 2H), 3.59 (t, J=4.4 Hz, 2H), 2.67-2.63 (m, 2H), 2.58-2.49 (m, 4H), 2.29 (dt, J=17.3, 7.4 Hz, 4H), 1.65-1.57 (m, 4H), 1.56-1.44 (m, 8H), 1.32-1.20 (m, 35H), 0.87 (t, J=7.1 Hz, 6H); ¹³C NMR (150 MHz, CDCl₃) δ 173.9, 173.3, 74.4, 60.2, 58.2, 55.9, 54.0, 53.6, 34.4, 34.2, 31.9, 29.6, 29.6, 29.4, 29.3, 29.3, 29.1, 27.3, 25.4, 25.0, 22.9, 22.7, 14.3, 14.2.

### Propyl 9-((5-(heptadecan-9-yloxy)-5-oxopentyl) (2-hydroxyethyl)amino) nonanoate (Compound 24)

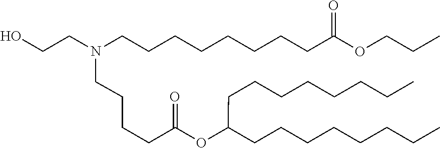 [SMILES: CCCCCCCCC(CCCCCCCC)OC(=O)CCCCN(CCO)CCCCCCCCC(=O)OCCC]

Compound 24 was synthesized according to Representative Procedure 1 and general Methods A, B, C, and G. Product was obtained as a pale yellow oil (401 mg, 0.67 mmol, 87%). MS (ESI): m/z [M+H]⁺ 599.0 for C₃₆H₇₂NO₅; ¹H NMR (600 MHz, CDCl₃) δ 4.86 (p, J=6.2 Hz, 1H), 4.02 (t, J=6.7 Hz, 2H), 3.58 (t, J=5.1 Hz, 2H), 2.65 (t, J=4.5 Hz, 2H), 2.58-2.48 (m, 4H), 2.30 (dt, J=10.7, 7.4 Hz, 4H), 1.67-1.58 (m, 6H), 1.56-1.44 (m, 8H), 1.32-1.21 (m, 32H), 0.93 (t, J=7.4 Hz, 3H), 0.87 (t, J=7.1 Hz, 6H); ¹³C NMR (150 MHz, CDCl₃) δ 174.0, 173.3, 74.4, 66.9, 58.2, 55.9, 54.0, 53.6, 34.4, 34.2, 31.9, 29.6, 29.6, 29.4, 29.3, 29.3, 29.2, 27.4, 25.4, 25.0, 22.9, 22.7, 22.0, 14.2, 10.5.

### Butyl 9-((2-(heptadecan-9-yloxy)-2-oxoethyl) (2-hydroxyethyl)amino) nonanoate (Compound 25)

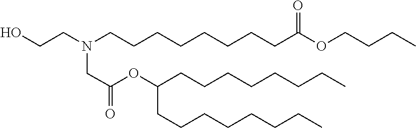 [SMILES: CCCCCCCCC(CCCCCCCC)OC(=O)CN(CCO)CCCCCCCCC(=O)OCCCC]

Compound 25 was synthesized according to Representative Procedure 1 and general Methods A, B, C, and G. Product was obtained as a colorless oil (200 mg, 0.351 mmol, 41%). MS (ESI): m/z [M+H]⁺ 570.4 for C₃₄H₆₈NO₅; ¹H NMR (400 MHz, CDCl₃) δ 4.95-4.89 (m, 1H), 4.07 (t, J=6.7 Hz, 2H), 3.56-3.53 (m, 2H), 3.34 (s, 2H), 2.79-2.76 (m, 2H), 2.64-2.60 (m, 2H), 2.30-2.27 (m, 2H), 1.64-1.57 (m, 4H), 1.53-1.52 (m, 4H), 1.46-1.35 (m, 4H), 1.29-1.26 (m, 35H), 0.93 (t, J=7.4 Hz, 3H), 0.88 (t, J=6.9 Hz, 6H); ¹³C NMR (100 MHz, CDCl₃) δ 174.1, 172.0, 75.2, 64.2, 59.1, 57.0, 55.4, 54.7, 34.5, 34.2, 32.0, 30.8, 29.6, 29.5, 29.4, 29.4, 29.3, 27.9, 27.3, 25.5, 25.1, 22.8, 19.3, 14.2, 13.8.

### Butyl 9-((3-(heptadecan-9-yloxy)-3-oxopropyl) (2-hydroxyethyl)amino) nonanoate (Compound 26)

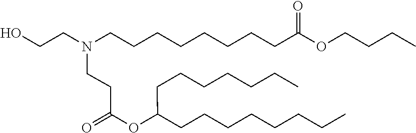 [SMILES: CCCCCCCCC(CCCCCCC)OC(=O)CCN(CCO)CCCCCCCCC(=O)OCCCC]

Compound 26 was synthesized according to Representative Procedure 1 and general Methods A, B, C, and G. Product was obtained as a colorless oil (100 mg, 0,171 mmol, 34%). MS (ESI): m/z [M+H]⁺ 584.9 for C₃₅H₇₀NO₅; ¹H-NMR (400 MHz, CDCl₃) δ 4.88 (qu, J=6.3 Hz, 1H), 4.07 (t, J=6.7 Hz, 2H), 3.58 (t, J=4.5 Hz, 2H), 2.82 (t, J=6.1 Hz, 2H), 2.61 (bs, 2H), 2.46 (bs, 4H), 2.29 (t, J=7.6 Hz, 2H), 1.64-1.57 (m, 4H), 1.52-1.50 (m, 5H), 1.41-1.33 (m, 3H), 1.30-1.26 (m, 32H), 0.93 (t, J=7.4 Hz, 3H), 0.88 (t, J=6.8 Hz, 6H); ¹³C NMR (100 MHz, CDCl₃) δ 173.9, 172.5, 74.8, 64.1, 58.7, 55.8, 54.0, 49.4, 34.4, 34.0 (2×), 31.8 (2×), 30.7, 29.5 (2×), 29.5 (2×), 29.4, 29.2 (3×), 29.1, 27.3, 25.3 (2×), 25.0, 22.6 (2×), 19.1, 14.1 (2×), 13.7.

### Butyl 9-((5-(heptadecan-9-yloxy)-5-oxopentyl) (2-hydroxyethyl)amino) nonanoate (Compound 27)

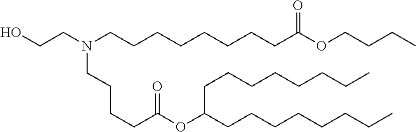 [SMILES: CCCCCCCCC(CCCCCCCC)OC(=O)CCCCN(CCO)CCCCCCCCC(=O)OCCCC]

Compound 27 was synthesized according to Representative Procedure 1 and general Methods A, B, C, and G. Product was obtained as a colorless oil (172 mg, 0.281 mmol, 59%). MS (ESI): m/z [M+H]⁺ 613.1 for C₃₇H₇₄NO₅; ¹H NMR (400 MHz, CDCl₃) δ 4.90-4.84 (m, 1H), 4.07 (t, J=6.7 Hz, 2H), 3.52 (t, J=5.4 Hz, 2H), 2.57 (t, J=5.4 Hz, 2H), 2.49-2.41 (m, 4H), 2.32-2.27 (m, 4H), 1.66-1.57 (m, 6H), 1.51-1.35 (m, 10H), 1.29-1.26 (m, 35H), 0.93 (t, J=7.4 Hz, 3H), 0.88 (t, J=6.9 Hz, 6H); ¹³C NMR (150 MHz, CDCl₃) δ 174.1, 173.5, 74.4, 64.2, 58.5, 55.6, 53.9, 53.6, 34.6, 34.5, 34.3, 32.0, 30.8, 29.7, 29.6, 29.4, 29.4, 29.3, 27.5, 27.3, 26.8, 25.5, 25.1, 23.1, 22.8, 19.3, 14.2, 13.8.

### Methyl 10-((5-(heptadecan-9-yloxy)-5-oxopentyl) (2-hydroxyethyl)amino) decanoate (Compound 28)

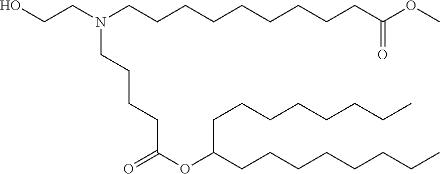 [SMILES: CCCCCCCCC(CCCCCCCC)OC(=O)CCCCN(CCO)CCCCCCCCCC(=O)OC]

Compound 28 was synthesized according to Representative Procedure 1 and general Methods A, B, C, and G. Product was obtained as a pale yellow oil (140 mg, 0.237 mmol, 39%). MS (ESI): m/z [M+H]⁺ 584.9 for C₃₅H₇NO₅; ¹H NMR (400 MHz, CDCl₃) δ 4.86 (p, J=6.3 Hz, 1H), 3.66 (bs, 5H), 2.47-2.63 (m, 5H), 2.34-2.28 (m, 4H), 1.63-1.40 (m, 12H), 1.28-1.25 (m, 35H), 0.87 (t, J=6.9 Hz, 6H); ¹³C NMR (100 MHz, CDCl₃) δ 174.4, 173.2, 74.6, 58.0, 54.2, 53.8, 51.6, 34.3, 34.2, 32.0, 29.7, 29.6, 29.5, 29.4, 29.3, 29.2, 27.3, 25.5, 25.1, 22.8, 14.2.

### Methyl 10-((4-(heptadecan-9-yloxy)-4-oxobutyl) (2-hydroxyethyl)amino) decanoate (Compound 29)

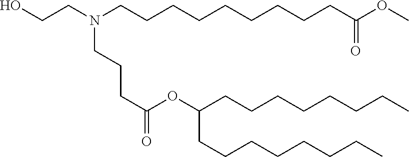 [SMILES: CCCCCCCCC(CCCCCCCC)OC(=O)CCCN(CCO)CCCCCCCCCC(=O)OC]

Compound 29 was synthesized according to Representative Procedure 1 and general Methods A, B, C, and G. Product was obtained as a pale yellow oil (140 mg, 0.243 mmol, 40%). MS (ESI): m/z [M+H]⁺ 570.7 for C₃₄H₆₈NO₅; ¹H NMR (400 MHz, CDCl₃) δ 4.86 (qu, J=6.3 Hz, 1H), 3.66 (s, 3H), 3.64 (bs, 2H), 2.72-2.59 (m, 5H), 2.35-2.28 (m, 4H), 1.87-1.84 (m, 2H), 1.63-1.57 (m, 2H), 1.51-1.40 (m, 6H), 1.28-1.25 (m, 35H), 0.87 (t, J=6.9 Hz, 6H); ¹³C NMR (100 MHz, CDCl₃) δ 174.4, 173.1, 74.8, 58.2, 56.4, 54.1, 53.3, 51.6, 34.2, 32.1, 32.0, 29.7, 29.6, 29.5, 29.4, 29.3, 29.2, 27.4, 25.5, 25.1, 22.8, 14.8.

### Ethyl 10-((5-(heptadecan-9-yloxy)-5-oxopentyl) (2-hydroxyethyl)amino) decanoate (Compound 30)

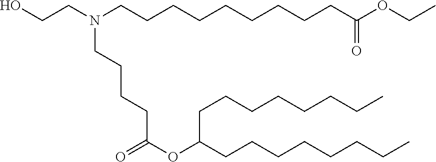 [SMILES: CCCCCCCCC(CCCCCCCC)OC(=O)CCCCN(CCO)CCCCCCCCCC(=O)OCC]

Compound 30 was synthesized according to Representative Procedure 1 and general Methods A, B, C, and G. Product was obtained as a pale yellow oil (200 mg, 0.331 mmol, 57%). MS (ESI): m/z [M+H]⁺ 598.3 for C₃₆H₇₂NO₅; ¹H NMR (600 MHz, CDCl₃) δ 4.88-4.84 (m, 1H), 4.12 (q, J=7.1 Hz, 2H), 3.68 (bs, 2H), 2.76-2.66 (m, 4H), 2.33-2.31 (m, 2H), 2.29-2.27 (m, 2H), 1.63-1.50 (m, 11H), 1.31-1.24 (m, 38H), 0.87 (t, J=7.0 Hz, 6H); ¹³C NMR (150 MHz, CDCl₃) δ 174.0, 173.2, 74.6, 60.3, 57.9, 56.5, 54.2, 53.8, 34.5, 34.3, 32.0, 29.7, 29.6, 29.5, 29.5, 29.4, 29.3, 29.2, 25.5, 25.1, 22.8, 14.4, 14.2.

### Ethyl 10-((4-(heptadecan-9-yloxy)-4-oxobutyl) (2-hydroxyethyl)amino) decanoate (Compound 31)

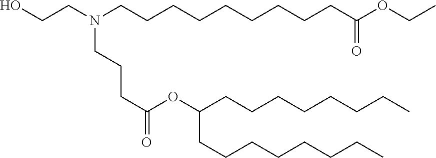 [SMILES: CCCCCCCCC(CCCCCCCC)OC(=O)CCCN(CCO)CCCCCCCCCC(=O)OCC]

Compound 31 was synthesized according to Representative Procedure 1 and general Methods A, B, C, and G. Product was obtained as a pale yellow oil (190 mg, 0.322 mmol, 56%). MS (ESI): m/z [M+H]⁺ 584.9 for C₃₅H₇₀NO₅; ¹H NMR (600 MHz, CDCl₃) δ 4.88-4.84 (m, 1H), 4.12 (q, J=7.1 Hz, 2H), 3.68-3.62 (m, 2H), 2.75-2.62 (m, 4H), 2.34 (t, J=6.8 Hz, 2H), 2.29-2.27 (m, 2H), 1.89 (bs, 2H), 1.63-1.59 (m 2H), 1.52-1.50 (m, 5H), 1.31-1.24 (m, 38H), 0.87 (t, J=7.1 Hz, 6H); ¹³C NMR (150 MHz, CDCl₃) δ 174.0, 173.0, 74.9, 60.3, 58.1, 54.2, 53.3, 34.5, 34.2, 32.0, 29.7, 29.5, 29.4, 29.3, 29.2, 25.5, 25.1, 22.8, 14.4, 14.2.

### Propyl 10-((5-(heptadecan-9-yloxy)-5-oxopentyl) (2-hydroxyethyl)amino) decanoate (Compound 32)

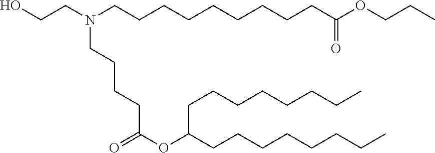 [SMILES: CCCCCCCCC(CCCCCCCC)OC(=O)CCCCN(CCO)CCCCCCCCCC(=O)OCCC]

Compound 32 was synthesized according to Representative Procedure 1 and general Methods A, B, C, and G. Product was obtained as a pale yellow oil (170 mg, 0.275 mmol, 50%). MS (ESI): m/z [M+H]⁺ 612.3 for C₃₇H₇₄NO₅; ¹H NMR (600 MHz, CDCl₃) δ 4.88-4.84 (m, 1H), 4.02 (t, J=6.7 Hz, 2H), 3.66-3.64 (m, 2H), 2.73-2.60 (m, 5H), 2.32 (t, J=7.3 Hz, 2H), 2.30-2.28 (m, 2H), 1.67-1.59 (m, 7H), 1.52-1.50 (m, 6H), 1.30-1.25 (m, 37H), 0.94 (t, J=7.4 Hz, 3H), 0.87 (t, J=7.0 Hz, 6H); ¹³C NMR (150 MHz, CDCl₃) δ 174.0, 173.2, 74.5, 65.9, 58.0, 56.2, 54.1, 53.7, 34.4, 34.3, 34.2, 31.9, 29.6, 29.6, 29.4, 29.3, 29.3, 29.2, 27.3, 25.4, 25.1, 22.8, 22.7, 22.1, 14.2, 10.5.

### Propyl 10-((4-(heptadecan-9-yloxy)-4-oxobutyl) (2-hydroxyethyl)amino) decanoate (Compound 33)

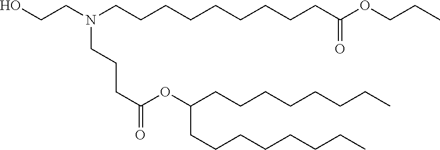 [SMILES: CCCCCCCCC(CCCCCCCC)OC(=O)CCCN(CCO)CCCCCCCCCC(=O)OCCC]

Compound 33 was synthesized according to Representative Procedure 1 and general Methods A, B, C, and G. Product was obtained as a pale yellow oil (170 mg, 0.281 mmol, 51%). MS (ESI): m/z [M+H]⁺ 598.3 for C₃₆H₇₂NO₅; ¹H NMR (600 MHz, CDCl₃) δ 4.88-4.84 (m, 1H), 4.02 (t, J=6.7 Hz, 2H), 3.62 (bs, 2H), 2.72-2.57 (m, 5H), 2.32 (t, J=7.1 Hz, 2H), 2.30-2.28 (m, 2H), 1.84 (bs, 2H), 1.67-1.59 (m, 4H), 1.51-1.50 (m, 6H), 1.30-1.25 (m, 37H), 0.94 (t, J=7.4 Hz, 3H), 0.87 (t, J=7.0 Hz, 6H); ¹³C NMR (150 MHz, CDCl₃) δ 174.1, 173.2, 74.8, 66.0, 58.3, 56.3, 54.1, 53.3, 34.5, 34.2, 32.1, 32.0, 29.7, 29.6, 29.5, 29.4, 29.3, 29.3, 27.4, 25.5, 25.1, 22.8, 22.2, 14.2, 10.5.

### Butyl 10-((5-(heptadecan-9-yloxy)-5-oxopentyl) (2-hydroxyethyl)amino) decanoate (Compound 34)

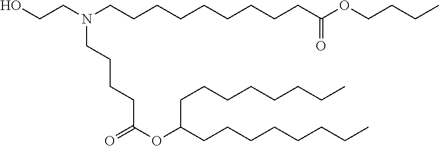 [SMILES: CCCCCCCCC(CCCCCCCC)OC(=O)CCCCN(CCO)CCCCCCCCCC(=O)OCCCC]

Compound 34 was synthesized according to Representative Procedure 1 and general Methods A, B, C, and G. Product was obtained as a pale yellow oil (450 mg, 0.712 mmol, 68.2%). MS (ESI): m/z [M+H]⁺ 626.6 for C₃₈H₇₆NO₅; ¹H NMR (400 MHz, CDCl₃) δ 4.86 (qu, J=6.2 Hz, 1H), 4.06 (t, J=6.7 Hz, 2H), 3.61 (t, J=5.0 Hz, 2H), 2.69-2.68 (m, 2H), 2.57 (dt, J=15.0, 7.5 Hz, 4H), 2.32 (t, J=5.6 Hz, 2H), 2.28 (t, J=6.0 Hz, 2H), 1.67-1.49 (m, 14H), 1.42-1.33 (m, 2H), 1.28-1.25 (m, 34H), 0.93 (t, J=7.4 Hz, 3H), 0.87 (t, J=6.8 Hz, 6H); ¹³C NMR (100 MHz, CDCl₃) δ 174.1, 173.3, 74.5, 64.3, 58.1, 56.1, 54.1, 53.7, 34.5, 34.4, 34.3, 32.0, 30.9, 29.7, 29.7, 29.5, 29.5, 29.4, 27.4 25.5, 25.1, 22.9, 22.8, 19.3, 14.2, 13.9.

### Butyl 11-((5-(heptadecan-9-yloxy)-5-oxopentyl) (2-hydroxyethyl)amino) undecanoate (Compound 35)

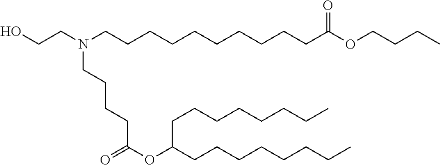 [SMILES: CCCCCCCCC(CCCCCCCC)OC(=O)CCCCN(CCO)CCCCCCCCCCC(=O)OCCCC]

Compound 35 was synthesized according to Representative Procedure 1 and general Methods A, B, C, and G. Product was obtained as a pale yellow oil (387 mg, 0.61 mmol, 91%). MS (ESI): m/z [M+H]⁺ 641.1 for C₃₉H₇₈NO₅; ¹H NMR (400 MHz, CDCl₃) δ 4.86 (qu, J=6.3 Hz, 1H), 4.06 (t, J=6.7 Hz, 2H), 3.54 (t, J=5.4 Hz, 2H), 2.59 (t, J=5.4 Hz, 2H), 2.53-2.42 (m, 4H), 2.29 (q, J=7.4 Hz, 4H), 1.66-1.55 (m, 6H), 1.54-1.46 (m, 6H), 1.44-1.33 (m, 4H), 1.32-1.19 (m, 36H), 0.92 (t, J=7.4 Hz, 3H), 0.87 (t, J=6.9 Hz, 6H); ¹³C NMR (100 MHz, CDCl₃) δ 174.1, 173.3, 74.4, 64.5, 58.2, 55.9, 54.0, 53.6, 34.5, 34.4, 34.2, 31.9, 29.6, 29.6, 29.5, 29.3, 29.2, 28.4, 28.2, 27.4, 25.4, 25.1, 22.9, 22.7, 14.2, 14.0.

### Butyl 11-((4-(heptadecan-9-yloxy)-4-oxobutyl) (2-hydroxyethyl)amino) undecanoate (Compound 36)

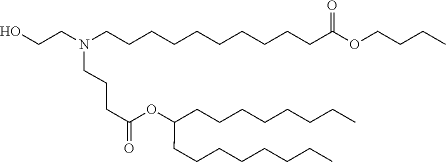 [SMILES: CCCCCCCCC(CCCCCCCC)OC(=O)CCCN(CCO)CCCCCCCCCCC(=O)OCCCC]

Compound 36 was synthesized according to Representative Procedure 1 and general Methods A, B, C, and G. Product was obtained as a pale yellow oil (331 mg, 0.53 mmol, 80%). MS (ESI): m/z [M+H]⁺ 627.1 for C₃₈H₇₆NO₅; ¹H NMR (400 MHz, CDCl₃) δ 4.86 (qu, J=6.3 Hz, 1H), 4.06 (t, J=6.7 Hz, 2H), 3.58 (t, J=5.2 Hz, 2H), 2.65 (t, J=5.1 Hz, 2H), 2.59-2.47 (m, 4H), 2.29 (dt, J=12.8, 7.5 Hz, 4H), 1.81 (qu, J=7.2 Hz, 2H), 1.64-1.54 (m, 4H), 1.55-1.45 (m, 6H), 1.43-1.33 (m, 2H), 1.33-1.19 (m, 36H), 0.93 (t, J=7.4 Hz, 3H), 0.87 (t, J=6.9 Hz, 6H); ¹³C NMR (100 MHz, CDCl₃) δ 174.1, 173.2, 74.6, 64.2, 58.4, 55.9, 54.0, 53.9, 53.2, 34.5, 34.2, 32.2, 31.9, 30.8, 29.6, 29.6, 29.6, 29.3, 29.2, 27.4, 25.4, 25.1, 22.8, 19.2, 14.2, 13.8.

### Butyl 12-((5-(heptadecan-9-yloxy)-5-oxopentyl) (2-hydroxyethyl)amino) dodecanoate (Compound 37)

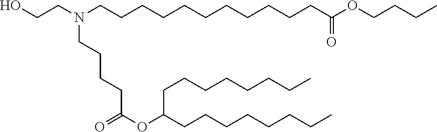 [SMILES: CCCCCCCCC(CCCCCCCC)OC(=O)CCCCN(CCO)CCCCCCCCCCCC(=O)OCCCC]

Compound 37 was synthesized according to Representative Procedure 1 and general Methods A, B, C, and G. Product was obtained as a pale yellow oil (137 mg, 0.209 mmol, 33%). MS (ESI): m/z [M+H]⁺ 655.0 for C₄₀H₈₀NO₅; ¹H NMR (400 MHz, CDCl₃-CD₃OD=2:1) δ 4.87 (qu, J=6.2 Hz, 1H), 4.08 (t, J=6.6 Hz, 2H), 3.61 (t, J=5.9 Hz, 2H), 2.68-2.60 (m, 2H), 2.57-2.47 (m, 4H), 2.33 (dt, J=13.1, 7.4 Hz, 4H), 1.67-1.58 (m, 6H), 1.57-1.47 (m, 8H), 1.46-1.36 (m, 4H), 1.35-1.20 (m, 37H), 0.95 (t, J=7.4 Hz, 3H), 0.89 (t, J=6.8 Hz, 6H); ¹³C NMR (100 MHz, CDCl₃-CD₃OD=2:1) δ 174.5, 173.7, 74.5, 64.1, 58.5, 55.4, 53.9, 53.5, 34.2, 34.1, 33.8, 31.6, 30.4, 29.3, 29.3, 29.3, 29.2, 29.2, 29.2, 29.0, 28.9, 28.8, 27.2, 26.2, 25.7, 25.1, 24.7, 22.7, 22.4, 18.8, 13.6, 13.2.

### Butyl 12-((4-(heptadecan-9-yloxy)-4-oxobutyl) (2-hydroxyethyl)amino) dodecanoate (Compound 38)

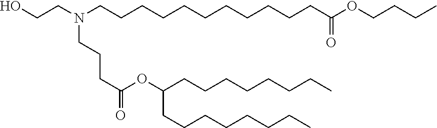 [SMILES: CCCCCCCCC(CCCCCCCC)OC(=O)CCCN(CCO)CCCCCCCCCCCC(=O)OCCCC]

Compound 38 was synthesized according to Representative Procedure 1 and general Methods A, B, C, and G. Product was obtained as a pale yellow oil (260 mg, 0.406 mmol, 64%). MS (ESI): m/z [M+H]⁺ 641.0 for C₃₉H₇₈NO₅; ¹H NMR (600 MHz, CDCl₃-CD₃OD=2:1) δ 4.88 (qu, J=6.2 Hz, 1H), 4.08 (t, J=6.6 Hz, 2H), 3.65 (t, J=4.8 Hz, 2H), 2.79-2.70 (m, 2H), 2.69-2.55 (m, 4H), 2.36 (t, J=7.1 Hz, 2H), 2.31 (t, J=7.5 Hz, 2H), 1.88-1.79 (m, 2H), 1.66-1.59 (m, 4H), 1.58-1.46 (m, 6H), 1.44-1.36 (m, 2H), 1.35-1.23 (m, 38H), 0.95 (t, J=7.4 Hz, 3H), 0.89 (t, J=7.0 Hz, 6H); ¹³C NMR (150 MHz, CDCl₃-CD₃OD=2:1) δ 174.5, 173.6, 74.6, 64.0, 58.6, 55.5, 54.0, 53.1, 34.1, 33.8, 32.0, 31.6, 30.4, 29.3, 29.3, 29.2, 29.2, 29.2, 29.1, 28.9, 28.8, 27.2, 26.3, 25.0, 24.7, 22.3, 21.8, 18.8, 13.6, 13.2.

### Butyl 15-((5-(heptadecan-9-yloxy)-5-oxopentyl) (2-hydroxyethyl)amino) pentadecanoate (Compound 39)

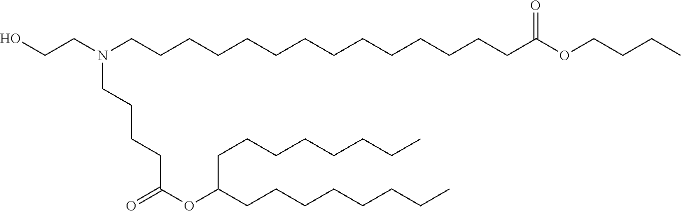 [SMILES: CCCCCCCCC(CCCCCCCC)OC(=O)CCCCN(CCO)CCCCCCCCCCCCCCC(=O)OCCCC]

Compound 39 was synthesized according to Representative Procedure 1 and general Methods A, B, C, and G. Product was obtained as a pale yellow oil (193 mg, 0.28 mmol, 83%). MS (ESI): m/z [M+H]⁺ 697.3 for C₄₃H₈₅NO₅; ¹H NMR (600 MHz, CDCl₃) δ 4.86 (qu, J=6.2 Hz, 1H), 4.06 (t, J=6.7 Hz, 2H), 3.62-3.57 (m, 2H), 2.69-2.63 (m, 2H), 2.60-2.50 (m, 4H), 2.29 (dt, J=16.8, 7.4 Hz, 4H), 1.66-1.57 (m, 6H), 1.57-1.45 (m, 8H), 1.42-1.33 (m, 2H), 1.32-1.20 (m, 44H), 0.93 (t, J=7.4 Hz, 3H), 0.87 (t, J=7.0 Hz, 6H); ¹³C NMR (150 MHz, CDCl₃) δ 174.1, 173.3, 74.4, 64.2, 58.2, 55.9, 54.0, 53.6, 34.5, 34.4, 34.2, 31.9, 30.8, 29.7, 29.7, 29.6, 29.6, 29.5, 29.3, 29.3, 29.2, 27.4, 25.4, 25.1, 22.9, 22.7, 19.2, 14.2, 13.8.

### Butyl 15-((4-(heptadecan-9-yloxy)-4-oxobutyl) (2-hydroxyethyl)amino) pentadecanoate (Compound 40)

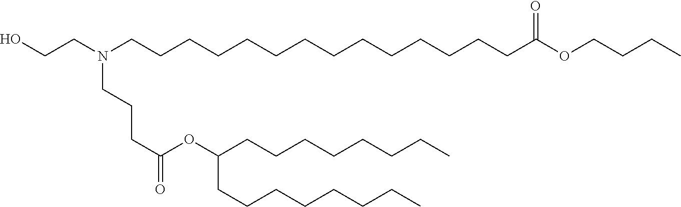 [SMILES: CCCCCCCCC(CCCCCCCC)OC(=O)CCCN(CCO)CCCCCCCCCCCCCCC(=O)OCCCC]

Compound 40 was synthesized according to Representative Procedure 1 and general Methods A, B, C, and G. Product was obtained as a pale yellow oil (299 mg, 0.44 mmol, 78%). MS (ESI): m/z [M+H]⁺ 683.2 for C₄₂H₈₄NO₅; ¹H NMR (600 MHz, CDCl₃) δ 4.86 (qu, J=6.2 Hz, 1H), 4.06 (t, J=6.7 Hz, 2H), 3.58 (t, J=6.0 Hz, 2H), 2.68-2.61 (m, 2H), 2.60-2.47 (m, 4H), 2.30 (dt, J=19.5, 7.4 Hz, 4H), 1.85-1.77 (m, 2H), 1.64-1.57 (m, 4H), 1.55-1.43 (m, 6H), 1.41-1.33 (m, 2H), 1.32-1.19 (m, 44H), 0.93 (t, J=7.4 Hz, 3H), 0.87 (t, J=7.0 Hz, 6H); ¹³C NMR (150 MHz, CDCl₃) δ 174.1, 173.2, 74.6, 64.2, 58.3, 56.0, 54.0, 53.2, 34.5, 34.2, 32.2, 31.9, 30.8, 29.7, 29.7, 29.6, 29.6, 29.6, 29.3, 29.3, 29.2, 27.5, 25.4, 25.1, 22.7, 19.2, 14.2, 13.8.

### Pentyl 9-((2-hydroxyethyl) (5-oxo-5-(pentadecan-8-yloxy) pentyl)amino) nonanoate (Compound 41)

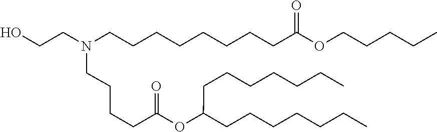 [SMILES: CCCCCCCC(CCCCCCC)OC(=O)CCCCN(CCO)CCCCCCCCC(=O)OCCCCC]

Compound 41 was synthesized according to Representative Procedure 1 and general Methods A, B, C, and G. Product was obtained as a pale yellow oil (278 mg, 0.465 mmol, 67%). MS (ESI): m/z [M+H]⁺ 599.1 for C₃₆H₇₂NO₅; ¹H NMR (600 MHz, CDCl₃-CD₃OD=2:1) δ 4.91-4.83 (m, 1H), 4.07 (t, J=6.7 Hz, 2H), 3.60 (t, J=5.9 Hz, 2H), 2.64 (t, J=5.9 Hz, 2H), 2.57-2.47 (m, 4H), 2.33 (dt, J=17.4, 7.4 Hz, 4H), 1.67-1.59 (m, 6H), 1.58-1.49 (m, 8H), 1.49-1.44 (m, 2H), 1.37-1.25 (m, 30H), 0.92 (t, J=7.0 Hz, 3H), 0.89 (t, J=7.0 Hz, 6H); ¹³C NMR (150 MHz, CDCl₃-CD₃OD=2:1) δ 174.4, 173.7, 74.4, 64.3, 58.5, 55.4, 53.9, 53.5, 34.2, 34.1, 33.8, 31.5, 29.2, 29.1, 29.0, 28.9, 28.8, 28.0, 27.8, 27.2, 26.3, 25.7, 25.0, 24.7, 22.7, 22.3, 22.0, 13.6, 13.4.

### Pentyl 9-((2-hydroxyethyl) (5-(nonadecan-10-yloxy)-5-oxopentyl)amino) nonanoate (Compound 42)

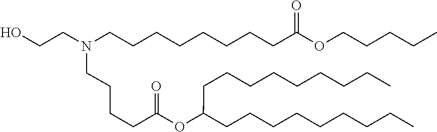 [SMILES: CCCCCCCCCC(CCCCCCCCC)OC(=O)CCCCN(CCO)CCCCCCCCC(=O)OCCCCC]

Compound 42 was synthesized according to Representative Procedure 1 and general Methods A, B, C, and G. Product was obtained as a pale yellow oil (295 mg, 0.451 mmol, 65%). MS (ESI): m/z [M+H]⁺ 655.1 for C₄₀H₃₀NO₅; ¹H NMR (600 MHz, CDCl₃-CD:OD=2:1) δ 4.87 (qu, J=6.3 Hz, 1H), 4.07 (t, J=6.7 Hz, 2H), 3.60 (t, J=5.9 Hz, 2H), 2.64 (t, J=5.9 Hz, 2H), 2.57-2.47 (m, 4H), 2.33 (dt, J=17.0, 7.4 Hz, 4H), 1.67-1.58 (m, 6H), 1.56-1.49 (m, 8H), 1.48-1.43 (m, 2H), 1.38-1.22 (m, 38H), 0.92 (t, J=7.0 Hz, 3H), 0.89 (t, J=7.0 Hz, 6H); ¹³C NMR (150 MHz, CDCl₃-CD₃OD=2:1) δ 174.4, 173.7, 74.5, 64.4, 58.5, 55.4, 53.9, 53.5, 34.2, 34.1, 33.8, 31.6, 29.2, 29.2, 29.1, 29.0, 29.0, 28.8, 28.0, 27.8, 27.2, 26.3, 25.7, 25.0, 24.7, 22.7, 22.4, 22.0, 13.6, 13.5.

### Butyl 10-((2-hydroxyethyl) (5-oxo-5-(pentadecan-8-yloxy) pentyl)amino) decanoate (Compound 43)

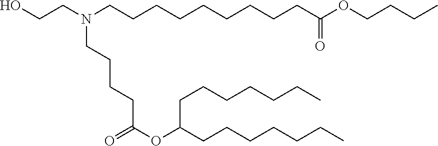 [SMILES: CCCCCCCC(CCCCCCC)OC(=O)CCCCN(CCO)CCCCCCCCCC(=O)OCCCC]

Compound 43 was synthesized according to Representative Procedure 1 and general Methods A, B, C, and G. Product was obtained as a pale yellow oil (206 mg, 0.341 mmol, 53%). MS (ESI): m/z [M+H]⁺ 598.2 for C₃₆H₇₂NO₅; ¹H NMR (400 MHz, CDCl₃) δ 4.86 (qu, J=6.3 Hz, 1H), 4.05 (t, J=6.8 Hz, 2H), 3.58 (t, J=4.8 Hz, 2H), 2.64 (m, 2H), 2.56-2.49 (m, 4H), 2.33-2.27 (m, 4H), 1.66-1.56 (m, 6H), 1.51-1.50 (m, 8H), 1.34-1.26 (m, 34H), 0.92-0.86 (m, 9H); ¹³C NMR (100 MHz, CDCl₃) δ 174.1, 173.4, 74.5, 64.5, 58.3, 55.9, 54.0, 53.7, 34.5, 34.5, 34.3, 31.9, 29.6, 29.6, 29.6, 29.4, 29.4, 29.3, 28.5, 28.2, 27.5, 25.5, 25.1, 23.0, 22.8, 22.5, 14.2, 14.1.

### Pentyl 10-((2-hydroxyethyl) (5-oxo-5-(pentadecan-8-yloxy) pentyl)amino) decanoate (Compound 44)

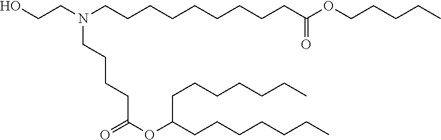 [SMILES: CCCCCCCC(CCCCCCC)OC(=O)CCCCN(CCO)CCCCCCCCCC(=O)OCCCCC]

Compound 44 was synthesized according to Representative Procedure 1 and general Methods A, B, C, and G. Product was obtained as a pale yellow oil (174 mg, 0.281 mmol, 44%). MS (ESI): m/z [M+H]⁺ 612.2 for C₃₇H₇₄NO₅; ¹H NMR (400 MHz, CDCl₃) δ 4.86 (p, J=6.3 Hz, 1H), 4.06 (t, J=6.7 Hz, 2H), 3.56 (t, J=5.2 Hz, 2H), 2.61 (t, J=5.2 Hz, 2H), 2.53-2.46 (m, 4H), 2.32-2.26 (m, 4H), 1.66-1.57 (m, 6H), 1.54-1.33 (m, 10H), 1.27-1.26 (m, 31H), 0.93 (t, J=7.4 Hz, 3H), 0.87 (t, J=6.8 Hz, 6H); ¹³C NMR (100 MHz, CDCl₃) δ 174.1, 173.4, 74.5, 64.2, 58.4, 55.8, 54.0, 53.6, 34.5, 34.5, 31.9, 30.9, 29.6, 29.6, 29.4, 29.4, 29.3, 27.5, 27.1, 27.0, 26.5, 25.5, 25.1, 23.1, 22.8, 19.3, 14.2, 13.9.

### Pentyl 10-((2-hydroxyethyl) (5-(nonadecan-10-yloxy)-5-oxopentyl)amino) decanoate (Compound 45)

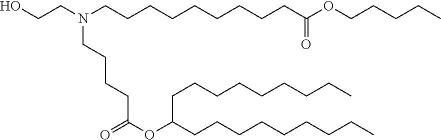 [SMILES: CCCCCCCCCC(CCCCCCCCC)OC(=O)CCCCN(CCO)CCCCCCCCCC(=O)OCCCCC]

Compound 45 was synthesized according to Representative Procedure 1 and general Methods A, B, C, and G. Product was obtained as a (mg, mmol, %). MS (ESI): m/z [M+H]⁺ 669.0 for C₄₁H₈₂NO₅;

### Pentyl 9-((2-hydroxyethyl) (4-(nonadecan-10-yloxy)-4-oxobutyl)amino) nonanoate (Compound 46)

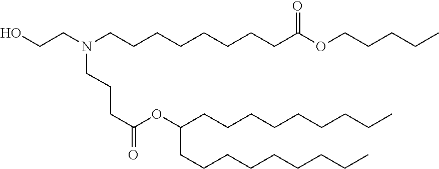 [SMILES: CCCCCCCCCC(CCCCCCCCC)OC(=O)CCCN(CCO)CCCCCCCCC(=O)OCCCCC]

Compound 46 was synthesized according to Synthesis Scheme 3 and general Methods A, B, C, and G. Product was obtained as a pale yellow oil (100 mg, 0.155 mmol, 24%). MS (ESI): m/z [M+H]⁺ 640.3 for C₃₉H₇₈NO₅; ¹H NMR (400 MHz, CDCl₃) δ 4.86 (qu, J=6.2 Hz, 1H), 4.05 (t, J=6.8 Hz, 2H), 3.62 (t, J=5.2 Hz, 2H), 2.70 (t, J=5.1 Hz, 2H), 2.63-2.54 (m, 4H), 2.34-2.26 (m, 4H), 1.84 (p, J=7.2 Hz, 2H), 1.65-1.57 (m, 4H), 1.51-1.50 (m, 4H), 1.34-1.25 (m, 41H), 0.92-0.85 (m, 9H); ¹³C NMR (100 MHz, CDCl₃) δ 174.1, 173.2, 74.8, 64.5, 58.2, 56.1, 54.1, 53.3, 34.5, 34.2, 32.1, 32.0, 29.7, 29.4, 29.4, 29.4, 29.2, 28.5, 28.2, 27.4, 26.4, 25.5, 25.1, 22.8, 22.5, 21.9, 14.2, 14.1.

### Pentyl 10-((2-hydroxyethyl) (4-(nonadecan-10-yloxy)-4-oxobutyl)amino) decanoate (Compound 47)

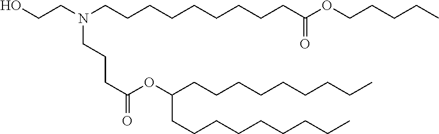 [SMILES: CCCCCCCCCC(CCCCCCCCC)OC(=O)CCCN(CCO)CCCCCCCCCC(=O)OCCCCC]

Compound 47 was synthesized according to Representative Procedure 1 and general Methods A, B, C, and G. Product was obtained as a pale yellow oil (210 mg, 0.318 mmol, 49%). MS (ESI): m/z [M+H]⁺ 654.6 for C₄₀H₈₀NO₅; ¹H NMR (400 MHz, CDCl₃) δ 4.86 (qu, J=6.2 Hz, 1H), 4.05 (t, J=6.8 Hz, 2H), 3.60 (bs, 2H), 2.68 (bs, 2H), 2.59-2.54 (m, 4H), 2.34-2.27 (m, 4H), 1.85-1.81 (m, 2H), 1.65-1.58 (m, 4H), 1.51-1.50 (m, 6H), 1.34-1.25 (m, 43H), 0.93-0.86 (m, 9H); ¹³C NMR (100 MHz, CDCl₃) δ 174.1, 173.2, 74.8, 64.5, 58.3, 56.2, 54.1, 53.3, 34.5, 34.2, 32.2, 32.0, 29.7, 29.6, 29.6, 29.5, 29.4, 29.3, 28.5, 28.2, 27.5, 25.5, 25.1, 22.8, 22.5, 14.3, 14.1.

### Pentan-3-yl 9-((5-(heptadecan-9-yloxy)-5-oxopentyl) (2-hydroxyethyl)amino) nonanoate (Compound 48) (also referred to as (I) (d))

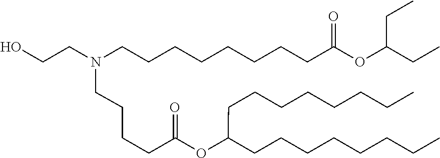 [SMILES: CCCCCCCCC(CCCCCCCC)OC(=O)CCCCN(CCO)CCCCCCCCC(=O)OC(CC)CC]

Compound 48 was synthesized according to Representative Procedure 1 and general Methods A, B, C, and G. Product was obtained as a pale yellow oil (365 mg, 0.583 mmol, 56%). MS (ESI): m/z [M+H]⁺ 627.0 for C₃₈H₇₆NO₅; ¹H NMR (600 MHz, CDCl₃) δ 4.56 (qu, J=6.2 Hz, 1H), 4.78-4.72 (m, 1H) p, 3.51 (t, J=5.4 Hz, 2H), 2.56 (t, J=5.4 Hz, 2H), 2.46 (dd, J=15.2, 7. Hz, 2H), 2.42 (dd, J=15.4, 7.8 Hz, 2H), 2.29 (td, J=7.5, 4.9 Hz, 4H), 1.66-1.59 (m, 4H), 1.59-1.52 (m, 4H), 1.52-1.45 (m, 7H), 1.45-1.38 (m, 2H), 1.35-1.18 (m, 38H), 0.87 (t, J=7.5 Hz, 12H); ¹³C NMR (150 MHz, CDCl₃) δ 173.9, 173.5, 76.5, 74.4, 58.5, 55.7, 54.0, 53.6, 34.8, 34.6, 34.3, 32.0, 29.7, 29.6, 29.6, 29.4, 29.4, 29.3, 27.5, 27.3, 26.8, 26.6, 25.5, 25.3, 23.1, 22.8, 14.2, 9.7.

### Isopentyl 9-((5-(heptadecan-9-yloxy)-5-oxopentyl) (2-hydroxyethyl)amino) nonanoate (Compound 49)

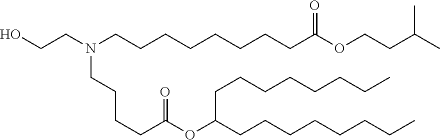 [SMILES: CCCCCCCCC(CCCCCCCC)OC(=O)CCCCN(CCO)CCCCCCCCC(=O)OCCC(C)C]

Compound 49 was synthesized according to Representative Procedure 1 and general Methods A, B, C, and G. Product was obtained as a pale yellow oil (398 mg, 0.636 mmol, 61%). MS (ESI): m/z [M+H]⁺ 627.0 for C₃₈H₇₆NO₅; ¹H NMR (600 MHz, CDCl₃) δ 4.86 (qu, J=6.2 Hz, 1H), 4.08 (t, J=6.9 Hz, 1H), 3.51 (t, J=5.4 Hz, 2H), 2.56 (t, J=5.4 Hz, 2H), 2.46 (dd, J=13.3, 5.9 Hz, 2H), 2.44-2.39 (m, 2H), 2.28 (dt, J=11.7, 7.5 Hz, 4H), 1.66-1.59 (m, 4H), 1.59-1.52 (m, 4H), 1.52-1.45 (m, 8H), 1.45-1.38 (m, 2H), 1.35-1.18 (m, 38H), 0.91 (d, J=6.7 Hz, 6H), 0.87 (t, J=7.5 Hz, 6H); ¹³C NMR (150 MHz, CDCl₃) δ 174.0, 173.4, 74.4, 63.0, 58.5, 55.7, 53.9, 53.6, 37.5, 34.6, 34.5, 34.3, 32.0, 30.0, 29.6, 29.5, 29.4, 29.4, 29.3, 27.5, 27.3, 26.8, 25.5, 25.2, 25.1, 23.1, 22.8, 22.6, 14.2.

### Pentyl 9-((5-(heptadecan-9-yloxy)-5-oxopentyl) (2-hydroxypropyl)amino) nonanoate (Compound 50)

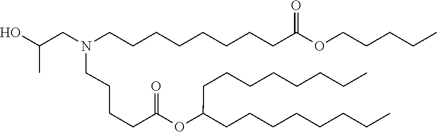 [SMILES: CCCCCCCCC(CCCCCCCC)OC(=O)CCCCN(CCCCCCCCC(=O)OCCCCC)CC(C)O]

Compound 50 was synthesized according to Representative Procedure 1 and general Methods A, B, C, and G. Product was obtained as a colorless oil (410 mg, 0,641 mmol, 64%). MS (ESI); m/z [M+H]⁺ 641.3 for C₃₉H₇₈NO₅; ¹H-NMR (600 MHz, CDCl₃) δ 4.87 (qu, J=6.2 Hz, 1H), 4.06 (t, J=6.8 Hz, 2H), 3.71 (n, J=3.0 Hz, 1H), 3.59 (bs, 1H), 2.56-2.48 (m, 2H), 2.43-2.38 (m, 2H), 2.36 (dd, J=12.5, 2.9 Hz, 1H), 2.29 (t, J=7.3 Hz, 4H), 2.21 (t, J=11.5 Hz, 1H), 1.66-1.57 (m, 6H), 1.51-1.48 (m, 8H), 1.36-1.26 (m, 36H), 1.11 (d, J=6.1 Hz, 3H), 0.91 (t, J=7.0 Hz, 3H), 0.88 (t, J=7.1 Hz, 6H); ¹³C NMR (150 MHz, CDCl₃) δ 173.9, 173.3, 74.2, 64.4 63.0, 62.4, 54.2, 53.9, 34.5, 34.4, 34.1 (2×), 31.8 (2×), 29.5 (2×), 29.5 (2×), 29.4, 29.3, 29.2 (2×), 29.1, 28.3, 28.1, 27.4, 27.2, 26.7, 25.3 (2×), 25.0, 23.0, 22.6 (2×), 22.3, 19.8, 14.1 (2×), 13.9.

### Pentyl 9-((5-(heptadecan-9-yloxy)-5-oxopentyl)((2R)-2-hydroxypropyl)amino) nonanoate (Compound 51)

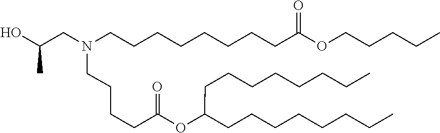 [SMILES: CCCCCCCCC(CCCCCCCC)OC(=O)CCCCN(CCCCCCCCC(=O)OCCCCC)C[C@@H](C)O]

Compound 51 was synthesized according to Representative Procedure 1 and general Methods A, B, C, and G. Product was obtained as a colorless oil (150 mg, 0,234 mmol, 71%). MS (ESI): m/z [M+H]⁺ 641.1 for C₃₉H₇₈NO₅; [α]_{D}²⁵=−23.8 (c 6.15, MTBE); ¹H NMR (600 MHz, 4.87 (qu, J=6.2 Hz, 1H), 4.06 (t, J=6.8 Hz, 2H), 3.71 (dtd, J=16.5, 6.1, 3.0 Hz, 1H), 3.59 (bs, 1H), 2.56-2.48 (m, 2H), 2.43-2.38 (m, 2H), 2.36 (dd, J=12.5, 2.9 Hz, 1H), 2.30 (t, J=7.3 Hz, 2H), 2.29 (t, J=7.3 Hz, 2H), 2.21 (dd, J=12.6, 10.5 Hz, 1H), 1.66-1.57 (m, 6H), 1.51-1.48 (m, 8H), 1.36-1.26 (m, 36H), 1.11 (d, J=6.1 Hz, 3H), 0.91 (t, J=7.0 Hz, 3H), 0.88 (t, J=7.0 Hz, 6H); ¹³C NMR (150 MHz, CDCl₃) δ 173.9, 173.3, 74.2, 64.3, 63.0, 62.4, 54.2, 53.9, 34.5, 34.3, 34.1 (2×), 31.8 (2×), 29.5 (2×), 29.5 (2×), 29.4, 29.2, 29.2 (2×), 29.1, 28.3, 28.1, 27.4, 27.2, 26.7, 25.3 (2×), 25.0, 23.0, 22.6 (2×), 22.3, 19.8, 14.1 (2×), 13.9.

### Pentyl 9-((5-(heptadecan-9-yloxy)-5-oxopentyl)((2S)-2-hydroxypropyl)amino) nonanoate (Compound 52)

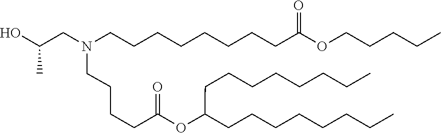 [SMILES: CCCCCCCCC(CCCCCCCC)OC(=O)CCCCN(CCCCCCCCC(=O)OCCCCC)C[C@H](C)O]

Compound 52 was synthesized according to Representative Procedure 1 and general Methods A, B, C, and G. Product was obtained as a colorless oil (150 mg, 0,234 mmol, 71%). MS (ESI): m/z [M+H]⁺ 641.1 for C₃₉H₇₈NO₅; [α]_{D}²⁵=+23.8 (c 7.15, MTBE); ¹H NMR (600 MHz, CDCl₃) δ 4.87 (qu, J=6.2 Hz, 1H), 4.06 (t, J=6.8 Hz, 2H), 3.71 (dtd, J=16.5, 6.1, 3.0 Hz, 1H), 3.59 (bs, 1H), 2.56-2.48 (m, 2H), 2.43-2.38 (m, 2H), 2.36 (dd, J=12.5, 2.9 Hz, 1H), 2.30 (t, J=7.3 Hz, 2H), 2.29 (t, J=7.3 Hz, 2H), 2.21 (dd, J=12.6, 10.5 Hz, 1H), 1.66-1.57 (m, 6H), 1.51-1.48 (m, 8H), 1.36-1.26 (m, 36H), 1.11 (d, J=6.1 Hz, 3H), 0.91 (t, J=7.0 Hz, 3H), 0.88 (t, J=7.0 Hz, 6H); ¹³C NMR (150 MHz, CDCl₃) δ 173.9, 173.3, 74.2, 64.4, 63.0, 62.4, 54.2, 53.9, 34.5, 34.4, 34.1 (2×), 31.8 (2×), 29.5 (2×), 29.5 (2×), 29.4, 29.3, 29.2 (2×), 29.1, 28.3, 28.1, 27.4, 27.2, 26.7, 25.3 (2×), 25.0, 23.0, 22.6 (2×), 22.3, 19.8, 14.1 (2×), 13.9.

### Pentyl 9-((4-aminobutyl) (5-(heptadecan-9-yloxy)-5-oxopentyl)amino) nonanoate (Compound 53) (Method H)

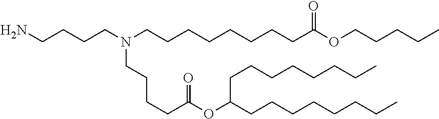 [SMILES: CCCCCCCCC(CCCCCCCC)OC(=O)CCCCN(CCCCN)CCCCCCCCC(=O)OCCCCC]

To a solution of pentyl 9-((4-((tert-butoxycarbonyl)amino)butyl) (5-(heptadecan-9-yloxy)-5-oxopentyl)amino) nonanoate (Intermediate E) (0.95 g, 1.26 mmol, 1 eq) in dioxane (1 mL) cooled to 0° C. a 4N HCl solution in dioxane (11.0 mL, 44.1 mmol, 35 eq) was added for 10 minutes. The reaction mixture was stirred for 20 hours at room temperature. Then the solvent and volatiles were evaporated to dryness. Product was purified by silica gel chromatography (Phase A: t-BuOMe; Phase B t-BuOMe:MeOH:NH₄OH=50:50:1; 100% A to A:B=50:50) to obtain pentyl 9-((4-aminobutyl) (5-(heptadecan-9-yloxy)-5-oxopentyl)amino) nonanoate (700 mg, 1.07 mmol, 85%) as a white wax. MS (ESI): m/z [M+H]⁺ 654.0 for C₄₀H₈₁N₂O₄; ¹H NMR (600 MHz, CDCl₃) δ 6.08 (bs, 2H), 4.85 (p, J=6.2 Hz, 1H), 4.05 (t, J=6.8 Hz, 2H), 2.93 (t, J=6.1 Hz, 2H), 2.60-2.60 (m, 6H, 3×CH2N), 2.34 (t, J=7.2 Hz, 2H), 2.29 (t, J=7.5 Hz, 2H), 1.82 (p, J=6.2 Hz, 2H), 1.71 (p, J=6.3 Hz, 2H), 1.61 (m, 8H), 1.51 (m, 6H), 1.26-1.26 (m, 36H), 0.91 (t, J=7.1 Hz, 3H), 0.88 (t, J=7.0 Hz, 6H); ¹³C NMR (150 MHz, CDCl₃) δ 174.0, 173.2, 74.5, 64.4, 53.3, 52.8, 52.3, 40.0, 34.3, 34.2, 34.1, 31.8, 29.5, 29.5, 29.2, 29.2, 29.2, 29.0, 28.3, 28.1, 27.5, 27.4, 25.3, 24.9, 24.8, 24.6, 24.4, 23.0, 22.6, 22.3, 14.1 (2×), 13.9.

### Pentyl 9-((5-(heptadecan-9-yloxy)-5-oxopentyl) (4-((2-(methylamino)-3,4-dioxocyclobut-1-en-1-yl)amino)butyl)amino) nonanoate (Compound 54) (Method I)

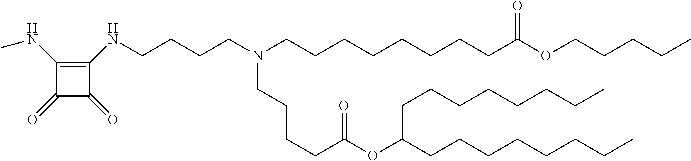 [SMILES: CCCCCCCCC(CCCCCCCC)OC(=O)CCCCN(CCCCCCCCC(=O)OCCCCC)CCCCNc1c(NC)c(=O)c1=O]

To a solution of pentyl 9-((4-aminobutyl) (5-(heptadecan-9-yloxy)-5-oxopentyl)amino) nonanoate (Compound 53) (300 mg, 0.459 mmol, 1 eq) in ethanol (10 mL) 3-methoxy-4-(methylamino)cyclobut-3-ene-1,2-dione (Intermediate F) (79 mg, 0.505 mmol, 1.1 eq) was added. The reaction mixture was stirred for 20 hours at 40° C. A solvent was evaporated to dryness and the crude product was purified by silica gel chromatography (Phase A: t-BuOMe; Phase B t-BuOMe:MeOH:NH₄OH=50:50:1; 100% A to A:B=50:50) to obtain pentyl 9-((5-(heptadecan-9-yloxy)-5-oxopentyl) (4-((2-(methylamino)-3,4-dioxocyclobut-1-en-1-yl)amino)butyl)amino) nonanoate (Compound 54) (220 mg, 0.289 mmol, 63%) as yellow wax. MS (ESI): m/z [M+H]⁺ 763.2 for C₄₅H₈₄N₃O₆; ¹H NMR (600 MHz, benzene-d₆) δ 8.47 (bs, 1), 8.16 (bs, 1), 5.19-5.14 (m, 1H), 4.08 (t, J=6.7 Hz, 2H), 3.86 (bs, 2H), 3.74 (bs, 1H), 3.32 (d, J=4.7 Hz, 2H), 2.50 (t, J=6.8 Hz, 2H), 2.43 (t, J=7.2 Hz, 4H), 2.37 (t, J=7.4 Hz, 2H), 2.27 (t, J=7.5 Hz, 2H), 1.85-1.85 (m, 2H), 1.77-1.77 (m, 2H), 1.67-1.67 (m, 6H), 1.54-1.54 (m, 8H), 1.33-1.33 (m, 32H), 1.23-1.23 (m, 4H), 0.97 (t, J=7.1 Hz, 6H), 0.87 (t, J=7.1 Hz, 3H); ¹³C NMR (150 MHz, benzene-d₆+CD₃OD) δ 183.8, 183.5, 174.7, 174.7, 174.3, 174.3, 169.3, 169.0, 128.7, 75.1, 65.1, 60.3, 54.7, 54.2, 44.7, 35.1, 35.0, 34.9, 32.7, 32.7, 31.0, 30.3, 30.3, 30.3, 30.2, 30.1, 30.1, 30.1, 29.8, 29.8, 29.1, 28.8, 28.3, 27.2, 26.6, 26.2, 25.7, 24.4, 23.9, 23.4, 23.0, 14.6, 14.5, 14.3.

### Pentyl 9-(3-aminopropyl-(5-(1-octylnonoxy)-5-oxo-pentyl)amino) nonanoate (Compound 55)

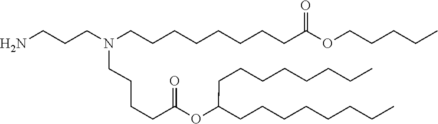 [SMILES: CCCCCCCCC(CCCCCCCC)OC(=O)CCCCN(CCCN)CCCCCCCCC(=O)OCCCCC]

Compound 55 was synthesized according to Representative Procedure 2 and general Methods A, C, D, E, and H. Product was obtained as a colorless oil (470 mg, 0,735 mmol, 86%). MS (ESI): m/z [M+H]⁺ 640.1 for C₃₉H₇₉N₂O₄; ¹H NMR (600 MHz, CDCl₃) δ 4.86 (qu, 1H, J=6.3, 1.0 Hz), 4.06 (t, J=6.8 Hz, 2H), 2.74 (t, J=6.8 Hz, 2H), 2.45 (t, J=7.1 Hz, 2H), 2.41 (t, J=7.5 Hz, 2H), 2.37 (t, J=7.6 Hz, 2H), 2.29 (t, 7.2 Hz, 2H), 2.28 (t, J=7.2 Hz, 4H), 1.78 (s, 2H), 1.55-1.68 (m, 8H), 1.37-1.55 (m, 8H), 1.17-1.37 (m, 36H), 0.91 (t, J=7.1 Hz, 3H), 0.88 (t, J=7.1 Hz, 6H); ¹³C NMR (150 MHz, CDCl₃) δ 173.9, 173.4, 74.2, 64.4, 54.1, 53.7, 52.0, 40.9, 34.6, 34.4, 34.1, 31.8, 30.7, 29.5, 29.5, 29.4, 29.3, 29.2, 29.1, 28.3, 28.1, 27.5, 27.0, 26.5, 25.3, 25.0, 23.1, 22.6, 22.3, 14.1, 13.9.

### Pentyl 9-((5-(heptadecan-9-yloxy)-5-oxopentyl) (3-((2-(methylamino)-3,4-dioxocyclobut-1-en-1-yl)amino) propyl)amino) nonanoate (Compound 56)

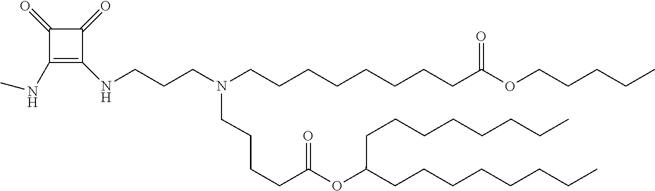 [SMILES: CCCCCCCCC(CCCCCCCC)OC(=O)CCCCN(CCCCCCCCC(=O)OCCCCC)CCCNc1c(NC)c(=O)c1=O]

Compound 56 was synthesized according to Representative Procedure 3 and general Method I. Product was obtained as a yellow wax (250 mg, 0,334 mmol, 97%). MS (ESI): m/z [M+H]⁺ 749.2 for C₄₄H₈₂N₃O₆; ¹H NMR (400 MHz, benzene-d₆) δ 8.40 (bs, 1H), 8.10 (bs, 1H), 5.20-5.10 (m, 1H), 4.08 (t, J=6.7 Hz, 2H), 3.82 (bs, 2H), 3.32 (d, J=4.7 Hz, 2H), 2.59 (t, J=6.2 Hz, 2H), 2.45 (t, J=7.0 Hz, 4H), 2.36 (t, J=7.3 Hz, 2H), 2.28 (t, J=7.5 Hz, 2H), 1.95 (dd, J=5.4 Hz, 2H), 1.13-1.84 (m, 50H), 0.96 (t, J=6.8 Hz, 6H), 0.86 (t, J=7.0 Hz, 3H); ¹³C NMR (100 MHz, benzene-d₆) δ 183.4, 183.0, 174.3, 173.9, 168.8, 168.5, 74.6, 64.6, 54.1, 53.7, 51.3, 42.9, 34.6, 34.5, 34.4, 32.2 (2×), 30.6, 29.9 (2×), 29.9 (2×), 29.8, 29.6, 29.6 (2×), 29.4, 28.6 (2×), 28.6, 28.3 (2×), 27.9, 26.7, 26.2, 25.8 (2×), 25.3, 23.4, 23.0 (2×), 22.6, 14.1 (2×), 13.9.

### Pentyl 9-((3-aminopropyl) (4-(heptadecan-9-yloxy)-4-oxobutyl)amino) nonanoate (Compound 57)

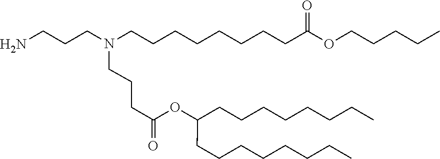 [SMILES: CCCCCCCCC(CCCCCCCC)OC(=O)CCCN(CCCN)CCCCCCCCC(=O)OCCCCC]

Compound 57 was synthesized according to Representative Procedure 2 and general Methods A, C, D, E, and H. Product was obtained as a colorless oil (1140 mg, 1.82 mmol, 83%). MS (ESI): m/z [M+H]⁺ 626.0 for C₃₈H₇₇N₂O₄; ¹H NMR (600 MHz, CDCl₃) δ 4.86 (qu, J=6.3, 1.0 Hz, 1H), 4.06 (t, J=6.8 Hz, 2H), 2.73 (t, J=6.8 Hz, 2H), 2.45 (t, J=7.1 Hz, 2H), 2.42 (t, J=7.3 Hz, 2H), 2.38 (t, J=7.6 Hz, 2H), 2.28 (t, J=7.7 Hz, 1H), 2.27 (t, J=7.7 Hz, 1H), 1.83 (bs, 2H), 1.74 (q, J=7.4, 2H), 1.65-1.56 (m, 6H), 1.51-1.50 (m, 4H), 1.43-1.38 (m, 2H), 1.35-1.26 (m, 36H), 0.89 (t, J=7.1, 3H), 0.86 (t, J=7.1, 6H); ¹³C NMR (150 MHz, CDCl₃) δ 173.9, 173.5, 74.2, 64.3, 54.1, 53.3, 51.9, 50.5, 40.7, 34.3, 34.1 (2×), 32.4, 31.8 (2×), 30.8, 29.5 (2×), 29.5 (2×), 29.4, 29.3, 29.2 (2×), 29.1, 28.3, 28.0, 27.5, 27.0, 25.3 (2×), 25.0, 22.6 (2×), 22.6, 22.3, 14.0 (2×), 13.9.

### Pentyl 9-((4-(heptadecan-9-yloxy)-4-oxobutyl) (3-((2-(methylamino)-3,4-dioxocyclobut-1-en-1-yl)amino) propyl)amino) nonanoate (Compound 58)

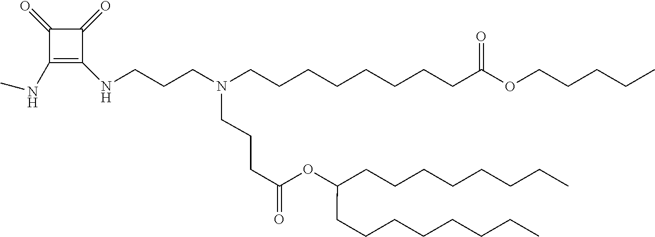 [SMILES: CCCCCCCCC(CCCCCCCC)OC(=O)CCCN(CCCCCCCCC(=O)OCCCCC)CCCNc1c(NC)c(=O)c1=O]

Compound 58 was synthesized according to Representative Procedure 3 and general Method I. Product was obtained as a yellow wax (345 mg, 0,470 mmol, 98%). MS (ESI): m/z [M+H]⁺ 735.1 for C₄₃H₈₀N₃O₆; ¹H NMR (600 MHz, CDCl₃) δ 7.21 (bs, 1H), 6.76 (bs, 1H), 4.82 (qu, J=6.3 Hz, 1H), 4.05 (t, J=6.8 Hz, 2H), 3.72 (bs, 2H), 3.31 (d, J=5.0 Hz, 3H), 2.52 (t, J=6.3 Hz, 2H), 2.42 (t, J=6.9 Hz, 2H), 2.39 (t, J=7.7 Hz, 2H), 2.36 (t, J=7.3 Hz, 2H), 2.29 (t, J=7.5 Hz, 2H), 1.79-1.74 (m, 4H), 1.65-1.59 (m, 4H), 1.53-1.52 (m, 4H), 1.41-1.26 (m, 38H), 0.91 (t, J=7.1 Hz, 3H), 0.88 (t, J=7.0 Hz, 6H); ¹³C NMR (150 MHz, CDCl₃-CD₃OD=2:1) δ 183.4, 182.9, 175.2, 174.6, 169.0, 168.7, 75.4, 65.1, 54.4, 53.5, 51.5, 43.2, 34.8, 34.6 (2×), 32.9, 32.3 (2×), 31.2, 30.0 (2×), 29.9, 29.7 (2×), 29.7, 29.5, 28.8, 28.6, 28.0, 26.9, 25.8 (2×), 25.4, 23.1 (2×), 22.7, 22.5, 14.3 (2×), 14.1.

### Pentyl 6-((5-(heptadecan-9-yloxy)-5-oxopentyl) (3-((2-(methylamino)-3,4-dioxocyclobut-1-en-1-yl)amino) propyl)amino) hexanoate (Compound 59)

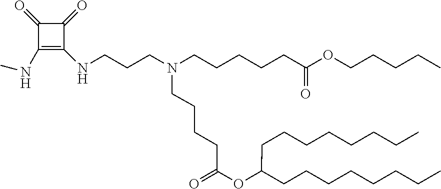 [SMILES: CCCCCCCCC(CCCCCCCC)OC(=O)CCCCN(CCCCCC(=O)OCCCCC)CCCNc1c(NC)c(=O)c1=O]

Compound 59 was synthesized according to Representative Procedures 2 and 3 and general Methods A, C, D, E, H, and I. Product was obtained as a pale yellow oil (590 mg, 0.827 mmol, 93%). MS (ESI): m/z [M+H]⁺ 707.1 for C₄₁H₇₆N₃O₆; ¹H NMR (600 MHz, CDCl₃-CD₃OD=2:1) δ 4.89-4.85 (m, 1H), 4.08 (t, J=6.8 Hz, 2H), 3.70 (t, J=6.3 Hz, 2H), 3.30 (bs, 3H), 3.16 (bs, 2H), 3.07 (bs, 4H), 2.41 (t, J=7.1 Hz, 2H), 2.36 (t, J=7.3 Hz, 2H), 2.06-2.01 (m, 2H), 1.76-1.62 (m, 10H), 1.55-1.53 (m, 4H), 1.44-1.38 (m, 2H), 1.36-1.33 (m, 4H), 1.32-1.25 (m, 26H), 0.92 (t, J=7.1 Hz, 3H), 0.89 (t, J=7.1 Hz, 6H); ¹³C NMR (150 MHz, CDCl₃-CD₃OD=2:1) δ 183.0, 182.9, 174.3, 173.6, 169.8, 167.9, 75.5, 65.2, 53.2, 53.0, 51.1, 41.2, 34.4, 34.1, 33.9, 32.2, 31.1, 29.8, 29.8, 29.6, 28.6, 28.4, 26.4, 26.2, 25.7, 24.6, 23.8, 23.5, 23.0, 22.6, 22.3, 14.2, 14.1.

### Pentyl 6-((5-(heptadecan-9-yloxy)-5-oxopentyl) (4-((2-(methylamino)-3,4-dioxocyclobut-1-en-1-yl)amino)butyl)amino) hexanoate (Compound 60)

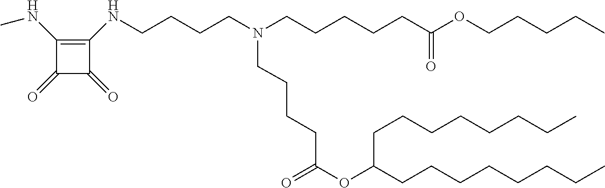 [SMILES: CCCCCCCCC(CCCCCCCC)OC(=O)CCCCN(CCCCCC(=O)OCCCCC)CCCCNc1c(NC)c(=O)c1=O]

Compound 60 was synthesized according to Representative Procedures 2 and 3 and general Methods A, C, D, E, H, and I. Product was obtained as a pale yellow oil (521 mg, 0.716 mmol, 100%). MS (ESI): m/z [M+H]⁺ 721.2 for C₄₂H₇₈N₃O₆; ¹H NMR (600 MHz, CDCl₃-CD₃OD=2:1) δ 4.90-4.86 (m, 1H), 4.08 (t, J=6.8 Hz, 2H), 3.67 (t, J=6.4 Hz, 2H), 3.29 (bs, 3H), 3.12-3.04 (m, 6H), 2.41 (t, J=7.1 Hz, 2H), 2.37 (t, J=7.3 Hz, 2H), 1.84-1.79 (m, 2H), 1.77-1.62 (m, 12H), 1.55-1.53 (m, 4H), 1.44-1.39 (m, 2H), 1.38-1.33 (m, 4H), 1.33-1.25 (m, 26H), 0.92 (t, J=7.1 Hz, 3H), 0.89 (t, J=7.1 Hz, 6H); ¹³C NMR (150 MHz, CDCl₃-CD₃OD=2:1) δ 182.9, 182.8, 174.3, 173.6, 169.6, 168.2, 75.5, 65.2, 53.2, 53.1, 53.0, 43.0, 34.4, 34.1, 33.9, 32.2, 31.0, 29.8, 29.8, 29.6, 28.6, 28.4, 28.2, 26.4, 25.7, 24.6, 23.8, 23.5, 23.0, 22.6, 22.3, 21.2, 14.2, 14.1;

### Butyl 11-((5-(heptadecan-9-yloxy)-5-oxopentyl) (3-((2-(methylamino)-3,4-dioxocyclobut-1-en-1-yl)amino) propyl)amino) undecanoate (Compound 61)

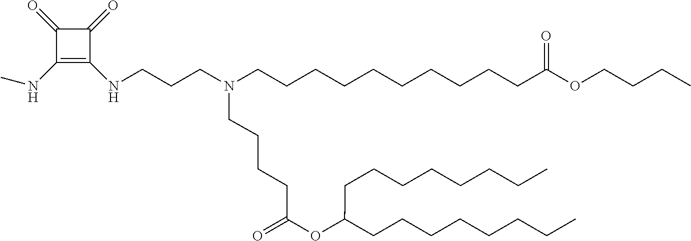 [SMILES: CCCCCCCCC(CCCCCCCC)OC(=O)CCCCN(CCCCCCCCCCC(=O)OCCCC)CCCNc1c(NC)c(=O)c1=O]

Compound 61 was synthesized according to Representative Procedures 2 and 3 and general Methods A, C, D, E, H, and I. Product was obtained as a white solid (230 mg, 0.30 mmol, 45%). MS (ESI): m/z [M+H]⁺ 763.3 for C₄₅H₈₄N₃O₆; ¹H NMR (400 MHz, CDCl₃) δ 4.81 (p, J=6.3 Hz, 1H), 4.06 (t, J=6.7 Hz, 2H), 3.80-3.65 (m, 2H), 3.29 (d, J=5.1 Hz, 3H), 2.53 (t, J=5.3 Hz, 2H), 2.45 (t, J=6.7 Hz, 2H), 2.38 (dt, J=14.0, 7.1 Hz, 4H), 2.28 (t, J=6.7 Hz, 4H), 1.81-1.72 (m, 2H), 1.68-1.57 (m, 6H), 1.56-1.45 (m, 6H), 1.44-1.35 (m, 4H), 1.33-1.19 (m, 36H), 0.93 (t, J=7.4 Hz, 3H), 0.87 (t, J=6.9 Hz, 6H); ¹³C NMR (100 MHz, CDCl₃-CD₃OD=2:1) δ 182.8, 182.7, 174.0, 169.0, 167.9, 74.9, 64.2, 53.4, 53.1, 34.4, 34.1, 31.9, 31.3, 29.6, 29.6, 29.6, 29.5, 29.4, 29.3, 29.3, 29.2, 28.4, 28.4, 27.3, 25.4, 25.1, 22.7, 19.2, 14.2, 13.8.

### Pentyl 11-((5-(heptadecan-9-yloxy)-5-oxopentyl) (3-((2-(methylamino)-3,4-dioxocyclobut-1-en-1-yl)amino) propyl)amino) undecanoate (Compound 62)

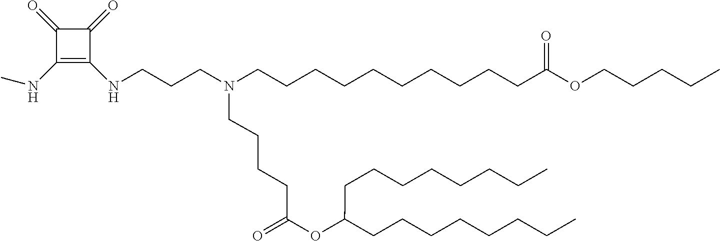 [SMILES: CCCCCCCCC(CCCCCCCC)OC(=O)CCCCN(CCCCCCCCCCC(=O)OCCCCC)CCCNc1c(NC)c(=O)c1=O]

Compound 62 was synthesized according to Representative Procedures 2 and 3 and general Methods A, C, D, E, H, and I. Product was obtained as a pale yellow solid (349 mg, 0.45 mmol, 94%). MS (ESI): m/z [M+H]⁺ 777.3 for C₄₆H₈₆N₃O₆; ¹H NMR (400 MHz, CDCl₃) δ 4.82 (p, J=6.2 Hz, 1H), 4.05 (t, J=6.8 Hz, 2H), 3.78-3.65 (m, 2H), 3.29 (d, J=5.0 Hz, 3H), 2.62 (t, J=5.8 Hz, 2H), 2.57-2.43 (m, 4H), 2.34 (t, J=7.0 Hz, 2H), 2.28 (t, J=7.5 Hz, 2H), 1.84-1.78 (m, 2H), 1.69-1.57 (m, 6H), 1.54-1.40 (m, 8H), 1.33-1.18 (m, 40H), 0.90 (t, J=5.9 Hz, 3H), 0.87 (t, J=6.7 Hz, 6H); ¹³C NMR (100 MHz, CDCl₃-CD₃OD=2:1) δ 183.3, 183.2, 174.1, 168.8, 168.1, 75.0, 64.5, 53.4, 53.2, 34.5, 34.2, 34.1, 31.9, 31.3, 29.6, 29.6, 29.6, 29.5, 29.3, 29.3, 29.2, 28.4, 28.4, 28.2, 27.6, 25.8, 25.4, 25.1, 22.9, 22.7, 22.4, 20.2, 14.2, 14.0.

### Butyl 12-((5-(heptadecan-9-yloxy)-5-oxopentyl) (3-((2-(methylamino)-3,4-dioxocyclobut-1-en-1-yl)amino) propyl)amino) dodecanoate (Compound 63)

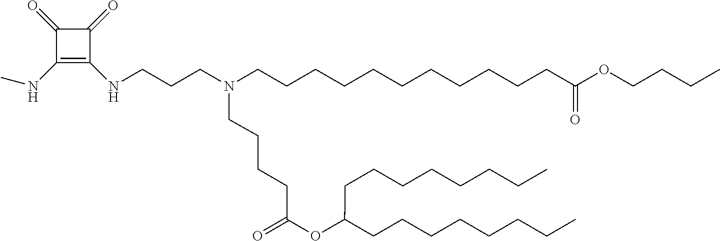 [SMILES: CCCCCCCCC(CCCCCCCC)OC(=O)CCCCN(CCCCCCCCCCCC(=O)OCCCC)CCCNc1c(NC)c(=O)c1=O]

Compound 63 was synthesized according to Representative Procedures 2 and 3 and general Methods A, C, D, E, H, and I. Product was obtained as a pale yellow oil (832 mg, 1.072 mmol, 70%). MS (ESI): m/z [M+H]⁺ 776.7 for C₄₆H₈₆N₃O₆; ¹H NMR (600 MHz, CDCl₃—CD₃OD=2:1) δ 4.90-4.84 (m, 1H), 4.08 (t, J=6.7 Hz, 2H), 3.63 (bs, 2H), 3.28 (s, 3H), 2.55-2.51 (m, 2H), 2.51-2.47 (m, 2H), 2.47-2.42 (m, 2H), 2.35 (t, J=7.4 Hz, 2H), 2.31 (td, J=7.6, 3.4 Hz, 2H), 1.80-1.73 (m, 2H), 1.66-1.58 (m, 6H), 1.57-1.48 (m, 6H), 1.48-1.36 (m, 4H), 1.34-1.23 (m, 40H), 0.95 (t, J=7.4 Hz, 3H), 0.89 (t, J=7.1 Hz, 6H); ¹³C NMR (150 MHz, CDCl₃-CD₃OD=2:1) δ 183.3, 182.8, 175.1, 174.5, 168.9, 168.5, 75.2, 64.7, 54.2, 53.8, 51.4, 43.1, 34.8, 34.7, 34.4, 32.2, 31.1, 31.0, 30.0, 29.9, 29.8, 29.8, 29.8, 29.6, 29.6, 29.4, 28.5, 28.0, 26.5, 26.0, 25.7, 25.3, 23.4, 23.0, 19.4, 14.2, 13.8.

### Pentyl 12-((5-(heptadecan-9-yloxy)-5-oxopentyl) (3-((2-(methylamino)-3,4-dioxocyclobut-1-en-1-yl)amino) propyl)amino) dodecanoate (Compound 64)

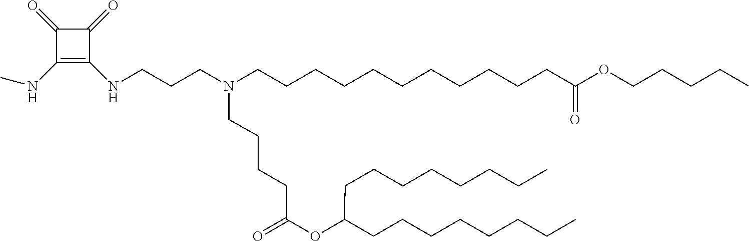 [SMILES: CCCCCCCCC(CCCCCCCC)OC(=O)CCCCN(CCCCCCCCCCCC(=O)OCCCCC)CCCNc1c(NC)c(=O)c1=O]

Compound 64 was synthesized according to Representative Procedures 2 and 3 and general Methods A, C, D, E, H, and I. Product was obtained as a pale yellow oil (246 mg, 0.311 mmol, 85%). MS (ESI): m/z [M+H]⁺ 790.6 for C₄₇H₈₈N₃O₆; ¹H NMR (600 MHz, CDCl₃-CD₃OD=2:1) δ 4.80 (qu, J=6.2 Hz, 1H), 4.05 (t, J=6.8 Hz, 2H), 3.74 (bs, 2H), 3.29 (d, J=5.0 Hz, 3H), 2.53-2.47 (m, 2H), 2.43 (t, J=6.5 Hz, 2H), 2.36 (t, J=7.0 Hz, 4H), 2.28 (td, J=7.6, 2.8 Hz, 2H), 1.75 (dt, J=11.1, 5.6 Hz, 2H), 1.67-1.57 (m, 6H), 1.55-1.50 (m, 4H), 1.49-1.43 (m, 2H), 1.43-1.37 (m, 2H), 1.36-1.20 (m, 44H), 0.92-0.86 (m, 9H).

### Pentyl 9-((5-(heptadecan-9-yloxy)-5-oxopentyl)((4-((1H-imidazol-5-yl) formamido)butyl)) amino) nonanoate (Compound 65) (Method J)

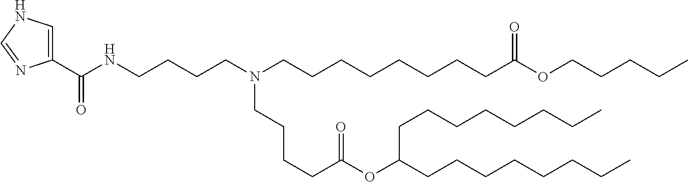 [SMILES: CCCCCCCCC(CCCCCCCC)OC(=O)CCCCN(CCCCCCCCC(=O)OCCCCC)CCCCNC(=O)c1c[nH]cn1]

A mixture of 4-imidazolecarboxylic acid (33 mg, 0.286 mmol, 1.1 eq), oxalyl chloride (0.57 mL, 6.51 mmol, 25 eq) and one drop of dimethylformamide was stirred for 3 hours at room temperature. Then, the volatiles were evaporated to dryness and to the residue dichloromethane (2 mL) and a solution of pentyl 9-((4-aminobutyl) (5-(heptadecan-9-yloxy)-5-oxopentyl)amino) nonanoate (Compound 53) (170 mg, 0.26 mmol, 1.0 eq) in dichloromethane (2 mL) and DIPEA (0.227 mL, 1.30 mmol, 5.0 eq) were added. The reaction mixture was stirred for 20 hours at room temperature. Then, to the reaction mixture water (40 mL) and ethyl acetate (10 mL) were added. Phases were separated and the aqueous phase was extracted with ethyl acetate (2×20 mL). Combined organic phases were dried with sodium sulfate and evaporated to dryness to obtain crude yellow oil (230 mg). The product was purified by silica gel chromatography (Phase A: t-BuOMe; Phase B: t-BuOMe:MeOH:NH₄OH=50:50:1; 100% A to A:B=50:50) to obtain pentyl 9-((5-(heptadecan-9-yloxy)-5-oxopentyl)((4-((1H-imidazol-5-yl) formamido)butyl)) amino) nonanoate (Compound 65) (160 mg, 0.214 mmol, 82%) as a yellow oil. MS (ESI): m/z [M+H]⁺ 748.2 for C₄₄H₈₃N₄O₅; ¹H NMR (600 MHz, CDCl₃) δ11.37 (bs, 1H), 7.58 (s, 1H), 7.57 (s, 1H), 7.28 (bs, 1H), 4.88-4.83 (m, 1H), 4.06 (t, J=6.8 Hz, 2H), 3.44 (dt, J=6.8, 6.3 Hz, 2H), 2.41 (t, J=7.9 Hz, 2H), 2.40 (t, J=7.9 Hz, 2H), 2.38-2.34 (m, 2H), 2.30-2.26 (m, 4H), 1.25-1.25 (m, 54H), 0.90 (t, J=7.1 Hz, 3H), 0.87 (t, J=7.0 Hz, 6H); ¹³C NMR (150 MHz, CDCl₃) δ 174.1, 173.5, 163.2, 136.7, 135.0, 118.7, 74.2, 64.4, 54.1, 53.7, 53.6, 38.9, 34.6, 34.4, 34.1, 31.8, 29.5, 29.5, 29.4, 29.3, 29.2, 29.1, 28.3, 28.1, 27.7, 27.6, 27.0, 26.5, 25.3, 25.0, 24.6, 23.2, 22.6, 22.3, 14.1, 13.9.

### Pentyl 9-((4-(heptadecan-9-yloxy)-4-oxobutyl)((3-((1H-imidazol-4-yl) formamido) propyl))amino) nonanoate (Compound 66)

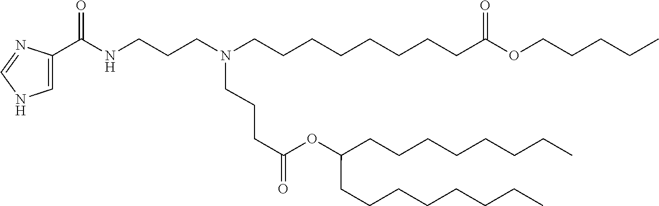 [SMILES: CCCCCCCCC(CCCCCCCC)OC(=O)CCCN(CCCCCCCCC(=O)OCCCCC)CCCNC(=O)c1c[nH]cn1]

Compound 66 was synthesized according to Representative Procedures 2 and 4 and general Methods A, C, D, E, H, and J. Product was obtained as a yellow tight oil (210 mg, 0,292 mmol, 91%). MS (ESI): m/z [M+H]⁺ 720.2 for C₄₂H₇₉N₄O₅; ¹H NMR (600 MHz, CDCl₃) δ 11.3 (bs, 1H), 8.10 (s, 1H), 7.71 (bs, 1H), 7.58 (s, 1H), 7.56 (s, 0.9H), 4.88-4.83 (m, 1H), 4.30-4.25 (m, 1H), 4.06 (t, J=6.8 Hz, 2H), 3.48 (dt, J=6.3, 6.5 Hz, 2H), 2.53 (t, J=6.5 Hz, 2H), 2.45 (t, J=7.3 Hz, 2H), 2.40 (t, J=7.6 Hz, 2H), 2.32 (t, J=7.4 Hz, 2H), 2.28 (t, J=7.6 Hz, 2H), 1.78-1.78 (m, 4H), 1.61-1.61 (m, 4H), 1.50-1.42 (m, 6H), 1.33-1.33 (m, 4H), 1.25-1.25 (m, 32H), 0.91 (t, 3H), 0.86 (t, 6H); ¹³C NMR (150 MHz, CDCl₃) δ 174.1, 173.5, 165.9, 135.1, 134.2, 129.5, 74.3, 67.8, 64.4, 54.0, 53.3, 52.3, 38.9, 34.4, 34.1, 32.4, 31.8, 30.6, 29.5, 29.5, 29.4, 29.2, 29.2, 29.1, 28.3, 28.1, 27.5, 25.3, 25.0, 24.0, 22.9, 22.6, 22.3, 14.1, 13.9.

### Butyl 11-((5-(heptadecan-9-yloxy)-5-oxopentyl)((3-((1H-imidazol-4-yl) formamido) propyl))amino) undecanoate (Compound 67)

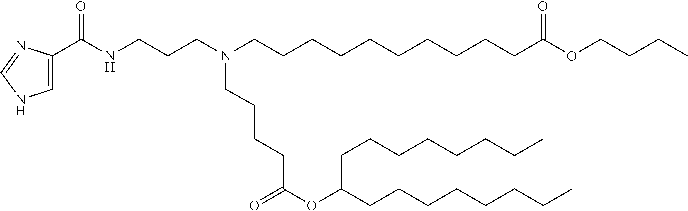 [SMILES: CCCCCCCCC(CCCCCCCC)OC(=O)CCCCN(CCCCCCCCCCC(=O)OCCCC)CCCNC(=O)c1c[nH]cn1]

Compound 67 was synthesized according to Representative Procedures 2 and 4 and general Methods A, C, D, E, H, and J. Product was obtained as a yellow tight oil (150 mg, 0,201 mmol, 66%). MS (ESI): m/z [M+H]⁺ 748.2 for C₄₄H₈₃N₄O₅; ¹H NMR (600 MHz, CDCl₃) δ 11.31 (bs, 1H), 7.73 (bs, 1H), 7.57 (bs, 1H), 7.54 (bs, 1H), 4.88-4.83 (m, 1H), 4.07 (t, J=6.7 Hz, 2H), 3.48 (dt, J=6.3, 6.3 Hz, 2H), 2.52 (t, J=6.2 Hz, 2H), 2.43 (t, J=7.3 Hz, 2H), 2.39 (t, J=7.5 Hz, 2H), 2.29 (t, J=7.5 Hz, 4H), 1.74 (q, J=6.7 Hz, 2H), 1.64-1.58 (m, 6H), 1.52-1.46 (m, 8H), 1.40-1.33 (m, 2H), 1.30-1.25 (m, 36H), 0.93 (t, J=7.4 Hz, 3H), 0.87 (t, J=7.1 Hz, 6H); ¹³C NMR (150 MHz, CDCl₃) δ 174.3, 173.6, 163.3, 136.9, 135.1, 118.7, 74.3, 64.2, 54.2, 53.8, 52.4, 38.2, 34.7, 34.5, 34.2, 31.9, 30.8, 29.7, 29.6, 29.6, 29.6, 29.5, 29.3, 29.2, 27.7, 27.0, 26.5, 25.4, 25.1, 23.3, 22.7, 19.2, 14.2, 13.8.

### Pentyl 6-((5-(heptadecan-9-yloxy)-5-oxopentyl) (2-hydroxyethyl)amino) hexanoate (Compound 68)

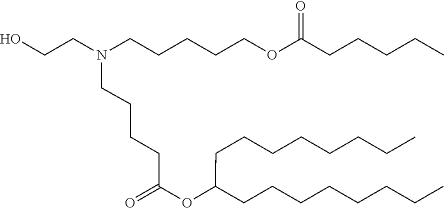 [SMILES: CCCCCCCCC(CCCCCCCC)OC(=O)CCCCN(CCO)CCCCCOC(=O)CCCCC]

Compound 68 was synthesized according to Representative Procedure 1 with using Intermediate G and general Methods F, B, C, and G. Product was obtained as a pale yellow oil (70 mg, 0.114 mmol, 23%). MS (ESI): m/z [M+H]⁺ 584.9 for C₃₅H₇₀NO₅; ¹H NMR (600 MHz, CDCl₃-CD₃OD=2:1) δ 4.90-4.85 (m, 1H), 4.08 (t, J=6.6 Hz, 2H), 3.65 (t, J=5.7 Hz, 2H), 2.74 (bs, 2H), 2.63 (bs, 3H), 2.36 (t, J=7.3 Hz, 2H), 2.36 (t, J=7.3 Hz, 2H), 1.70-1.61 (m, 6H), 1.59-1.52 (m, 8H), 1.40-1.34 (m, 2H), 1.34-1.25 (m, 29H), 0.91 (t, J=7.1 Hz, 3H), 0.89 (t, J=7.0 Hz, 6H); ¹³C NMR (150 MHz, CDCl₃-CD₃OD=2:1) δ 175.0, 174.1, 75.3, 64.5, 55.7, 54.2, 54.0, 34.6, 34.5, 34.4, 32.2, 31.6, 29.8, 29.8, 29.6, 28.7, 25.7, 25.0, 24.2, 23.9, 23.0, 22.9, 14.2, 14.0.

### Pentan-3-yl 9-((5-(heptadecan-9-yloxy)-5-oxopentyl) (3-((2-(methylamino)-3,4-dioxocyclobut-1-en-1-yl)amino) propyl)amino) nonanoate (Compound 69)

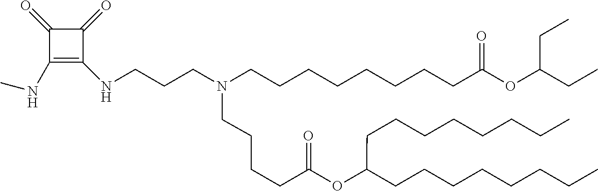 [SMILES: CCCCCCCCC(CCCCCCCC)OC(=O)CCCCN(CCCCCCCCC(=O)OC(CC)CC)CCCNc1c(NC)c(=O)c1=O]

Compound 69 was synthesized according to Representative Procedures 2 and 3 and general Methods A, C, D, E, H and I. Product was obtained as a pale yellow oil (390 mg, 0,521 mmol, 70%). MS (ESI): m/z [M+H]⁺ 748.9 for C₄₄H₈₂N₃O₆; ¹H NMR (600 MHz, CDCl₃) δ 4.81 (qu, J=6.2 Hz, 1H), 4.74 (tt, J=7.1, 5.3 Hz, 1H), 3.70 (bs, 2H), 3.29 (d, J=5.0 Hz, 3H), 2.48 (t, J=6.1 Hz, 2H), 2.41 (t, J=6.9 Hz, 2H), 2.35 (t, J=7.5 Hz, 2H), 2.33 (t, J=7.2 Hz, 2H), 2.28 (t, J=7.5 Hz, 2H), 1.75 (qu, J=6.2 Hz, 2H), 1.64-1.57 (m, 4H), 1.57-1.48 (m, 8H), 1.47-1.41 (m, 2H), 1.41-1.34 (m, 2H), 1.32-1.19 (m, 34H), 0.86 (t, J=7.4 Hz, 12H); NMR (151 MHz,) δ 183.4, 183.2, 174.0, 168.5, 168.3, 76.6, 75.0, 53.8, 53.6, 34.8, 34.8, 34.5, 34.2, 32.0, 32.0, 32.0, 31.3, 29.7, 29.7, 29.7, 29.6, 29.6, 29.6, 29.6, 29.4, 29.4, 29.4, 29.4, 29.3, 28.1, 27.7, 26.6, 26.6, 26.6, 26.6, 26.6, 26.6, 26.6, 25.5, 25.3, 23.1, 22.8, 22.8, 14.2, 14.2, 9.8, 9.7, 9.7, 9.7.

### Isopentyl 9-((5-(heptadecan-9-yloxy)-5-oxopentyl) (3-((2-(methylamino)-3,4-dioxocyclobut-1-en-1-yl)amino) propyl)amino) nonanoate (Compound 70)

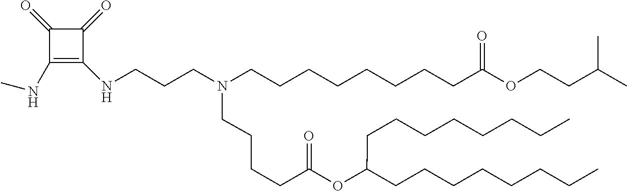 [SMILES: CCCCCCCCC(CCCCCCCC)OC(=O)CCCCN(CCCCCCCCC(=O)OCCC(C)C)CCCNc1c(NC)c(=O)c1=O]

Compound 70 was synthesized according to Representative Procedures 2 and 3 and general Methods A, C, D, E, H and I. Product was obtained as a pale yellow oil (550 mg, 0,735 mmol, 71%). MS (ESI): m/z [M+H]⁺ 749.0 for C₄₄H₈₂N₃O₆; ¹H NMR (600 MHz, CDCl₃+CD₃OD) δ 4.76 (qu, J=6.3 Hz, 1H), 4.01 (t, J=6.9 Hz, 2H), 3.59 (bs, 1H), 3.47 (bs, 1H), 3.20 (s, 3H), 3.00 (q, J=7.3 Hz, 3H), 2.88-2.80 (m, 2H), 2.79-2.65 (m, 2H), 2.30-2.24 (m, 2H), 2.21 (t, J=7.5 Hz, 2H), 1.90-1.80 (m, 2H), 1.65-1.45 (m, 10H), 1.45-1.39 (m, 7H), 1.30-1.10 (m, 42H), 0.83 (t, J=3.7 Hz, 6H), 0.79 (t, J=7.1 Hz, 6H); ¹³C NMR (150 MHz, CDCl₃+CD₃OD) δ 182.6, 174.0, 173.2, 169.1, 168.0, 162.4, 162.1, 117.9, 115.9, 74.8, 63.0, 63.0, 53.2, 52.9, 51.1, 46.1, 37.5, 34.4, 34.4, 34.4, 34.4, 34.4, 34.1, 34.0, 32.0, 32.0, 32.0, 31.2, 29.7, 29.6, 29.6, 29.6, 29.6, 29.4, 29.4, 29.3, 29.3, 29.3, 29.3, 29.2, 29.2, 29.1, 29.0, 27.1, 25.4, 25.2, 25.0, 25.0, 22.8, 22.8, 22.8, 22.8, 22.6, 22.6, 22.6, 22.6, 22.5, 22.5, 14.2, 14.2, 9.1.

### Pentyl 9-((5-(heptadecan-9-yloxy)-5-oxopentyl) (3-hydroxypropyl)amino) nonanoate (Compound 71)

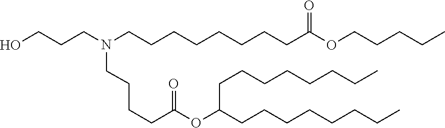 [SMILES: CCCCCCCCC(CCCCCCCC)OC(=O)CCCCN(CCCO)CCCCCCCCC(=O)OCCCCC]

Compound 71 was synthesized according to Representative Procedure 1 general Methods A, B, C and G. Product was obtained as a colorless oil (560 mg, 0.875 mmol, 80%). MS (ESI): m/z [M+H]⁺ 640.5 for C₃₉H₇₇NO₅; ¹H-NMR (400 MHz, CDCl₃) δ 5.47 (bs, 1H), 4.85 (qu, J=6.3 Hz, 1H), 4.04 (t, J=6.8 Hz, 2H), 3.77 (t, J=5.1 Hz, 2H), 2.61 (t, J=5.6 Hz, 2H), 2.47-2.36 (m, 4H), 2.31 (t, J=7.5 Hz, 2H), 2.29 (t, 7.3 Hz, 2H), 1.70-1.57 (m, 8H), 1.56-1.40 (m, 8H), 1.38-1.20 (m, 36H), 0.91 (t, J=7.0 Hz, 3H), 0.88 (t, J=6.9 Hz, 6H); ¹³C NMR (101 MHz, CDCl₃) δ 173.9, 173.2, 74.2, 64.7, 64.3, 55.1, 54.2, 53.7, 34.4, 34.3, 34.1, 31.8, 29.5, 29.5, 29.4, 29.2, 29.1, 28.3, 28.1, 27.8, 27.4, 26.8, 26.2, 25.3, 24.9, 23.0, 22.6, 22.3, 14.1, 13.9

### Pentyl 9-((5-(heptadecan-9-yloxy)-5-oxopentyl) (4-hydroxybutyl)amino) nonanoate (Compound 72)

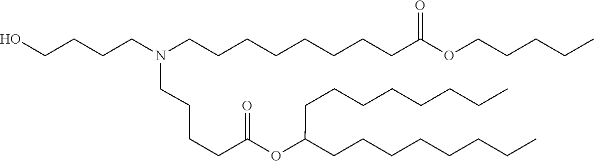 [SMILES: CCCCCCCCC(CCCCCCCC)OC(=O)CCCCN(CCCCO)CCCCCCCCC(=O)OCCCCC]

Compound 72 was synthesized according to Representative Procedure 1 general Methods A, B, C and G. Product was obtained as a colorless oil (340 mg, 0.520 mmol, 75%). MS (ESI): m/z [M+H]⁺ 654.6 for C₄₀H₇₉NO₅; ¹H NMR (400 MHz, CDCl₃) δ 6.27 (bs, 1H), 4.85 (qu, J=6.3 Hz, 1H), 4.04 (t, J=6.8 Hz, 2H), 3.53 (t, J=4.7 Hz, 2H), 2.46-2.36 (m, 6H), 2.28 (t, J=7.8, 2H), 2.26 (t, J=7.4 Hz, 2H), 1.65-1.53 (m, 10H), 1.53-1.38 (m, 8H), 1.33-1.20 (m, 36H), 0.88 (t, J=7.0, 3H), 0.85 (t, J=6.9 Hz, 6H); ¹³C NMR (101 MHz, CDCl₃) δ 173.9, 173.3, 74.2, 64.3, 62.7, 54.6, 53.7, 53.2, 34.4, 34.3, 34.1, 32.7, 31.8, 29.5, 29.5, 29.3, 29.2, 29.2, 29.1, 28.3, 28.1, 27.6, 26.3, 26.0, 25.4, 25.3, 25.0, 23.2, 22.6, 22.3, 14.1, 13.9.

### 2-Methylbutyl 9-((5-(heptadecan-9-yloxy)-5-oxopentyl) (2-hydroxyethyl)amino) nonanoate (Compound 73)

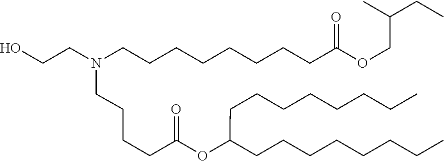 [SMILES: CCCCCCCCC(CCCCCCCC)OC(=O)CCCCN(CCO)CCCCCCCCC(=O)OCC(C)CC]

Compound 73 was synthesized according to Representative Procedure 1 and general Methods A, B, C and G. Product was obtained as a colorless oil (510 mg, 0.815 mmol, 78%). MS (ESI): m/z [M+H]⁺ 627.3 for C₃₈H₇₆NO₅; ¹H NMR (600 MHz, CDCl₃) δ 4.89-4.82 (m, 1H), 3.95 (dd, J=10.7, 6.0 Hz, 1H), 3.88-3.83 (m, 1H), 3.52 (t, J=5.4 Hz, 2H), 2.56 (t, J=5.4 Hz, 2H), 2.44 (ddd, J=25.1, 16.2, 8.7 Hz, 4H), 2.29 (td, J=7.6, 1.0 Hz, 4H), 1.74-1.65 (sept, J=6.6 Hz, 1H), 1.65-1.55 (m, 4H), 1.55-1.37 (m, 9H), 1.34-1.12 (m, 33H), 0.93-0.82 (m, 12H); ¹³C NMR (151 MHz, CDCl₃) δ 174.1, 173.5, 74.4, 69.0, 58.5, 55.6, 53.9, 53.6, 34.6, 34.5, 34.3, 34.3, 32.0, 29.7, 29.6, 29.6, 29.4, 29.4, 29.3, 27.5, 27.3, 26.8, 26.2, 25.5, 25.2, 23.1, 22.8, 16.5, 14.2, 11.4.

### Pentan-3-yl 9-((2-hydroxyethyl) (5-(nonadecan-10-yloxy)-5-oxopentyl)amino) nonanoate (Compound 74)

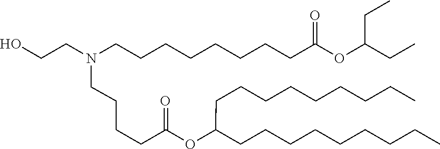 [SMILES: CCCCCCCCCC(CCCCCCCCC)OC(=O)CCCCN(CCO)CCCCCCCCC(=O)OC(CC)CC]

Compound 74 was synthesized according to Representative Procedure 1 and general Methods A, B, C and G. Product was obtained as a colorless oil (560 mg, 0.856 mmol, 82%). MS (ESI): m/z [M+H]⁺ 655.3 for C₄₀H₈₀NO₅; ¹H NMR (600 MHz, CDCl₃) δ 4.89-4.83 (m, 1H), 4.75 (tt, J=7.1, 5.3 Hz, 1H), 3.52 (t, J=5.4 Hz, 2H), 2.57 (t, J=5.4 Hz, 2H), 2.49-2.40 (m, 4H), 2.29 (td, J=7.5, 5.1 Hz, 4H), 1.65-1.39 (m, 16H), 1.34-1.20 (m, 36H), 0.87 (t, J=7.3 Hz, 12H); ¹³C NMR (151 MHz, CDCl₃) δ 173.9, 173.5, 76.5, 74.4, 58.5, 55.6, 53.9, 53.6, 34.8, 34.6, 34.3, 32.0, 29.7, 29.7, 29.6, 29.4, 29.4, 29.3, 27.5, 27.3, 26.8, 26.6, 25.5, 25.3, 23.1, 22.8, 14.2, 9.7.

### Pentan-3-yl 10-((2-hydroxyethyl) (5-(nonadecan-10-yloxy)-5-oxopentyl)amino) decanoate (Compound 75)

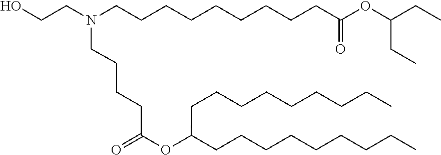 [SMILES: CCCCCCCCCC(CCCCCCCCC)OC(=O)CCCCN(CCO)CCCCCCCCCC(=O)OC(CC)CC]

Compound 75 was synthesized according to Representative Procedure 1 and general Methods A, B, C and G. Product was obtained as a colorless oil (510 mg, 0.763 mmol, 76%). MS (ESI): m/z [M+H]⁺ 668.7 for C₄₁H₈₂NO₅; ¹H NMR (600 MHz, CDCl₃) δ 4.89-4.83 (m, 1H), 4.75 (tt, J=7.1, 5.3 Hz, 1H), 3.52 (t, J=5.4 Hz, 2H), 2.88 (bs, 1H), 2.57 (t, J=5.4 Hz, 2H), 2.47 (t, J=7.8 Hz, 2H), 2.44 (t, J=7.8 Hz, 2H), 2.30 (t, J=7.5 Hz, 2H), 2.29 (t, J=7.5 Hz, 2H), 1.65-1.39 (m, 16H), 1.33-1.19 (m, 38H), 0.87 (t, J=7.4 Hz, 12H); ¹³C NMR (151 MHz, CDCl₃) δ 173.9, 173.4, 76.5, 74.4, 58.5, 55.7, 54.0, 53.6, 34.8, 34.6, 34.3, 32.0, 29.7, 29.7, 29.6, 29.4, 29.4, 29.3, 27.6, 27.3, 26.8, 26.6, 25.5, 25.3, 23.1, 22.8, 14.2, 9.7.

### 3-Methylbut-2-en-1-yl 9-((5-(heptadecan-9-yloxy)-5-oxopentyl) (2-hydroxyethyl)amino) nonanoate (Compound 76)

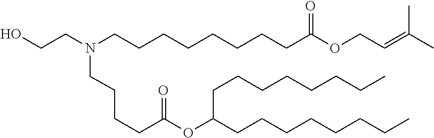 [SMILES: CCCCCCCCC(CCCCCCCC)OC(=O)CCCCN(CCO)CCCCCCCCC(=O)OCC=C(C)C]

Compound 76 was synthesized according to Representative Procedure 1 and general Methods A, B, C and G. Product was obtained as a colorless oil (240 mg, 0.385 mmol, 55%). MS (ESI): m/z [M+H]⁺ 624.5 for C₃₈H₇₄NO₅; ¹H NMR (600 MHz, CDCl₃) δ 5.33 (tsept, J=7.2, 1.2 Hz, 1H), 4.89-4.83 (m, 1H), 4.56 (d, J=7.2 Hz, 2H), 3.56 (t, J=5.3 Hz, 2H), 3.07 (bs, 1H), 2.62 (t, J=5.4 Hz, 2H), 2.52 (t, J=7.5 Hz, 2H), 2.48 (t, J=7.5 Hz, 2H), 2.30 (t, J=7.2 Hz, 2H), 2.29 (t, J=7.5 Hz, 2H), 1.77-1.74 (m, 3H), 1.70 (d, J=0.9 Hz, 3H), 1.65-1.58 (m, 4H), 1.55-1.42 (m, 8H), 1.33-1.20 (m, 32H), 0.87 (t, J=6.9 Hz, 6H); ¹³C NMR (151 MHz, CDCl₃) δ 174.0, 173.4, 139.0, 118.9, 74.5, 61.3, 58.3, 55.7, 54.0, 53.7, 34.5, 34.5, 34.3, 32.0, 29.7, 29.6, 29.5, 29.4, 29.4, 29.2, 27.5, 26.9, 26.5, 25.9, 25.5, 25.1, 23.1, 22.8, 18.1, 14.2.

### 3-Methylbut-2-en-1-yl 9-((2-hydroxyethyl) (5-(nonadecan-10-yloxy)-5-oxopentyl)amino) nonanoate (Compound 77)

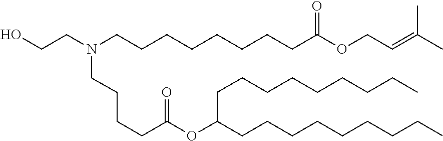 [SMILES: CCCCCCCCCC(CCCCCCCCC)OC(=O)CCCCN(CCO)CCCCCCCCC(=O)OCC=C(C)C]

Compound 77 was synthesized according to Representative Procedure 1 and general Methods A, B, C and G. Product was obtained as a colorless oil (240 mg, 0.368 mmol, 53%). MS (ESI): m/z [M+H]⁺ 652.6 for C₄₀H₇₈NO₅; ¹H NMR (600 MHz, CDCl₃) δ 5.33 (tsept, J=7.2, 1.2 Hz, 1H), 4.89-4.83 (m, 1H), 4.56 (d, J=7.2 Hz, 2H), 3.56 (t, J=5.3 Hz, 2H), 3.07 (bs, 1H), 2.62 (t, J=5.4 Hz, 2H), 2.52 (t, J=7.5 Hz, 2H), 2.48 (t, J=7.5 Hz, 2H), 2.30 (t, J=7.2 Hz, 2H), 2.29 (t, J=7.5 Hz, 2H), 1.77-1.74 (m, 3H), 1.70 (d, J=0.9 Hz, 3H), 1.65-1.58 (m, 4H), 1.55-1.42 (m, 8H), 1.33-1.20 (m, 32H), 0.87 (t, J=6.9 Hz, 6H); ¹³C NMR (151 MHz, CDCl₃) δ 174.0, 173.4, 139.0, 118.9, 74.5, 61.3, 58.3, 55.7, 54.0, 53.7, 34.5, 34.5, 34.3, 32.0, 29.7, 29.6, 29.5, 29.4, 29.4, 29.2, 27.5, 26.9, 26.5, 25.9, 25.5, 25.1, 23.1, 22.8, 18.1, 14.2.

### Pent-3-yn-1-yl 9-((5-(heptadecan-9-yloxy)-5-oxopentyl) (2-hydroxyethyl)amino) nonanoate (Compound 78)

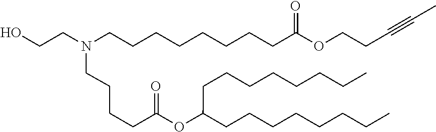 [SMILES: CC#CCCOC(=O)CCCCCCCCN(CCO)CCCCC(=O)OC(CCCCCCCC)CCCCCCCC]

Compound 78 was synthesized according to Representative Procedures 1 and general Methods A, B, C and G. Product was obtained as a colorless oil (350 mg, 0,563 mmol, 80%). MS (ESI): m/z [M+H]⁺ 623.1 for C₃₈H₇₂NO₅; ¹H NMR (600 MHz, CDCl₃) δ 4.87 (qu, J=6.3 Hz, 1H), 4.13 (t, J=7.0 Hz, 2H), 3.56 (t, J=5.3 Hz, 2H), 3.13 (bs, 1H), 2.62 (t, J=5.3 Hz, 2H), 2.52 (t, J=7.5 Hz, 2H), 2.48 (t, J=7.3 Hz, 2H), 2.45 (m, J=2.6 Hz, 2H), 2.31 (t, J=7.4 Hz, 4H), 1.78 (t, J=2.5 Hz, 3H), 1.62 (qu, J=7.4 Hz, 4H), 1.54-1.48 (m, 6H), 1.45 (m, J=7.2 Hz, 2H), 1.30-1.26 (m, 32H), 0.88 (t, J=7.0 Hz, 6H); ¹³C NMR (151 MHz, CDCl₃) δ 173.6, 173.2, 77.2, 74.7, 74.3, 62.6, 58.2, 55.5, 53.8, 53.5, 34.4, 34.2, 34.1, 31.8, 29.5, 29.5, 29.3, 29.2, 29.0, 27.3, 26.8, 26.3, 25.3, 24.9, 22.9, 22.6, 19.2, 14.1, 3.4.

### Pentan-3-yl 10-((4-aminobutyl) (5-(heptadecan-9-yloxy)-5-oxopentyl)amino) decanoate (Compound 79)

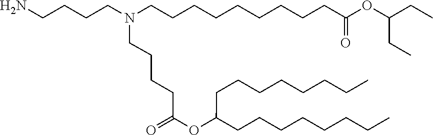 [SMILES: CCCCCCCCC(CCCCCCCC)OC(=O)CCCCN(CCCCN)CCCCCCCCCC(=O)OC(CC)CC]

Compound 79 was synthesized according to Representative Procedure 2 and general Methods A, C, D, E and H. Product was obtained as a colorless oil (580 mg, 0.869 mmol, 93%). MS (ESI): m/z [M+H]⁺ 667.7 for C₄₁H₈₂N₂O₄; ¹H NMR (400 MHz, CDCl₃) δ 4.85 (qu J=6.3 Hz, 1H), 4.74 (tt, J=7.0, 5.4 Hz, 1H), 2.68 (t, J=6.6 Hz, 2H), 2.42-2.31 (m, 6H), 2.28 (t, J=7.6 Hz, 2H), 2.28 (t, J=7.6 Hz, 2H), 1.66-1.11 (m, 56H), 0.86 (t, J=7.4 Hz, 12H); ¹³C NMR (101 MHz, CDCl₃) δ 173.9, 173.6, 76.5, 74.3, 54.3, 54.2, 53.8, 42.4, 34.8, 34.8, 34.3, 32.1, 32.0, 29.7, 29.7, 29.6, 29.4, 29.4, 29.3, 27.7, 27.3, 26.8, 26.6, 25.5, 25.3, 24.7, 23.3, 22.8, 14.2, 9.7.

### Pentan-3-yl 10-((5-(heptadecan-9-yloxy)-5-oxopentyl) (4-((2-(methylamino)-3,4-dioxocyclobut-1-en-1-yl)amino)butyl)amino) decanoate (Compound 80)

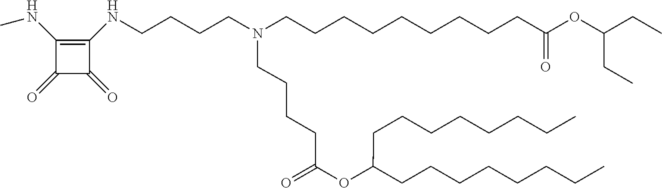 [SMILES: CCCCCCCCC(CCCCCCCC)OC(=O)CCCCN(CCCCCCCCCC(=O)OC(CC)CC)CCCCNc1c(NC)c(=O)c1=O]

Compound 80 was synthesized according to Representative Procedures 2 and 3 and general Methods A, C, D, E, H and I. Product was obtained as a colorless waxy amorphous solid (310 mg, 0.399 mmol, 89%). MS (ESI): m/z [M+H]⁺ 777.3 for C₄₆H₈₆N₃O₆; ¹H NMR (600 MHz, CDCl₃) δ0 7.61 (bs, 1H), 7.37 (bs, 1H), 4.83 (qu, J=6.3 Hz, 1H), 4.73 (tt, J=7.1, 5.3 Hz, 1H), 3.65 (bs, 2H), 3.37-3.27 (m, 3H), 2.42-2.32 (m, 6H), 2.31-2.25 (m, 4H), 1.66-1.33 (m, 20H), 1.33-1.17 (m, 34H), 0.88-0.84 (m, 12H); ¹³C NMR (151 MHz, CDCl₃) δ 183.0, 182.4, 173.9, 173.9, 168.4, 168.2, 76.5, 74.5, 54.2, 53.7, 44.6, 34.8, 34.7, 34.2, 32.0, 31.4, 29.7, 29.7, 29.6, 29.6, 29.4, 29.4, 29.4, 29.3, 29.2, 27.8, 27.1, 26.6, 26.6, 25.5, 25.3, 24.3, 23.3, 22.8, 14.2, 9.7.

### Pentan-3-yl 10-((4-aminobutyl) (5-(nonadecan-10-yloxy)-5-oxopentyl)amino) decanoate (Compound 81)

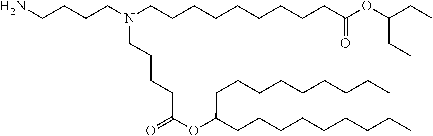 [SMILES: CCCCCCCCCC(CCCCCCCCC)OC(=O)CCCCN(CCCCN)CCCCCCCCCC(=O)OC(CC)CC]

Compound 81 was synthesized according to Representative Procedure 2 and general Methods A, C, D, E and H. Product was obtained as a colorless oil (570 mg, 0.820 mmol, 91%). MS (ESI): m/z [M+H]⁺ 695.7 for C₄₃H₈₇N₂O₄; ¹H NMR (400 MHz, CDCl₃) δ 4.85 (qu J=6.2 Hz, 1H), 4.74 (tt, J=7.0, 5.5 Hz, 1H), 2.68 (t, J=6.6 Hz, 2H), 2.43-2.31 (m, 6H), 2.28 (t, J=7.6 Hz, 2H), 2.28 (t, J=7.4 Hz, 2H), 1.66-1.15 (m, 60H), 0.86 (t, J=7.4 Hz, 12H); ¹³C NMR (101 MHz, CDCl₃) δ 173.9, 173.6, 76.5, 74.3, 54.3, 54.2, 53.8, 42.4, 34.8, 34.8, 34.3, 32.0, 32.0, 29.7, 29.7, 29.7, 29.7, 29.6, 29.4, 29.4, 29.3, 27.7, 27.3, 26.8, 26.6, 25.5, 25.3, 24.7, 23.3, 22.8, 14.2, 9.7.

### Pentan-3-yl 10-((4-((2-(methylamino)-3,4-dioxocyclobut-1-en-1-yl)amino)butyl) (5-(nonadecan-10-yloxy)-5-oxopentyl)amino) decanoate (Compound 82)

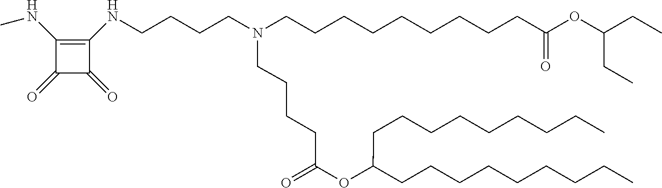 [SMILES: CCCCCCCCCC(CCCCCCCCC)OC(=O)CCCCN(CCCCCCCCCC(=O)OC(CC)CC)CCCCNc1c(NC)c(=O)c1=O]

Compound 82 was synthesized according to Representative Procedures 2 and 3 and general Methods A, C, D, E, H and I. Product was obtained as a colorless waxy amorphous solid (330 mg, 0.410 mmol, 95%). MS (ESI): m/z [M+H]⁺ 805.4 for C₄₈H₉₀N₃O₆; ¹H NMR (600 MHz, CDCl₃) δ 7.61 (bs, 1H), 7.36 (bs, 1H), 4.83 (qu, J=6.3 Hz, 1H), 4.73 (tt, J=7.1, 5.3 Hz, 1H), 3.65 (bs, 2H), 3.31 (d, J=5.1 Hz, 3H), 2.42-2.32 (m, 6H), 2.31-2.25 (m, 4H), 1.67-1.33 (m, 20H), 1.32-1.17 (m, 38H), 0.86 (t, J=7.3 Hz, 12H); ¹³C NMR (151 MHz, CDCl₃) δ 183.0, 182.4, 173.9, 173.9, 168.4, 168.2, 76.5, 74.5, 54.2, 53.7, 44.6, 34.8, 34.7, 34.2, 32.0, 31.4, 29.7, 29.7, 29.7, 29.7, 29.4, 29.4, 29.4, 29.3, 29.2, 27.8, 27.1, 26.7, 26.6, 25.5, 25.3, 24.3, 23.3, 22.8, 14.2, 9.7.

### Pentan-3-yl 10-((3-aminopropyl) (5-(heptadecan-9-yloxy)-5-oxopentyl)amino) decanoate (Compound 83)

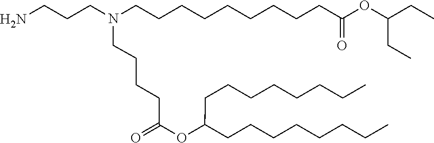 [SMILES: CCCCCCCCC(CCCCCCCC)OC(=O)CCCCN(CCCN)CCCCCCCCCC(=O)OC(CC)CC]

Compound 83 was synthesized according to Representative Procedure 2 and general Methods A, C, D, E and H. Product was obtained as a colorless oil (540 mg, 0.827 mmol, 94%). MS (ESI): m/z [M+H]⁺ 653.7 for C₄₀H₈₁N₂O₄; ¹H NMR (400 MHz, CDCl₃) δ 4.90-4.80 (qu J=4.6 Hz, 1H), 4.74 (tt, J=7.0, 5.4 Hz, 1H), 2.70 (t, J=7.0 Hz, 2H), 2.46-2.24 (m, 6H), 2.28 (t, J=7.4 Hz, 2H), 2.28 (t, J=7.6 Hz, 2H), 1.66-1.17 (m, 54H), 0.86 (t, J=7.4 Hz, 12H); 13C NMR (101 MHz, CDCl₃) δ 173.9, 173.6, 76.5, 74.3, 54.3, 53.9, 52.0, 41.0, 34.8, 34.7, 34.3, 32.0, 31.2, 29.7, 29.7, 29.7, 29.6, 29.4, 29.4, 29.3, 27.7, 27.2, 26.7, 26.6, 25.5, 25.3, 23.3, 22.8, 14.2, 9.7.

### Pentan-3-yl 10-((5-(heptadecan-9-yloxy)-5-oxopentyl) (3-((2-(methylamino)-3,4-dioxocyclobut-1-en-1-yl)amino) propyl)amino) decanoate (Compound 84)

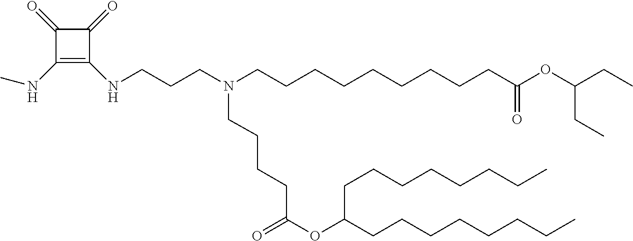 [SMILES: CCCCCCCCC(CCCCCCCC)OC(=O)CCCCN(CCCCCCCCCC(=O)OC(CC)CC)CCCNc1c(NC)c(=O)c1=O]

Compound 84 was synthesized according to Representative Procedures 2 and 3 and general Methods A, C, D, E, H and I. Product was obtained as a yellowish waxy amorphous solid (320 mg, 0.420 mmol, 91%). MS (ESI): m/z [M+H]⁺ 763.1 for C₄₅H₈₄N₃O₆; ¹H NMR (600 MHz, CDCl₃) δ 7.38 (bs, 1H), 7.00 (bs, 1H), 4.81 (qu J=6.3 Hz, 1H), 4.73 (tt, J=7.1, 5.3 Hz, 1H), 3.69 (bs, 2H), 3.29 (d, J=5.0 Hz, 3H), 2.48 (t, J=6.4 Hz, 2H), 2.40 (t, J=7.0 Hz, 2H), 2.35 (t, J=7.5 Hz, 2H), 2.31 (t, J=7.2 Hz, 2H), 2.27 (t, J=7.5 Hz, 2H), 1.75 (qu J=6.4 Hz, 2H), 1.64-1.47 (m, 12H), 1.47-1.34 (m, 4H), 1.33-1.17 (m, 34H), 0.86 (t, J=7.4 Hz, 12H); ¹³C NMR (151 MHz, CDCl₃) δ 183.3, 183.0, 174.3, 173.9, 168.5, 168.3, 76.5, 74.8, 53.9, 53.6, 51.3, 43.4, 34.8, 34.5, 34.2, 32.0, 31.3, 29.7, 29.6, 29.6, 29.6, 29.4, 29.3, 29.3, 28.2, 27.7, 26.7, 26.6, 26.4, 25.4, 25.3, 23.1, 22.8, 14.2, 9.7.

### Pentan-3-yl 10-((3-aminopropyl) (5-(nonadecan-10-yloxy)-5-oxopentyl)amino) decanoate (Compound 85)

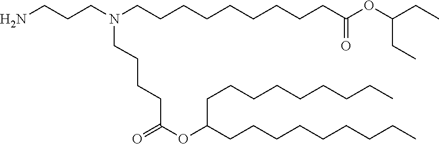 [SMILES: CCCCCCCCCC(CCCCCCCCC)OC(=O)CCCCN(CCCN)CCCCCCCCCC(=O)OC(CC)CC]

Compound 85 was synthesized according to Representative Procedure 2 and general Methods A, C, D, E and H. Product was obtained as a colorless oil (540 mg, 0.793 mmol, 97%). MS (ESI): m/z [M+H]⁺ 681.7 for C₄₂H₈₅N₂O₄; ¹H NMR (400 MHz, CDCl₃) δ 4.84 (qu J=6.3 Hz, 1H), 4.74 (tt, J=7.0, 5.4 Hz, 1H), 2.70 (t, J=6.8 Hz, 2H), 2.46-2.31 (m, 6H), 2.28 (t, J=7.6 Hz, 2H), 2.27 (t, J=7.6 Hz, 2H), 1.66-1.14 (m, 58H), 0.86 (t, J=7.4 Hz, 12H); ¹³C NMR (101 MHz, CDCl₃) δ 173.9, 173.6, 76.5, 74.3, 54.3, 53.9, 52.0, 41.0, 34.8, 34.7, 34.3, 32.0, 31.2, 29.7, 29.7, 29.7, 29.7, 29.6, 29.4, 29.4, 29.3, 27.7, 27.2, 26.7, 26.6, 25.5, 25.3, 23.3, 22.8, 14.2, 9.7.

### Pentan-3-yl 10-((3-((2-(methylamino)-3,4-dioxocyclobut-1-en-1-yl)amino) propyl) (5-(nonadecan-10-yloxy)-5-oxopentyl)amino) decanoate (Compound 86)

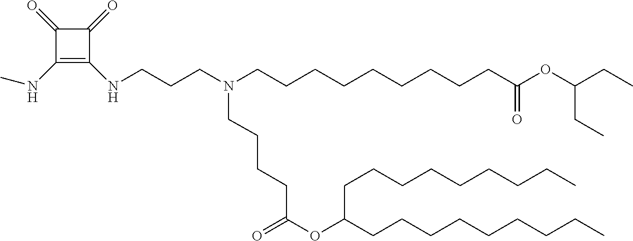 [SMILES: CCCCCCCCCC(CCCCCCCCC)OC(=O)CCCCN(CCCCCCCCCC(=O)OC(CC)CC)CCCNc1c(NC)c(=O)c1=O]

Compound 86 was synthesized according to Representative Procedures 2 and 3 and general Methods A, C, D, E, H and I. Product was obtained as a colorless waxy amorphous solid (320 mg, 0.405 mmol, 92%). MS (ESI): m/z [M+H]⁺ 791.1 for C₄₇H₈₈N₃O₆; ¹H NMR (600 MHz, CDCl₃) δ 7.32 (bs, 1H), 6.92 (bs, 1H), 4.81 (qu, J=6.2 Hz, 1H), 4.73 (tt, J=7.1, 5.3 Hz, 1H), 3.70 (bs, 2H), 3.29 (d, J=5.0 Hz, 3H), 2.48 (t, J=6.4 Hz, 2H), 2.40 (t, J=7.0 Hz, 2H), 2.35 (t, J=7.5 Hz, 2H), 2.32 (t, J=7.2 Hz, 2H), 2.28 (t, J=7.5 Hz, 2H), 1.75 (qu, J=6.3 Hz, 2H), 1.63-1.47 (m, 12H), 1.47-1.34 (m, 4H), 1.33-1.17 (m, 38H), 0.88-0.82 (m, 12H); ¹³C NMR (151 MHz, CDCl₃) δ 183.3, 183.0, 174.3, 173.9, 168.5, 168.3, 76.5, 74.9, 53.9, 53.6, 51.3, 43.4, 34.8, 34.5, 34.2, 32.0, 31.3, 29.7, 29.7, 29.6, 29.6, 29.4, 29.4, 29.3, 28.1, 27.7, 26.7, 26.6, 26.5, 25.5, 25.3, 23.1, 22.8, 14.2, 9.7.

### Pentyl 10-((4-aminobutyl) (5-(heptadecan-9-yloxy)-5-oxopentyl)amino) decanoate (Compound 87)

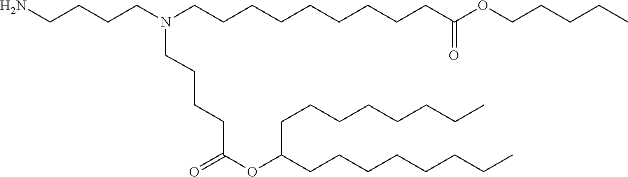 [SMILES: CCCCCCCCC(CCCCCCCC)OC(=O)CCCCN(CCCCN)CCCCCCCCCC(=O)OCCCCC]

Compound 87 was synthesized according to Representative Procedure 2 and general Methods A, C, D, E and H. Product was obtained as a colorless oil (560 mg, 0.839 mmol, 90%). MS (ESI): m/z [M+H]⁺ 667.7 for C₄₁H₈₃N₂O₄; ¹H NMR (400 MHz, CDCl₃) δ 4.84 (qu J=6.3 Hz, 1H), 4.04 (t, J=6.8 Hz, 2H), 2.68 (t, J=6.6 Hz, 2H), 2.43-2.32 (m, 6H), 2.28 (t, J=7.6 Hz, 2H), 2.27 (t, J=7.4 Hz, 2H), 1.67-1.16 (m, 58H), 0.94-0.81 (m, 9H); ¹³C NMR (101 MHz, CDCl₃) δ 174.1, 173.6, 74.3, 64.5, 54.3, 54.2, 53.8, 42.4, 34.8, 34.5, 34.3, 32.1, 32.0, 29.7, 29.7, 29.6, 29.6, 29.4, 29.4, 29.3, 28.5, 28.2, 27.7, 27.2, 26.7, 25.5, 25.2, 24.7, 23.3, 22.8, 22.4, 14.2, 14.1.

### Pentyl 10-((5-(heptadecan-9-yloxy)-5-oxopentyl) (4-((2-(methylamino)-3,4-dioxocyclobut-1-en-1-yl)amino)butyl)amino) decanoate (Compound 88)

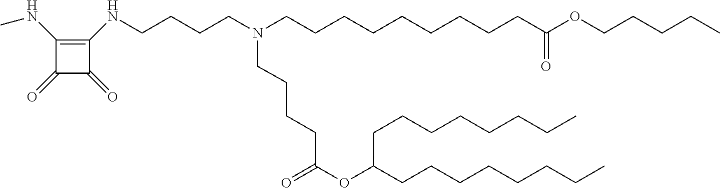 [SMILES: CCCCCCCCC(CCCCCCCC)OC(=O)CCCCN(CCCCCCCCCC(=O)OCCCCC)CCCCNc1c(NC)c(=O)c1=O]

Compound 88 was synthesized according to Representative Procedures 2 and 3 and general Methods A, C, D, E, H and I. Product was obtained as a colorless waxy amorphous solid (260 mg, 0.335 mmol, 75%). MS (ESI): m/z [M+H]⁺ 777.2 for C₄₆H₈₆N₃O₆; ¹H NMR (600 MHz, CDCl₃) δ 7.62 (bs, 1H), 7.39 (bs, 1H), 4.83 (qu J=6.2 Hz, 1H), 4.03 (t, J=6.8 Hz, 2H), 3.65 (d, J=5.0 Hz, 2H), 3.31 (d, J=5.0 Hz, 3H), 2.45-2.31 (m, 6H), 2.31-2.23 (m, 4H), 1.68-1.54 (m, 8H), 1.53-1.15 (m, 48H), 0.89 (t, J=7.2 Hz, 3H), 0.86 (t, J=7.0 Hz, 6H); ¹³C NMR (151 MHz, CDCl₃) δ 183.0, 182.3, 174.1, 173.9, 168.4, 168.2, 74.5, 64.5, 54.2, 53.7, 44.6, 34.7, 34.5, 34.2, 32.0, 31.4, 31.2, 29.7, 29.6, 29.6, 29.4, 29.4, 29.3, 29.2, 28.5, 28.2, 27.8, 27.1, 26.6, 25.4, 25.1, 24.2, 23.3, 22.8, 22.4, 14.2, 14.1.

### Pentyl 10-((3-aminopropyl) (5-(heptadecan-9-yloxy)-5-oxopentyl)amino) decanoate (Compound 89)

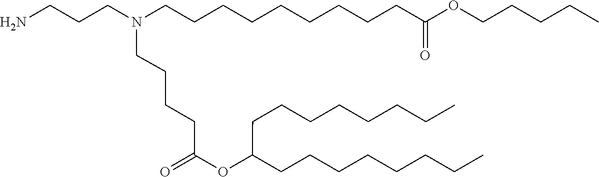 [SMILES: CCCCCCCCC(CCCCCCCC)OC(=O)CCCCN(CCCN)CCCCCCCCCC(=O)OCCCCC]

Compound 89 was synthesized according to Representative Procedure 2 and general Methods A, C, D, E and H. Product was obtained as a colorless oil (500 mg, 0.766 mmol, 92%). MS (ESI): m/z [M+H]⁺ 653.7 for C₄₀H₈₁N₂O₄; ¹H NMR (600 MHz, CDCl₃) δ 4.85 (qu J=6.3 Hz, 1H), 4.04 (t, J=6.8 Hz, 2H), 2.70 (t, J=6.8 Hz, 2H), 2.42 (t, J=6.9 Hz, 2H), 2.38 (t, J=7.5 Hz, 2H), 2.35 (t, J=7.5 Hz, 2H), 2.28 (t, J=7.2 Hz, 2H), 2.27 (t, J=7.5 Hz, 2H), 1.65-1.10 (m, 56H), 0.89 (t, J=7.2 Hz, 3H), 0.86 (t, J=7.2 Hz, 6H); ¹³C NMR (151 MHz, CDCl₃) δ 174.1, 173.6, 74.3, 64.5, 54.3, 53.9, 52.0, 41.0, 34.7, 34.5, 34.3, 32.0, 31.2, 29.7, 29.7, 29.6, 29.6, 29.4, 29.4, 29.3, 28.5, 28.2, 27.7, 27.2, 26.7, 25.4, 25.1, 23.3, 22.8, 22.4, 14.2, 14.1.

### Pentyl 10-((5-(heptadecan-9-yloxy)-5-oxopentyl) (3-((2-(methylamino)-3,4-dioxocyclobut-1-en-1-yl)amino) propyl)amino) decanoate (Compound 90)

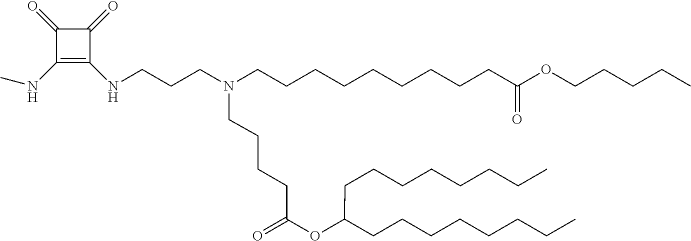 [SMILES: CCCCCCCCC(CCCCCCCC)OC(=O)CCCCN(CCCCCCCCCC(=O)OCCCCC)CCCNc1c(NC)c(=O)c1=O]

Compound 90 was synthesized according to Representative Procedures 2 and 3 and general Methods A, C, D, E, H and I. Product was obtained as a colorless waxy amorphous solid (210 mg, 0.276 mmol, 60%). MS (ESI): m/z [M+H]⁺ 763.3 for C₄₅H₈₄N₃O₆; ¹H NMR (400 MHz, CDCl₃) δ 7.45 (bs, 1H), 7.12 (bs, 1H), 4.81 (qu, J=6.3 Hz, 1H), 4.03 (t, J=6.8 Hz, 2H), 3.68 (bs, 2H), 3.29 (d, J=5.0 Hz, 3H), 2.47 (t, J=6.5 Hz, 2H), 2.43-2.23 (m, 8H), 1.75 (qu, J=6.4 Hz, 2H), 1.66-1.13 (m, 52H), 0.92-0.79 (m, 9H); ¹³C NMR (101 MHz, CDCl₃) δ 183.2, 182.8, 174.2, 174.1, 168.4, 168.3, 74.7, 64.5, 53.9, 53.6, 51.3, 43.3, 34.5, 34.5, 34.2, 31.9, 31.3, 29.7, 29.6, 29.6, 29.6, 29.4, 29.3, 29.3, 28.4, 28.3, 28.2, 27.7, 26.7, 26.5, 25.4, 25.1, 23.1, 22.8, 22.4, 14.2, 14.1.

### Pentyl 9-((3-((2-(methylamino)-3,4-dioxocyclobut-1-en-1-yl)amino) propyl) (5-(nonadecan-10-yloxy)-5-oxopentyl)amino) nonanoate (Compound 91)

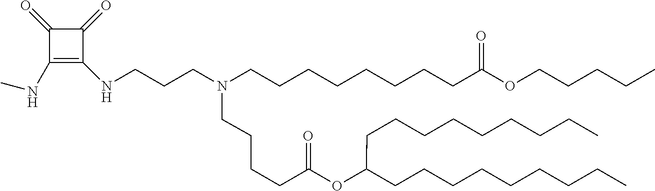 [SMILES: CCCCCCCCCC(CCCCCCCCC)OC(=O)CCCCN(CCCCCCCCC(=O)OCCCCC)CCCNc1c(NC)c(=O)c1=O]

Compound 91 was synthesized according to Representative Procedures 2 and 3 and general Methods A, C, D, E, H and I. Product was obtained as a pale yellow oil (810 mg, 1.040 mmol, 80%). MS (ESI): m/z [M+H]⁺ 777.2 for C₄₆H₈₆N₃O₆; ¹H NMR (600 MHz, CDCl₃) δ 7.19 (bs, 1H), 6.74 (bs, 1H), 4.82 (qu, J=6.2 Hz, 1H), 4.05 (t, J=6.8 Hz, 2H), 3.72 (bs, 2H), 3.30 (d, J=5.0 Hz, 3H), 2.50 (bt, J=6.1 Hz, 2H), 2.42 (bt, J=6.9 Hz, 2H), 2.37 (bt, J=6.5 Hz, 2H), 2.34 (t, J=7.2 Hz, 2H), 2.29 (t, J=7.5 Hz, 2H), 2.01 (bs, 1H), 1.76 (qu, J=6.2 Hz, 2H), 1.62 (sext, J=7.1 Hz, 6H), 1.53-1.52 (m, 4H), 1.48-1.37 (m, 4H), 1.36-1.30 (m, 4H), 1.29-1.25 (m, 36H), 0.91 (t, J=7.1 Hz, 3H), 0.88 (t, J=7.1 Hz, 6H); ¹³C NMR (151 MHz, CDCl₃) δ 183.2, 183.1, 174.4, 174.0, 168.4, 168.1, 74.9, 64.4, 53.6, 53.4, 51.1, 43.2, 34.3, 34.3, 34.0, 31.8, 31.2, 29.5, 29.5, 29.4, 29.3, 29.1, 28.3, 28.0, 27.8, 27.5, 26.3, 25.3, 24.9, 22.9, 22.6, 22.3, 14.1, 13.9.

### Pentyl 9-((4-((2-(methylamino)-3,4-dioxocyclobut-1-en-1-yl)amino)butyl) (5-(nonadecan-10-yloxy)-5-oxopentyl)amino) nonanoate (Compound 92)

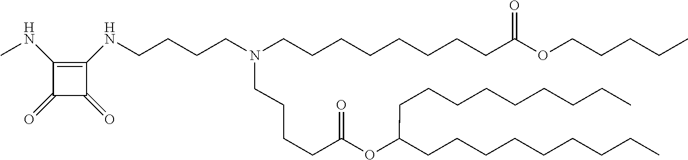 [SMILES: CCCCCCCCCC(CCCCCCCCC)OC(=O)CCCCN(CCCCCCCCC(=O)OCCCCC)CCCCNc1c(NC)c(=O)c1=O]

Compound 92 was synthesized according to Representative Procedures 2 and 3 and general Methods A, C, D, E, H and I. Product was obtained as a pale yellow oil (1.17 g, 1.480 mmol, 80%). MS (ESI): m/z [M+H]⁺ 791.2 for C₄₈H₈₇N₃O₆; ¹H NMR (400 MHz, CDCl₃) δ 7.46 (bs, 1H), 7.21 (bs, 1H), 4.84 (qu, J=6.2 Hz, 1H), 4.05 (t, J=6.8 Hz, 2H), 3.66 (bd, J=4.9 Hz, 2H), 3.33 (d, J=5.0 Hz, 3H), 2.42-2.36 (m, 6H), 2.34-2.26 (m, 4H), 1.67-1.59 (m, 8H), 1.52-1.45 (m, 8H), 1.39-1.25 (m, 42H), 0.91 (t, J=6.9 Hz, 3H), 0.87 (t, J=6.8 Hz, 6H); ¹³C NMR (101 MHz, CDCl₃) δ 182.9, 182.4, 174.0, 173.9, 168.2, 168.2, 74.5, 64.4, 54.0, 53.5, 44.3, 34.5, 34.3, 34.1, 31.9, 31.2, 29.5, 29.4, 29.3, 29.1, 29.0, 28.3, 28.1, 27.5, 26.8, 26.3, 25.3, 25.0, 24.0, 23.1, 22.6, 22.3, 14.1, 13.9.

### pentyl 10-((3-aminopropyl) (5-(nonadecan-10-yloxy)-5-oxopentyl)amino) decanoate (Compound 93)

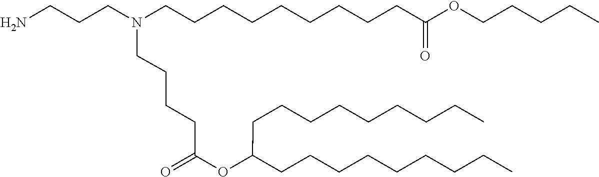 [SMILES: CCCCCCCCCC(CCCCCCCCC)OC(=O)CCCCN(CCCN)CCCCCCCCCC(=O)OCCCCC]

Compound 93 was synthesized according to Representative Procedure 2 and general Methods A, C, D, E and H. Product was obtained as a colorless oil (510 mg, 0.749 mmol, 93%). MS (ESI): m/z [M+H]⁺ 681.7 for C₄₂H₈₈N₂O₄; ¹H NMR (400 MHz, CDCl₃) δ 4.85 (qu J=6.2 Hz, 1H), 4.04 (t, J=6.8 Hz, 2H), 2.70 (t, J=6.6 Hz, 2H), 2.42 (t, J=7.2 Hz, 2H), 2.38 (t, J=7.5 Hz, 2H), 2.35 (t, J=7.5 Hz, 2H), 2.28 (t, J=7.5 Hz, 2H), 2.27 (t, J=7.8 Hz, 2H), 1.66-1.07 (m, 60H), 0.89 (t, J=7.2 Hz, 3H), 0.86 (t, J=7.2 Hz, 6H); ¹³C NMR (101 MHz, CDCl₃) δ 174.1, 173.6, 74.3, 64.5, 54.3, 53.9, 52.0, 41.0, 34.7, 34.5, 34.3, 32.0, 31.2, 29.7, 29.7, 29.7, 29.7, 29.6, 29.4, 29.4, 29.3, 28.5, 28.2, 27.7, 27.2, 26.7, 25.5, 25.1, 23.3, 22.8, 22.4, 14.2, 14.1.

### Pentyl 10-((3-((2-(methylamino)-3,4-dioxocyclobut-1-en-1-yl)amino) propyl) (5-(nonadecan-10-yloxy)-5-oxopentyl)amino) decanoate (Compound 94)

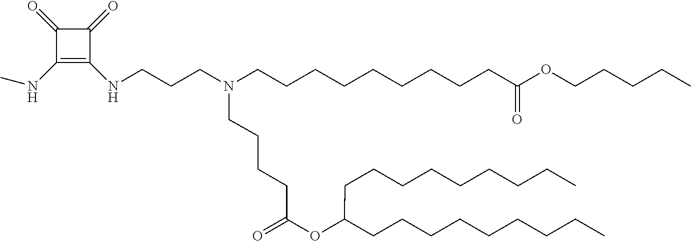 [SMILES: CCCCCCCCCC(CCCCCCCCC)OC(=O)CCCCN(CCCCCCCCCC(=O)OCCCCC)CCCNc1c(NC)c(=O)c1=O]

Compound 94 was synthesized according to Representative Procedures 2 and 3 and general Methods A, C, D, E, H and I. Product was obtained as a colorless waxy amorphous solid (260 mg, 0.329 mmol, 75%). MS (ESI): m/z [M+H]⁺ 791.4 for C₄₇H₈₈N₃O₆; ¹H NMR (400 MHz, CDCl₃) δ 7.34 (bs, 1H), 6.98 (bs, 1H), 4.81 (qu J=6.2 Hz, 1H), 4.04 (t, J=6.8 Hz, 2H), 3.69 (bs, 2H), 3.29 (d, J=5.0 Hz, 3H), 2.48 (t, J=6.4 Hz, 2H), 2.40 (t, J=7.0 Hz, 2H), 2.35 (t, J=7.4 Hz, 2H), 2.32 (t, J=7.2 Hz, 2H), 2.27 (t, J=7.6 Hz, 2H), 1.75 (qu J=6.3 Hz, 2H), 1.66-1.14 (m, 56H), 0.89 (t, J=6.8 Hz, 3H), 0.86 (t, J=7.0 Hz, 6H); ¹³C NMR (101 MHz, CDCl₃) δ 183.3, 183.0, 174.3, 174.1, 168.5, 168.3, 74.9, 64.5, 53.9, 53.6, 51.3, 43.3, 34.5, 34.2, 32.0, 31.3, 29.7, 29.7, 29.6, 29.6, 29.4, 29.4, 29.3, 28.5, 28.2, 28.2, 27.7, 26.7, 26.4, 25.4, 25.1, 23.1, 22.8, 22.4, 14.2, 14.1.

### Pentyl 10-((4-aminobutyl) (5-(nonadecan-10-yloxy)-5-oxopentyl)amino) decanoate (Compound 95)

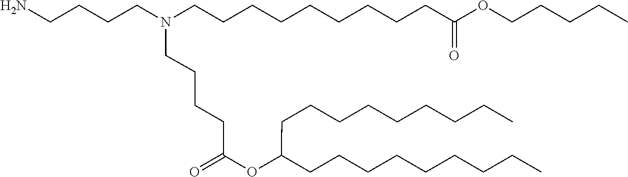 [SMILES: CCCCCCCCCC(CCCCCCCCC)OC(=O)CCCCN(CCCCN)CCCCCCCCCC(=O)OCCCCC]

Compound 95 was synthesized according to Representative Procedure 2 and general Methods A, C, D, E and H. Product was obtained as a colorless oil (600 mg, 0.863 mmol, 95%). MS (ESI): m/z [M+H]⁺ 695.7 for C₄₃H₈₇N₂O₄; ¹H NMR (400 MHz, CDCl₃) δ 4.84 (qu J=6.3 Hz, 1H), 4.04 (t, J=6.8 Hz, 2H), 2.68 (t, J=6.6 Hz, 2H), 2.44-2.31 (m, 6H), 2.28 (t, J=7.6 Hz, 2H), 2.27 (t, J=7.6 Hz, 2H), 1.66-1.14 (m, 60H), 0.92-0.82 (m, 9H); ¹³C NMR (101 MHz, CDCl₃) δ 174.1, 173.6, 74.3, 64.5, 54.2, 54.1, 53.8, 42.3, 34.7, 34.5, 34.3, 32.0, 31.9, 29.7, 29.7, 29.7, 29.7, 29.6, 29.4, 29.4, 29.3, 28.5, 28.2, 27.7, 27.2, 26.7, 25.4, 25.1, 24.7, 23.3, 22.8, 22.4, 14.2, 14.1.

### Pentyl 10-((4-((2-(methylamino)-3,4-dioxocyclobut-1-en-1-yl)amino)butyl) (5-(nonadecan-10-yloxy)-5-oxopentyl)amino) decanoate (Compound 96)

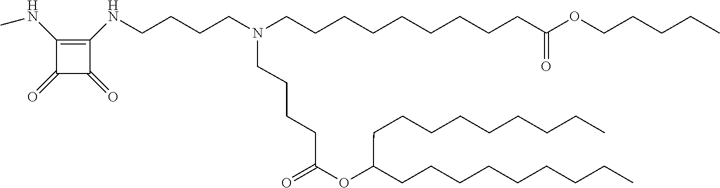 [SMILES: CCCCCCCCCC(CCCCCCCCC)OC(=O)CCCCN(CCCCCCCCCC(=O)OCCCCC)CCCCNc1c(NC)c(=O)c1=O]

Compound 96 was synthesized according to Representative Procedures 2 and 3 and general Methods A, C, D, E, H and I. Product was obtained as a colorless waxy amorphous solid (240 mg, 0.299 mmol, 69%). MS (ESI): m/z [M+H]⁺ 805.4 for C₄₈H₉₀N₃O₆; ¹H NMR (600 MHz, CDCl₃) δ 7.54 (bs, 1H), 7.29 (bs, 1H), 4.83 (qu J=6.3 Hz, 1H), 4.04 (t, J=6.6 Hz, 2H), 3.65 (bs, 2H), 3.31 (d, J=5.1 Hz, 3H), 2.45-2.32 (m, 6H), 2.29 (t, J=7.5 Hz, 2H), 2.27 (t, J=7.5 Hz, 2H), 1.68-1.55 (m, 8H), 1.54-1.11 (m, 52H), 0.89 (t, J=7.2 Hz, 3H), 0.86 (t, J=7.2 Hz, 6H); ¹³C NMR (151 MHz, CDCl₃) δ 183.0, 182.5, 174.1, 174.0, 168.4, 168.3, 74.6, 64.5, 54.2, 53.7, 44.6, 34.7, 34.5, 34.2, 32.0, 31.4, 29.7, 29.7, 29.7, 29.6, 29.4, 29.4, 29.3, 29.2, 28.5, 28.2, 27.8, 25.5, 25.1, 24.2, 23.3, 22.8, 22.4, 14.2, 14.1.

### 9-[(2-hydroxyethyl) [5-(nonadecan-10-yloxy)-5-oxopentyl]amino]nonyl hexanoate (Compound 97)

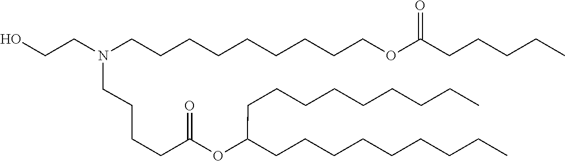 [SMILES: CCCCCCCCCC(CCCCCCCCC)OC(=O)CCCCN(CCO)CCCCCCCCCOC(=O)CCCCC]

Compound 97 was synthesized according to Representative Procedure 1 using Intermediate H and general Methods F, B, C and G. Product was obtained as a pale yellow oil (240 mg, 0.498 mmol, 71%). MS (ESI): m/z [M+H]⁺ 669.3 for C₄₁H₈₁NO₅; ¹H NMR (600 MHz, CDCl₃-CD₃OD=2:1 v/v) δ 4.90-4.85 (m, 1H), 4.07 (t, J=6.7 Hz, 2H), 3.60 (t, J=5.9 Hz, 2H), 2.63 (t, J=5.9 Hz, 2H), 2.54-2.50 (m, 2H), 2.49-2.47 (m, 2H), 2.34 (t, J=7.4 Hz, 2H), 2.31 (t, J=7.5 Hz, 2H), 1.66-1.61 (m, 6H), 1.54-1.50 (m, 6H), 1.49-1.44 (m, 2H), 1.38-1.27 (m, 43H), 0.91 (t, J=7.1 Hz, 3H), 0.89 (t, J=7.0 Hz, 6H); ¹³C NMR (151 MHz, CDCl₃-CD₃OD=2:1 v/v) δ 175.1, 174.4, 75.1, 65.0, 59.2, 56.1, 54.6, 54.1, 34.8, 34.7, 34.4, 32.2, 31.6, 29.9, 29.9, 29.8, 29.8, 29.6, 29.5, 28.9, 27.8, 27.0, 26.4, 26.3, 25.7, 25.0, 23.4, 23.0, 22.6, 14.2, 14.0.

### 11-{[5-(heptadecan-9-yloxy)-5-oxopentyl](2-hydroxyethyl)amino}undecyl hexanoate (Compound 98)

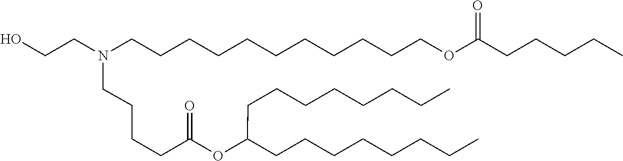 [SMILES: CCCCCCCCC(CCCCCCCC)OC(=O)CCCCN(CCO)CCCCCCCCCCCOC(=O)CCCCC]

Compound 98 was synthesized according to Representative Procedure 1 using Intermediate H and general Methods F, B, C and G. Product was obtained as a pale yellow oil (260 mg, 0.455 mmol, 84%). MS (ESI): m/z [M+H]⁺ 669.1 for C₄₁H₈₁NO₅; ¹H NMR (600 MHz, CDCl₃-CD₃OD=2:1 v/v) δ 4.90-4.85 (m, 1H), 4.07 (t, J=6.7 Hz, 2H), 3.61 (t, J=5.9 Hz, 2H), 2.64 (t, J=5.9 Hz, 2H), 2.55-2.53 (m, 2H), 2.52-2.49 (m, 2H), 2.35 (t, J=7.4 Hz, 2H), 2.31 (t, J=7.5 Hz, 2H), 1.66-1.61 (m, 6H), 1.55-1.51 (m, 6H), 1.49-1.44 (m, 2H), 1.37-1.28 (m, 43H), 0.91 (t, J=7.1 Hz, 3H), 0.89 (t, J=7.0 Hz, 6H); ¹³C NMR (151 MHz, CDCl₃-CD₃OD=2:1 v/v) δ 175.1, 174.4, 75.1, 65.0, 59.1, 56.1, 54.5, 54.1, 34.8, 34.7, 34.4, 32.2, 31.6, 29.9, 29.9, 29.9, 29.8, 29.8, 29.6, 28.9, 27.9, 26.9, 26.3, 26.3, 25.7, 25.0, 23.3, 23.0, 22.6, 14.2, 14.0.

### 11-[(2-hydroxyethyl) [5-(nonadecan-10-yloxy)-5-oxopentyl]amino]undecyl hexanoate (Compound 99)

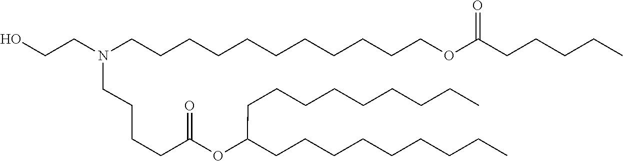 [SMILES: CCCCCCCCCC(CCCCCCCCC)OC(=O)CCCCN(CCO)CCCCCCCCCCCOC(=O)CCCCC]

Compound 99 was synthesized according to Representative Procedure 1 using Intermediate H and general Methods F, B, C and G. Product was obtained as a pale yellow oil (230 mg, 0.455 mmol, 72%). MS (ESI): m/z [M+H]⁺ 697.4 for C₄₃H₈₈NO₅; ¹H NMR (600 MHz, CDCl₃-CD₃OD=2:1 v/v) δ 4.90-4.85 (m, 1H), 4.07 (t, J=6.7 Hz, 2H), 3.60 (t, J=5.9 Hz, 2H), 2.63 (t, J=5.9 Hz, 2H), 2.54-2.52 (m, 2H), 2.50-2.48 (m, 2H), 2.34 (t, J=7.4 Hz, 2H), 2.31 (t, J=7.5 Hz, 2H), 1.66-1.61 (m, 6H), 1.54-1.50 (m, 6H), 1.49-1.44 (m, 2H), 1.37-1.27 (m, 47H), 0.91 (t, J=7.1 Hz, 3H), 0.89 (t, J=7.1 Hz, 6H); ¹³C NMR (150 MHz, CDCl₃-CD₃OD=2:1 v/v) δ 175.1, 174.3, 75.1, 65.0, 59.2, 56.1, 54.5, 54.1, 34.8, 34.7, 34.4, 32.2, 31.6, 29.9, 29.9, 29.9, 29.8, 29.6, 29.6, 28.9, 27.9, 26.9, 26.4, 26.3, 25.7, 25.0, 23.4, 23.0, 22.6, 14.2, 14.0.

### 9-[(2-hydroxyethyl) [5-(nonadecan-10-yloxy)-5-oxopentyl]amino]nonyl hexanoate (Compound 100)

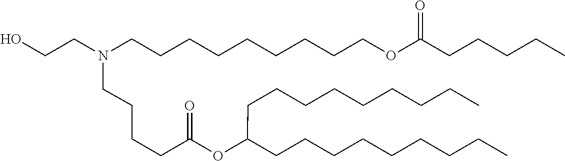 [SMILES: CCCCCCCCCC(CCCCCCCCC)OC(=O)CCCCN(CCO)CCCCCCCCCOC(=O)CCCCC]

Compound 100 was synthesized according to Representative Procedure 1 using Intermediate H and general Methods F, B, C, and G. Product was obtained as a pale yellow oil (240 mg, 0.356 mmol, 71%). MS (ESI): m/z [M+H]⁺ 669.3 for C₄₁H₈₂NO₅; ¹H NMR (600 MHz, CDCl₃-CD₃OD=2:1 v/v) δ 4.90-4.85 (m, 1H), 4.07 (t, J=6.7 Hz, 2H), 3.60 (t, J=5.9 Hz, 2H), 2.63 (t, J=5.9 Hz, 2H), 2.54-2.50 (m, 2H), 2.49-2.47 (m, 2H), 2.34 (t, J=7.4 Hz, 2H), 2.31 (t, J=7.5 Hz, 2H), 1.66-1.61 (m, 6H), 1.54-1.50 (m, 6H), 1.49-1.44 (m, 2H), 1.38-1.27 (m, 43H), 0.91 (t, J=7.1 Hz, 3H), 0.89 (t, J=7.0 Hz, 6H); ¹³C NMR (151 MHz, CDCl₃-CD₃OD=2:1 v/v) δ 175.1, 174.4, 75.1, 65.0, 59.2, 56.1, 54.6, 54.1, 34.8, 34.7, 34.4, 32.2, 31.6, 29.9, 29.9, 29.8, 29.8, 29.6, 29.5, 28.9, 27.8, 27.0, 26.4, 26.3, 25.7, 25.0, 23.4, 23.0, 22.6, 14.2, 14.0.

### 5-{[5-(heptadecan-9-yloxy)-5-oxopentyl](3-{[2-(methylamino)-3,4-dioxocyclobut-1-en-1-yl]amino}propyl)amino}pentyl hexanoate (Compound 101)

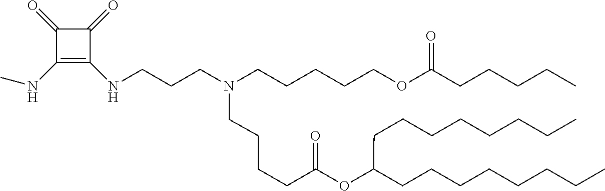 [SMILES: CCCCCCCCC(CCCCCCCC)OC(=O)CCCCN(CCCCCOC(=O)CCCCC)CCCNc1c(NC)c(=O)c1=O]

Compound 101 was synthesized according to Representative Procedures 2 and 3 and general Methods A, C, D, E, G and I. Product was obtained as a pale yellow oil (50 mg, 0.070 mmol, 55%). MS (ESI): m/z [M+H]⁺ 706.1 for C₄₁H₇₆N₃O₆; ¹H NMR (600 MHz, CDCl₃-CD₃OD=2:1 v/v) δ 4.84-4.80 (m, 1H), 4.04 (t, J=6.5 Hz, 2H), 3.65-3.62 (m, 2H), 3.25 (bs, 3H), 2.96-2.87 (m, 5H), 2.34 (t, J=6.1 Hz, 2H), 2.27 (t, J=7.6 Hz, 2H), 1.93 (bs, 2H), 1.67-1.54 (m, 9H), 1.49-1.48 (m, 4H), 1.39-1.34 (m, 2H), 1.31-1.23 (m, 30H), 0.86-0.83 (m, 9H); ¹³C NMR (150 MHz, CDCl₃-CD₃OD=2:1 v/v) δ 182.4, 182.0, 174.2, 173.1, 168.7, 167.5, 74.7, 63.6, 52.8, 52.6, 50.5, 41.2, 33.9, 33.7, 33.5, 31.5, 31.0, 30.9, 30.5, 29.2, 29.2, 28.9, 28.0, 26.2, 25.0, 24.3, 23.8, 23.6, 23.1, 22.3, 22.0, 22.0, 13.6, 13.4.

### 9-[(3-{[2-(methylamino)-3,4-dioxocyclobut-1-en-1-yl]amino}propyl) [5-(nonadecan-10-yloxy)-5-oxopentyl]amino]nonyl hexanoate (Compound 102)

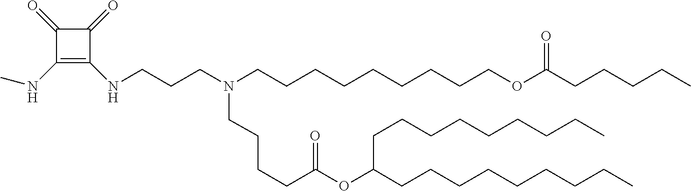 [SMILES: CCCCCCCCCC(CCCCCCCCC)OC(=O)CCCCN(CCCCCCCCCOC(=O)CCCCC)CCCNc1c(NC)c(=O)c1=O]

Compound 102 was synthesized according to Representative Procedures 2 and 3 and general Methods A, C, D, E, H and I. Product was obtained as a pale yellow oil (520 mg, 0.858 mmol, 61%). MS (ESI): m/z [M+H]⁺ 791.4 for C₄₇H₈₇N₃O₆; ¹H NMR (600 MHz, CDCl₃-CD₃OD=2:1 v/v) δ 4.90-4.84 (m, 1H), 4.07 (t, J=6.7 Hz, 2H), 3.63 (bs, 2H), 3.28 (bs, 3H), 2.54-2.42 (m, 6H), 2.37-2.29 (m, 4H), 1.80-1.73 (m, 2H), 1.67-1.59 (m, 6H), 1.56-1.41 (m, 8H), 1.39-1.25 (m, 45H), 0.93-0.87 (m, 9H); ¹³C NMR (151 MHz, CDCl₃-CD₃OD=2:1 v/v) δ 182.6, 182.4, 174.7, 173.2, 169.4, 167.6, 75.1, 64.5, 53.0, 52.6, 50.7, 40.8, 34.3, 34.0, 33.5, 31.8, 31.2, 30.7, 29.5, 29.5, 29.4, 29.2, 29.2, 29.0, 28.5, 26.6, 25.8, 25.3, 24.6, 23.6, 23.1, 22.6, 22.2, 21.9, 13.8, 13.6.

### 11-[(3-{[2-(methylamino)-3,4-dioxocyclobut-1-en-1-yl]amino}propyl)[5-(nonadecan-10-yloxy)-5-oxopentyl]amino]undecyl hexanoate (Compound 103)

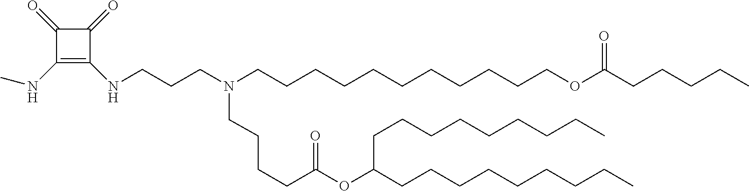 [SMILES: CCCCCCCCCC(CCCCCCCCC)OC(=O)CCCCN(CCCCCCCCCCCOC(=O)CCCCC)CCCNc1c(NC)c(=O)c1=O]

Compound 103 was synthesized according to Representative Procedures 2 and 3 and general Methods A, C, D, E, H and I. Product was obtained as a pale yellow oil (580 mg, 0.748 mmol, 77%). MS (ESI): m/z [M+H]⁺ 819.2 for C₄₉H₉₁N₃O₆; ¹H NMR (600 MHz, CDCl₃-CD₃OD=2:1 v/v) δ 4.88-4.84 (m, 1H), 4.07 (t, J=6.7 Hz, 2H), 3.63 (m, 2H), 3.28 (bs, 3H), 2.52-2.42 (m, 6H), 2.35 (t, J=7.4 Hz, 2H), 2.31 (t, J=7.5 Hz, 2H), 1.78-1.73 (m, 2H), 1.66-1.59 (m, 6H), 1.55-1.48 (m, 6H), 1.47-1.41 (m, 2H), 1.37-1.27 (m, 49H), 0.91 (t, J=7.1 Hz, 3H), 0.88 (t, J=7.1 Hz, 6H); ¹³C NMR (150 MHz, CDCl₃-CD₃OD=2:1 v/v) δ 182.9, 182.7, 175.2, 173.6, 169.7, 167.9, 75.5, 64.9, 53.3, 52.9, 51.0, 41.2, 34.7, 34.3, 33.8, 32.2, 31.6, 31.1, 29.9, 29.9, 29.8, 29.8, 29.7, 29.6, 29.5, 29.5, 28.9, 27.0, 26.2, 26.2, 25.7, 25.0, 24.0, 23.5, 23.0, 22.6, 22.2, 14.2, 14.0.

The compounds listed above correspond to the exemplary structures of Formula I according to Table 1:

TABLE 1

| | Compound |
|---|---|
| Exemplary Structures of Formula I | number |
| 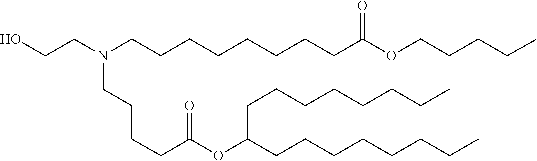 [SMILES: CCCCCCCCC(CCCCCCCC)OC(=O)CCCCN(CCO)CCCCCCCCC(=O)OCCCCC] (I)(a) | Compound 1 |
| 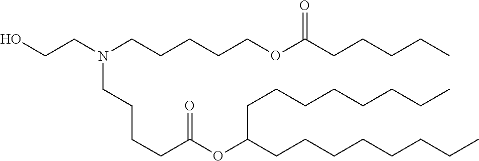 [SMILES: CCCCCCCCC(CCCCCCCC)OC(=O)CCCCN(CCO)CCCCCOC(=O)CCCCC] (I)(b) | Compound 68 |
| 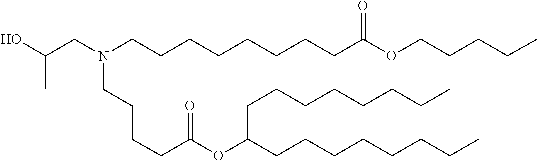 [SMILES: CCCCCCCCC(CCCCCCCC)OC(=O)CCCCN(CCCCCCCCC(=O)OCCCCC)CC(C)O] (I)(c) | Compound 50 |
| 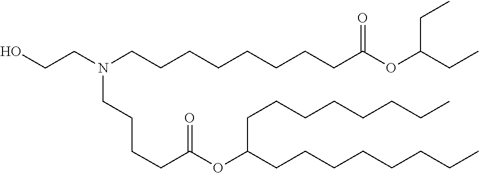 [SMILES: CCCCCCCCC(CCCCCCCC)OC(=O)CCCCN(CCO)CCCCCCCCC(=O)OC(CC)CC] (I)(d) | Compound 48 |
| 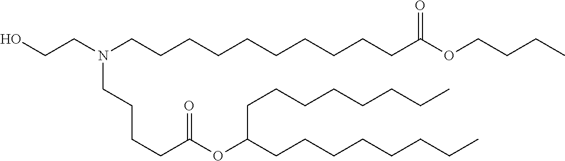 [SMILES: CCCCCCCCC(CCCCCCCC)OC(=O)CCCCN(CCO)CCCCCCCCCCC(=O)OCCCC] (I)(e) | Compound 35 |
| 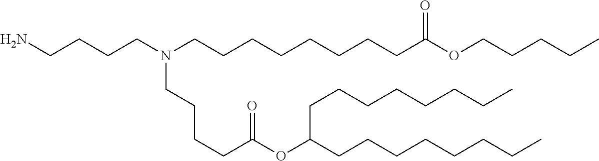 [SMILES: CCCCCCCCC(CCCCCCCC)OC(=O)CCCCN(CCCCN)CCCCCCCCC(=O)OCCCCC] (I)(f) | Compound 53 |
| 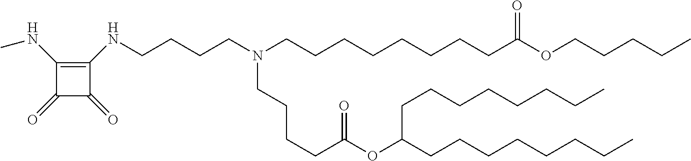 [SMILES: CCCCCCCCC(CCCCCCCC)OC(=O)CCCCN(CCCCCCCCC(=O)OCCCCC)CCCCNc1c(NC)c(=O)c1=O] (I)(g) | Compound 54 |
| 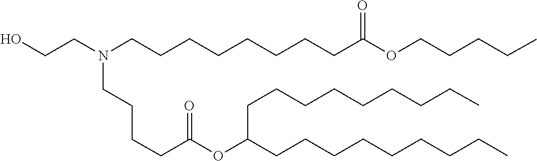 [SMILES: CCCCCCCCCC(CCCCCCCCC)OC(=O)CCCCN(CCO)CCCCCCCCC(=O)OCCCCC] (I)(h) | Compound 42 |
| 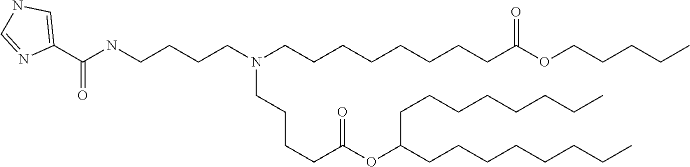 (I)(i) | Compound 65 |
| 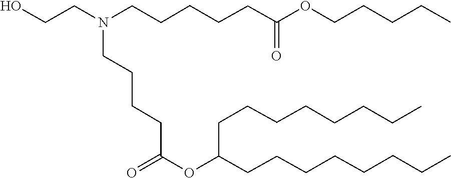 [SMILES: CCCCCCCCC(CCCCCCCC)OC(=O)CCCCN(CCO)CCCCCC(=O)OCCCCC] (I)(n) | Compound 2 |
| 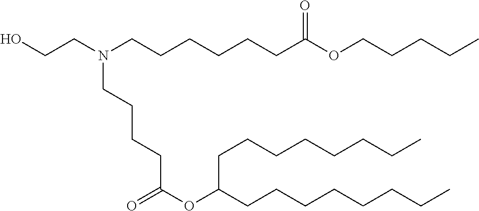 [SMILES: CCCCCCCCC(CCCCCCCC)OC(=O)CCCCN(CCO)CCCCCCC(=O)OCCCCC] (I)(o) | Compound 3 |
| 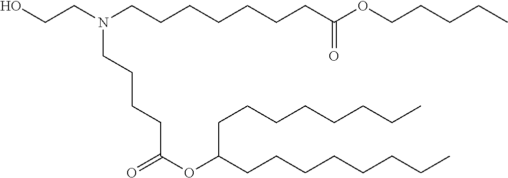 [SMILES: CCCCCCCCC(CCCCCCCC)OC(=O)CCCCN(CCO)CCCCCCCC(=O)OCCCCC] (I)(p) | Compound 4 |
| 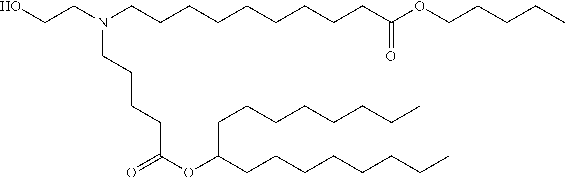 [SMILES: CCCCCCCCC(CCCCCCCC)OC(=O)CCCCN(CCO)CCCCCCCCCC(=O)OCCCCC] (I)(q) | Compound 5 |
| 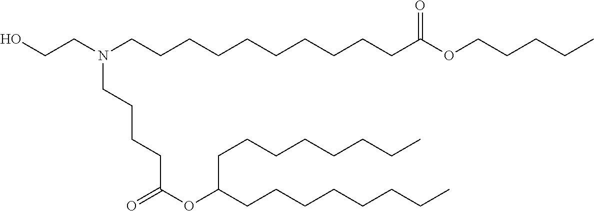 [SMILES: CCCCCCCCC(CCCCCCCC)OC(=O)CCCCN(CCO)CCCCCCCCCCC(=O)OCCCCC] (I)(r) | Compound 6 |
| 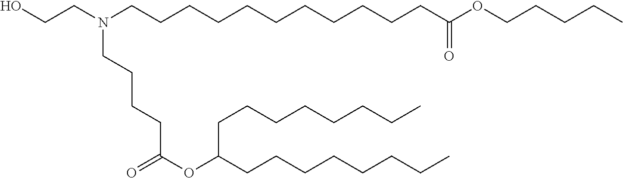 [SMILES: CCCCCCCCC(CCCCCCCC)OC(=O)CCCCN(CCO)CCCCCCCCCCCC(=O)OCCCCC] (I)(s) | Compound 7 |
| 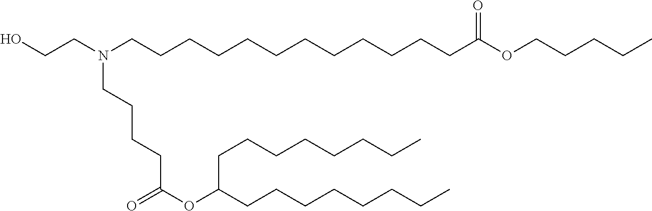 [SMILES: CCCCCCCCC(CCCCCCCC)OC(=O)CCCCN(CCO)CCCCCCCCCCCCC(=O)OCCCCC] (I)(t) | Compound 8 |
| 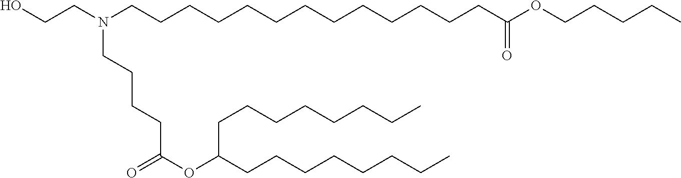 [SMILES: CCCCCCCCC(CCCCCCCC)OC(=O)CCCCN(CCO)CCCCCCCCCCCCCC(=O)OCCCCC] (I)(u) | Compound 9 |
| 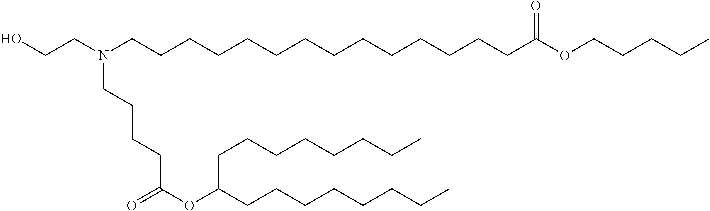 [SMILES: CCCCCCCCC(CCCCCCCC)OC(=O)CCCCN(CCO)CCCCCCCCCCCCCCC(=O)OCCCCC] (I)(v) | Compound 10 |
| 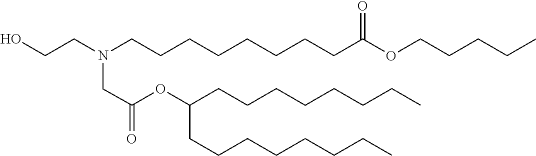 [SMILES: CCCCCCCCC(CCCCCCCC)OC(=O)CN(CCO)CCCCCCCCC(=O)OCCCCC] (I)(w) | Compound 11 |
| 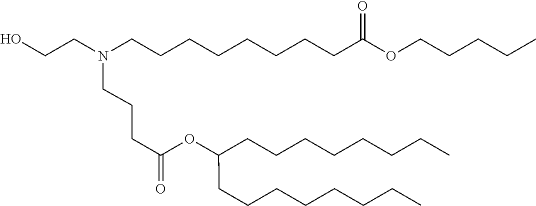 [SMILES: CCCCCCCCC(CCCCCCCC)OC(=O)CCCN(CCO)CCCCCCCCC(=O)OCCCCC] (I)(x) | Compound 12 |
| 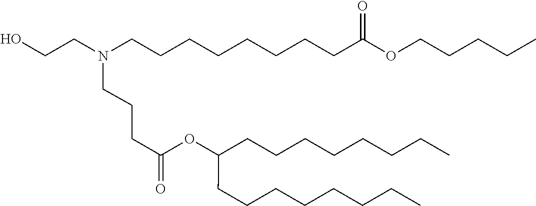 [SMILES: CCCCCCCCC(CCCCCCCC)OC(=O)CCCN(CCO)CCCCCCCCC(=O)OCCCCC] (I)(y) | Compound 13 |
| 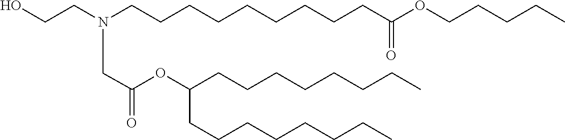 [SMILES: CCCCCCCCC(CCCCCCCC)OC(=O)CN(CCO)CCCCCCCCCC(=O)OCCCCC] (I)(z) | Compound 14 |
| 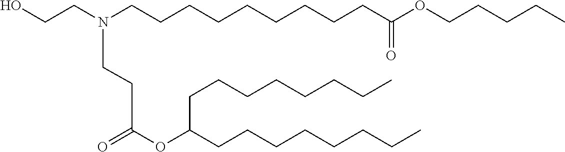 [SMILES: CCCCCCCCC(CCCCCCCC)OC(=O)CCN(CCO)CCCCCCCCCC(=O)OCCCCC] (I)(aa) | Compound 15 |
| 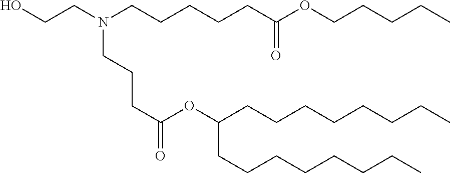 [SMILES: CCCCCCCCC(CCCCCCCC)OC(=O)CCCN(CCO)CCCCCC(=O)OCCCCC] (I)(bb) | Compound 16 |
| 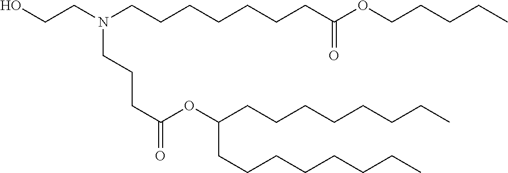 [SMILES: CCCCCCCCC(CCCCCCCC)OC(=O)CCCN(CCO)CCCCCCCC(=O)OCCCCC] (I)(cc) | Compound 17 |
| 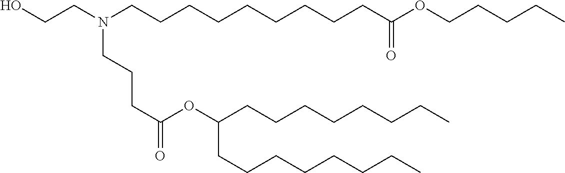 [SMILES: CCCCCCCCC(CCCCCCCC)OC(=O)CCCN(CCO)CCCCCCCCCC(=O)OCCCCC] (I)(dd) | Compound 18 |
| 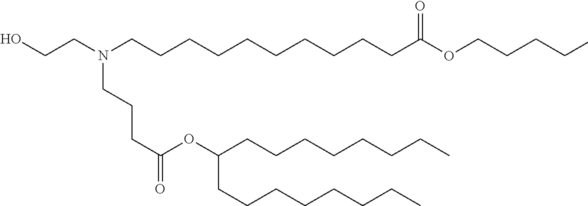 [SMILES: CCCCCCCCC(CCCCCCCC)OC(=O)CCCN(CCO)CCCCCCCCCCC(=O)OCCCCC] (I)(ee) | Compound 19 |
| 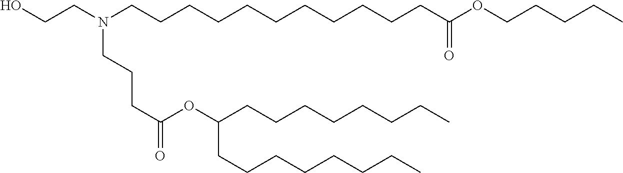 [SMILES: CCCCCCCCC(CCCCCCCC)OC(=O)CCCN(CCO)CCCCCCCCCCCC(=O)OCCCCC] (I)(ff) | Compound 20 |
| 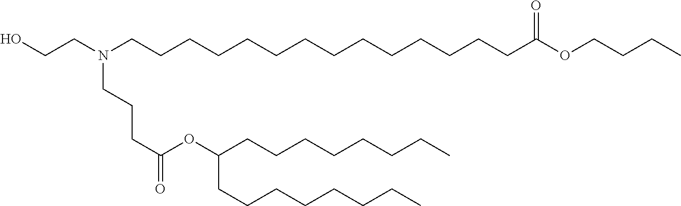 [SMILES: CCCCCCCCC(CCCCCCCC)OC(=O)CCCN(CCO)CCCCCCCCCCCCCCC(=O)OCCCC] (I)(gg) | Compound 21 |
| 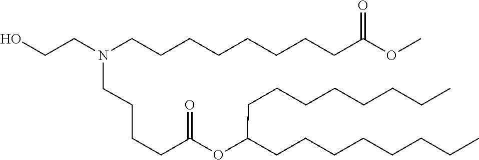 [SMILES: CCCCCCCCC(CCCCCCCC)OC(=O)CCCCN(CCO)CCCCCCCCC(=O)OC] (I)(hh) | Compound 22 |
| 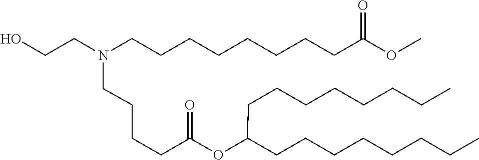 [SMILES: CCCCCCCCC(CCCCCCCC)OC(=O)CCCCN(CCO)CCCCCCCCC(=O)OC] (I)(ii) | Compound 23 |
| 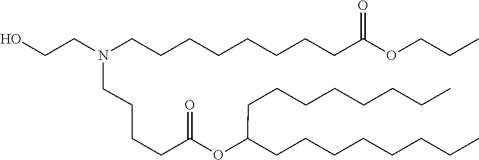 [SMILES: CCCCCCCCC(CCCCCCCC)OC(=O)CCCCN(CCO)CCCCCCCCC(=O)OCCC] (I)(jj) | Compound 24 |
| 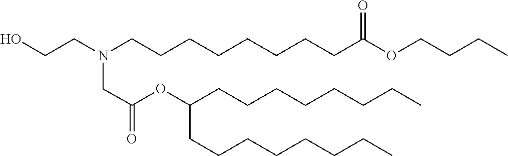 [SMILES: CCCCCCCCC(CCCCCCCC)OC(=O)CN(CCO)CCCCCCCCC(=O)OCCCC] (I)(kk) | Compound 25 |
| 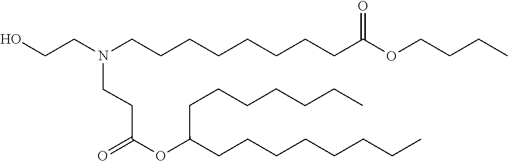 [SMILES: CCCCCCCCC(CCCCCCC)OC(=O)CCN(CCO)CCCCCCCCC(=O)OCCCC] (I)(ll) | Compound 26 |
| 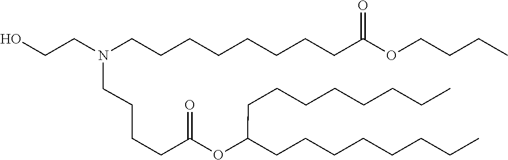 [SMILES: CCCCCCCCC(CCCCCCCC)OC(=O)CCCCN(CCO)CCCCCCCCC(=O)OCCCC] (I)(mm) | Compound 27 |
| 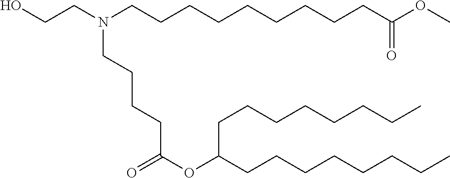 [SMILES: CCCCCCCCC(CCCCCCCC)OC(=O)CCCCN(CCO)CCCCCCCCCC(=O)OC] (I)(nn) | Compound 28 |
| 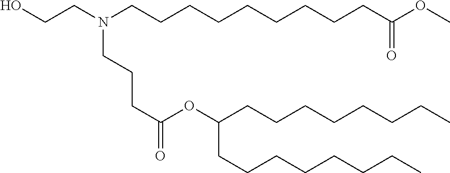 [SMILES: CCCCCCCCC(CCCCCCCC)OC(=O)CCCN(CCO)CCCCCCCCCC(=O)OC] (I)(oo) | Compound 29 |
| 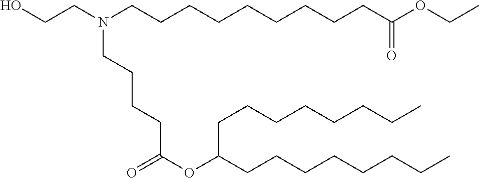 [SMILES: CCCCCCCCC(CCCCCCCC)OC(=O)CCCCN(CCO)CCCCCCCCCC(=O)OCC] (I)(pp) | Compound 30 |
| 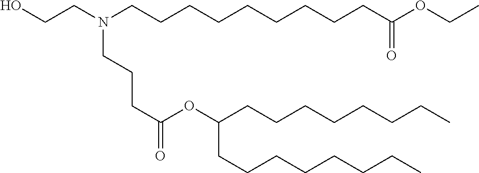 [SMILES: CCCCCCCCC(CCCCCCCC)OC(=O)CCCN(CCO)CCCCCCCCCC(=O)OCC] (I)(qq) | Compound 31 |
| 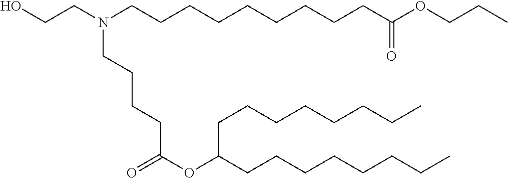 [SMILES: CCCCCCCCC(CCCCCCCC)OC(=O)CCCCN(CCO)CCCCCCCCCC(=O)OCCC] (I)(rr) | Compound 32 |
| 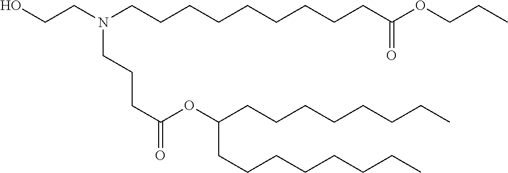 [SMILES: CCCCCCCCC(CCCCCCCC)OC(=O)CCCN(CCO)CCCCCCCCCC(=O)OCCC] (I)(ss) | Compound 33 |
| 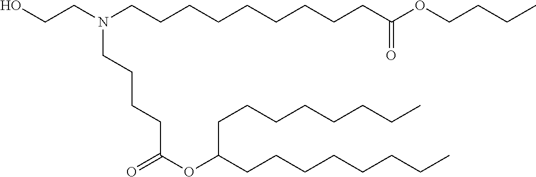 [SMILES: CCCCCCCCC(CCCCCCCC)OC(=O)CCCCN(CCO)CCCCCCCCCC(=O)OCCCC] (I)(tt) | Compound 34 |
| 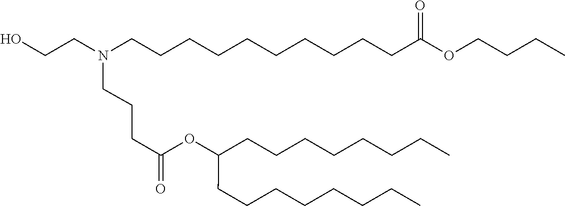 [SMILES: CCCCCCCCC(CCCCCCCC)OC(=O)CCCN(CCO)CCCCCCCCCCC(=O)OCCCC] (I)(uu) | Compound 36 |
| 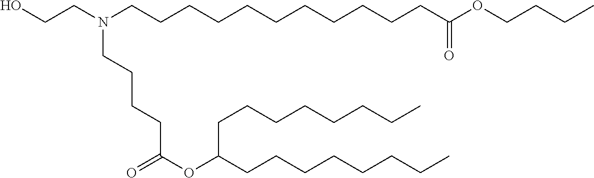 [SMILES: CCCCCCCCC(CCCCCCCC)OC(=O)CCCCN(CCO)CCCCCCCCCCCC(=O)OCCCC] (I)(vv) | Compound 37 |
| 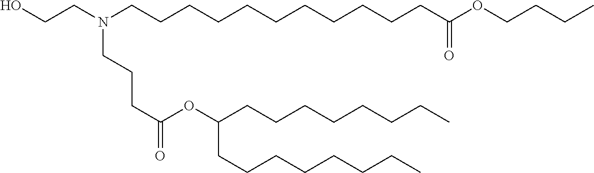 [SMILES: CCCCCCCCC(CCCCCCCC)OC(=O)CCCN(CCO)CCCCCCCCCCCC(=O)OCCCC] (I)(ww) | Compound 38 |
| 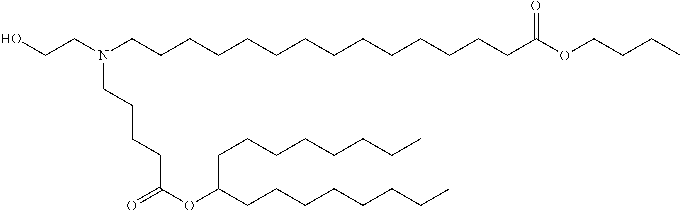 [SMILES: CCCCCCCCC(CCCCCCCC)OC(=O)CCCCN(CCO)CCCCCCCCCCCCCCC(=O)OCCCC] (I)(xx) | Compound 39 |
| 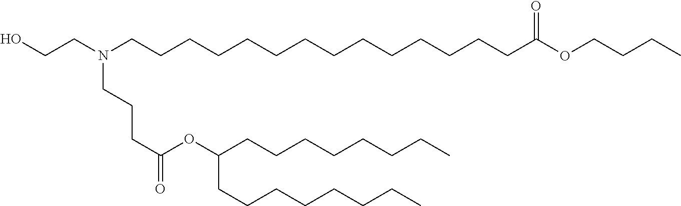 [SMILES: CCCCCCCCC(CCCCCCCC)OC(=O)CCCN(CCO)CCCCCCCCCCCCCCC(=O)OCCCC] (I)(yy) | Compound 40 |
| 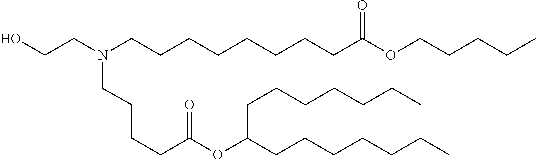 [SMILES: CCCCCCCC(CCCCCCC)OC(=O)CCCCN(CCO)CCCCCCCCC(=O)OCCCCC] (I)(zz) | Compound 41 |
| 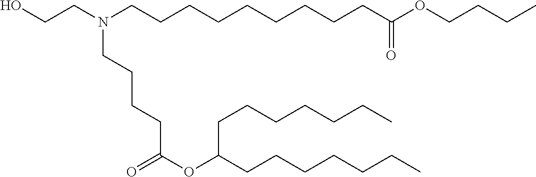 [SMILES: CCCCCCCC(CCCCCCC)OC(=O)CCCCN(CCO)CCCCCCCCCC(=O)OCCCC] (I)(aaa) | Compound 43 |
| 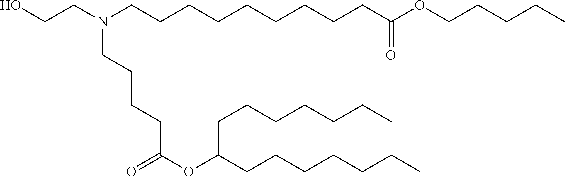 [SMILES: CCCCCCCC(CCCCCCC)OC(=O)CCCCN(CCO)CCCCCCCCCC(=O)OCCCCC] (I)(bbb) | Compound 44 |
| 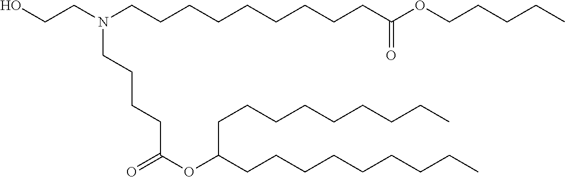 [SMILES: CCCCCCCCCC(CCCCCCCCC)OC(=O)CCCCN(CCO)CCCCCCCCCC(=O)OCCCCC] (I)(ccc) | Compound 45 |
| 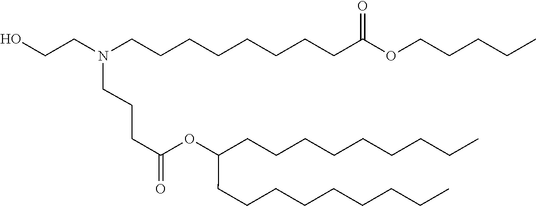 [SMILES: CCCCCCCCCC(CCCCCCCCC)OC(=O)CCCN(CCO)CCCCCCCCC(=O)OCCCCC] (I)(ddd) | Compound 46 |
| 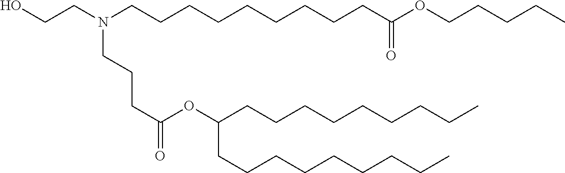 [SMILES: CCCCCCCCCC(CCCCCCCCC)OC(=O)CCCN(CCO)CCCCCCCCCC(=O)OCCCCC] (I)(eee) | Compound 47 |
| 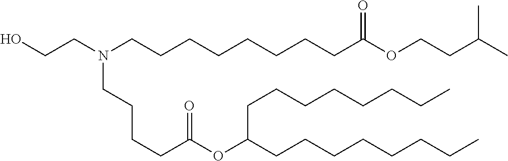 [SMILES: CCCCCCCCC(CCCCCCCC)OC(=O)CCCCN(CCO)CCCCCCCCC(=O)OCCC(C)C] (I)(fff) | Compound 49 |
| 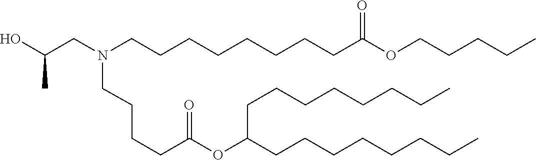 [SMILES: CCCCCCCCC(CCCCCCCC)OC(=O)CCCCN(CCCCCCCCC(=O)OCCCCC)C[C@@H](C)O] (I)(ggg) | Compound 51 |
| 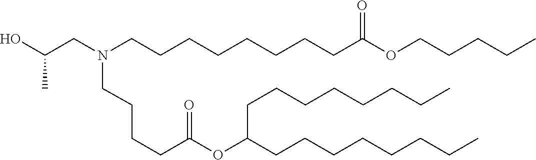 [SMILES: CCCCCCCCC(CCCCCCCC)OC(=O)CCCCN(CCCCCCCCC(=O)OCCCCC)C[C@H](C)O] (I)(hhh) | Compound 52 |
| 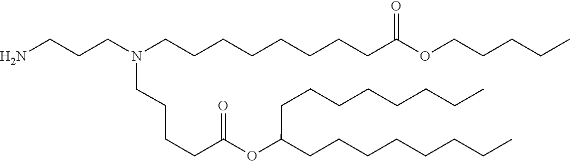 [SMILES: CCCCCCCCC(CCCCCCCC)OC(=O)CCCCN(CCCN)CCCCCCCCC(=O)OCCCCC] (I)(iii) | Compound 55 |
| 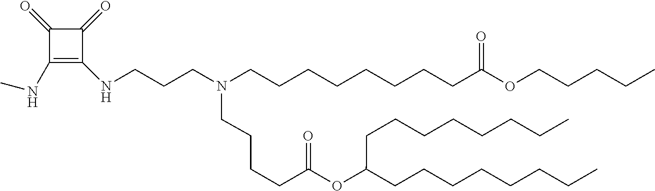 [SMILES: CCCCCCCCC(CCCCCCCC)OC(=O)CCCCN(CCCCCCCCC(=O)OCCCCC)CCCNc1c(NC)c(=O)c1=O] (I)(jjj) | Compound 56 |
| 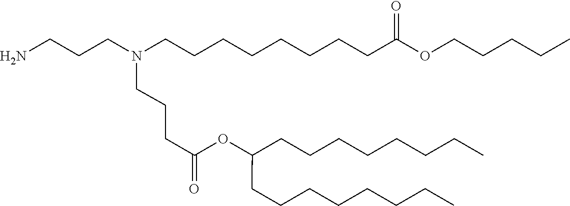 [SMILES: CCCCCCCCC(CCCCCCCC)OC(=O)CCCN(CCCN)CCCCCCCCC(=O)OCCCCC] (I)(kkk) | Compound 57 |
| 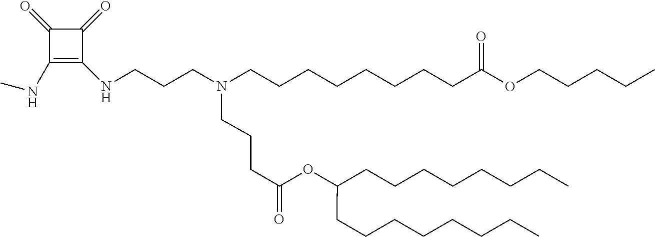 [SMILES: CCCCCCCCC(CCCCCCCC)OC(=O)CCCN(CCCCCCCCC(=O)OCCCCC)CCCNc1c(NC)c(=O)c1=O] (I)(lll) | Compound 58 |
| 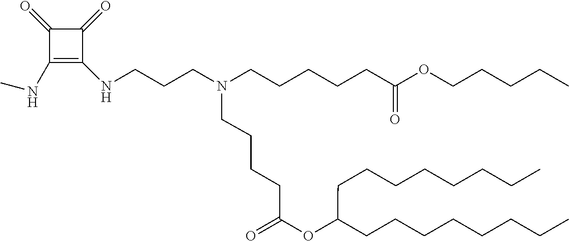 [SMILES: CCCCCCCCC(CCCCCCCC)OC(=O)CCCCN(CCCCCC(=O)OCCCCC)CCCNc1c(NC)c(=O)c1=O] (I)(mmm) | Compound 59 |
| 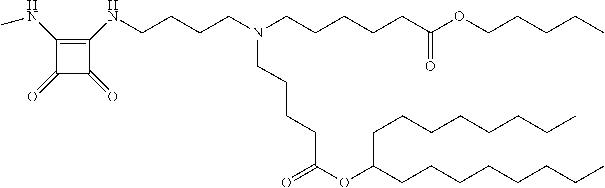 [SMILES: CCCCCCCCC(CCCCCCCC)OC(=O)CCCCN(CCCCCC(=O)OCCCCC)CCCCNc1c(NC)c(=O)c1=O] (I)(nnn) | Compound 60 |
| 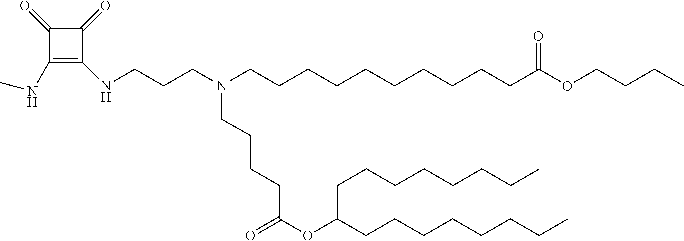 [SMILES: CCCCCCCCC(CCCCCCCC)OC(=O)CCCCN(CCCCCCCCCCC(=O)OCCCC)CCCNc1c(NC)c(=O)c1=O] (I)(ooo) | Compound 61 |
| 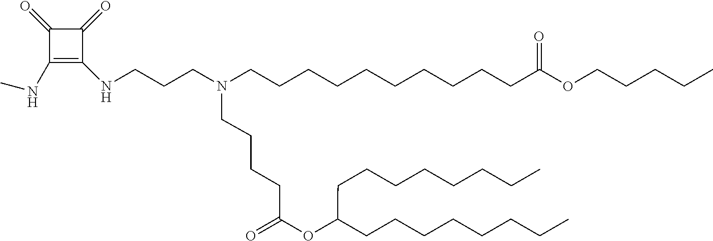 [SMILES: CCCCCCCCC(CCCCCCCC)OC(=O)CCCCN(CCCCCCCCCCC(=O)OCCCCC)CCCNc1c(NC)c(=O)c1=O] (I)(ppp) | Compound 62 |
| 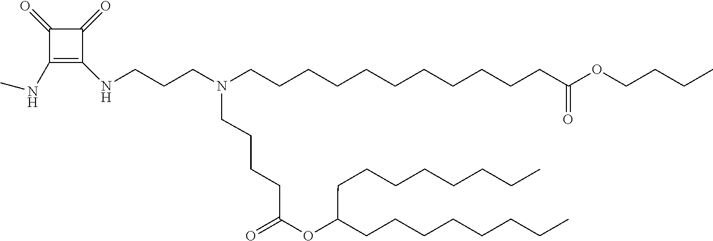 [SMILES: CCCCCCCCC(CCCCCCCC)OC(=O)CCCCN(CCCCCCCCCCCC(=O)OCCCC)CCCNc1c(NC)c(=O)c1=O] (I)(qqq) | Compound 63 |
| 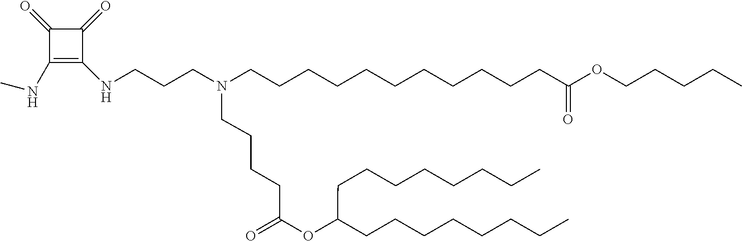 [SMILES: CCCCCCCCC(CCCCCCCC)OC(=O)CCCCN(CCCCCCCCCCCC(=O)OCCCCC)CCCNc1c(NC)c(=O)c1=O] (I)(rrr) | Compound 64 |
| 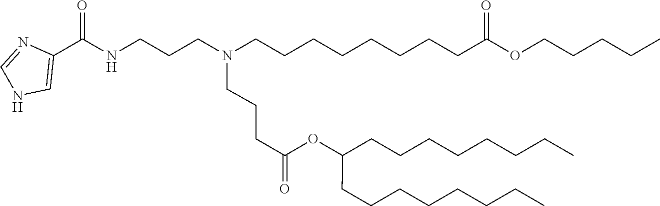 [SMILES: CCCCCCCCC(CCCCCCCC)OC(=O)CCCN(CCCCCCCCC(=O)OCCCCC)CCCNC(=O)c1c[nH]cn1] (I)(sss) | Compound 66 |
| 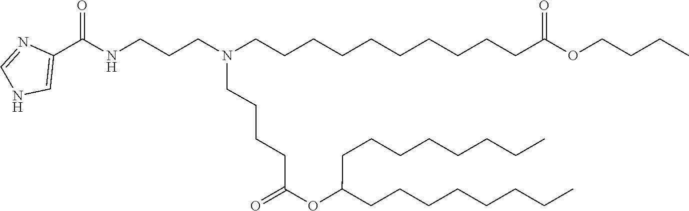 [SMILES: CCCCCCCCC(CCCCCCCC)OC(=O)CCCCN(CCCCCCCCCCC(=O)OCCCC)CCCNC(=O)c1c[nH]cn1] (I)(ttt) | Compound 67 |
| 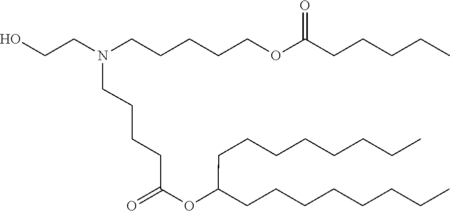 [SMILES: CCCCCCCCC(CCCCCCCC)OC(=O)CCCCN(CCO)CCCCCOC(=O)CCCCC] (I)(uuu) | Compound 68 |
| 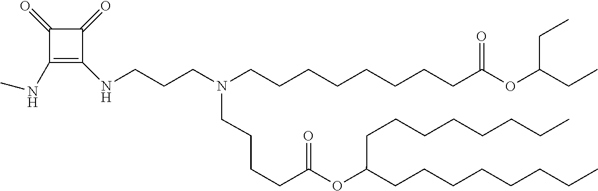 [SMILES: CCCCCCCCC(CCCCCCCC)OC(=O)CCCCN(CCCCCCCCC(=O)OC(CC)CC)CCCNc1c(NC)c(=O)c1=O] (I)(vvv) | Compound 69 |
| 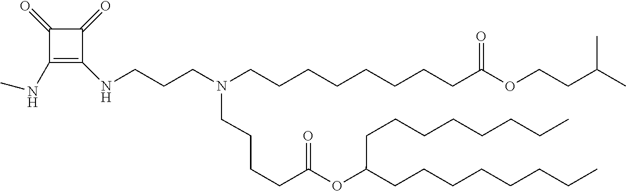 [SMILES: CCCCCCCCC(CCCCCCCC)OC(=O)CCCCN(CCCCCCCCC(=O)OCCC(C)C)CCCNc1c(NC)c(=O)c1=O] (I)(www) | Compound 70 |
| 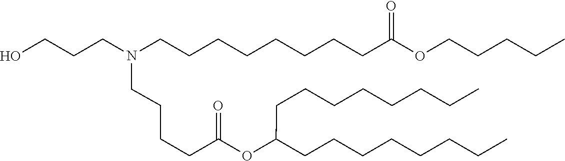 [SMILES: CCCCCCCCC(CCCCCCCC)OC(=O)CCCCN(CCCO)CCCCCCCCC(=O)OCCCCC] (I)(xxx) | Compound 71 |
| 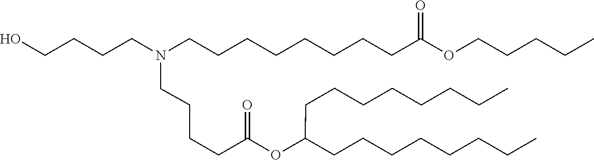 [SMILES: CCCCCCCCC(CCCCCCCC)OC(=O)CCCCN(CCCCO)CCCCCCCCC(=O)OCCCCC] (I)(yyy) | Compound 72 |
| 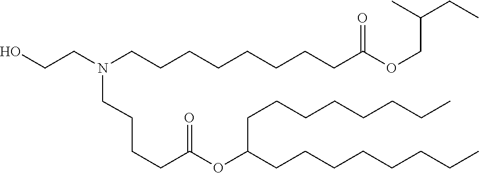 [SMILES: CCCCCCCCC(CCCCCCCC)OC(=O)CCCCN(CCO)CCCCCCCCC(=O)OCC(C)CC] (I)(zzz) | Compound 73 |
| 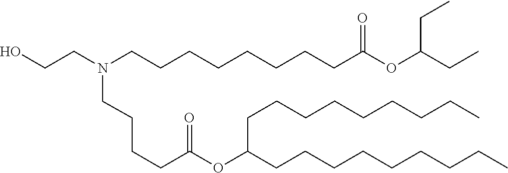 [SMILES: CCCCCCCCCC(CCCCCCCCC)OC(=O)CCCCN(CCO)CCCCCCCCC(=O)OC(CC)CC] (I)(aaaa) | Compound 74 |
| 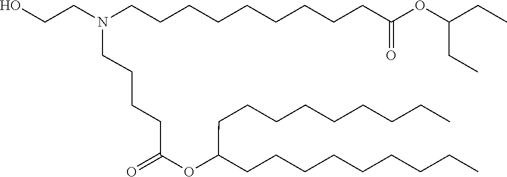 [SMILES: CCCCCCCCCC(CCCCCCCCC)OC(=O)CCCCN(CCO)CCCCCCCCCC(=O)OC(CC)CC] (I)(bbbb) | Compound 75 |
| 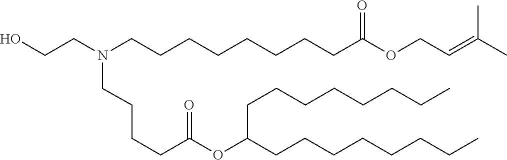 [SMILES: CCCCCCCCC(CCCCCCCC)OC(=O)CCCCN(CCO)CCCCCCCCC(=O)OCC=C(C)C] (I)(cccc) | Compound 76 |
| 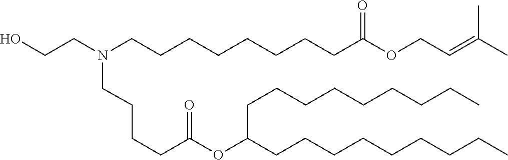 [SMILES: CCCCCCCCCC(CCCCCCCCC)OC(=O)CCCCN(CCO)CCCCCCCCC(=O)OCC=C(C)C] (I)(dddd) | Compound 77 |
| 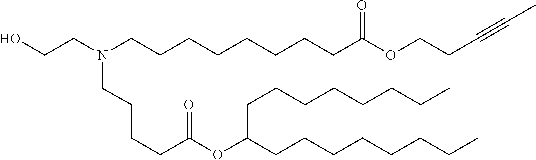 [SMILES: CC#CCCOC(=O)CCCCCCCCN(CCO)CCCCC(=O)OC(CCCCCCCC)CCCCCCCC] (I)(eeee) | Compound 78 |
| 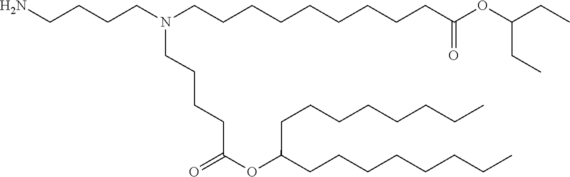 [SMILES: CCCCCCCCC(CCCCCCCC)OC(=O)CCCCN(CCCCN)CCCCCCCCCC(=O)OC(CC)CC] (I)(ffff) | Compound 79 |
| 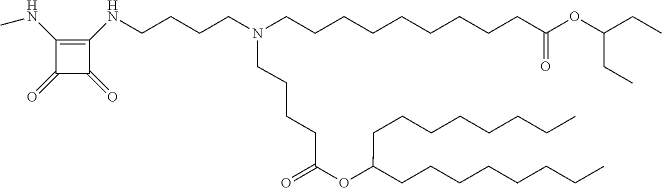 [SMILES: CCCCCCCCC(CCCCCCCC)OC(=O)CCCCN(CCCCCCCCCC(=O)OC(CC)CC)CCCCNc1c(NC)c(=O)c1=O] (I)(gggg) | Compound 80 |
| 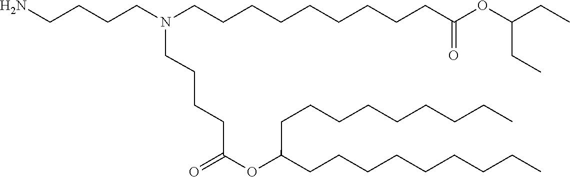 [SMILES: CCCCCCCCCC(CCCCCCCCC)OC(=O)CCCCN(CCCCN)CCCCCCCCCC(=O)OC(CC)CC] (I)(hhhh) | Compound 81 |
| 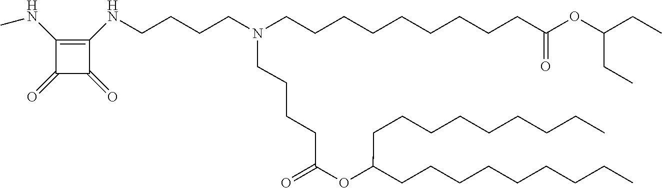 [SMILES: CCCCCCCCCC(CCCCCCCCC)OC(=O)CCCCN(CCCCCCCCCC(=O)OC(CC)CC)CCCCNc1c(NC)c(=O)c1=O] (I)(iiii) | Compound 82 |
| 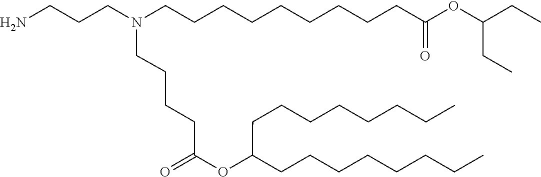 [SMILES: CCCCCCCCC(CCCCCCCC)OC(=O)CCCCN(CCCN)CCCCCCCCCC(=O)OC(CC)CC] (I)(jjjj) | Compound 83 |
| 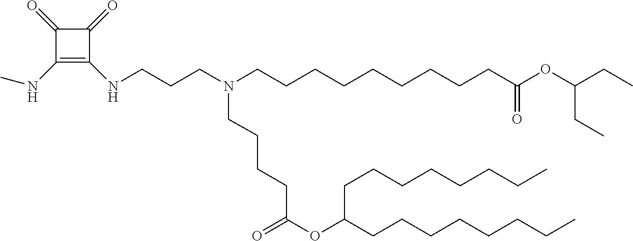 [SMILES: CCCCCCCCC(CCCCCCCC)OC(=O)CCCCN(CCCCCCCCCC(=O)OC(CC)CC)CCCNc1c(NC)c(=O)c1=O] (I)(kkkk) | Compound 84 |
| 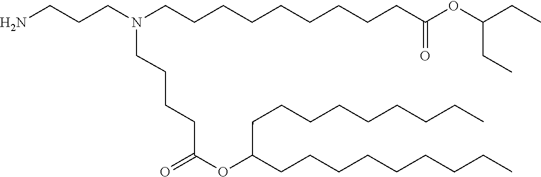 [SMILES: CCCCCCCCCC(CCCCCCCCC)OC(=O)CCCCN(CCCN)CCCCCCCCCC(=O)OC(CC)CC] (I)(llll) | Compound 85 |
| 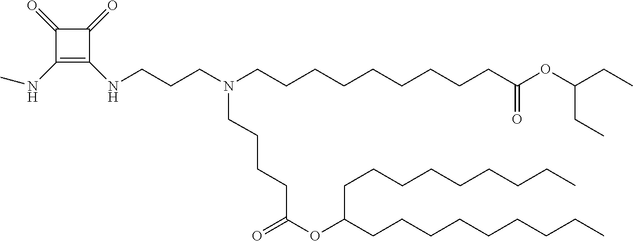 [SMILES: CCCCCCCCCC(CCCCCCCCC)OC(=O)CCCCN(CCCCCCCCCC(=O)OC(CC)CC)CCCNc1c(NC)c(=O)c1=O] (I)(mmmm) | Compound 86 |
| 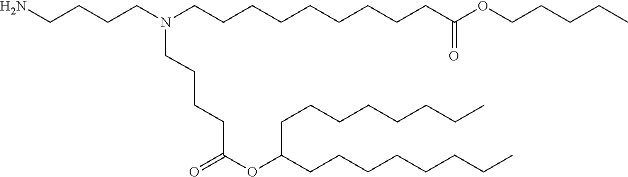 [SMILES: CCCCCCCCC(CCCCCCCC)OC(=O)CCCCN(CCCCN)CCCCCCCCCC(=O)OCCCCC] (I)(nnnn) | Compound 87 |
| 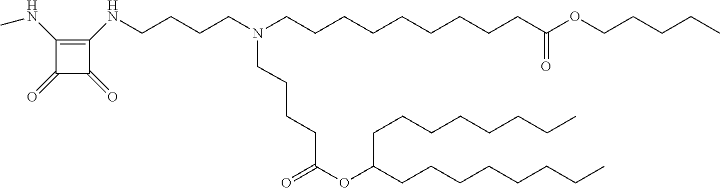 [SMILES: CCCCCCCCC(CCCCCCCC)OC(=O)CCCCN(CCCCCCCCCC(=O)OCCCCC)CCCCNc1c(NC)c(=O)c1=O] (I)(oooo) | Compound 88 |
| 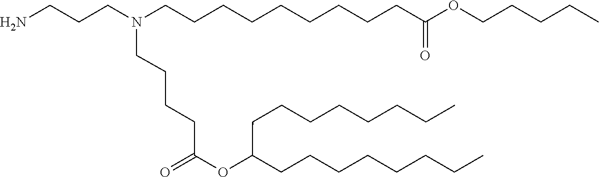 [SMILES: CCCCCCCCC(CCCCCCCC)OC(=O)CCCCN(CCCN)CCCCCCCCCC(=O)OCCCCC] (I)(pppp) | Compound 89 |
| 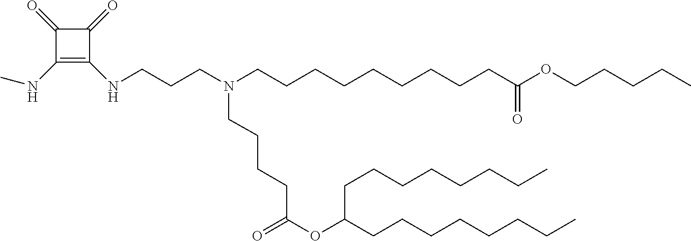 [SMILES: CCCCCCCCC(CCCCCCCC)OC(=O)CCCCN(CCCCCCCCCC(=O)OCCCCC)CCCNc1c(NC)c(=O)c1=O] (I)(qqqq) | Compound 90 |
| 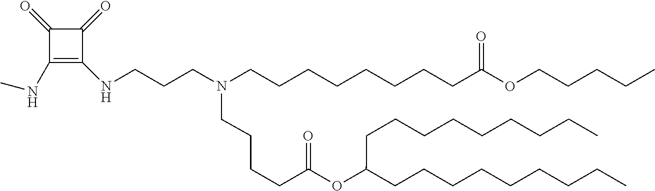 [SMILES: CCCCCCCCCC(CCCCCCCCC)OC(=O)CCCCN(CCCCCCCCC(=O)OCCCCC)CCCNc1c(NC)c(=O)c1=O] (I)(rrrr) | Compound 91 |
| 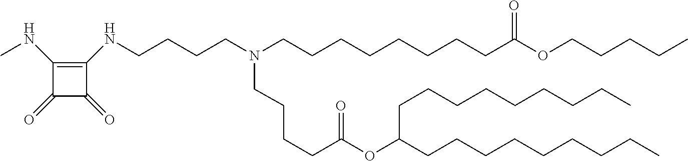 [SMILES: CCCCCCCCCC(CCCCCCCCC)OC(=O)CCCCN(CCCCCCCCC(=O)OCCCCC)CCCCNc1c(NC)c(=O)c1=O] (I)(ssss) | Compound 92 |
| 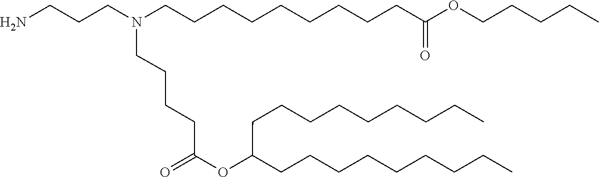 [SMILES: CCCCCCCCCC(CCCCCCCCC)OC(=O)CCCCN(CCCN)CCCCCCCCCC(=O)OCCCCC] (I)(tttt) | Compound 93 |
| 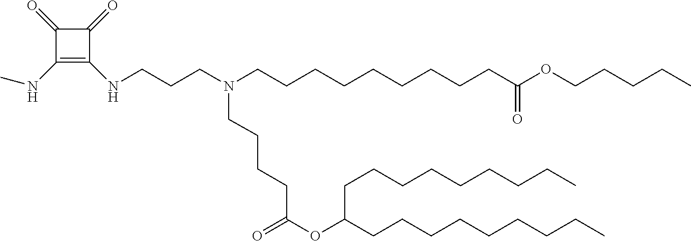 [SMILES: CCCCCCCCCC(CCCCCCCCC)OC(=O)CCCCN(CCCCCCCCCC(=O)OCCCCC)CCCNc1c(NC)c(=O)c1=O] (I)(uuuu) | Compound 94 |
| 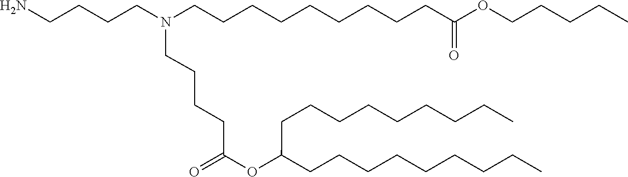 [SMILES: CCCCCCCCCC(CCCCCCCCC)OC(=O)CCCCN(CCCCN)CCCCCCCCCC(=O)OCCCCC] (I)(vvvv) | Compound 95 |
| 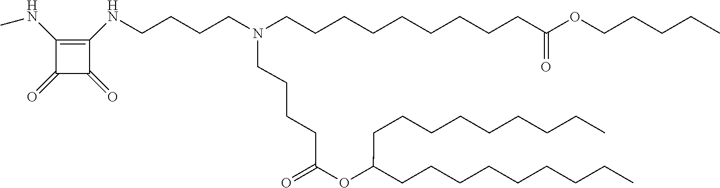 [SMILES: CCCCCCCCCC(CCCCCCCCC)OC(=O)CCCCN(CCCCCCCCCC(=O)OCCCCC)CCCCNc1c(NC)c(=O)c1=O] (I)(wwww) | Compound 96 |
| 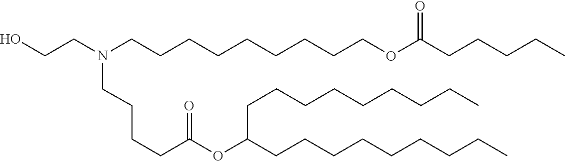 [SMILES: CCCCCCCCCC(CCCCCCCCC)OC(=O)CCCCN(CCO)CCCCCCCCCOC(=O)CCCCC] (I)(xxxx) | Compound 97 |
| 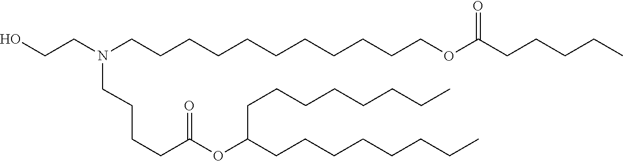 [SMILES: CCCCCCCCC(CCCCCCCC)OC(=O)CCCCN(CCO)CCCCCCCCCCCOC(=O)CCCCC] (I)(yyyy) | Compound 98 |
| 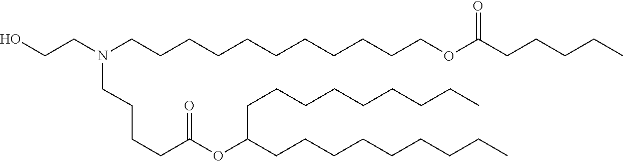 [SMILES: CCCCCCCCCC(CCCCCCCCC)OC(=O)CCCCN(CCO)CCCCCCCCCCCOC(=O)CCCCC] (I)(zzzz) | Compound 99 |
| 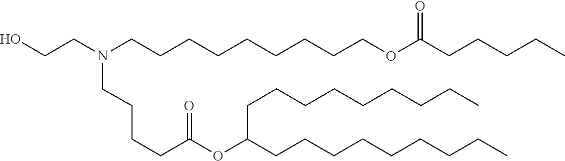 [SMILES: CCCCCCCCCC(CCCCCCCCC)OC(=O)CCCCN(CCO)CCCCCCCCCOC(=O)CCCCC] (I)(aaaaa) | Compound 100 |
| 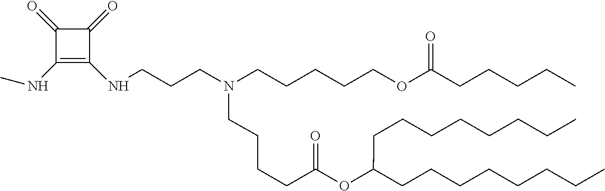 [SMILES: CCCCCCCCC(CCCCCCCC)OC(=O)CCCCN(CCCCCOC(=O)CCCCC)CCCNc1c(NC)c(=O)c1=O] (I)(bbbbb) | Compound 101 |
| 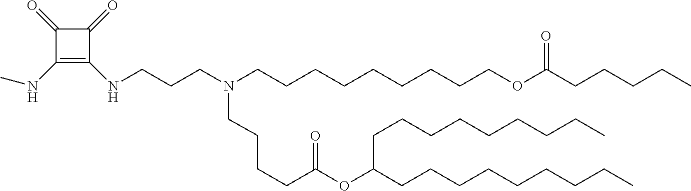 [SMILES: CCCCCCCCCC(CCCCCCCCC)OC(=O)CCCCN(CCCCCCCCCOC(=O)CCCCC)CCCNc1c(NC)c(=O)c1=O] (I)(ccccc) | Compound 102 |
| 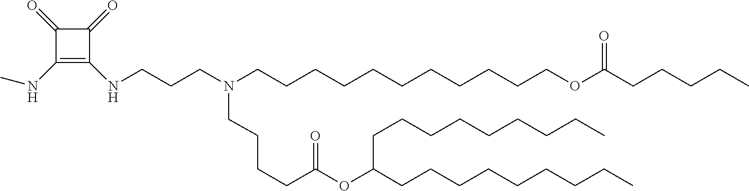 [SMILES: CCCCCCCCCC(CCCCCCCCC)OC(=O)CCCCN(CCCCCCCCCCCOC(=O)CCCCC)CCCNc1c(NC)c(=O)c1=O] (I)(ddddd) | Compound 103 |

In addition, it is to be understood that any particular embodiment of the present disclosure that falls within the prior art may be explicitly excluded from any one or more of the claims. Since such embodiments are deemed to be known to one of ordinary skill in the art, they may be excluded even if the exclusion is not set forth explicitly herein.

It is to be understood that while the present disclosure has been described in conjunction with the detailed description thereof, the foregoing description is intended to illustrate and not limit the scope of the present disclosure, which is defined by the scope of the appended claims. Other aspects, advantages, and alterations are within the scope of the following claims.

1. A compound of Formula I 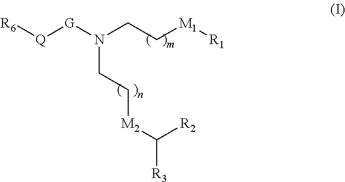
or a salt or isomer thereof, wherein
each m is independently an integer from 4-13;
each n is independently an integer from 1-3;
each R¹ is independently selected from C1-C5 alkyl, C2-C5 alkenyl, or C2-C5 alkynyl, wherein alkyl, alkenyl, or alkynyl is linear or branched;
each R₂ and R₃ is independently selected from C₁ to C₁₄ alkyl;
each R₆ is independently selected from H, 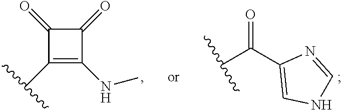
each M₁ and M₂ is independently selected from —C(O)O— and —OC(O)—, wherein at least one of M₁ or M₂ is —C(O)O—;
each Q is selected from —O— or —NH—;
and each G is —(CR₄R₅)ₖ—;
wherein each k is selected from an integer from 2-5,
and each R₄ and R₅ is independently selected from H, and C1-C3 alkyl.

2. The compound of claim 1, wherein the compound is: 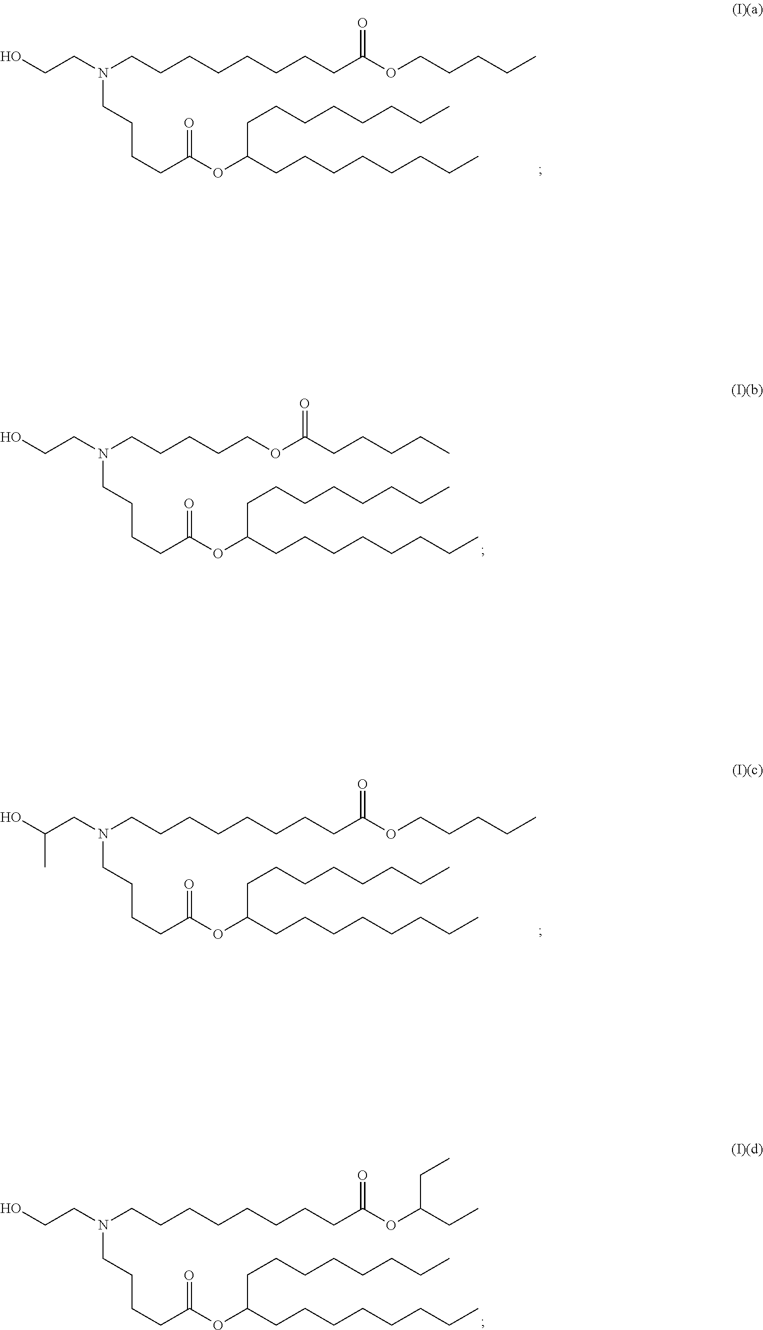 [SMILES: CCCCCCCCC(CCCCCCCC)OC(=O)CCCCN(CCO)CCCCCCCCC(=O)OCCCCC ; CCCCCCCCC(CCCCCCCC)OC(=O)CCCCN(CCO)CCCCCOC(=O)CCCCC ; CCCCCCCCC(CCCCCCCC)OC(=O)CCCCN(CCCCCCCCC(=O)OCCCCC)CC(C)O ; CCCCCCCCC(CCCCCCCC)OC(=O)CCCCN(CCO)CCCCCCCCC(=O)OC(CC)CC] 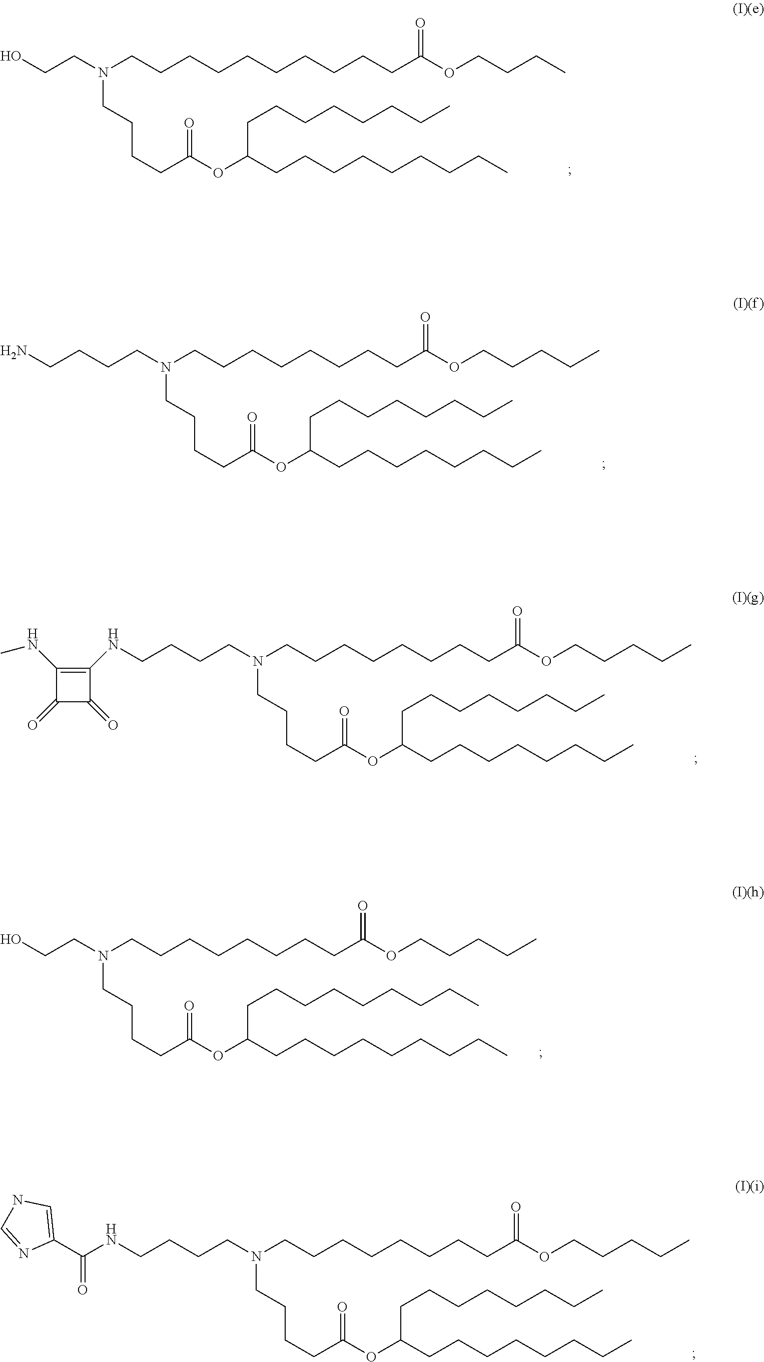 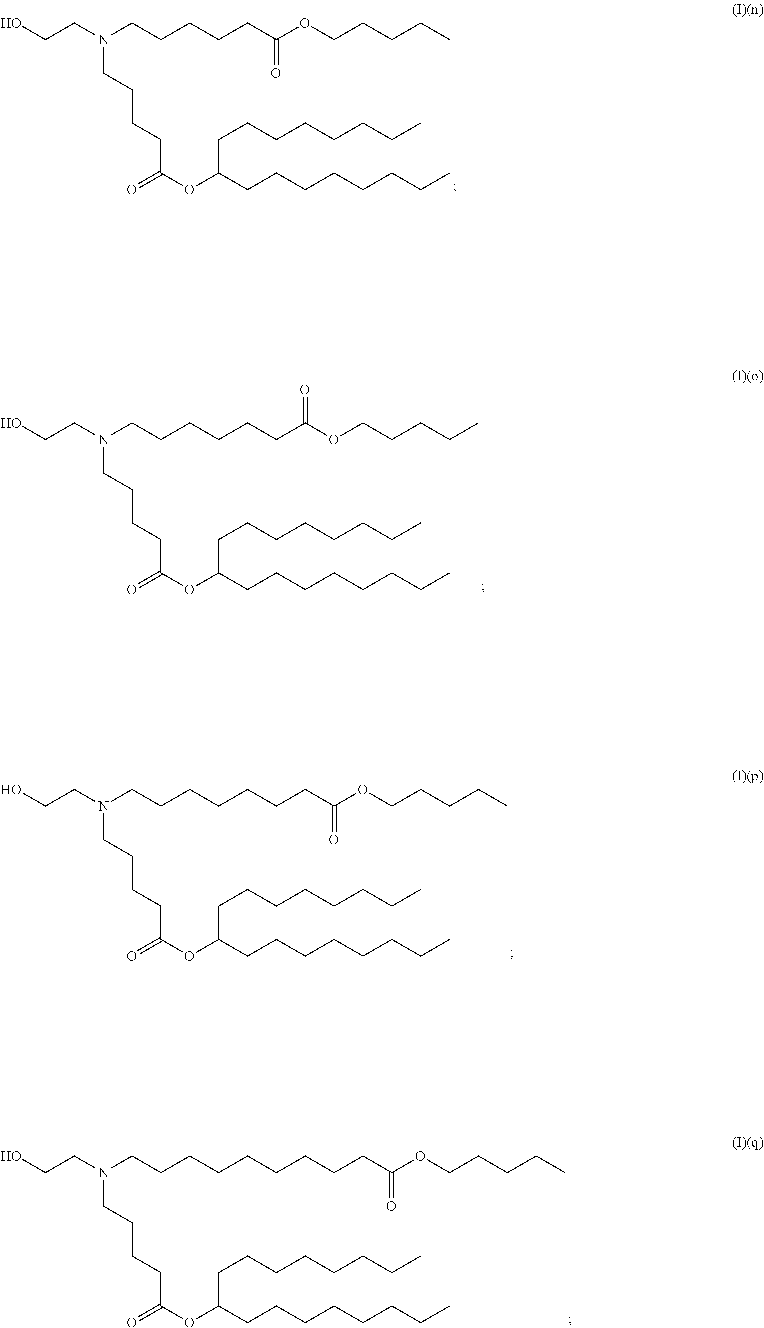 [SMILES: CCCCCCCCC(CCCCCCCC)OC(=O)CCCCN(CCO)CCCCCC(=O)OCCCCC ; CCCCCCCCC(CCCCCCCC)OC(=O)CCCCN(CCO)CCCCCCC(=O)OCCCCC ; CCCCCCCCC(CCCCCCCC)OC(=O)CCCCN(CCO)CCCCCCCC(=O)OCCCCC ; CCCCCCCCC(CCCCCCCC)OC(=O)CCCCN(CCO)CCCCCCCCCC(=O)OCCCCC] 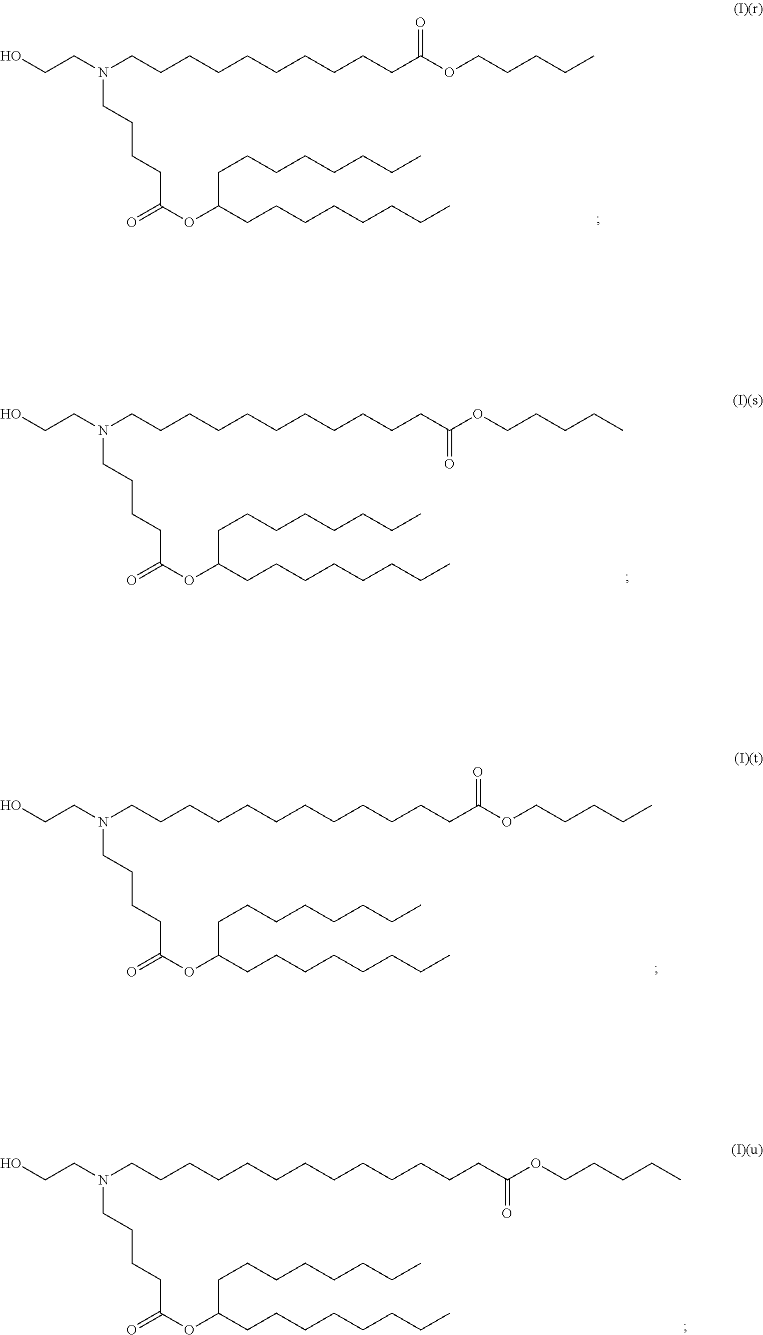 [SMILES: CCCCCCCCC(CCCCCCCC)OC(=O)CCCCN(CCO)CCCCCCCCCCC(=O)OCCCCC ; CCCCCCCCC(CCCCCCCC)OC(=O)CCCCN(CCO)CCCCCCCCCCCC(=O)OCCCCC ; CCCCCCCCC(CCCCCCCC)OC(=O)CCCCN(CCO)CCCCCCCCCCCCC(=O)OCCCCC ; CCCCCCCCC(CCCCCCCC)OC(=O)CCCCN(CCO)CCCCCCCCCCCCCC(=O)OCCCCC] 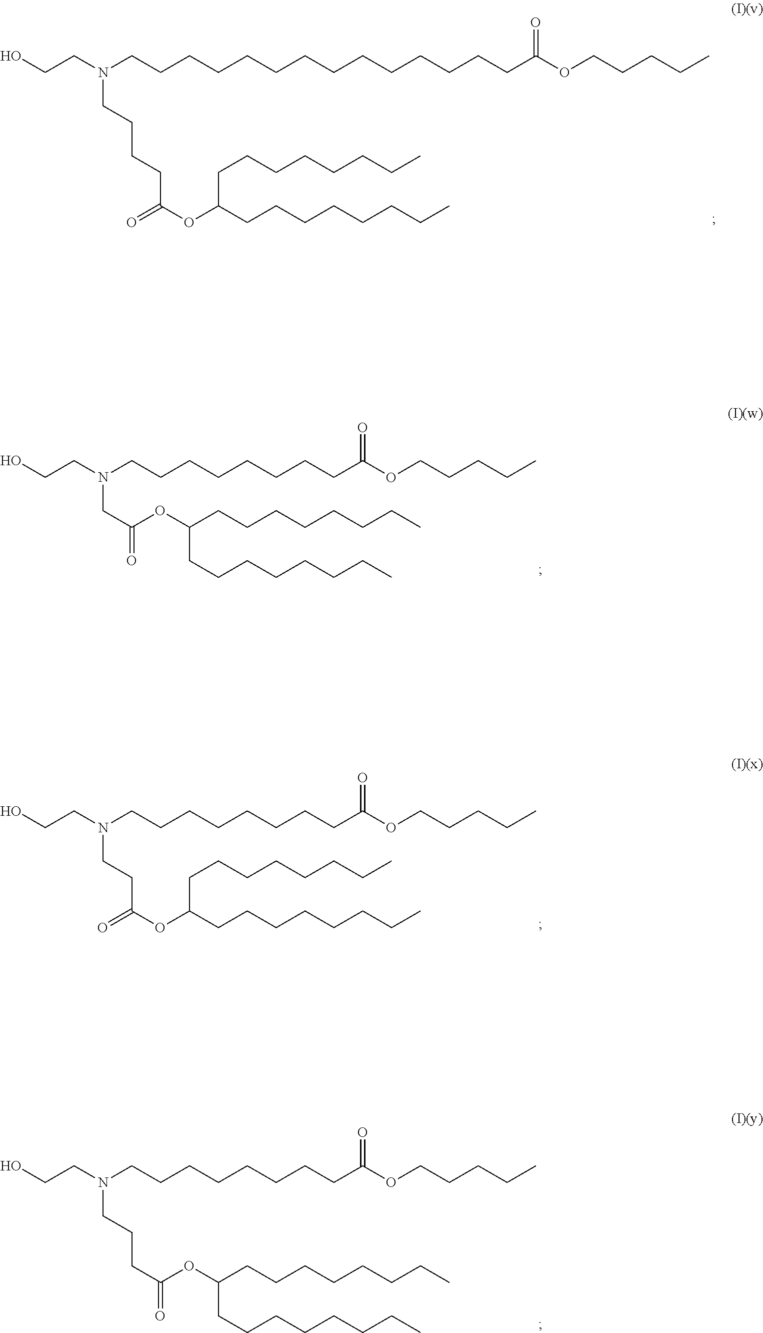 [SMILES: CCCCCCCCC(CCCCCCCC)OC(=O)CCCCN(CCO)CCCCCCCCCCCCCCC(=O)OCCCCC ; CCCCCCCCC(CCCCCCCC)OC(=O)CN(CCO)CCCCCCCCC(=O)OCCCCC ; CCCCCCCCC(CCCCCCCC)OC(=O)CCN(CCO)CCCCCCCCC(=O)OCCCCC ; CCCCCCCCC(CCCCCCCC)OC(=O)CCCN(CCO)CCCCCCCCC(=O)OCCCCC] 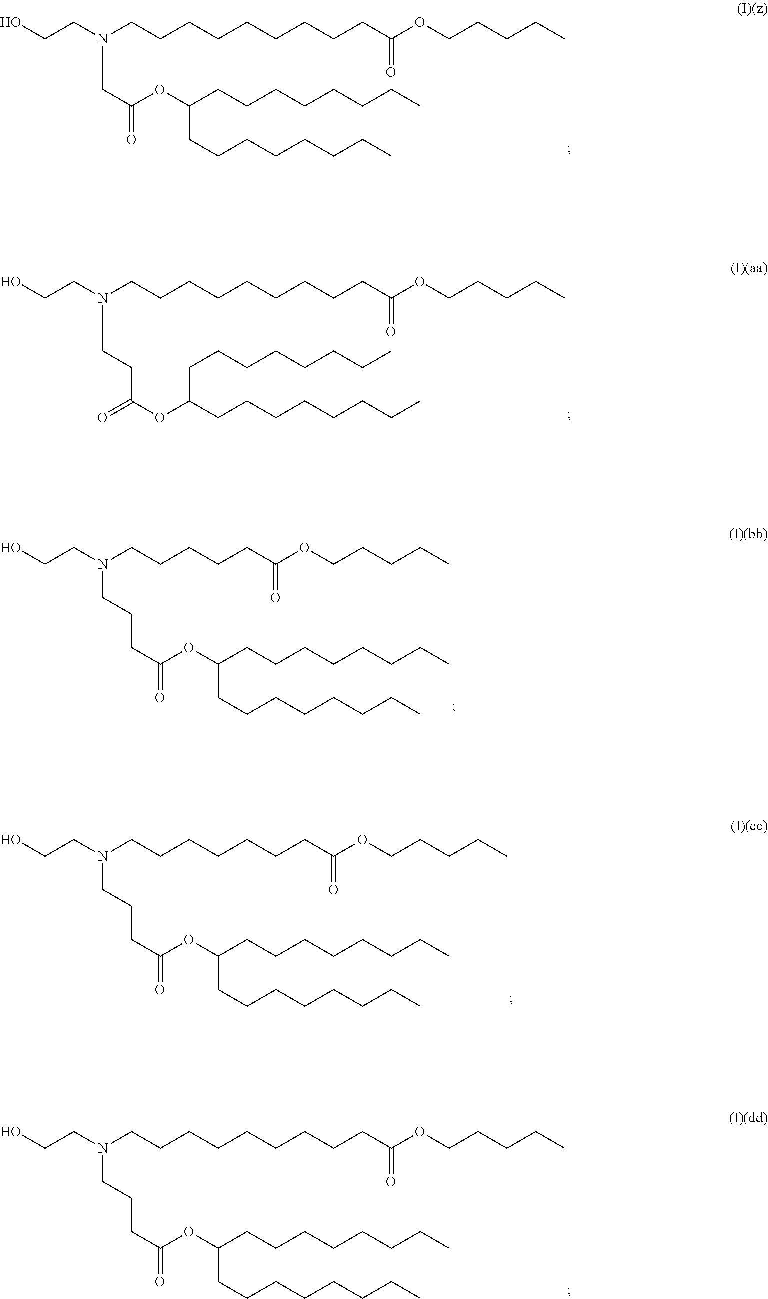 [SMILES: CCCCCCCCC(CCCCCCCC)OC(=O)CN(CCO)CCCCCCCCCC(=O)OCCCCC ; CCCCCCCCC(CCCCCCCC)OC(=O)CCN(CCO)CCCCCCCCCC(=O)OCCCCC ; CCCCCCCCC(CCCCCCCC)OC(=O)CCCN(CCO)CCCCCC(=O)OCCCCC ; CCCCCCCCC(CCCCCCCC)OC(=O)CCCN(CCO)CCCCCCCC(=O)OCCCCC ; CCCCCCCCC(CCCCCCCC)OC(=O)CCCN(CCO)CCCCCCCCCC(=O)OCCCCC] 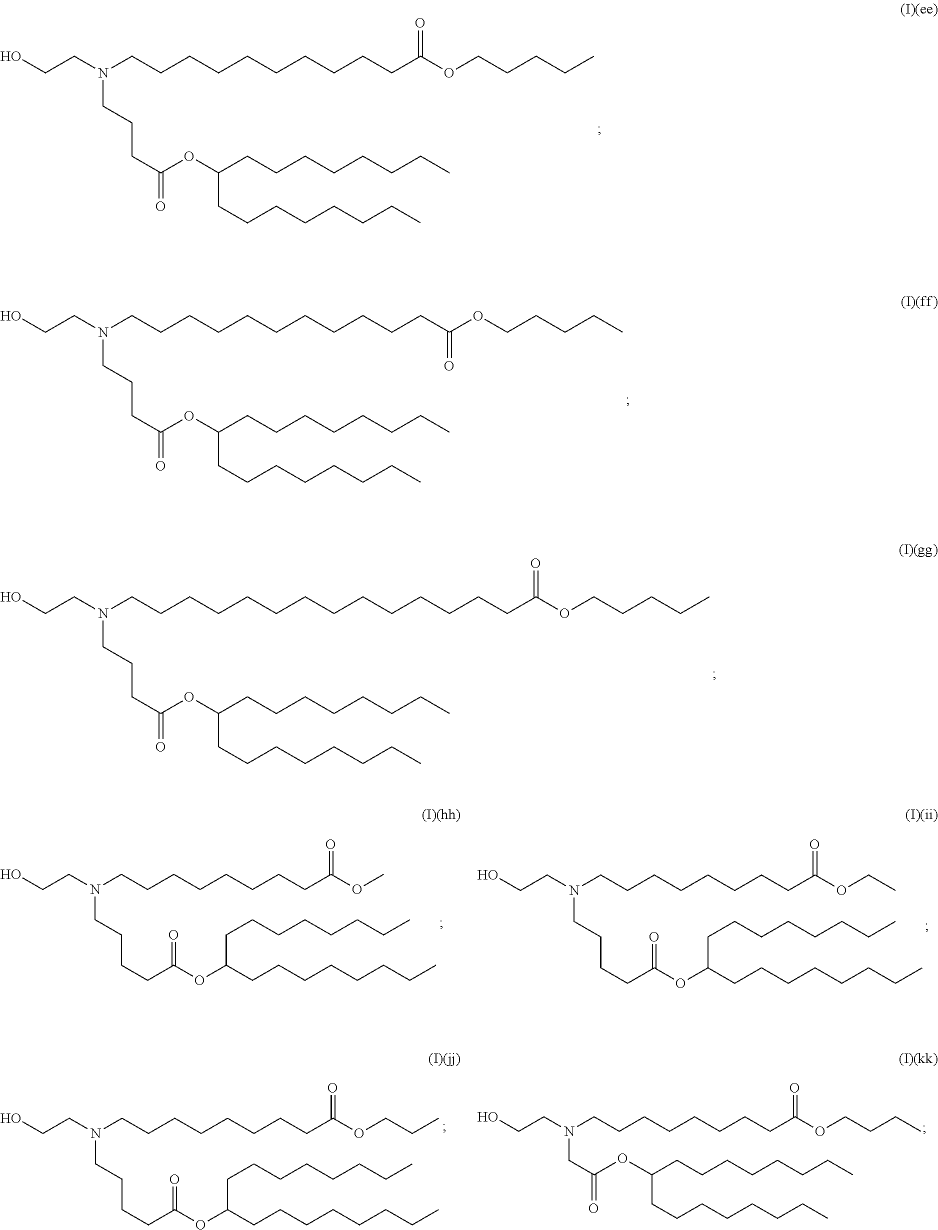 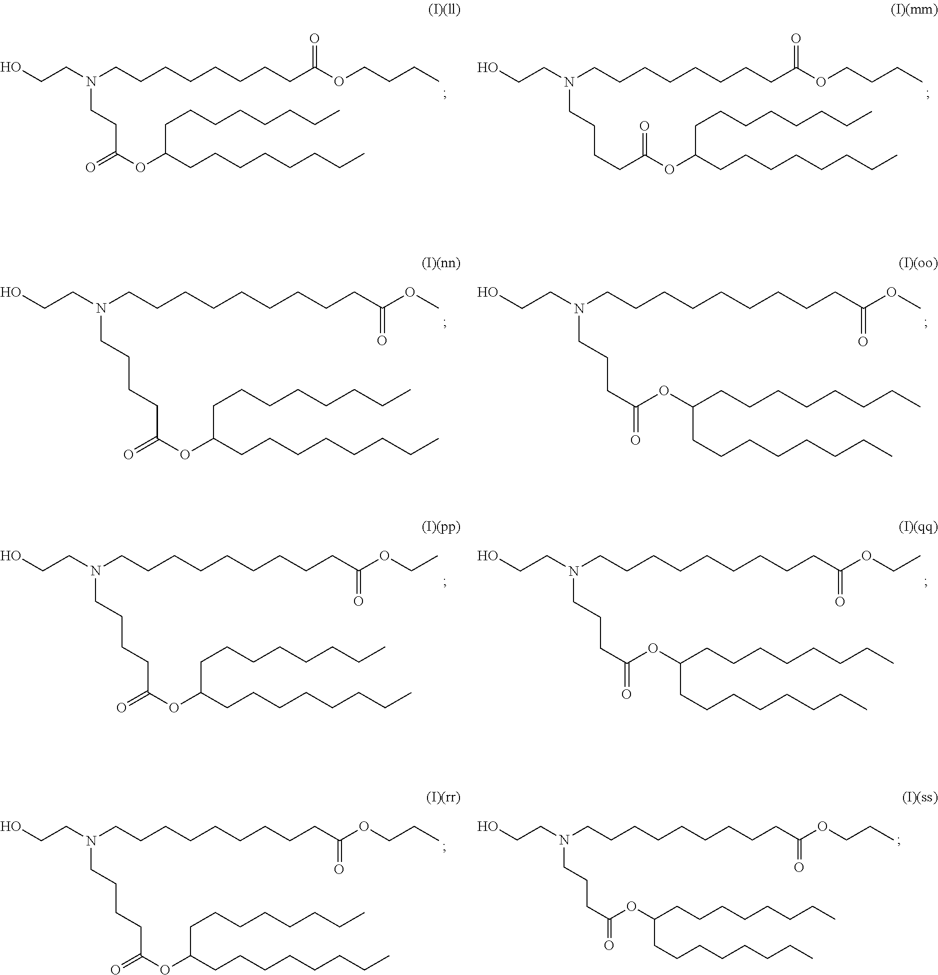 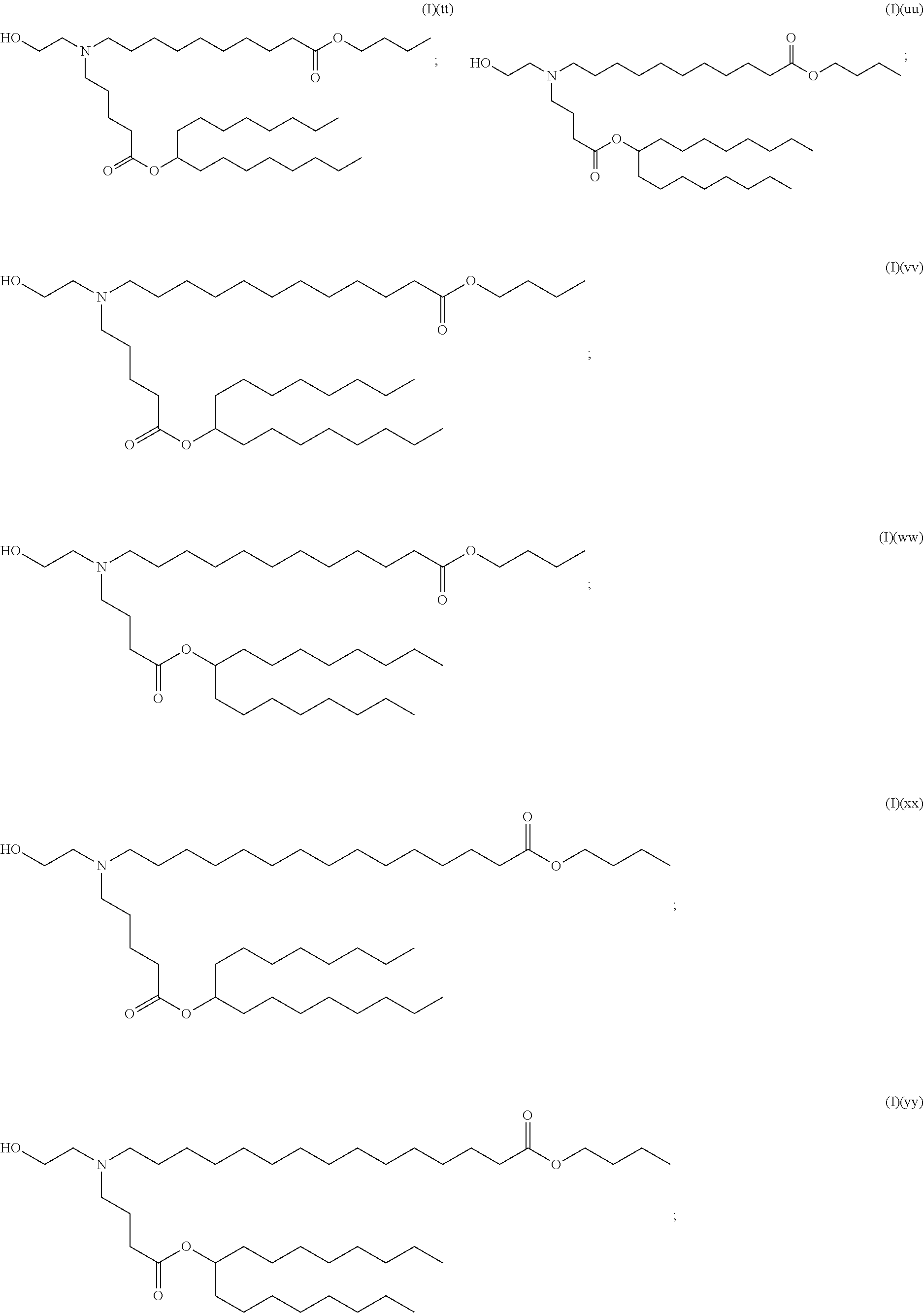 [SMILES: CCCCCCCCC(CCCCCCCC)OC(=O)CCCCN(CCO)CCCCCCCCCC(=O)OCCCC ; CCCCCCCCC(CCCCCCCC)OC(=O)CCCN(CCO)CCCCCCCCCCC(=O)OCCCC ; CCCCCCCCC(CCCCCCCC)OC(=O)CCCCN(CCO)CCCCCCCCCCCC(=O)OCCCC ; CCCCCCCCC(CCCCCCCC)OC(=O)CCCN(CCO)CCCCCCCCCCCC(=O)OCCCC ; CCCCCCCCC(CCCCCCCC)OC(=O)CCCCN(CCO)CCCCCCCCCCCCCCC(=O)OCCCC ; CCCCCCCCC(CCCCCCCC)OC(=O)CCCN(CCO)CCCCCCCCCCCCCCC(=O)OCCCC] 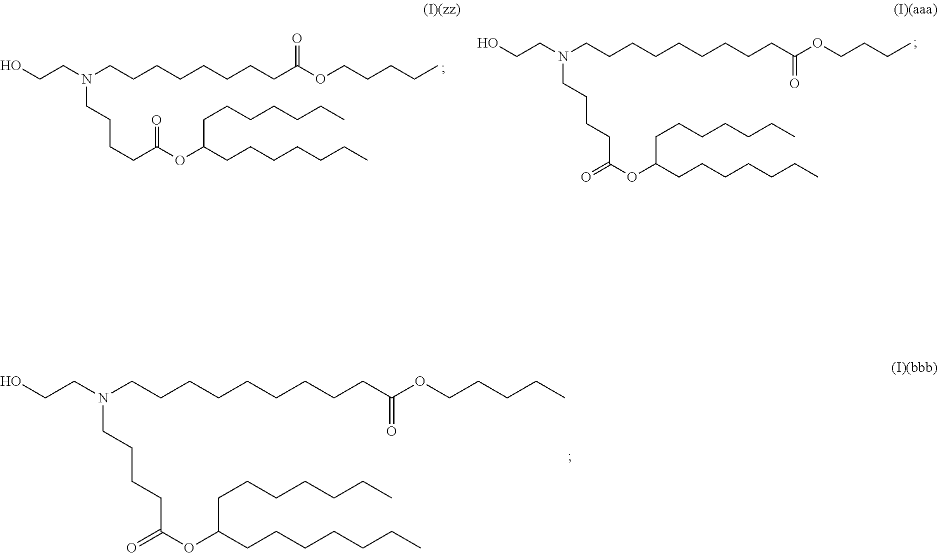 [SMILES: CCCCCCCC(CCCCCCC)OC(=O)CCCCN(CCO)CCCCCCCCC(=O)OCCCCC ; CCCCCCCC(CCCCCCC)OC(=O)CCCCN(CCO)CCCCCCCCCC(=O)OCCCC ; CCCCCCCC(CCCCCCC)OC(=O)CCCCN(CCO)CCCCCCCCCC(=O)OCCCCC] 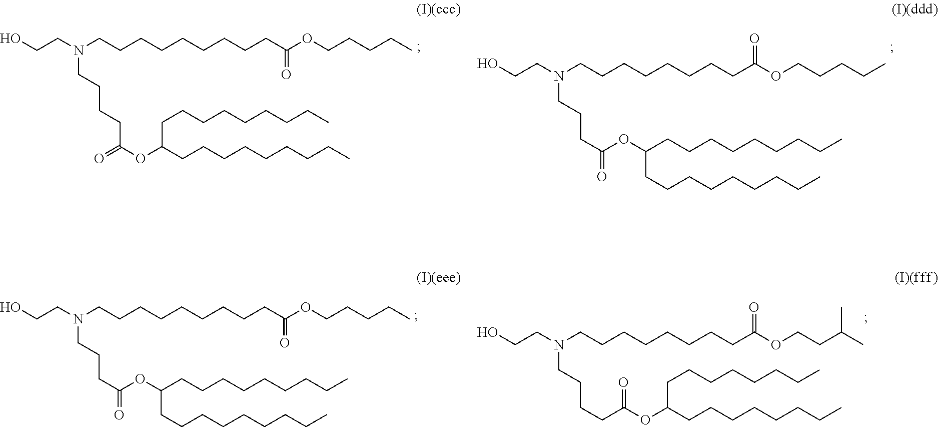 [SMILES: CCCCCCCCCC(CCCCCCCCC)OC(=O)CCCCN(CCO)CCCCCCCCCC(=O)OCCCCC ; CCCCCCCCCC(CCCCCCCCC)OC(=O)CCCN(CCO)CCCCCCCCC(=O)OCCCCC ; CCCCCCCCCC(CCCCCCCCC)OC(=O)CCCN(CCO)CCCCCCCCCC(=O)OCCCCC ; CCCCCCCCC(CCCCCCCC)OC(=O)CCCCN(CCO)CCCCCCCCC(=O)OCCC(C)C] 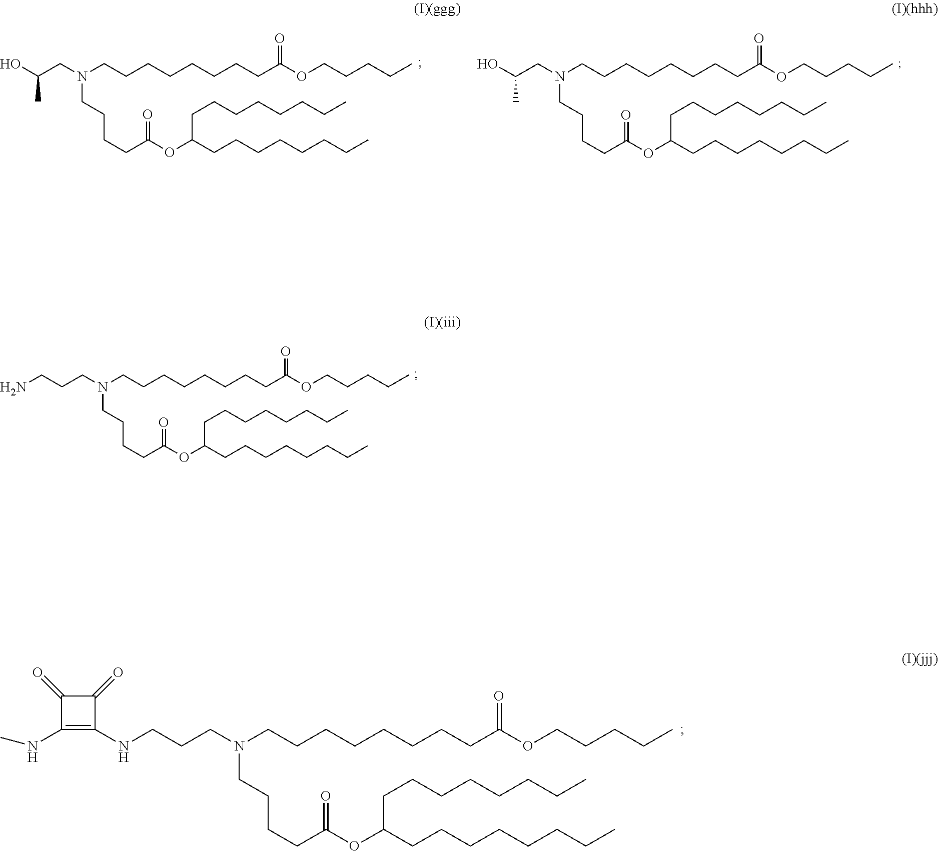 [SMILES: CCCCCCCCC(CCCCCCCC)OC(=O)CCCCN(CCCCCCCCC(=O)OCCCCC)C[C@@H](C)O ; CCCCCCCCC(CCCCCCCC)OC(=O)CCCCN(CCCCCCCCC(=O)OCCCCC)C[C@H](C)O ; CCCCCCCCC(CCCCCCCC)OC(=O)CCCCN(CCCN)CCCCCCCCC(=O)OCCCCC ; CCCCCCCCC(CCCCCCCC)OC(=O)CCCCN(CCCCCCCCC(=O)OCCCCC)CCCNc1c(NC)c(=O)c1=O] 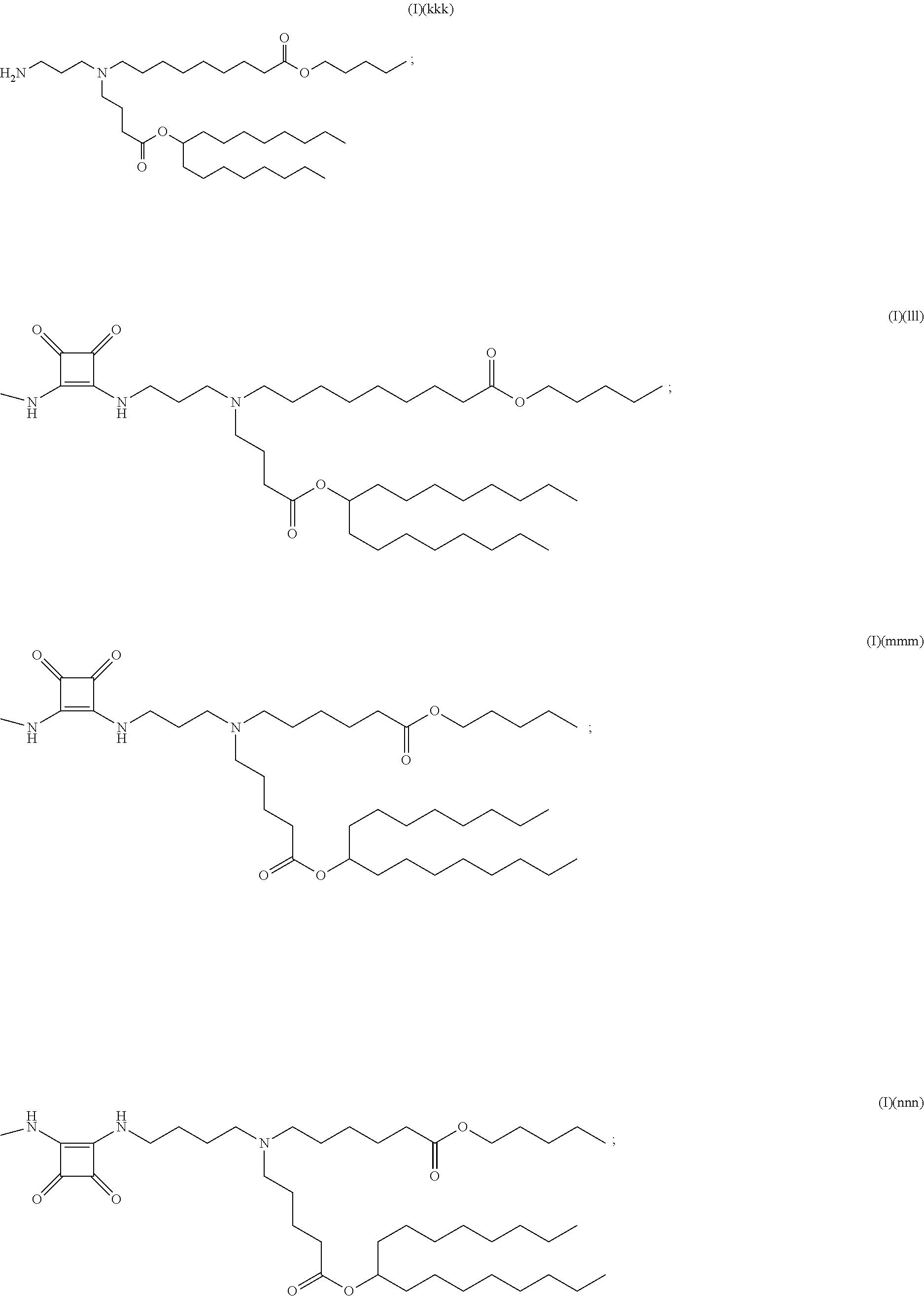 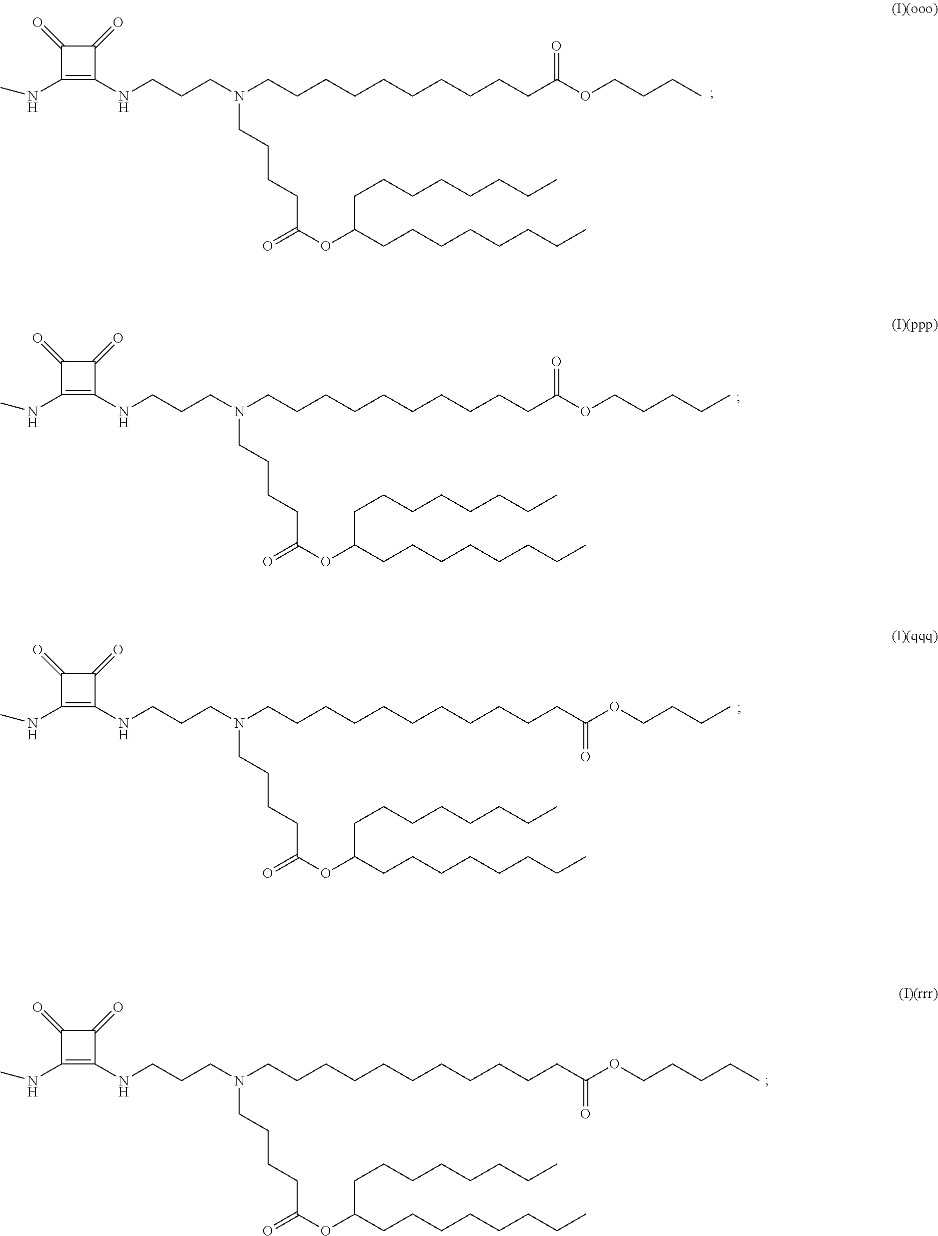 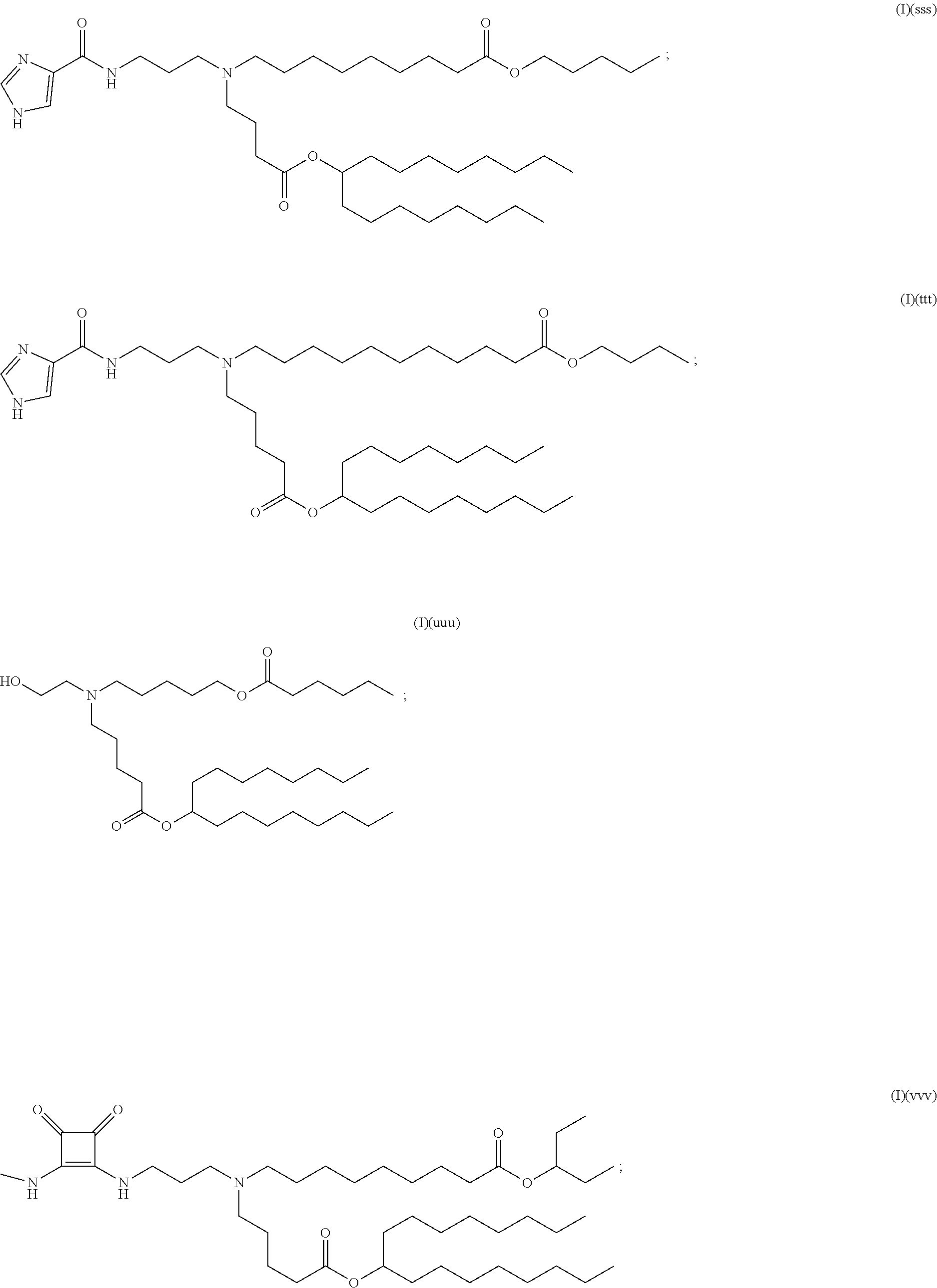 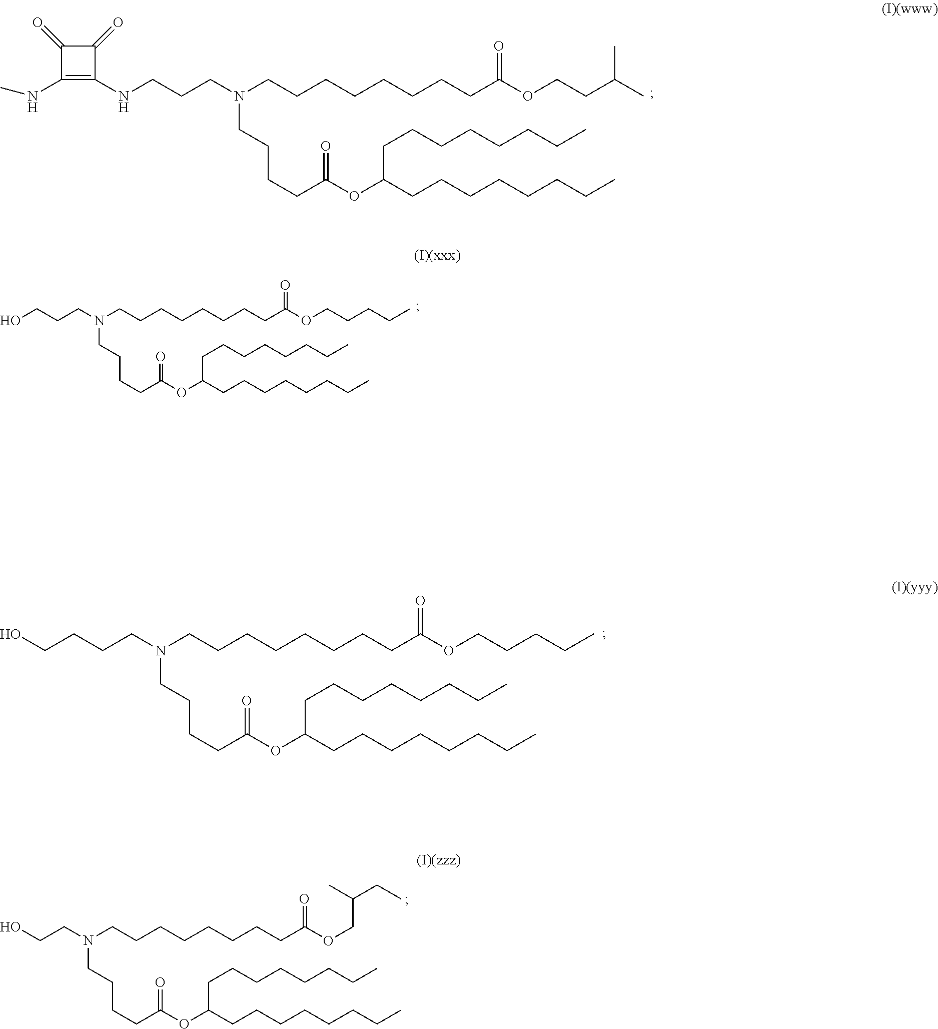 [SMILES: CCCCCCCCC(CCCCCCCC)OC(=O)CCCCN(CCCCCCCCC(=O)OCCC(C)C)CCCNc1c(NC)c(=O)c1=O ; CCCCCCCCC(CCCCCCCC)OC(=O)CCCCN(CCCO)CCCCCCCCC(=O)OCCCCC ; CCCCCCCCC(CCCCCCCC)OC(=O)CCCCN(CCCCO)CCCCCCCCC(=O)OCCCCC ; CCCCCCCCC(CCCCCCCC)OC(=O)CCCCN(CCO)CCCCCCCCC(=O)OCC(C)CC] 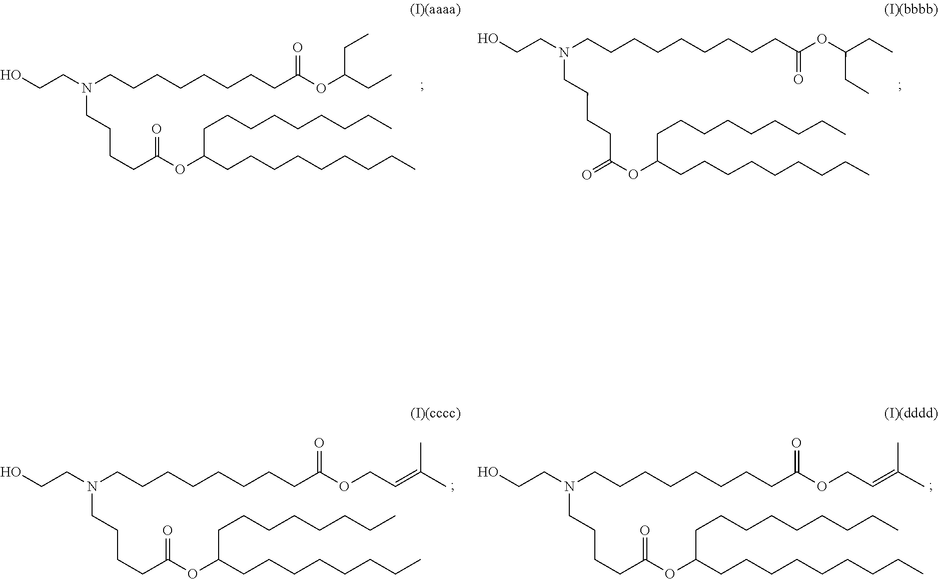 [SMILES: CCCCCCCCCC(CCCCCCCCC)OC(=O)CCCCN(CCO)CCCCCCCCC(=O)OC(CC)CC ; CCCCCCCCCC(CCCCCCCCC)OC(=O)CCCCN(CCO)CCCCCCCCCC(=O)OC(CC)CC ; CCCCCCCCC(CCCCCCCC)OC(=O)CCCCN(CCO)CCCCCCCCC(=O)OCC=C(C)C ; ";" ; CCCCCCCCCC(CCCCCCCCC)OC(=O)CCCCN(CCO)CCCCCCCCC(=O)OCC=C(C)C ; ";"] 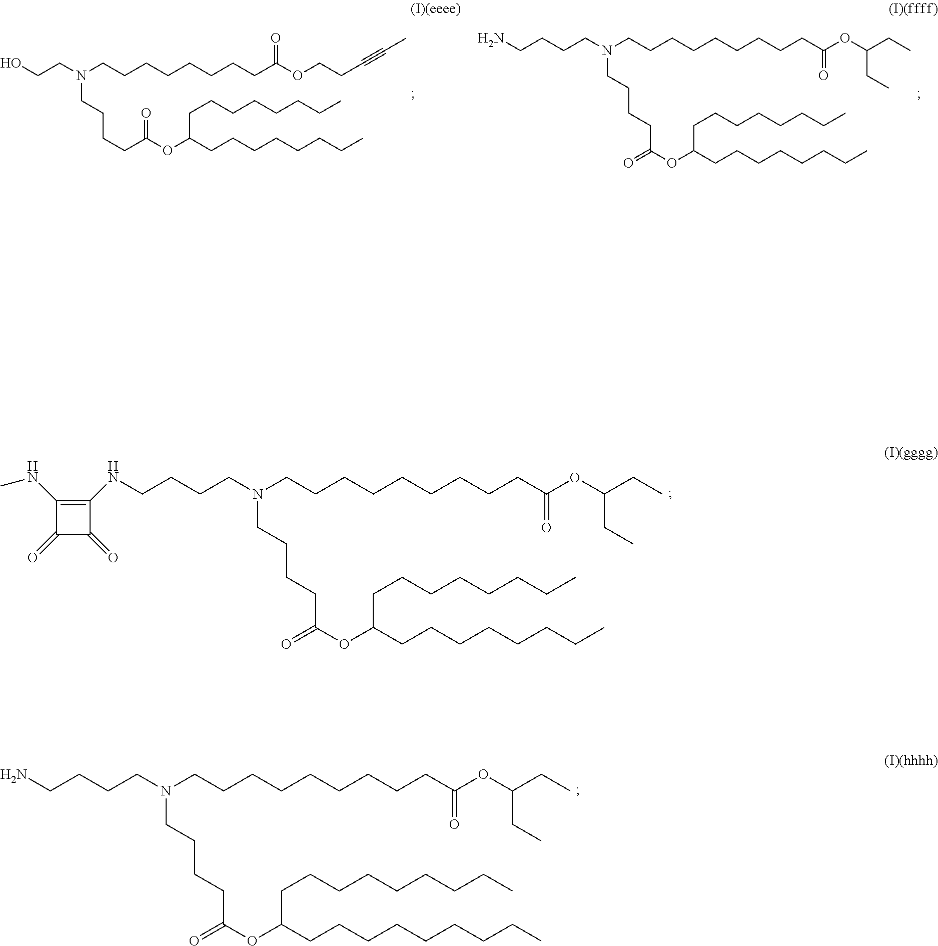 [SMILES: CC#CCCOC(=O)CCCCCCCCN(CCO)CCCCC(=O)OC(CCCCCCCC)CCCCCCCC ; CCCCCCCCC(CCCCCCCC)OC(=O)CCCCN(CCCCN)CCCCCCCCCC(=O)OC(CC)CC ; CCCCCCCCC(CCCCCCCC)OC(=O)CCCCN(CCCCCCCCCC(=O)OC(CC)CC)CCCCNc1c(NC)c(=O)c1=O ; ";" ; CCCCCCCCCC(CCCCCCCCC)OC(=O)CCCCN(CCCCN)CCCCCCCCCC(=O)OC(CC)CC ; ";"] 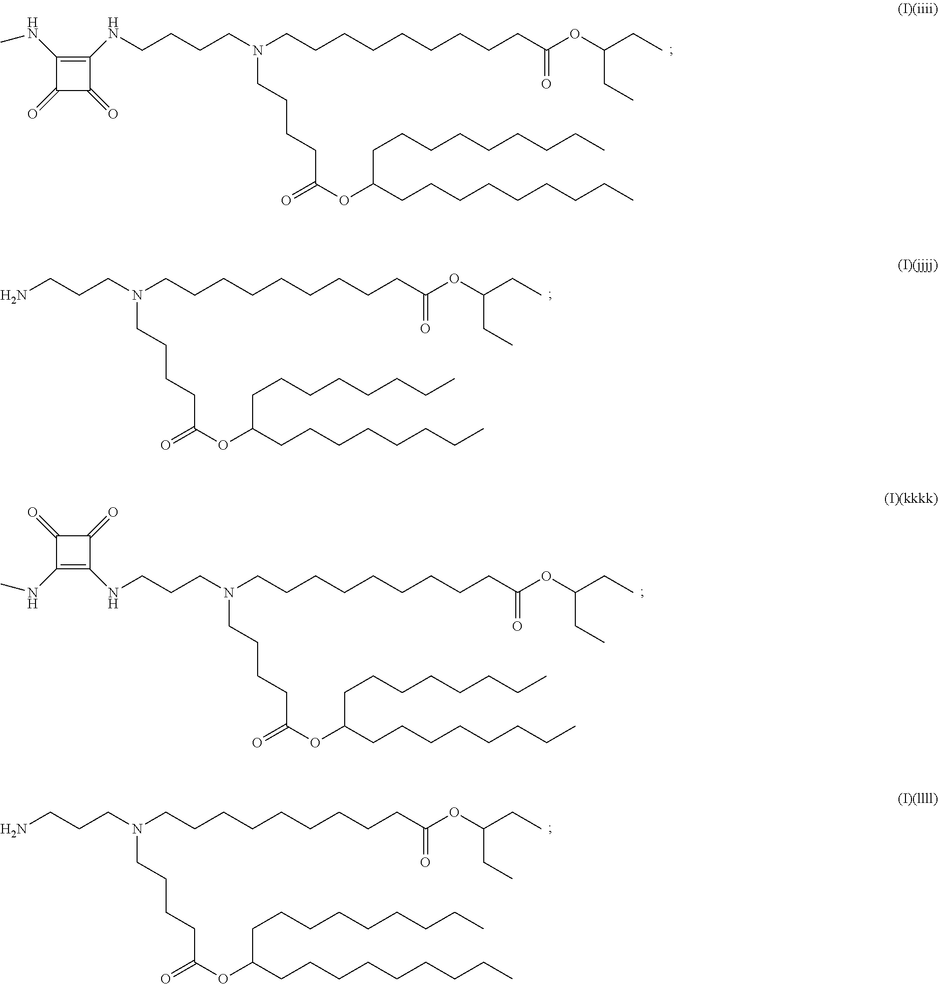 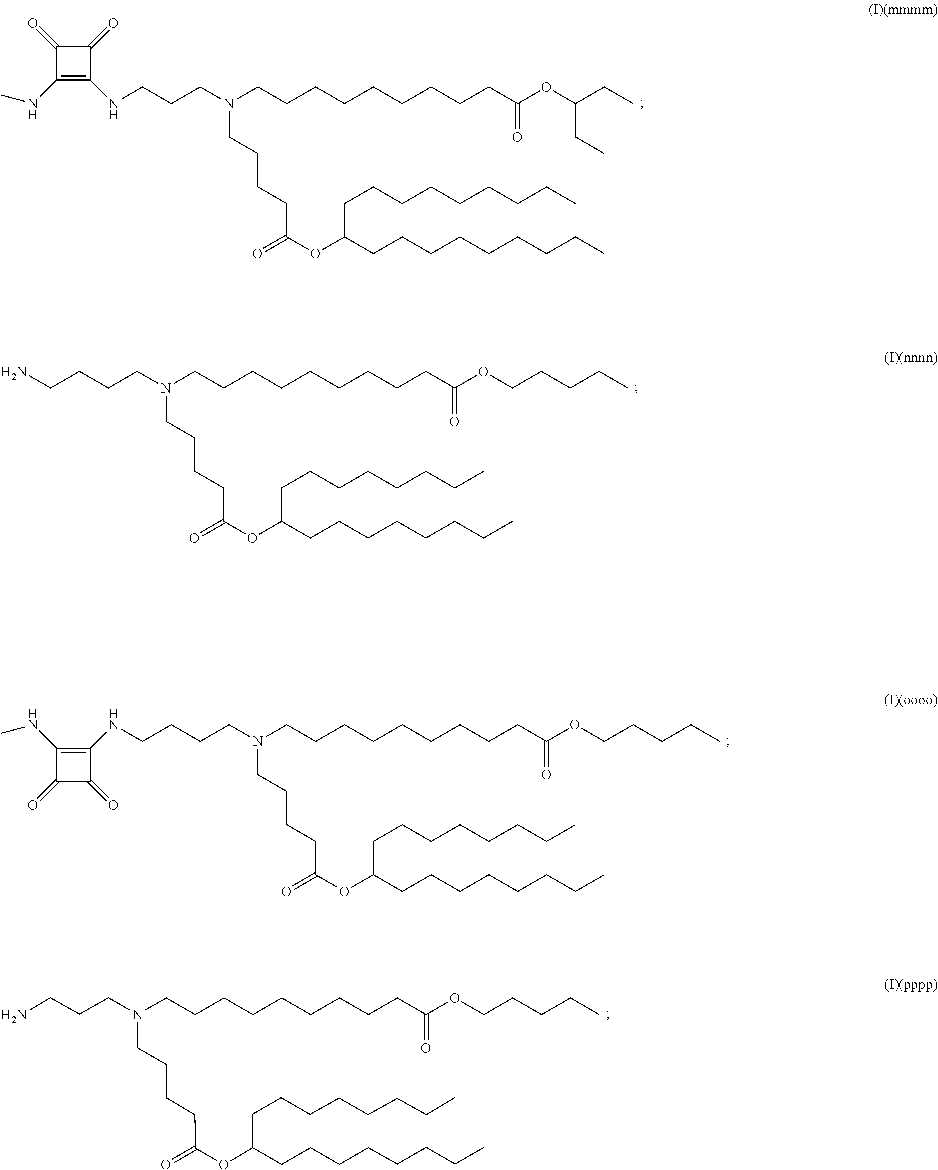 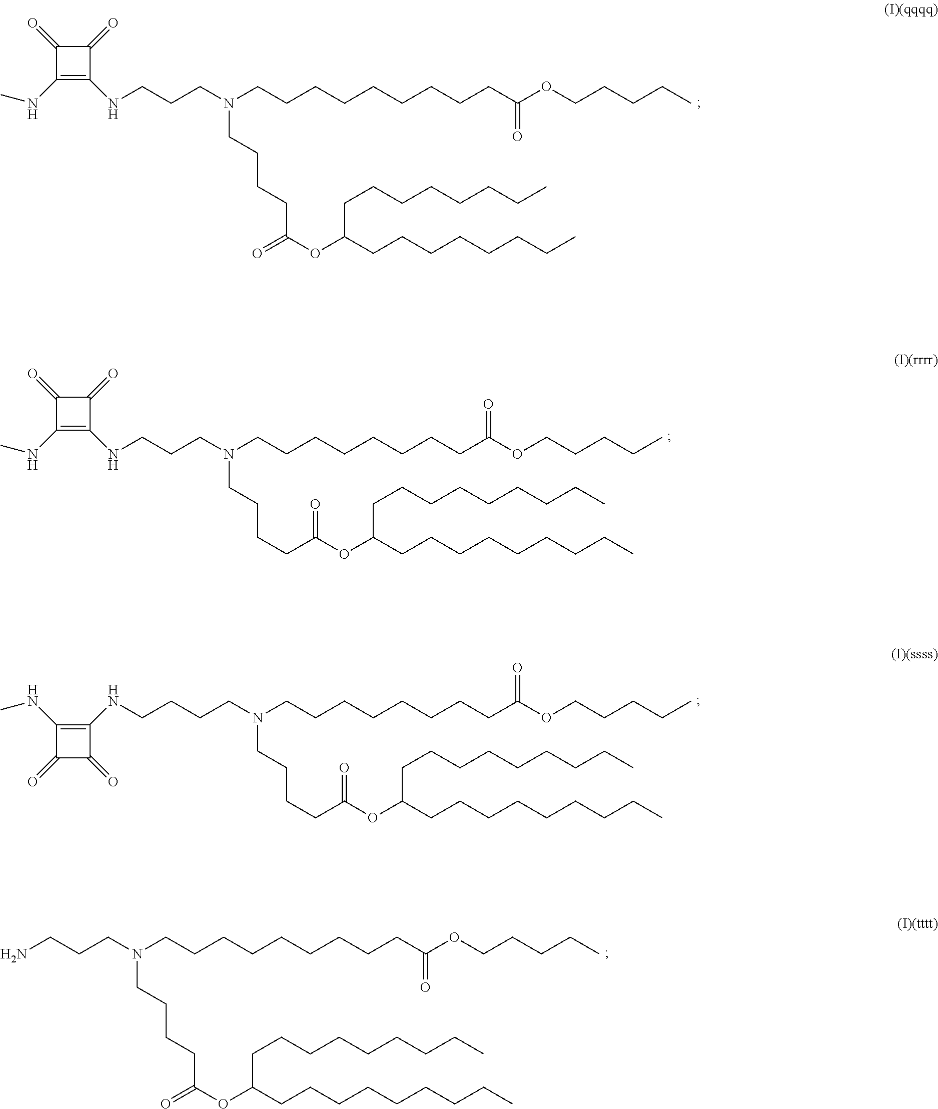 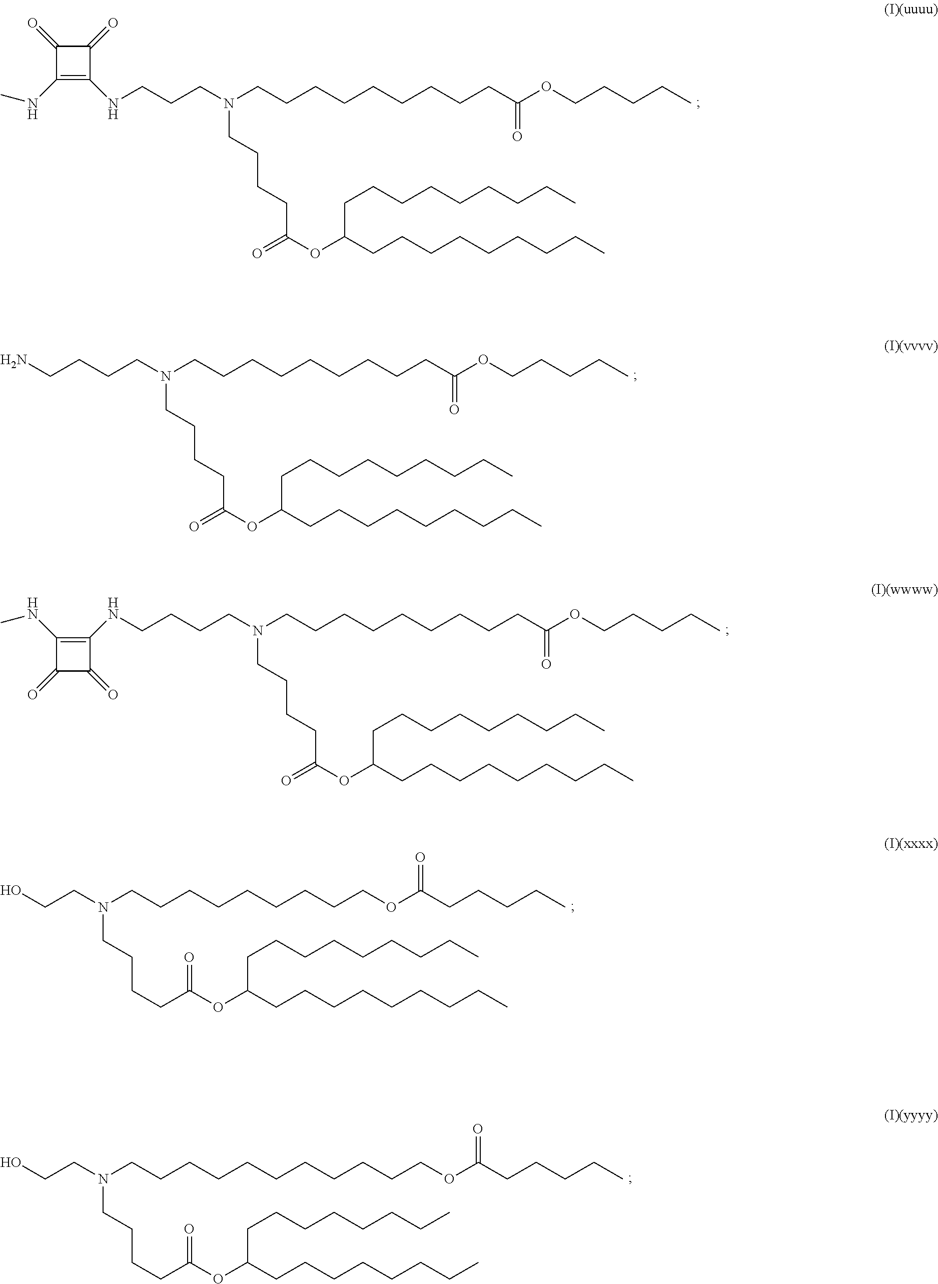 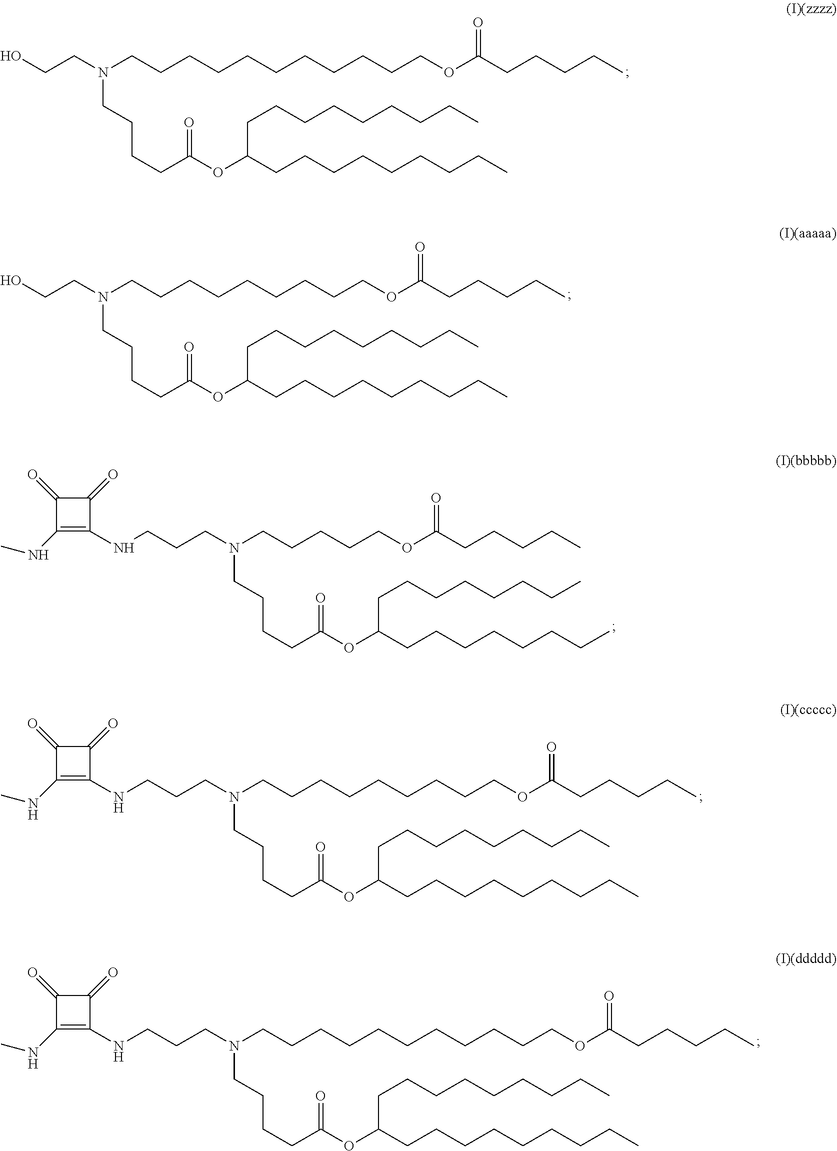
or a salt or isomer thereof.

3. The compound of claim 1, wherein the compound is: 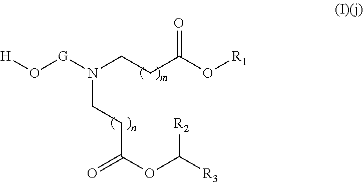
or a salt or isomer thereof, wherein
each m is independently an integer from 4-13;
each n is independently an integer from 1-3;
each R¹ is independently selected from C1-C5 alkyl, C2-C5 alkenyl, C2-C5 alkynyl, wherein the alkyl, the alkenyl, or the alkynyl is linear or branched;
each R₂ and R₃ is independently selected from C₁ to C₁₄ alkyl;
and each G is —(CR₄R₅)ₖ—;
wherein each k is selected from an integer from 2-5;
and each R₄ and R₅ is independently selected from H, and C₁-C₃ alkyl.

4. The compound of claim 1, wherein the compound is: 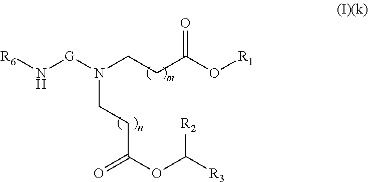
or a salt or isomer thereof, wherein
each m is independently an integer from 4-13;
each n is independently an integer from 1-3;
each R¹ is independently selected from C1-C5 alkyl, C2-C5 alkenyl, C2-C5 alkynyl, wherein the alkyl, the alkenyl, or the alkynyl is linear or branched;
each R₂ and R₃ is independently selected from C₁ to C₁₄ alkyl;
each R₆ is H,
and each G is —(CR₄R₅)ₖ—;
wherein each k is selected from an integer from 2-5;
and each R₄ and R₅ is independently selected from H, and C₁-C₃ alkyl.

5. The compound of claim 1, wherein the compound is: 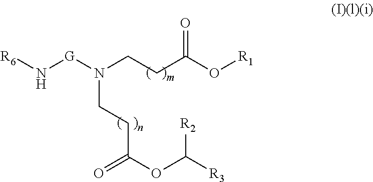
or a salt or isomer thereof, wherein
each m is independently an integer from 4-13;
each n is independently an integer from 1-3;
each R¹ is independently selected from C1-C5 alkyl, C2-C5 alkenyl, C2-C5 alkynyl, wherein the alkyl, the alkenyl, or the alkynyl is linear or branched;
each R₂ and R₃ is independently selected from C₁ to C₁₄ alkyl;
each R₆ is 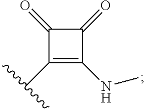 [SMILES: *c1c(NC)c(=O)c1=O]
and each G is —(CR₄R₅)ₖ—;
wherein each k is selected from an integer from 2-5;
and each R₄ and R₅ is independently selected from H, and C₁-C₃ alkyl.

6. The compound of claim 1, wherein the compound is: 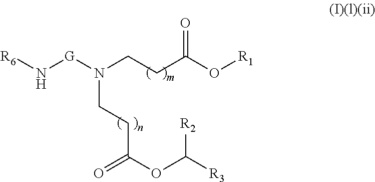
or a salt or isomer thereof, wherein
each m is independently an integer from 4-13;
each n is independently an integer from 1-3;
each R¹ is independently selected from C1-C5 alkyl, C2-C5 alkenyl, C2-C5 alkynyl, wherein the alkyl, the alkenyl, or the alkynyl is linear or branched;
each R₂ and R₃ is independently selected from C₁ to C₁₄ alkyl;
each R₆ is 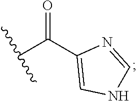 [SMILES: *C(=O)c1c[nH]cn1]
and each G is —(CR₄R₅)ₖ—;
wherein each k is selected from an integer from 2-5;
and each R₄ and R₅ is independently selected from H, and C₁-C₃ alkyl.

7. The compound of claim 1, wherein the compound is: 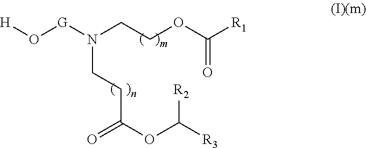
or a salt or isomer thereof, wherein
each m is independently an integer from 4-13;
each n is independently an integer from 1-3;
each R¹ is independently selected from C1-C5 alkyl, C2-C5 alkenyl, C2-C5 alkynyl, wherein the alkyl, the alkenyl, or the alkynyl is linear or branched;
each R₂ and R₃ is independently selected from C₁ to C₁₄ alkyl;
and each G is —(CR₄R₅)ₖ—;
wherein each k is selected from an integer from 2-5;
and each R₄ and R₅ is independently selected from H, and C₁-C₃ alkyl.

8. A nanoparticle composition comprising an ionizable lipid component comprising a compound of any one of claims 1-7.

9. The nanoparticle composition of claim 8, further comprising a biologically active agent.

10. The nanoparticle composition of any one of claims 8-9, further comprising one or more phospholipids.

11. The nanoparticle composition of claim 10, wherein the phospholipid comprises at least one of 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-an-glycero-3-phosphocholine (DPPC), 1,2-distearoyl-sn-glycero-3-phosphocholine (DSPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-en-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-dioleoyl-sn-glycero-3-phosphoethanol amine (DOPE), 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, and mixtures thereof.

12. The nanoparticle composition of any one of claims 8-11, further comprising one or more structural lipids.

13. The nanoparticle composition of claim 12, wherein the structural lipid comprises at least one of cholesterol, fecosterol, sitosterol, ergosterol, campesterol, stigmasterol, brassicasterol, tomatidine, ursolic acid, and alpha-tocopherol, and mixtures thereof.

14. The nanoparticle composition of any one of claims 8-13, further comprising one or more PEG lipids.

15. The nanoparticle composition of claim 14, wherein the PEG comprises at least one of PEG-modified phosphatidylethanolamine, PEG-modified phosphatidic acid, PEG-modified ceramide, a PEG-modified dialkylamine, a PEG-modified diacylglycerol, PEG-modified dialkylglycerol, and mixtures thereof.

16. The nanoparticle composition of any one of claims 8-15, further comprising a second ionizable lipid other than the ionizable lipid component.

17. A pharmaceutical composition comprising the nanoparticle composition of any one of claims 8-16 and a pharmaceutically acceptable carrier.

18. A method of delivering a biologically active agent to a cell, the method comprising administering to a subject the nanoparticle composition of any one of claims 8-16, said administering comprising contacting the cell with the nanoparticle composition, whereby the biologically active agent is delivered to the cell.

19. A method of delivering a biologically active agent to a cell, the method comprising administering the pharmaceutical composition of claim 17 to a subject.

20. A method of producing a polypeptide of interest in a cell, the method comprising contacting the cell with the nanoparticle composition of any one of claims 8-16, wherein the biologically active agent is a mRNA encoding the polypeptide of interest, whereby the mRNA is capable of being translated in the cell to produce the polypeptide of interest.

21. A method of modulating the expression of an endogenous nucleic acid in a cell, the method comprising contacting the cell with the nanoparticle composition of any one of claims 8-16, wherein the biologically active agent is a siRNA capable of binding to the endogenous nucleic acid, whereby the siRNA is capable of modulating the expression of the endogenous nucleic acid.

22. The method of claim 21, wherein the siRNA bas a sequence that is complementary to a portion of the endogenous nucleic acid.

23. A method of modulating the expression of an endogenous nucleic acid in a cell, the method comprising contacting the cell with the nanoparticle composition of any one of claims 8-16, wherein the biologically active agent is an antisense RNA capable of binding to the endogenous nucleic acid, whereby the siRNA is capable of modulating the expression of the endogenous nucleic acid.

24. The method of claim 23, wherein the antisense RNA has a sequence that is complementary to a portion of the endogenous nucleic acid.

25. A method of treating a subject having a disease or disorder, the method comprising administering to the subject a therapeutically effective amount of the nanoparticle composition of any one of claims 8-16.

26. A method for synthesizing the compound 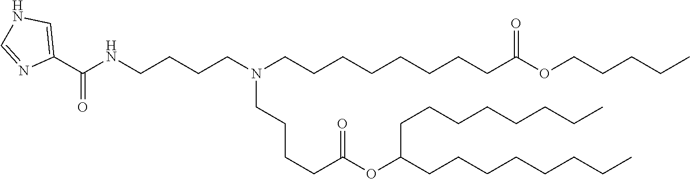 [SMILES: CCCCCCCCC(CCCCCCCC)OC(=O)CCCCN(CCCCCCCCC(=O)OCCCCC)CCCCNC(=O)c1c[nH]cn1]
(I) (i) comprising performing the following reaction:

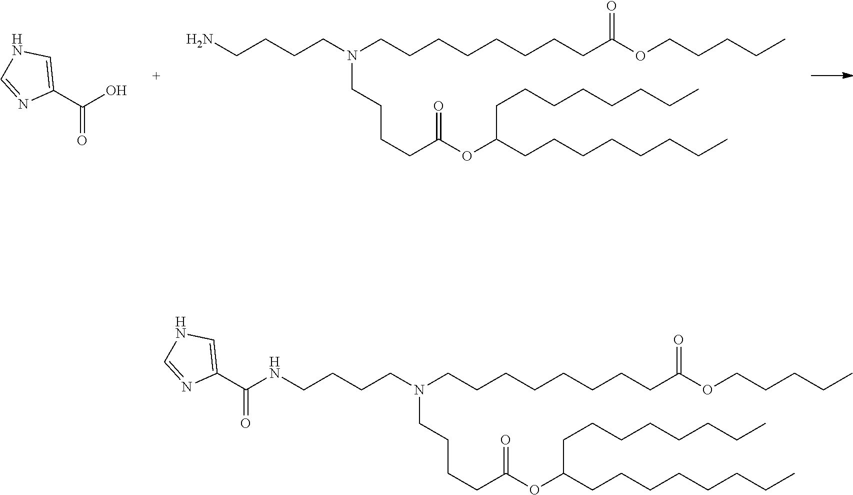 [SMILES: O=C(O)c1c[nH]cn1 ; CCCCCCCCC(CCCCCCCC)OC(=O)CCCCN(CCCCN)CCCCCCCCC(=O)OCCCCC ; CCCCCCCCC(CCCCCCCC)OC(=O)CCCCN(CCCCCCCCC(=O)OCCCCC)CCCCNC(=O)c1c[nH]cn1]

27. A method for synthesizing the compound 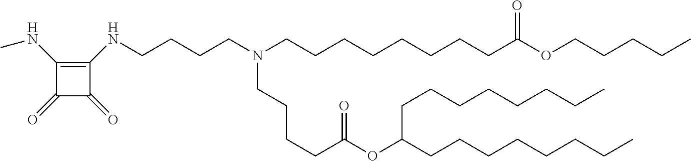 [SMILES: CCCCCCCCC(CCCCCCCC)OC(=O)CCCCN(CCCCCCCCC(=O)OCCCCC)CCCCNc1c(NC)c(=O)c1=O]
(I) (g) comprising performing the following reaction:

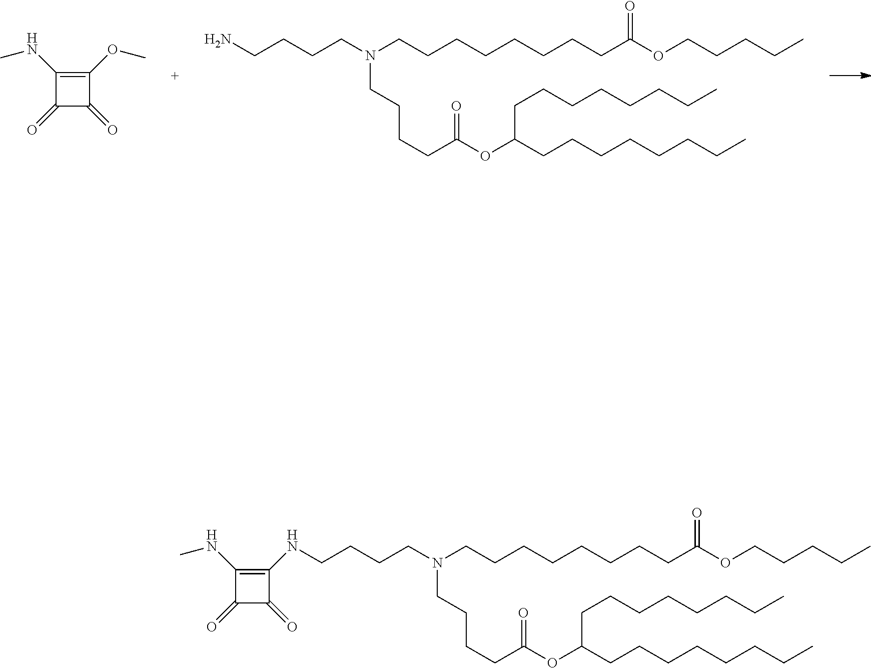

28. A method for synthesizing the compound 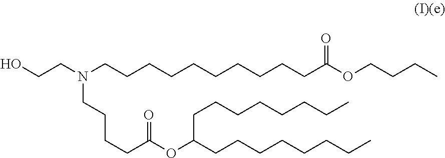 [SMILES: CCCCCCCCC(CCCCCCCC)OC(=O)CCCCN(CCO)CCCCCCCCCCC(=O)OCCCC]
comprising performing the following reaction:

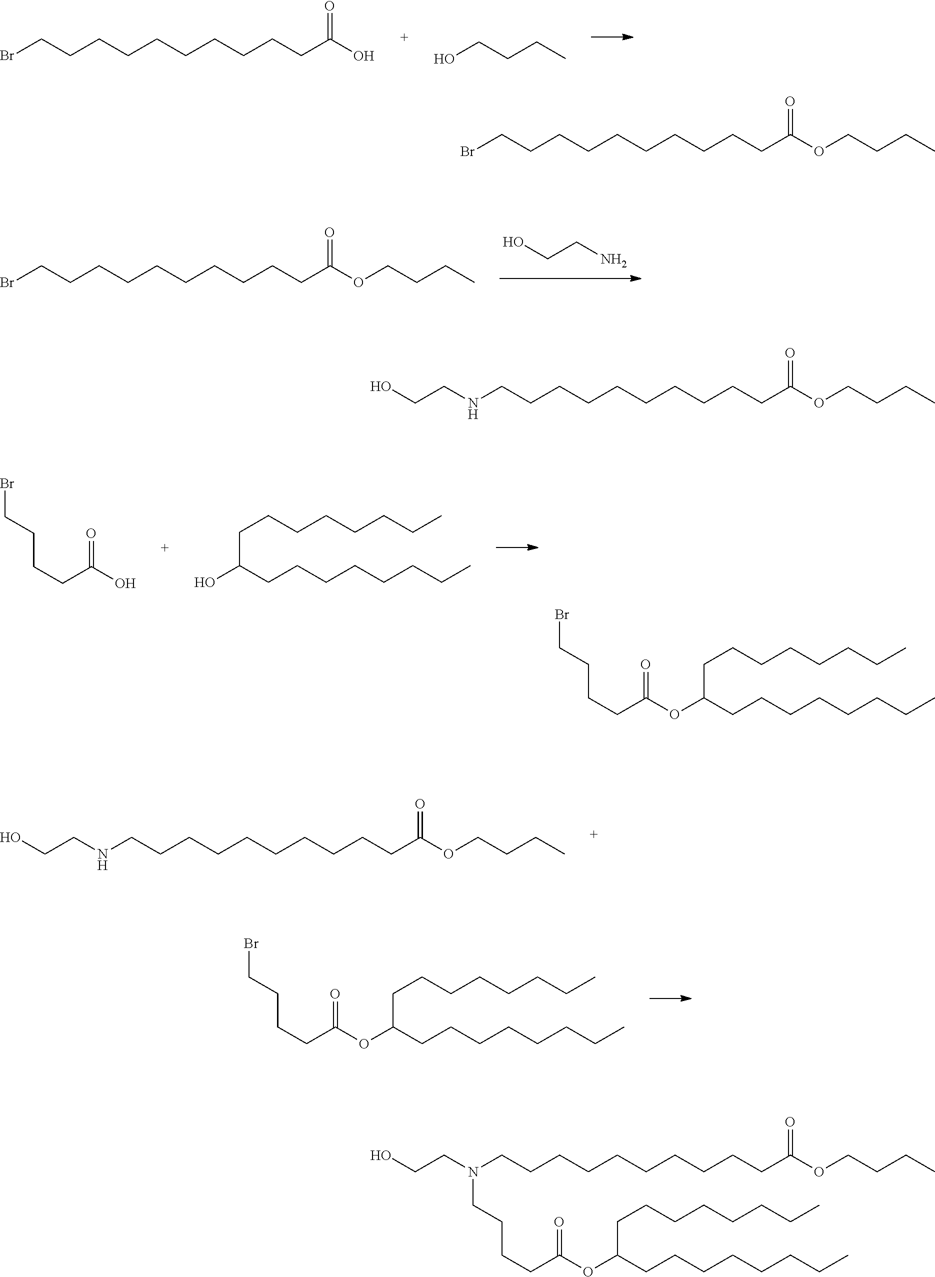

29. A method for synthesizing the compound 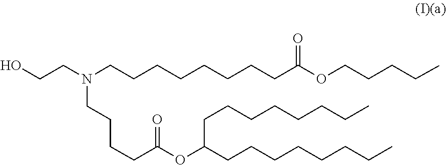 [SMILES: CCCCCCCCC(CCCCCCCC)OC(=O)CCCCN(CCO)CCCCCCCCC(=O)OCCCCC]
comprising performing the following reaction:

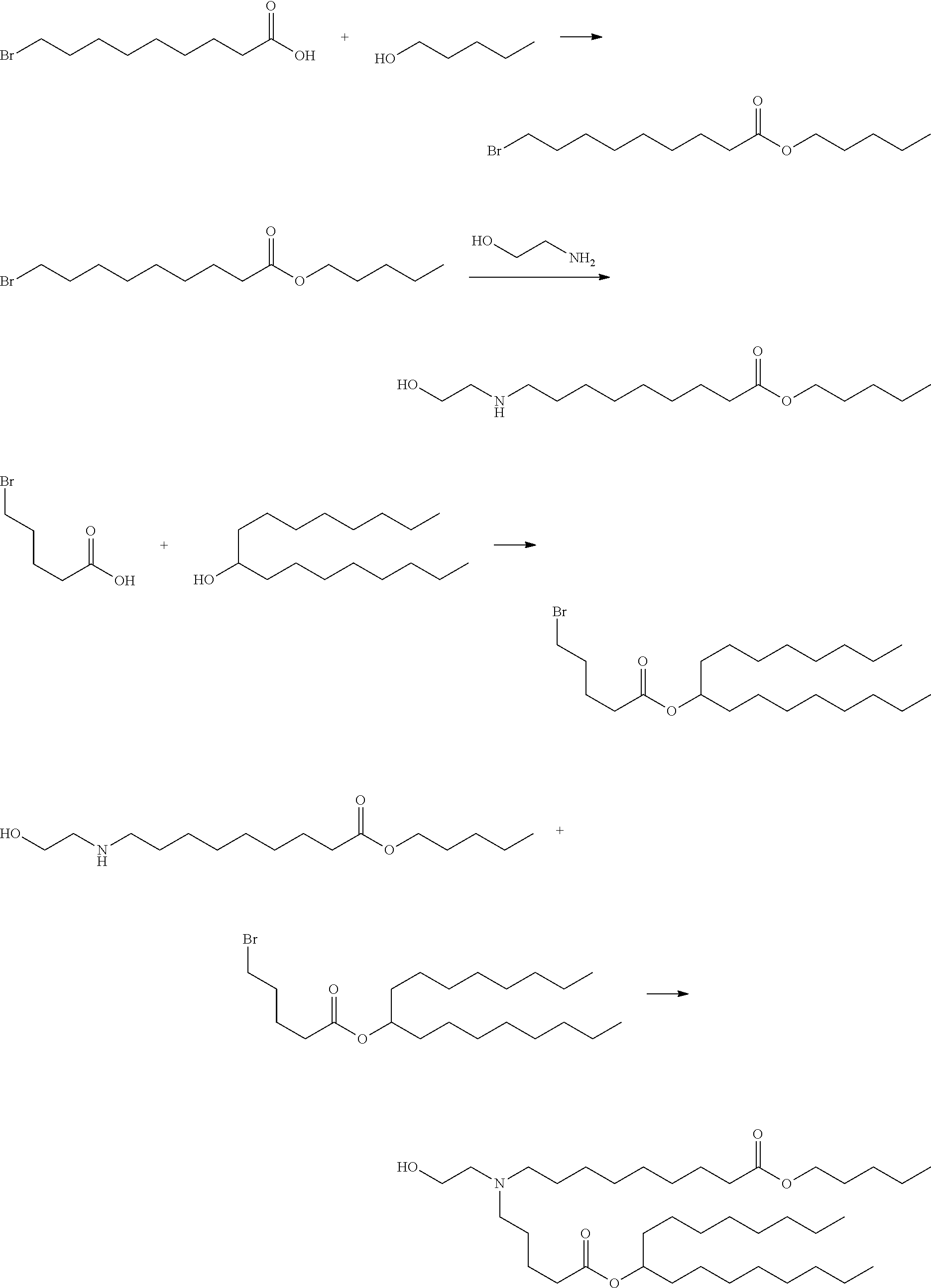

30. A method for synthesizing the compound 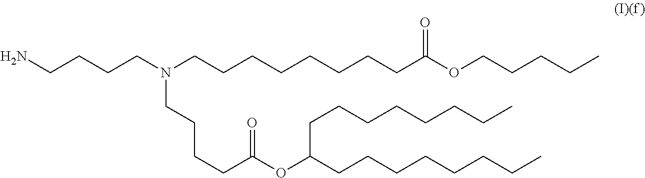 [SMILES: CCCCCCCCC(CCCCCCCC)OC(=O)CCCCN(CCCCN)CCCCCCCCC(=O)OCCCCC]
comprising performing the following reaction;

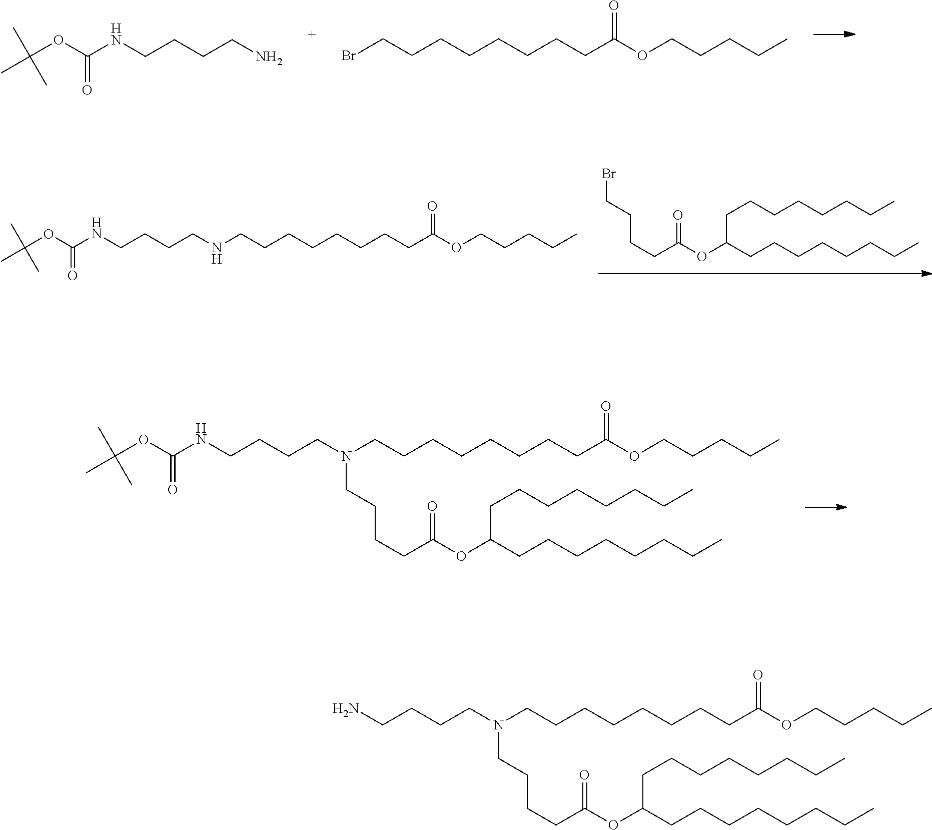

31. A method for synthesizing the compound of claim 3 comprising performing the following reaction: 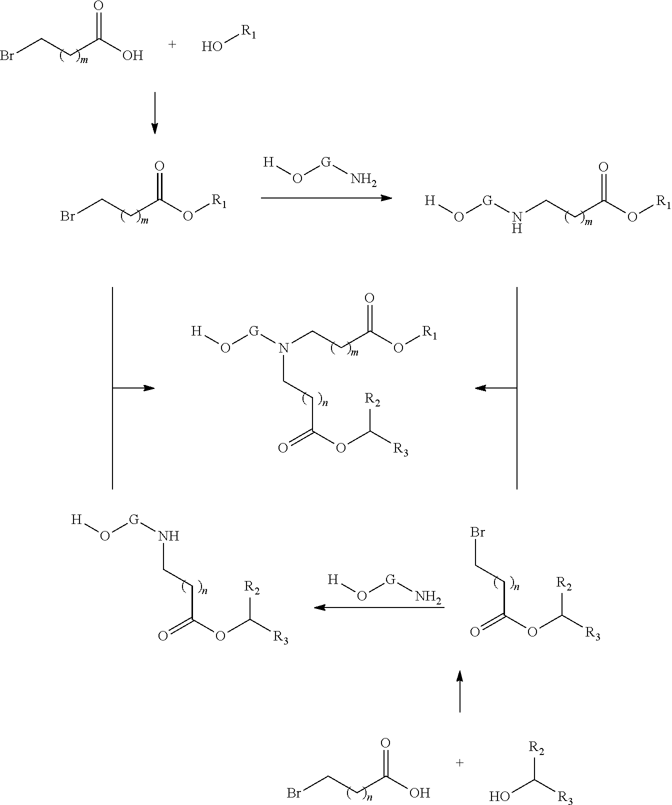
wherein
each m is independently an integer from 4-13;
each n is independently an integer from 1-3;
each R¹ is independently selected from C1-C5 alkyl, C2-C5 alkenyl, C2-C5 alkynyl, wherein the alkyl, the alkenyl, or the alkynyl is linear or branched;
each R₂ and R₃ is independently selected from C₁ to C₁₄ alkyl;
and each G is —(CR₄R₅)ₖ—;
wherein each k is selected from an integer from 2-5;
and each R₄ and R₅ is independently selected from H, and C₁-C₃ alkyl.

32. A method for synthesizing the compound of claim 4 comprising performing the following reaction: 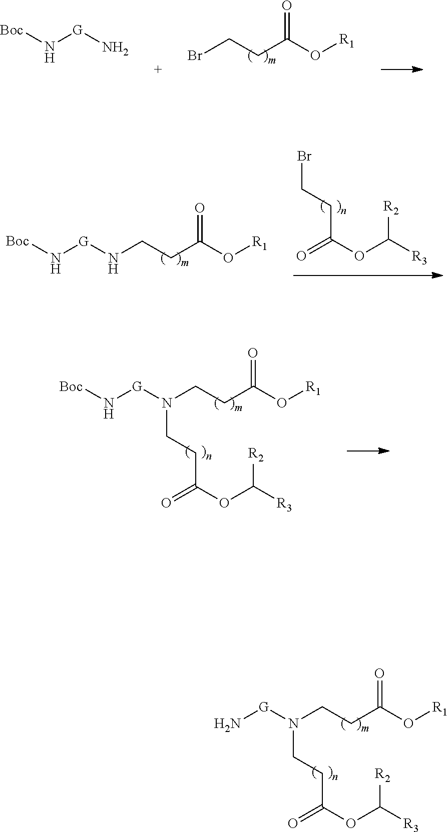
wherein
wherein
each m is independently an integer from 4-13;
each n is independently an integer from 1-3;
each R¹ is independently selected from C1-C5 alkyl, C2-C5 alkenyl, C2-C5 alkynyl, wherein the alkyl, the alkenyl, or the alkynyl is linear or branched;
each R₂ and R₃ is independently selected from C₁ to C₁₄ alkyl;
each R₆ is H,
and each G is —(CR₄R₅)ₖ—;
wherein each k is selected from an integer from 2-5;
and each R₄ and R₅ is independently selected from H, and C₁-C₃ alkyl.

33. A method for synthesizing the compounds of claim 5 comprising performing the following reaction: 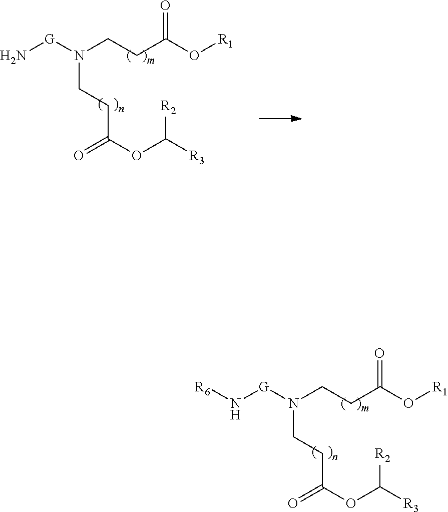
wherein
each m is independently an integer from 4-13;
each n is independently an integer from 1-3;
each R¹ is independently selected from C1-C5 alkyl, C2-C5 alkenyl, C2-C5 alkynyl, wherein the alkyl, the alkenyl, or the alkynyl is linear or branched;
each R₂ and R₃ is independently selected from C₁ to C₁₄ alkyl;
R₆ is 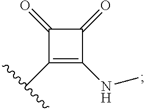 [SMILES: *c1c(NC)c(=O)c1=O]
and each G is —(CR₄R₅)ₖ—;
wherein each k is selected from an integer from 2-5;
and each R₄ and R₅ is independently selected from H, and C₁-C₃ alkyl.
and each G is —(CR₄R₅)₅—;
wherein each k is selected from an integer from 2-5;
and each R₄ and R₅ is independently selected from H, and C₁-C₃ alkyl.

34. A method for synthesizing the compounds of claim 6 comprising performing the following reaction: 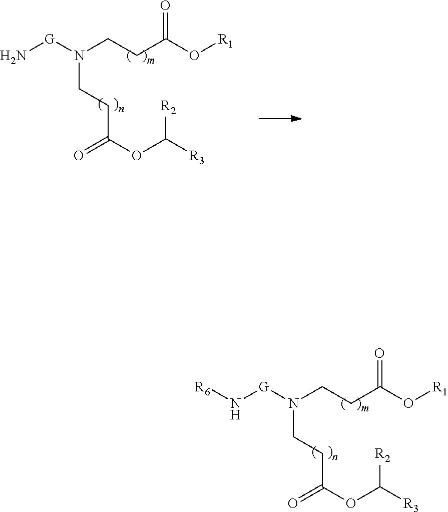
wherein
each m is independently an integer from 4-13;
each n is independently an integer from 1-3;
each R¹ is independently selected from C1-C5 alkyl, C2-C5 alkenyl, C2-C5 alkynyl, wherein the alkyl, the alkenyl, or the alkynyl is linear or branched;
each R₂ and R₃ is independently selected from C₁ to C₁₄ alkyl;
R₆ is 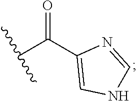 [SMILES: *C(=O)c1c[nH]cn1]
and each G is —(CR₄R₅)ₖ—;
wherein each k is selected from an integer from 2-5;
and each R₄ and R₅ is independently selected from H, and C₁-C₃ alkyl.

35. A method for synthesizing the compound of claim 7 comprising performing the following reaction: 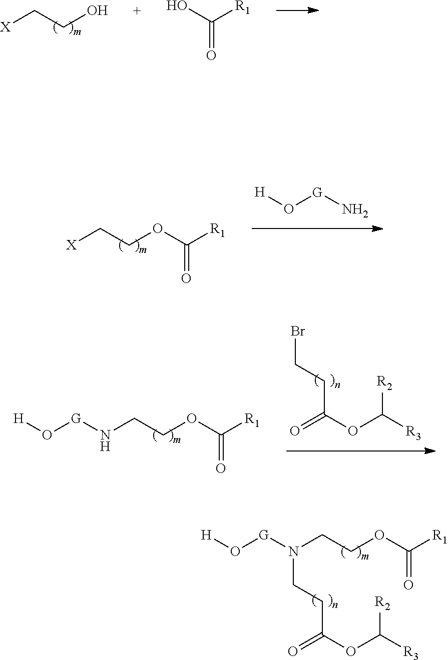
wherein
X is Cl, Br;
each m is independently an integer from 4-13;
each n is independently an integer from 1-3;
each R¹ is R₁ is C₁-C₅ alkyl, wherein the alkyl is linear or branched;
each R₂ and R₃ is independently selected from C₁ to C₁₄ alkyl;
and each G is —(CR₄R₅)₅—;
wherein each k is selected from an integer from 2-5.
